(19) United States
(12) Patent Application Publication
(10) Pub. No.: US 2009/0239845 A1
(43) Pub. Date: Sep. 24, 2009

(54) PYRAZOLE COMPOUNDS AS PROSTAGLANDIN RECEPTORS LIGANDS

(76) Inventors: Elizabeth Ann Conway, Essex (GB); Gerard Martin Paul Giblin, Essex (GB); Mairi Gibson, Essex (GB); Adrian Hall, Essex (GB); Thomas George Christopher Hayhow, Essex (GB); Mark Patrick Healy, Essex (GB); David Nigel Hurst, Essex (GB); Ian Reginald Kilford, Essex (GB); Stephen Carl McKeown, Essex (GB); Alan Naylor, Essex (GB); Helen Susanne Price, Essex (GB); Derek Anthony Rawlings, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/912,402

(22) PCT Filed: Apr. 24, 2006
(86) PCT No.: PCT/EP06/03919

§ 371 Date: Apr. 25, 2008

(30) Foreign Application Priority Data
Apr. 26, 2005 (GB) 0508472.8

(51) Int. Cl. A61K 31/55; C07D 231/40; A61K 31/415; A61K 31/5377; C07D 413/12; C07D 403/12; A61P 9/00

(52) U.S. Cl. 514/217.01; 548/372.5; 514/407; 514/236.5; 544/140; 540/594

(57) ABSTRACT

Compounds of formula (I) or a pharmaceutically acceptable derivative thereof: wherein Z, R¹, R^{2a}, R^{2b}, R¹⁰, R¹¹ and R^{x} are as defined in the specification, a process for the preparation of such compounds, pharmaceutical compositions comprising such compounds and the use of such compounds in medicine.

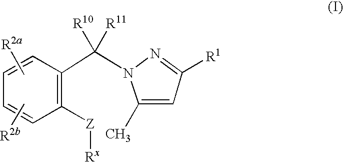

This invention relates to pyrazole compounds, to processes for their preparation, to pharmaceutical compositions containing them and to their use in medicine, in particular their use in the treatment of conditions mediated by the action of PGE₂ at the EP₁ receptor.

Prostaglandin receptors, including the EP₁₋₄, DP, FP, IP and TP receptors are the effector proteins for the products (prostaglandins) downstream of COX-1/2 activation (PGE₂, PGD2, PGF2a, PGI2 and thromboxane respectively). The NSAIDS (non-steroidal anti-inflammatory drugs) are indiscriminate cyclooxygenase inhibitors and reduce the levels of these prostaglandins. This in turn reduces the action of the prostaglandins at their respective receptors. In view of the relatively large number of receptors affected, the pharmacology of the NSAIDS is complex.

The EP₁ receptor is a 7-transmembrane receptor and its natural ligand is the prostaglandin PGE₂. PGE₂ also has affinity for the other EP receptors (types EP₂, EP₃ and EP₄). The E₁ receptor is associated with smooth muscle contraction, pain (in particular inflammatory, neuropathic and visceral), inflammation, allergic activities, renal regulation and gastric or enteric mucus secretion.

We have now found a novel group of compounds which bind with high affinity to the EP₁ receptor. These compounds are antagonists of the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S. Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. In The American Physiological Society (1994, 267, R289-R-294), studies suggest that PGE₂-induced hyperthermia in the rat is mediated predominantly through the EP₁ receptor.

The TP (also known as TxA₂) receptor is a prostanoid receptor subtype stimulated by the endogenous mediator thromboxane. Activation of this receptor results in various physiological actions primarily incurred by its platelet aggregatory and smooth muscle constricting effects, thus opposing those of prostacyclin receptor activation.

TP receptors have been identified in human kidneys (G. P. Brown et al, Prostaglandins and other lipid mediators, 1999, 57, 179-188) in the glomerulus and extraglomerular vascular tissue. Activation of TP receptors constricts glomerular capillaries and suppresses glomerular filtration rates (M. D. Breyer et al, Current Opinion in Nephrology and Hypertension, 2000, 9, 23-29), indicating that TP receptor antagonists could be useful for renal dysfunction in glomerulonephritis, diabetes mellitus and sepsis.

Activation of TP receptors induces bronchoconstriction, increase in microvascular permeability, formation of mucosal oedema and mucus secretion, typical characteristic features of bronchial asthma (T. Obata et al, Clinical Review of Allergy, 1994, 12(1), 79-93). TP antagonists have been investigated as potential asthma treatments resulting in, for example, orally active Seratrodast (AA-2414) (S. Terao et al, Yakugaku Zasshi, 1999, 119(5), 377-390). Ramatroban is another TP receptor antagonist currently undergoing phase III clinical trials as an anti-asthmatic compound.

Antagonists at the TP receptor have been shown to have a gastroprotective effect. In rats it has been shown that SQ 33961 and BM 13505 inhibit gastric lesions induced by taurocholate acid, aspirin or indomethacin (E. H. Ogletree et al, Journal of Pharmacology and Experimental Therapeutics, 1992, 263(1), 374-380.

Certain compounds of the present invention also exhibit antagonism at the TP receptor and are therefore indicated to be useful in treating conditions mediated by the action of thromboxane at the TP receptor. Such conditions include those disclosed in WO 2004/039807 (Merck Frosst Canada & Co) which is incorporated herein by reference, and include respiratory diseases e.g. asthma, allergic diseases, male erectile dysfunction, thrombosis, renal disorders and gastric lesions.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005) and WO2005/108369 (17 Nov. 2005) disdose compounds as being useful in the treatment of prostaglandin mediated diseases.

A. Hall et al, Bioorg. Med. Chem. Lett., 2006, 16, 2666-2671 discloses biaryl heterocyclic EP₁ receptor agonists.

P. Lacombe et al (220th National Meeting of The American Chemical Society, Washington D.C., USA, 20-24 Aug., 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} Aug. 2004) disclosed 2,3-diarylthiophenes as EP₁ receptor antagonists. Y. Ducharme et al, Biorg. Med. Chem. Lett., 2005, 15(4): 1155 also discloses 2,3-diarylthiophenes as selective EP₁ receptor antagonists.

Accordingly the present invention provides compounds of formula (I):

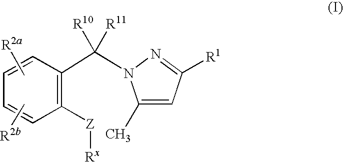

wherein:

Z is O, S, SO or SO₂;

R^{x} is optionally substituted C₂₋₁₀alkyl, optionally substituted C₂₋₁₀alkenyl, optionally substituted C₂₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;

R¹ is CONR³R⁴, NR³CO₂R⁵, NR³COR⁶, OCONR³R⁷, tetrazolyl, oxazolin-2-yl, oxazol-2-yl, benzoxazol-2-yl, pyrrolidinonyl, isoindoledionyl, dihydroisoindolonyl, or optionally substituted SO₂NHCOaryl; or R¹ is optionally substituted imidazolyl or optionally substituted 1,2,4-triazolyl wherein optionally the imidazole or 1,2,4-triazole ring is fused to give an optionally substituted bicyclic or tricyclic ring system;

### or R¹ is

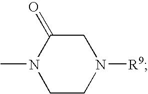

R^{2a} and R^{2b} are independently selected from hydrogen, halo, CN, SO₂alkyl, SR³, NO₂, optionally substituted alkyl, and optionally substituted alkoxy;

R³ is hydrogen or C₁₋₄alkyl;

R⁴ is hydrogen, OH, optionally substituted alkyl, optionally substituted aryl, optionally substituted heterocyclyl, optionally substituted bicyclic heterocyclyl, optionally substituted CQ^{c}Q^{d}aryl, optionally substituted CQ^{c}Q^{d}heterocyclyl, optionally substituted CQ^{c}Q^{d}bicyclic heterocyclyl, or SO₂R⁸;

R⁵ is C₁₋₄alkyl optionally substituted by SiMe₃, SO₂C₁₋₄alkyl, OC₁₋₄alkyl, N(C₁₋₄alkyl)₂, CO₂C₁₋₄alkyl, or CF₃; cyclohexyl substituted by CH₂NHC₁₋₄alkyl, CH₂pyrrolidinyl, CH₂morpholinyl or CH₂piperidinyl; phenyl; CQ^{c}Q^{d}phenyl; CQ^{c}Q^{d}pyridyl; CQ^{c}Q^{d}thienyl;

CQ^{c}Q^{d}tetrahydrofuryl; CQ^{c}Q^{d}furyl; CQ^{c}Q^{d}piperidinyl optionally substituted by C₁₋₄alkyl; CH₂CH₂pyrrolidinonyl; CQ^{c}Q^{d}CH₂morpholinyl; tetrahydropyranyl; tetrahydrofuryl; 2-pyrrolidinon-4-yl; tetrahydrothienyl-1,1-dioxide; piperidin-4-yl optionally substituted on the 1-position by CO₂C₁₋₄alkyl; or dihydroindenyl;

R⁶ is alkyl, optionally substituted aryl, optionally substituted heterocyclyl, optionally substituted bicyclic heterocyclyl, optionally substituted CQ^{c}Q^{d}-Y-aryl, optionally substituted CQ^{c}Q^{d}-Y-heterocyclyl or optionally substituted CQ^{c}Q^{d}-Y-bicyclic heterocyclyl;

R⁷ is optionally substituted alkyl, optionally substituted alkenyl, optionally substituted aryl, or optionally substituted CQ^{c}Q^{d}aryl;

R⁸ is optionally substituted alkyl, optionally substituted aryl or optionally substituted heterocyclyl;

R⁹ is alkyl optionally substituted by OH, CN, OC₁₋₃alkyl, CONH₂, CONHC₁₋₄alkyl, or SO₂phenyl; alkenyl; optionally substituted CQ^{c}Q^{d}-Y-aryl; optionally substituted CQ^{c}Q^{d}-Y-heterocyclyl; or optionally substituted CQ^{c}Q^{d}-Y-bicyclic heterocyclyl;

R¹⁰ and R¹¹ are independently selected from hydrogen, fluorine and alkyl; or R¹⁰ and R¹¹ together with the carbon to which they are attached form a cycloalkyl ring, optionally containing up to one heteroatom selected from O, S, NH or N-alkyl; and

Y is CH₂ or a bond;

Q^{a} and Q^{b} are each independently selected from hydrogen, CH₃ and fluorine;

Q^{c} and Q^{d} are each independently selected from hydrogen, CH₃ and fluorine; and derivatives thereof;

provided that:

R⁹ is not optionally substituted CH₂furan or optionally substituted CH₂imidazole;

when R^{x} is 2-methylpropyl, then R⁵ is not 1-methylethyl;
when R^{x} is optionally substituted CH₂cyclopropyl, then R⁹ is not 2-methylpropyl, CH₂cyclopropyl, CH₂cyclobutyl, CH₂CH₂OCH₃ or CH₂CH₂OH;
when R^{x} is CH₂tetrahydropyranyl or CH₂CH₂N(CH₃)₂, then R⁹ is not 2-methylpropyl.
when R¹ is benzimidazolyl it is unsubstituted on the 1-position; and
when R¹ is benzimidazole optional substituents on the 4 or 7 position are selected from CH₂OH or CO₂H.

Suitably Z is O.

In one aspect R¹ is CONR³R⁴, NR³CO₂R⁵, NR³COR⁶, OCONR³R⁷, tetrazolyl, oxazolin-2-yl, oxazol-2-yl, benzoxazol-2-yl, isoindole-1,3-dione or optionally substituted SO₂NHCOaryl; or R¹ is optionally substituted imidazolyl or optionally substituted 1,2,4-triazolyl wherein optionally the imidazole or 1,2,4-triazole ring is fused to give an optionally substituted bicyclic or tricyclic ring system; or R¹ is

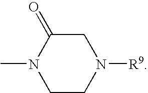

Examples of fused imidazole groups include benzimidazole, imidazo[1,2-a]pyridine, imidazo[1,2-a]pyrazine, imidazo[1,2-a]pyrimidine, imidazo[4,5-b]pyridine, imidazo[4,5-b]pyrazine, imidazo[4,5-c]pyridine and purine, all of which may be optionally substituted.

An example of a fused 1,2,4-triazolyl group is 1,2,4-triazolo[1,5-a]pyridine.

In another aspect R¹ is CONR³R⁴, NR³CO₂R⁵, NR³COR⁶, OCONR³R⁷, tetrazolyl, oxazolin-2-yl, oxazol-2-yl, benzoxazol-2-yl, isoindol-1,3-dione, optionally substituted SO₂NHCOaryl, imidazo-2-yl, optionally substituted imidazo-4-yl, optionally substituted 1,2,4-triazol-3-yl, optionally substituted benzimidazolyl, imidazo[1,2-a]pyridinyl, imidazo[1,2-a]pyrazinyl, imidazo[1,2-a]pyrimidinyl, imidazo[4,5-b]pyridinyl, imidazo[4,5-b]pyrazinyl, imidazo[4,5-c]pyridinyl, purinyl, and 1,2,4-triazolo[1,5-a]pyridinyl.

When R¹ is imidazo-4-yl suitable substituents include phenyl, pyridyl, imidazolyl, morpholinyl and pyrazinyl. In one aspect the substituents are on the 2-position of the imidazole ring.

When R¹ is 1,2,4-imidazo-3-yl suitable substituents include methyl.

When R¹ is benzimidazole suitable substituents include one or two substituents selected from F, CH₂OH, piperazinylmethyl, piperazinylethyl, morpholinyl, CH₂NHC₁₋₂alkyl, CH₂N(C₁₋₂alkyl)₂, CH₂morpholinyl, CH₂pyrrolidinyl, CH₂piperidinyl, CH₂piperazinylmethyl, and CH₂piperazinylethyl. If the benzimidazole is substituted, preferably it is substituted on the 5 and/or 6 positions.

When R¹ is benzimidazole, in one aspect it is attached to the pyrazole ring through the 2-position carbon atom.

Suitably R¹ is CONR³R⁴, NR³CO₂R⁵, NR³COR⁶, OCONR³R⁷, tetrazolyl, oxazolin-2-yl, oxazol-2-yl, benzoxazol-2-yl, pyrrolidinone, isoindoledione, dihydroisoindolone, 3-methyl-1,2,4-triazol-5-yl, or SO₂NR³COphenyl; or R¹ represents imidazolyl wherein optionally the imidazole ring is fused to give an optionally substituted bicyclic or tricyclic ring system;

### or R¹ is

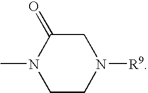

In one aspect R¹ is NR³CO₂R⁵, NR³COR⁶, or OCONR³R⁷. In a further aspect R¹ is NHCO₂R⁵, NHCOR⁶, or OCONHR⁷.

In another aspect R¹ is CONR³R⁴. In yet another aspect R¹ is optionally substituted SO₂NHCOphenyl.

In a further aspect R¹ is tetrazolyl, oxazolin-2-yl, oxazol-2-yl, benzoxazol-2-yl, pyrrolidinone, isoindoledione, dihydroisoindolone, or optionally substituted 1,2,4-triazolyi; or R¹ is imidazolyl wherein optionally the imidazole ring is fused to give an optionally substituted bicyclic or tricyclic ring system.

In yet another aspect R¹ is

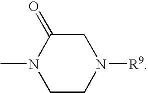

In one aspect R^{2a} is hydrogen.

Suitably R^{2b} is selected from halogen, e.g. Cl or Br.

Preferably R^{2b} is positioned 1,4-relative to the Z substituent and 1,3-relative to the pyrazole moiety.

In a particular aspect Z is O, R^{2a} is hydrogen, R^{2b} is Cl or Br and is positioned 1,4-relative to the Z substituent and 1,3-relative to the pyrazole moiety.

In a further aspect Z is O; R^{2a} is hydrogen; R^{2b} is Cl or Br and is positioned 1,4-relative to the Z substituent; and 1,3-relative to the pyrazole moiety; and R¹⁰ and R¹¹ are each hydrogen.

In one aspect when R^{x} represents optionally substituted C₂₋₁₀alkyl this group is suitably optionally substituted C₃₋₈alkyl, for example 2-methylpropyl, optionally substituted CH₂cyclopropyl, e.g. CH₂(CF₃)cyclopropyl and CH₂(CF₃)cyclopropyl.

When R^{x} represents optionally substituted C₂₋₁₀alkenyl, suitably it is C₃₋₆alkenyl e.g. CH₂CH(CH₃)═CH₂ or CH₂CH(Cl)═CH₂.

When R^{x} represents optionally substituted CQ^{a}Q^{b}-heterocyclyl, suitably this group is CH₂heterocyclyl, e.g. CH₂tetrahydrofuran, CH₂tetrahydropyran and CH₂pyridine.

When R^{x} represents optionally substituted CQ^{a}Q^{b}-bicyclic heterocyclyl or optionally substituted CQ^{a}Q^{b}-aryl, suitably R^{x} is optionally substituted CH₂-heterocyclyl, optionally substituted CH₂-bicyclic heterocyclyl or optionally substituted CH₂-aryl e.g. optionally substituted CH₂-phenyl. Optional substituents for R^{x} when CH₂-phenyl include one, two or three substituents each independently selected from Cl and F.

In one aspect R^{x} represents optionally substituted C₃₋₈alkyl, optionally substituted C₃₋₈alkenyl or optionally substituted CH₂phenyl.

In a further aspect R^{x} is optionally substituted CH₂phenyl.

Suitably R³ is hydrogen or CH₃. In one aspect R³ is hydrogen.

In one aspect R⁴ includes hydrogen, OH, optionally substituted C₁₋₄alkyl, tetrazolyl, optionally substituted phenyl, optionally substituted pyridinyl, optionally substituted pyrazinyl, optionally substituted piperidinyl, optionally substituted pyrrolidinyl, optionally substituted morpholinyl, optionally substituted tetrahydroisoquinolin-6-yl, optionally substituted CH₂phenyl, optionally substituted C(CH₃)₂phenyl, optionally substituted CH₂pyridinyl, phenylsulfonyl and 3,5-dimethyl-4-isoxazolylsulfonyl.

In another aspect R⁴ includes hydrogen, OH, C₁₋₄alkyl e.g. methyl or isopropyl, bis(ethyloxy)ethyl, tetrazolyl, optionally substituted phenyl, optionally substituted pyridinyl, optionally substituted pyrazinyl, piperidinyl, pyrrolidinyl, morpholinyl, tetrahydroisoquinolin-6-yl, CH₂phenyl, C(CH₃)₂phenyl, CH₂pyridinyl, phenylsulfonyl and 3,5-dimethyl-4-isoxazolylsulfonyl.

Optional substituents for phenyl when forming part of a group R⁴ include 1 to 3 substituents selected from Cl, F, CN, OC₁₋₄alkyl, CF₃, OCF₃, CH₂OH, CH₂NR^{x}R^{y} wherein R^{x} is hydrogen or C₁₋₃alkyl optionally substituted by OH, and R^{y} is C₁₋₃alkyl optionally substituted by OH; or R^{x} and R^{y} together with the nitrogen to which they are attached form a piperazinyl, morpholinyl, pyrrolidinyl or piperidinyl group wherein the pyrrolidinyl or piperidinyl group may be optionally substituted by OH and the piperazinyl ring may be optionally substituted by oxo.

In one aspect optional substituents for phenyl when forming part of a group R⁴ include 1 to 3 substituents selected from Cl, F, CN, OC₁₋₄alkyl e.g OMe, CF₃, OCF₃, CH₂OH, CH₂NHMe, CH₂NHEt, CH₂NH-isopropyl, CH₂NHCH₂CH₂OH, CH₂NMe₂, CH₂N(CH₂CH₂OH)₂, CH₂pyrrolidinyl optionally substituted by OH, CH₂piperidinyl optionally substituted by OH, CH₂pyrazinyl optionally substituted by oxo, and CH₂morpholinyl.

In a further aspect optional substituents for phenyl when forming part of a group R⁴ include 1 to 3 substituents selected from Cl, F, CN, OC₁₋₄alkyl, CF₃, OCF₃, CH₂OH, CH₂N(C₁₋₄alkyl)₂, and CH₂NHC₁₋₄alkyl.

Optional substituents for pyridinyl when forming part of a group R⁴ include ethenyl, CO₂H, CH₂OH, CH₂N(C₁₋₄alkyl)₂, e.g. CH₂NMe₂ and CH₂NEt₂, CH₂NHC₁₋₄alkyl e.g. CH₂NHMe and CH₂NHEt, CH₂morpholine, and CH₂pyrrolidine.

Optional substituents for pyrazinyl when forming part of a group R⁴ include CH₂N(C₁₋₄alkyl)₂ e.g. CH₂NMe₂, and CH₂NHC₁₋₄alkyl e.g. CH₂NHMe, CH₂morpholine, CH₂pyrrolidine, CH₂piperidine and CH₂piperazinylmethyl.

In one aspect R⁵ is methyl, ethyl, isopropyl, iso-butyl, or tert-butyl all of which may be substituted by SiMe₃, SO₂Me, OMe, NEt₂, CO₂Et, or CF₃;

cyclohexyl substituted by CH₂NHC₂H₅, CH₂pyrrolidinyl, CH₂morpholinyl or CH₂ piperidinyl; phenyl; CH₂phenyl; CMe₂phenyl; CH₂pyridyl; CH₂thienyl; CH₂tetrahydrofuryl, CH₂furyl, CH₂piperidin-4-yl optionally substituted on the 1-position by C₁₋₄alkyl e.g. C₃H₇; CH₂CH₂pyrrolidinonyl; CH₂CH₂morpholinyl; CH(CH₃)morpholinyl; tetrahydropyranyl; tetrahydrofuryl; 2-pyrrolidinon-4-yl; tetrahydrothienyl-1,1-dioxide; piperidinyl optionally substituted by CO₂C(CH₃)₃; or dihydroindenyl.

In a further aspect R⁵ is methyl; ethyl optionally substituted by SiMe₃, SO₂Me, CF₃ or OMe; isopropyl optionally substituted by NEt₂, CO₂Et, or CF₃; iso-butyl; tert-butyl; cyclohexyl substituted on the 3-position by CH₂NHC₂H₅, CH₂pyrrolidinyl, CH₂morpholinyl or CH₂ piperidinyl; phenyl; CH₂phenyl; CMe₂phenyl; CH₂pyridyl; CH₂thienyl; CH₂tetrahydrofuryl, CH₂furyl, CH₂piperidin-4-yl optionally substituted on the 1-position by C₃H₇; CH₂CH₂pyrrolidinonyl; CH₂CH₂morpholinyl; CH(CH₃)morpholinyl; tetrahydropyran-4-yl; tetrahydrofuran-3-yl; 2-pyrrolidinon-4-yl; tetrahydrothiophene-3-yl-1,1-dioxide; piperidin-4-yl optionally substituted on the 1-position by CO₂C(CH₃)₃; or 2,3-dihydro-1H-inden-2-yl.

Suitably R⁶ includes C₁₋₈alkyl, CF₃, optionally substituted naphthyl, optionally substituted phenyl, optionally substituted heterocyclyl, optionally substituted bicyclic heterocyclyl, optionally substituted CQ^{c}Q^{d}-Y-aryl, optionally substituted CQ^{c}Q^{d}-Y-heterocyclyl or optionally substituted CQ^{c}Q^{d}-Y-bicyclic heterocyclyl.

In one aspect R⁶ includes C₁₋₈alkyl, CF₃, optionally substituted naphthyl, optionally substituted phenyl, optionally substituted heterocyclyl, optionally substituted bicyclic heterocyclyl, optionally substituted CH₂phenyl, optionally substituted C(CH₃)₂phenyl, optionally substituted CF₂phenyl, optionally substituted CH₂heterocyclyl, optionally substituted C(CH₃)₂heterocyclyl, and optionally substituted CF₂heterocyclyl.

Substituents for phenyl and naphthyl when part of a group R⁶ include 1 to 3 substituents selected from Cl, F, C₁₋₄alkyl, C₂₋₄alkenyl, OCF₃, CN, OC₁₋₄alkyl, CH₂OH, CHO, COC₁₋₄alkyl, CH₂OCOphenyl, N(C₁₋₄alkyl)₂, NHC₁₋₄alkyl, CHR^{f}NR^{g}R^{h}, and pyrrolidinyl: wherein R^{f} is hydrogen or C₁₋₄alkyl, R^{g} is hydrogen or C₁₋₄alkyl, and R^{h} is hydrogen, or C₁₋₄alkyl optionally substituted by OCH₃; or R^{g} and R^{h} together with the nitrogen to which they are attached form a piperidine, morpholine, pyrrolidine, or piperazine ring.

In one aspect, substituents for phenyl and naphthyl when part of a group R⁶ include 1 to 3 substituents selected from Cl, F, C₁₋₄alkyl, ethenyl, OCF₃, CN, OC₁₋₄alkyl, CH₂OH, CHO, COC₁₋₄alkyl, CH₂OCOphenyl, N(C₁₋₄alkyl)₂, CH₂N(C₁₋₄alkyl)₂, CHCH₃N(C₁₋₄alkyl)₂, NHC₁₋₄alkyl, CH₂NHC₁₋₄alkyl, CHCH₃NHC₁₋₄alkyl, CH₂NHCH₂CH₂OCH₃, CH₂OH, COH, COC₁₋₄alkyl, CH₂OCOphenyl, pyrrolidinyl, CHCH₃pyrrolidine, CH₂piperidine, CH₂morpholine, CH₂pyrrolidine, CH₂piperazine, or CH₂piperazinone.

In a further aspect, substituents for phenyl and naphthyl when part of a group R⁶ include 1 to 3 substituents selected from Cl, F, C₁₋₄alkyl, OCF₃, CN, OC₁₋₄alkyl, N(C₁₋₄alkyl)₂, CH₂N(C₁₋₄alkyl)₂, NHC₁₋₄alkyl, CH₂NHC₁₋₄alkyl, CH₂OH, COH, CH₂OCOphenyl, CH₂piperidine, CH₂morpholine, CH₂pyrrolidine, CH₂piperazine, or CH₂piperazinone.

When R⁶ is optionally substituted heterocyclyl suitable heterocyclic groups include pyridine, thiophene, furan, isoxazole, pyrazine, tetrahydrofuran, and morpholine.

When R⁶ is optionally substituted bicyclic heterocyclyl suitable groups include benzofuran, benzthiadiazole, quinoline, isoquinoline, quinoxazoline, cinnoline, tetrahydroisoquinolin-6-yl and tetrahydrobenzazepine.

When part of an R⁶ group, heterocyclyl and bicyclic heterocyclyl groups may be substituted by one or more substituents selected from, for example, halogen, OH, NO₂, C₁₋₄alkyl, phenyl, SO₂C₁₋₄alkyl, CO₂C₁₋₄alkyl, 1-alkylpiperidine, morpholine, and pyrrolidine.

Suitably R⁷ is optionally substituted C₁₋₆alkyl, optionally substituted C₃₋₆alkenyl, optionally substituted phenyl, or optionally substituted CH₂phenyl.

In one aspect R⁷ is propyl, isopropyl, isobutyl, sec-butyl, tert-butyl, propenyl, optionally substituted phenyl or optionally substituted CH₂phenyl.

When part of an R⁷ group, a phenyl group may be substituted by one or more substituents, for example one or two substituents, independently selected from halogen e.g. Cl, NHCOC₁₋₄alkyl, CH₂NHCOC₁₋₄alkyl, piperidinylmethyl, morpholinyl, and N(C₁₋₄alkyl)₂. In one aspect substituents are selected from Cl, NHCOC(CH₃)₃, CH₂NHCOC(CH₃)₃, piperidinylmethyl, morpholinyl, and N(CH₃)₂.

Suitably R⁸ represents phenyl or optionally substituted heteroaryl e.g. dimethylisoxazolyl.

Suitably R⁹ includes C₁₋₈alkyl optionally substituted by OH, CN, OC₁₋₄alkyl, CONH₂, CONHC₁₋₄alkyl, or SO₂phenyl; C₂₋₈alkenyl; optionally substituted CH₂phenyl; optionally substituted CH₂heterocyclyl; CH₂CH₂heterocyclyl; or optionally substituted CH₂-bicyclic heterocyclyl.

In one aspect R⁹ includes C₁₋₈alkyl optionally substituted by OH, CN, OC₁₋₄alkyl e.g. OCH₃, OC₂H₅, or OCH(CH₃)₂, CONH₂; CONHC₁₋₄alkyl, or SO₂phenyl; C₂₋₈alkenyl, e.g. propenyl; optionally substituted CH₂phenyl; CH₂pyridine; CH₂tetrahydrofuran; CH₂CH₂-2-(1,3-dioxanyl); optionally substituted CH₂ isoxazole e.g. CH₂(5-methyl-3-isoxazole); CH₂CH₂pyrrole and CH₂quinoline.

Suitable substituents when R⁹ is optionally substituted phenyl include one or two substituents selected from F, Cl, Br, C₁₋₄alkyl e.g. methyl or isopropyl, CF₃, OCH₃, OCF₃, CN, SO₂NH₂, SO₂N(CH₃)₂, and SO₂CH₃.

Suitably R¹⁰ is hydrogen.

Suitably R¹¹ is hydrogen.

Suitably Q^{a} is hydrogen.

Suitably Q^{b} is hydrogen.

Suitably Q^{c} is hydrogen, CH₃ or F. In one aspect Q^{c} is hydrogen or CH₃.

Suitably Q^{d} is hydrogen, CH₃ or F. In one aspect Q^{d} is hydrogen or CH₃.

Compounds of formula (I) include the compounds of examples 1 to 496 and derivatives thereof.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine.

The term “alkyl” as a group or part of a group means a straight, branched or cyclic alkyl group or combinations thereof. Unless hereinbefore defined, examples of alkyl include C₁₋₈alkyl, for example methyl, ethyl, n-propyl, iso-propyl, n-butyl, sec-butyl, iso-butyl, t-butyl, pentyl, hexyl, 1,1-dimethylethyl, cyclopropyl, cyclopentyl or cyclohexyl or combinations thereof such as cyclopropylmethylene, cyclohexylmethylene and cyclopentylmethylene.

When used herein the term “cycloalkyl” means a cyclic alkyl group comprising up to eight carbon atoms in a ring.

The term “alkenyl” means linear or branched structures and combinations thereof, of the indicated number of carbon atoms, having at least one carbon-to-carbon double bond, wherein hydrogen may be replaced by an additional carbon to carbon double bond. C₃₋₈alkenyl, for example, includes 2-methyl-2-propenyl and the like.

The term “alkynyl” means linear or branched structures and combinations thereof, of the indicated number of carbon atoms, having at least one carbon-to-carbon triple bond. C₃₋₈alkynyl, for example, includes propynyl and the like.

The term “alkoxy” as a group or as part of a group means a straight, branched or cyclic chain alkoxy group. Unless hereinbefore defined “alkoxy” includes C₁₋₈alkoxy, e.g. methoxy, ethoxy, n-propoxy, iso-propoxy, n-butoxy, sec-butoxy, iso-butoxy, t-butoxy, pentoxy, hexyloxy, cyclopentoxy or cyclohexyloxy. In one aspect “alkoxy” is C₁₋₆alkoxy.

The term “heterocyclyl” as a group or as part of a group means an aromatic or non-aromatic five or six membered ring which contains from 1 to 4 heteroatoms selected from nitrogen, oxygen or sulfur and unsubstituted or substituted by, for example, up to three substituents, preferably one or two substituents. Examples of 5-membered heterocycles include furan, tetrahydrofuran, thiophene, tetrahydrothiophene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, and tetrazole. Examples of 6-membered heterocycles include pyran, tetrahydropyran, pyridine, piperidine, dioxane, morpholine, dithiane, thiomorpholine, pyridazine, pyrimidine, pyrazine, piperazine, and triazine.

The term “heterocyclyloxy” as a group or as part of a group refers to an “—O-heterocyclyl” group, wherein the term “heterocyclyl” is as defined above.

The term “aliphatic heterocyclyl” as a group or as part of a group means an aliphatic five or six membered ring which contains 1 or 2 heteroatoms selected from nitrogen, oxygen or sulfur and is unsubstituted or substituted by, for example, up to three substituents, preferably one or two substituents.

The term “aryl” as a group or part of a group means a 5- or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is phenyl.

The term “aryloxy” as a group or as part of a group refers to an “—O-aryl” group, wherein the term “aryl” is as defined above.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents, for example up to 3 or up to 2 substituents. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

When the heteroatom nitrogen replaces a carbon atom in an alkyl group, or when nitrogen is present in a heteroaryl, heterocyclyl or bicyclic heterocyclyl group, the nitrogen atom will, where appropriate be substituted by one or two substituents selected from hydrogen and C₁₋₈alkyl, preferably hydrogen and C₁₋₆alkyl, more preferably hydrogen.

Compounds of formula (I) can be prepared as set forth in the following scheme and in the examples. The following processes form another aspect of the present invention.

Compounds of formula (I) may be prepared from intermediates of formula (II).

Intermediates of Formula (II) may be prepared by the following general route:

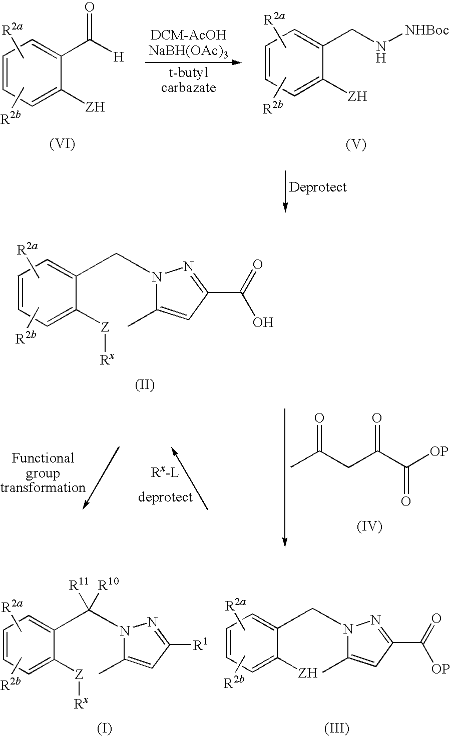

wherein L is a leaving group for example halo, e.g. bromo; P is a protecting group for example C₁₋₄ alkyl e.g. methyl or ethyl; and Z, R^{2a}, R^{2b}, R¹ and R^{x} are as defined above for compounds of formula (I). In one aspect Z is O.

Suitable reaction conditions for the reaction of a compound of formula (III) with a compound R^{x}-L are known to those skilled in the art and include the use of a solvent e.g. a C₁₋₄alcohol such as methanol or ethanol in the presence of a base, e.g. sodium hydroxide.

Suitable conditions for the deprotection of an ester to give the corresponding carboxylic acid are known to those skilled in the art.

Suitable reaction conditions for the reaction of a compound of formula (IV) with a compound of formula (V) to give a pyrazole of formula (III) will be apparent to the skilled person, and include treatment with trifluoroacetic acid in a solvent, e.g. dichloromethane, at room temperature to remove the protecting group on the compound of formula (V) followed by condensation with (IV) in a solvent, such as acetic acid, or an alcohol, such as methanol.

Suitable reaction conditions for the conversion of a salicylaldehyde of formula (VI) to a compound of formula (V) include reacting the salicylaldehyde with tert-butyl carbazate in the presence of acetic acid and sodium triacetoxyborohydride in a solvent such as dichloromethane.

Intermediates of formula R^{x}-L wherein L is OH, Br or Cl and R^{x} is as defined for compounds of formula (I) are commercially available, or may be readily prepared by known transformations of commercially available compounds.

Intermediates of formula (IV):

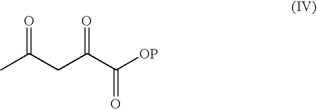

wherein P is C₁₋₄ alkyl e.g. methyl or ethyl, are commercially available.

Intermediates of formula (VI):

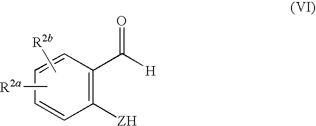

wherein R^{2a} and R^{2b} are as defined for compounds of formula (I) are commercially available, or may readily be prepared by methods known to those skilled in the art, for example from suitable commercially available starting materials using methods as described in the examples. The preparation of aldehydes is reviewed in The Chemistry of the Carbonyl Group, S. Patai (Ed), Interscience, New York, 1966, and references cited therein.

Compounds of formula (I) where R¹⁰ and R¹¹ are independently selected from alkyl; or R¹⁰ and R¹¹ together with the carbon to which they are attached form a cycloalkyl ring, optionally containing up to one heteroatom selected from O, S, NH or N-alkyl can be prepared from compounds of formula (I) wherein R¹⁰ and/or R¹¹ are hydrogen, by deprotonation with for example lithium diisopropylamide (LDA), and subsequent alkylation with a suitable reagent.

Compounds of formula (I) where R¹⁰ and R¹¹ are independently selected from alkyl; or R10 and R¹¹ together with the carbon to which they are attached form a cycloalkyl ring, optionally containing up to one heteroatom selected from O, S, NH or N-alkyl may also be prepared from compounds of formula (II) or formula (III), by deprotonation as described above followed by alkylation with a suitable reagent. Subsequently the resulting carboxylic ester group may be converted to a group R¹ by known functional group transformations to give a compound of formula (I).

Compounds of formula (I) where R¹⁰ and/or R¹¹ are fluorine may be prepared by an analogous method to that described in P. Kirsch & A. Taugerbeck, Eur. J. Org. Chem., 3923-3926 (2002).

It will be recognised to those skilled in the art that the compounds of formula (I) can be derived from the carboxylic acid intermediates of formula (II). Compounds of formula (I) wherein R¹ is CONR³R⁴, such as amides, can be prepared by activation of the carboxylic acid, for example by forming the acid chloride (for example by reaction of the carboxylic acid with thionyl chloride) followed by reaction with an amine or a sulfonamide respectively. Compounds of Formula (I) wherein R¹ is NR³CO₂R⁵ may be accessed by using the Curtius reaction (P. A. S. Smith, Org. React. 3, 337-449 (1946) and J. H. Saunders, R. J. Slocombe, Chem. Rev. 43, 205 (1948)). Compounds of Formula (I) wherein R¹ is NR³COR⁶ may also be accessed by using the aforementioned Curtius reaction followed by deprotection of the resulting carbamate and reaction of the amine with a carboxylic acid derivative such as an acid chloride. It will be recognised to those skilled in the art that a carboxylic acid group may be converted to a pyrazole, oxazole, triazole, benzoxazole or imidazole group by a sequence of well known functional group transformations such as those described in the Examples and those described in A. R. Katritzky, C. W. Rees ‘Comprehensive Heterocyclic Chemistry’, Pergamon (1984). Tetrazoles may be formed from carboxylic acids by converting the carboxylic acid to the primary amides, for example by reaction with oxalyl chloride followed by ammonia, followed by dehydration of the amide to the nitrile, for example by heating in phosphorous oxychloride, followed by reaction with azide.

Compounds of formula (I) wherein R¹ is OCONR³R⁷ [hereinafter referred to as compounds of formula Ia] may be prepared in accordance with the following scheme:

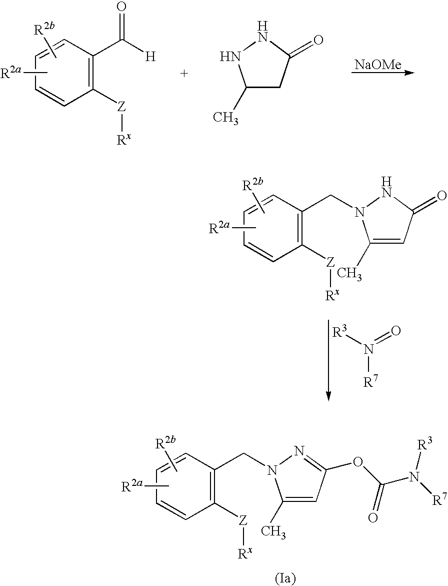

Compounds of formula (I) wherein R¹ is an imidazole moiety fused to give an optionally substituted bicyclic or tricyclic ring system [hereinafter referred to as compounds of formula Ib] may be prepared from compounds of formula (VII) following the methods described in, for example, A. Czarny et al, J. Het. Chem., 1996, 33(4), 1393-1398 and according to the following scheme:

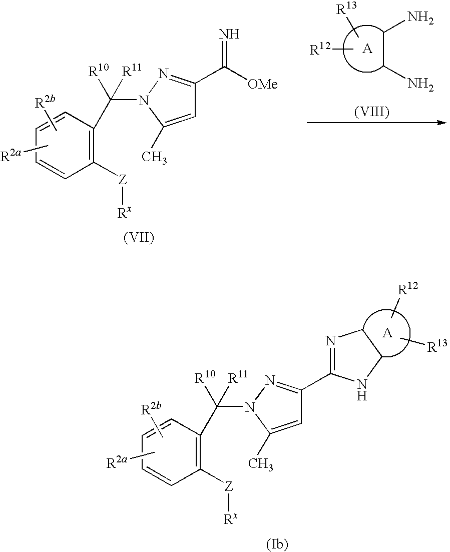

wherein Z, R^{2a}, R^{2b}, R¹⁰, R¹¹ and R^{x} are as defined for compounds of formula (I); A represents e.g. phenyl, pyridine, quinoline, or thiophene, and R¹² and R¹³ each represent hydrogen or a substituent.

Suitable reaction conditions for the preparation of a compound of formula (Ib) include heating the intermediates together in a suitable solvent e.g. ethanol.

Compounds of formula (VII) may be prepared from the corresponding carboxylic acid of formula (II) by known methods, for example as described in the examples. Suitable methods include the reaction of a compound of formula (II) with thionyl chloride then ammonia, then phosphorus oxychloride, then sodium methoxide in methanol.

Diamines of formula (VIII) are commercially available, or may be prepared by known methods.

Compounds of formula (I) wherein R¹ is a benzimidazole may also be prepared from the reaction of a diamine of formula (VIII) with a compound of formula (IX):

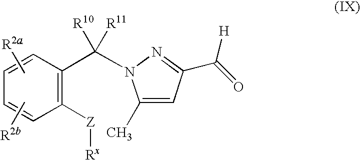

Compounds of formula (IX) may be prepared from compounds of formula (II) by known methods, for example by reaction of a compound of formula (II) with lithium aluminium hydride in a suitable solvent, e.g. THF to give the corresponding methanol, followed by conversion to the corresponding carbaldehyde using Dess-Martin periodinane.

Compounds of formula (I) wherein R¹ is benzimidazole may be functionalised on the benzimidazole ring using methods described in the Examples and in the scheme below:

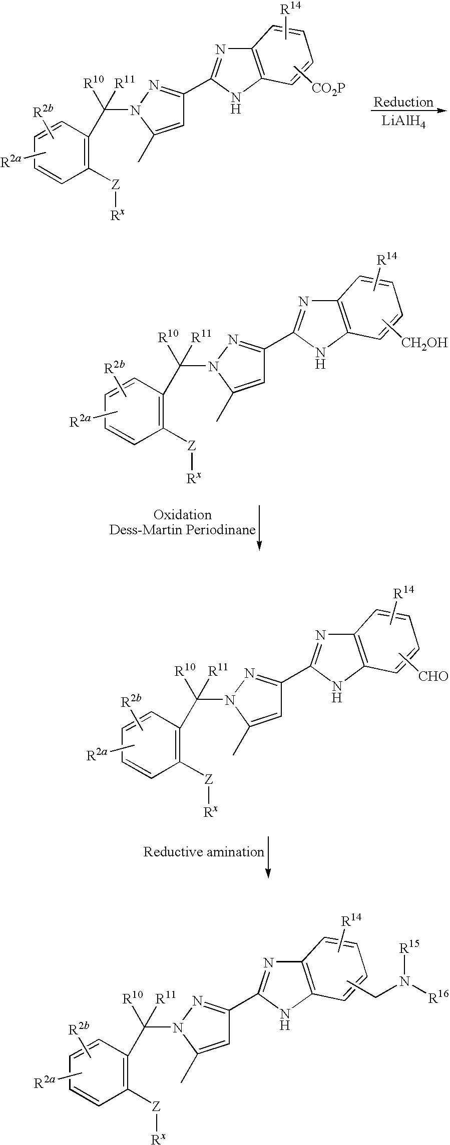

wherein X, Z, R^{2a}, R^{2b}, R³, R¹⁰, R¹¹ and R^{x} are as hereinbefore defined for compounds of formula (I), R¹⁴ is hydrogen or a substituent, R¹⁵ and R¹⁶ are independently selected from hydrogen and optionally substituted C₁₋₄alkyl, or R¹⁵ and R¹⁶ together with the nitrogen atom to which they are attached form a heterocyclyl ring optionally containing another heteroatom selected from O, NH, NC₁₋₄alkyl, or S.

Compounds of formula (I) wherein R¹ is CONR³R⁴ or NR³COR⁶ when R⁴ or R⁶ are optionally substituted aryl may be functionalised in accordance with the methods outlined in the scheme above.

Compounds of formula (I) wherein R¹ is a substituted piperazinone (compounds of formula Ic) may be prepared by methods as described in the examples and, for example, in S. A. Weissmann et al, Tet. Lett. 1998 39(41) 7459-7462, in accordance with the general route below:

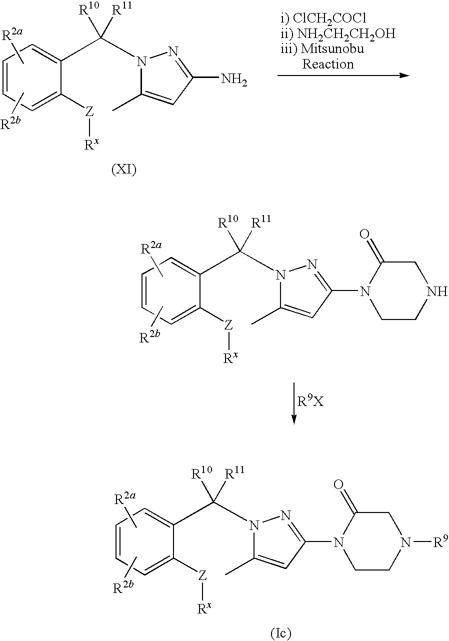

Compounds of formula (XI) may be prepared from compounds of formula (II) by known methods, for example by reaction of a compound of formula (II) with diphenylphosphoryl azide in tert-butanol in the presence of triethylamine to give the corresponding tert-butylcarbamate, followed by treatment with HCl in a suitable solvent such as 1,4-dioxan to give the amine of formula (XI).

Certain substituents in any of the reaction intermediates and compounds of formula (I) may be converted to other substituents by conventional methods known to those skilled in the art. Examples of such transformations include the reduction of a nitro group to give an amino group; alkylation and amidation of amino groups; hydrolysis of esters, alkylation of hydroxy and amino groups; and amidation and esterification of carboxylic acids. Such transformations are well known to those skilled in the art and are described in for example, Richard Larock, Comprehensive Organic Transformations, 2nd edition, Wiley-VCH, ISBN 0-471-19031-4.

The skilled person will recognise that one group R^{x} may be converted to another group R^{x} using known chemistry. For example when R^{x} is a benzyl group and Z is O, cleavage of the ether to give a phenol may be effected using for example HBr/acetic acid. The resulting phenol can then be converted to another group OR^{x} by reaction of the phenol with, for example, a suitable substituted benzyl bromide or alkyl bromide.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

Accordingly the present invention also provides a process for the preparation of a compound of formula (I) or a derivative thereof:

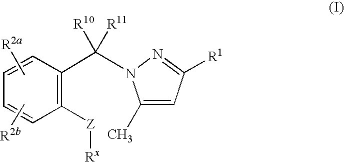

wherein:

Z is O, S, SO or SO₂;

R^{x} is optionally substituted C₂₋₁₀alkyl, optionally substituted C₂₋₁₀alkenyl, optionally substituted C₂₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;

R¹ is CONR³R⁴, NR³CO₂R⁵, NR³COR⁶, OCONR³R⁷, tetrazolyl, oxazolin-2-yl, oxazol-2-yl, benzoxazol-2-yl, pyrrolidinonyl, isoindoledionyl, dihydroisoindolonyl, or optionally substituted SO₂NHCOaryl; or R¹ is optionally substituted imidazolyl or optionally substituted 1,2,4-triazolyl wherein optionally the imidazole or 1,2,4-triazole ring is fused to give an optionally substituted bicyclic or tricyclic ring system;

### or R¹ is

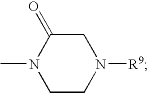

R^{2a} and R^{2b} are independently selected from hydrogen, halo, CN, SO₂alkyl, SR³, NO₂, optionally substituted alkyl, and optionally substituted alkoxy;

R³ is hydrogen or C₁₋₄alkyl;

R⁴ is hydrogen, OH, optionally substituted alkyl, optionally substituted aryl, optionally substituted heterocyclyl, optionally substituted bicyclic heterocyclyl, optionally substituted CQ^{c}Q^{d}aryl, optionally substituted CQ^{c}Q^{d}heterocyclyl, optionally substituted CQ^{c}Q^{d}bicyclic heterocyclyl, or SO₂R⁸;

R⁵ is C₁₋₄alkyl optionally substituted by SiMe₃, SO₂C₁₋₄alkyl, OC₁₋₄alkyl, N(C₁₋₄alkyl)₂, CO₂C₁₋₄alkyl, or CF₃; cyclohexyl substituted by CH₂NHC₁₋₄alkyl, CH₂pyrrolidinyl, CH₂morpholinyl or CH₂piperidinyl; phenyl; CQ^{c}Q^{d}phenyl; CQ^{c}Q^{d}pyridyl; CQ^{c}Q^{d}thienyl; CQ^{c}Q^{d}tetrahydrofuryl; CQ^{c}Q^{d}furyl; CQ^{c}Q^{d}piperidinyl optionally substituted by C₁₋₄alkyl; CH₂CH₂pyrrolidinonyl; CQ^{c}Q^{d}CH₂morpholinyl; tetrahydropyranyl; tetrahydrofuryl; 2-pyrrolidinon-4-yl; tetrahydrothienyl-1,1-dioxide; piperidin-4-yl optionally substituted on the 1-position by CO₂C₁₋₄alkyl; or dihydroindenyl;

R⁶ is alkyl, optionally substituted aryl, optionally substituted heterocyclyl, optionally substituted bicyclic heterocyclyl, optionally substituted CQ^{c}Q^{d}-Y-aryl, optionally substituted CQ^{c}Q^{d}-Y-heterocyclyl or optionally substituted CQ^{c}Q^{d}-Y-bicyclic heterocyclyl;

R⁷ is optionally substituted alkyl, optionally substituted alkenyl, optionally substituted aryl, or optionally substituted CQ^{c}Q^{d}aryl;

R⁸ is optionally substituted alkyl, optionally substituted aryl or optionally substituted heterocyclyl;

R⁹ is alkyl optionally substituted by OH, CN, OC₁₋₃alkyl, CONH₂, CONHC₁₋₄alkyl, or SO₂phenyl; alkenyl; optionally substituted CQ^{c}Q^{d}-Y-aryl; optionally substituted CQ^{c}Q^{d}-Y-heterocyclyl; or optionally substituted CQ^{c}Q^{d}-Y-bicyclic heterocyclyl;

R¹⁰ and R¹¹ are independently selected from hydrogen, fluorine and alkyl; or R¹⁰ and R¹¹ together with the carbon to which they are attached form a cycloalkyl ring, optionally containing up to one heteroatom selected from O, S, NH or N-alkyl; and

Y is CH₂ or a bond;

Q^{a} and Q^{b} are each independently selected from hydrogen, CH₃ and fluorine;

Q^{c} and Q^{d} are each independently selected from hydrogen, CH₃ and fluorine;

provided that:

R⁹ is not optionally substituted CH₂furan or optionally substituted CH₂imidazole;

when R^{x} is 2-methylpropyl, then R⁵ is not 1-methylethyl;
when R^{x} is optionally substituted CH₂cyclopropyl, then R⁹ is not 2-methylpropyl, CH₂cyclopropyl, CH₂cyclobutyl, CH₂CH₂OCH₃ or CH₂CH₂OH;
when R^{x} is CH₂tetrahydropyranyl or CH₂CH₂N(CH₃)₂, then R⁹ is not 2-methylpropyl.
when R¹ is benzimidazolyl it is unsubstituted on the 1-position; and
when R¹ is benzimidazole optional substituents on the 4 or 7 position are selected from CH₂OH or CO₂H.
comprising:
converting a compound of formula (II):

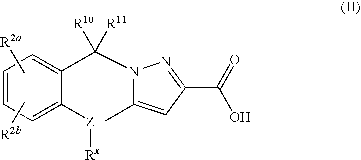

wherein R^{2a}, R^{2b}, R^{x}, R¹⁰, and R¹¹ are as defined for compounds of formula (I); to a compound of formula (I);
and if required, and in any order;
converting one group R¹⁰ to another group R¹⁰; and/or
converting one group R¹¹ to another group R¹¹; and/or
converting one group R¹ to another group R¹; and/or
forming a derivative thereof.

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0-471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opiods (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendonitis, bursitis, and Sjogren's syndrome.

Immunological diseases include autoimmune diseases, immunological deficiency diseases or organ transplantation. The compounds of formula (I) are also effective in increasing the latency of HIV infection

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendonitis and bursitis.

Cardiovascular diseases include hypertension or myocardiac ischemia; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X_{4/7}. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

In addition to activity at the EP₁ receptor, certain compounds of the present invention and pharmaceutically acceptable derivatives thereof exhibit antagonism of the TP receptor and are therefore indicated to be useful in treating conditions mediated by the action of thromboxane at the TP receptor. Conditions mediated by the action of thromboxane at the TP receptor include renal disorders, asthma, or gastric lesions.

In certain situations it is envisaged that the administration of a compound exhibiting antagonism of TP receptors in combination with a compound exhibiting antagonism of EP₁ receptors may be advantageous.

Certain compounds of the invention are selective for EP₁ over EP₃.

No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

It will be appreciated to those skilled in the art that where compounds are named as hydrochloride salts the stoichiometry of the isolated reaction products is undetermined due to the nature of their preparation. Compounds have therefore been named as hydrochlorides and denoted as xHCl, where x is 0-3 and represents the stoichiometry of said salt.

### Abbreviations

AcOH, acetic acid, Bn (benzyl), Bu, Pr, Me, Et (butyl, propyl, methyl, ethyl), DMSO (dimethyl sulfoxide), DCM/MDC (dichloromethane), DME (ethylene glycol dimethyl ether), DMF (N,N-dimethylformamide), EDTA (ethylenediaminetetraacetic acid), EtOAc (ethyl acetate), EtOH (ethanol), HPLC (High pressure liquid chromatography), IPA (isopropanol), LCMS (Liquid chromatography/Mass spectroscopy), MDAP (Mass Directed Auto Preparation), MeOH (methanol), ML (mother liquor), NMR (Nuclear Magnetic Resonance (spectrum)), NMP (n-methylpyrrolidone), Ph (phenyl), pTSA (para-toluene sulphonic acid), RT/Rt (retention time), SM (starting material), SPE (Solid Phase Extraction—silica cartridge chromatography), TBAF (tetrabutylammonium fluoride), TBME (tertiary butyl methyl ether), THF (tetrahydrofuran), s, d, dd, t, q, m, br (singlet, doublet, double doublet, triplet, quartet, multiplet, broad.)

### Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation, also referred to as mass directed LCMS purification). MDAP is described in e.g. W. Goetzinger et al, Int. J. Mass Spectrom., 2004, 238, 153-162.

Flash Master II is an automated chromatography system using commercial prepacked columns. Biotage is a chromatography system using commercial pre-packed silica gel cartridges. The term FLEX (Parallel Flex) when used herein refers to a parallel HPLC purification system.

### LCMS

The following conditions were used for LCMS in the preparation of the examples.
Column: 3.3 cm×4.6 mm ID, 3 μm ABZ+PLUS
Flow Rate: 3 ml/min
Injection Volume: 5 μl
Temp: Room temperature
UV Detection Range: 215 to 330 nm

| Solvents: | A: 0.1% Formic Acid + 10 mMolar Ammonium Acetate. | | |
|---|---|---|---|
| | B: 95% Acetonitrile + 0.05% Formic Acid | | |
| | Time | A % | B % |
| Gradient: | 0.00 | 100 | 0 |
| | 0.70 | 100 | 0 |
| | 4.20 | 0 | 100 |
| | 5.30 | 0 | 100 |
| | 5.50 | 100 | 0 |

All retention times are measured in minutes.

### Preparation of Intermediates

### 1,1-Dimethylethyl 2-[(5-bromo-2-hydroxyphenyl)methyl]hydrazinecarboxylate

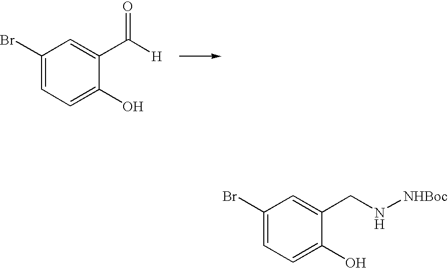

5-Bromo-2-hydroxybenzaldehyde (4.02 g, 20 mmol) was dissolved in dichloromethane (100 ml). tert-Butyl carbazate (2.64 g, 20 mmol) and acetic acid (1.14 ml, 1.2 g, 20 mmol) were added and the mixture was stirred under nitrogen for 30mins. Sodium triacetoxyborohydride (12.72 g, 60 mmol) was added portionwise and the resulting suspension was then stirred overnight under nitrogen. 2M hydrochloric acid (30 ml, 60 mmol) was added and the resulting solution was diluted with dichloromethane and water. The organic phase was separated, washed sequentially with brine and water then dried (Na₂SO₄) and evaporated to dryness to give the title compound as a white solid (6.01 g). LC/MS Rt==3.11 min, [MH+] 317, 319 [MH−] 315, 317.

### 1,1-Dimethylethyl 2-[(5-chloro-2-hydroxyphenyl)methyl]hydrazinecarboxylate

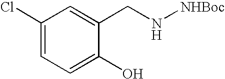

The title compound was prepared in a similar manner to 1,1-dimethylethyl 2-[(5-bromo-2-hydroxyphenyl)methyl]hydrazinecarboxylate from the appropriate intermediates.

¹H NMR (CDCl₃) δ: 1.50 (9H, s), 4.10 (2H, d), 4.40 (1H, brs), 6.15 (1H, brs), 6.80 (1H, d), 7.00 (1H, d), 7.20 (1H, dd), 9.25 (1H, br s).

### Ethyl 1-[(5-bromo-2-hydroxyphenyl)methyl]-5-methyl-1H-pyrazole-3-carboxylate

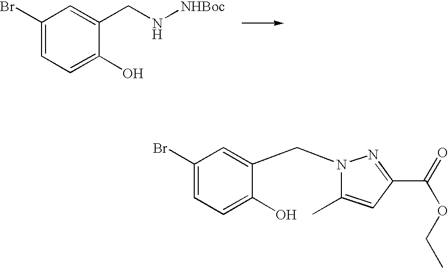

Trifluoroacetic acid (20 ml) was added to 1,1-dimethylethyl 2-[(5-bromo-2-hydroxyphenyl)methyl]hydrazinecarboxylate (3.2 g, 10 mmol) in dichloromethane (40 ml) and the reaction mixture stirred overnight at room temperature under nitrogen. The solvent was removed in vacuo and the residue dissolved in acetic acid (20 ml). The resulting solution was added dropwise to a solution of ethyl 2,4-dioxopentanoate (1.40 ml, 1.58 g, 10 mmol) in acetic acid (10 ml) and the reaction mixture was heated at reflux under nitrogen for 1 h. The title compound crystallized upon cooling, was filtered, washed with acetic acid and dried in vacuo to give the title compound as white crystals (1.85 g).

LC/MS Rt=3.17 min, [MH+] 339, 341.

### Ethyl 1-[(5-chloro-2-hydroxyphenyl)methyl]-5-methyl-1H-pyrazole-3-carboxylate

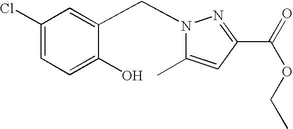

The title compound was prepared in a similar manner to ethyl 1-[(5-bromo-2-hydroxyphenyl)methyl]-5-methyl-1H-pyrazole-3-carboxylate from the appropriate intermediates. LC/MS Rt=2.60 min, [MH+] 295.

### Ethyl 1-({5-chloro-2-[(phenylmethyl)oxy]phenyl}methyl)-5-methyl-1H-pyrazole-3-carboxylate

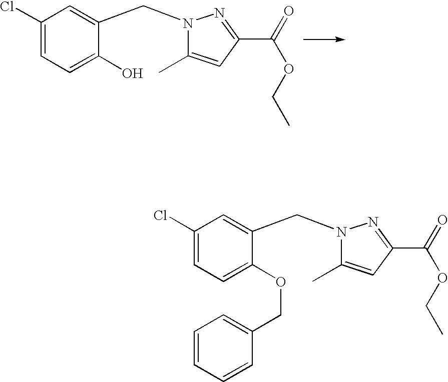

Ethyl 1-[(5-chloro-2-hydroxyphenyl)methyl]-5-methyl-1H-pyrazole-3-carboxylate (17.5 g, 59 mmol) was suspended in acetone (280 ml). To the mixture was added benzyl bromide (7.46 ml, 62 mmol) followed by potassium carbonate (16.40 g, 119 mmol). This was heated at reflux for 18 hours. The reaction was filtered and washed with acetone. The filtrate was evaporated to a white solid, which was triturated in hexane. The resulting white solid was collected by filtration (22.87 g). LC/MS Rt=3.14 min, [MH+] 385.

### Ethyl 1-({5-chloro-2-[(2-methylpropyl)oxy]phenyl}methyl)-5-methyl-1H-pyrazole-3-carboxylate

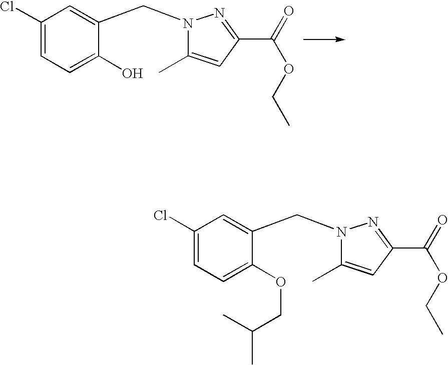

To a solution of ethyl 1-[(5-chloro-2-hydroxyphenyl)methyl]-5-methyl-1H-pyrazole-3-carboxylate (17.46 g, 59 mmol) in N,N-dimethylformamide (120 ml) was added 1-bromo-2-methylpropane (12.18 g, 89 mmol) and potassium carbonate (16.36 g, 119 mmol). This was heated to 80° C. for 8 hours. 1-bromo-2-methylpropane (4.04 g, 29.5 mmoll) and potassium carbonate (8.08 g, 59 mmol) was added to the mixture and heating continued for 16 hours. The reaction mixture was diluted with diethyl ether and water. The organic phase was separated. The aqueous phase was re-extracted with diethyl ether, the extracts combined and washed with water then dried (Na₂SO₄) and evaporated to an oil which crystallised. This was triturated with hexane and the resulting white solid was collected by filtration (20.68 g). LC/MS Rt=3.47 min, [MH+] 351.

The following compounds were prepared in a similar manner to ethyl 1-({5-chloro-2-[(2-methylpropyl)oxy]phenyl}methyl)-5-methyl-1H-pyrazole-3-carboxylate from the appropriate intermediates.

| Structure | Name | Data |
|---|---|---|
| 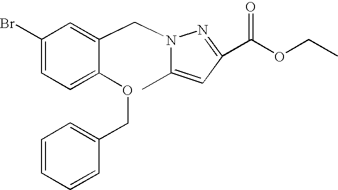 | Ethyl 1-({5-bromo-2- [(phenylmethyl)oxy]phenyl} methyl)-5-methyl-1H-pyrazole-3- carboxylate | LC/MS Rt = 3.48 min [MH⁺] 429, 431 |
| 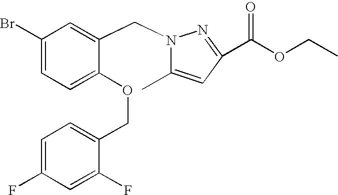 | Ethyl 1-[(5-bromo-2-{[(2,4- difluorophenyl)methyl]oxy} phenyl)methyl]-5-methyl-1H- pyrazole-3-carboxylate | LC/MS Rt = 3.51 min [MH⁺] 465 |
| 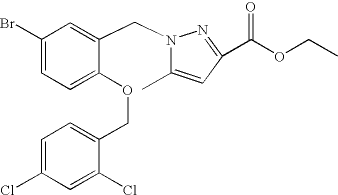 | Ethyl 1-[(5-bromo-2-{[(2,4- dichlorophenyl)methyl]oxy} phenyl)methyl]-5-methyl-1H- pyrazole-3-carboxylate | LC/MS Rt = 3.85 min [MH⁺] 497, 499 |
| 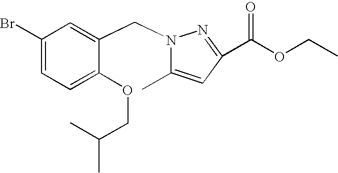 | Ethyl 1-({5-bromo-2-[(2- methylpropyl)oxy]phenyl}methyl)- 5-methyl-1H-pyrazole-3- carboxylate | LC/MS Rt = 3.64 min [MH⁺] 396, 398 |
| 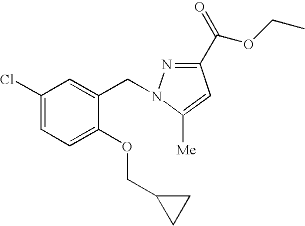 | Ethyl 1-({5-chloro-2- [(cyclopropylmethyl)oxy]phenyl} methyl)-5-methyl-1H-pyrazole- 3-carboxylate | LC/MS Rt = 3.53 min [MH⁺] 349.2 |
| 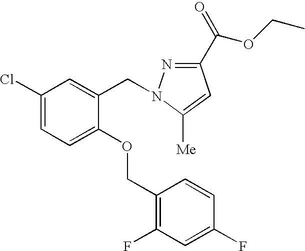 | Ethyl 1-[(5-chloro-2-{[(2,4- difluorophenyl)methyl]oxy}phenyl) methyl]-5-methyl-1H- pyrazole-3-carboxylate | |

### 1-({5-Chloro-2-[(phenylmethyl)oxy]phenyl}methyl)-5-methyl-1H-pyrazole-3-carboxylic acid

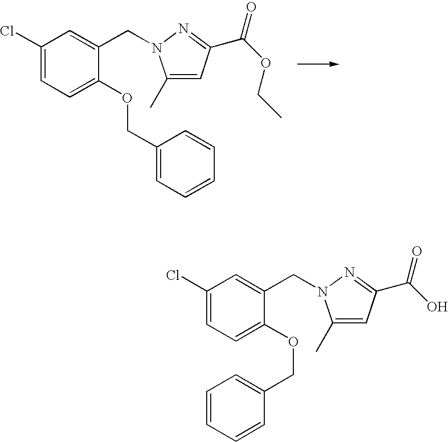

Ethyl 1-({5-chloro-2-[(phenylmethyl)oxy]phenyl}methyl)-5-methyl-1H-pyrazole-3-carboxylate (26.93 g, 70 mmol) was dissolved in ethanol (300 ml) and 2M sodium hydroxide (100 ml). This mixture was stirred at reflux for 30 minutes. The ethanol was evaporated and the mixture diluted with ethyl acetate and water. This was acidified with concentrated hydrochloric acid, and the phases separated. The aqueous phase was re-extracted with ethyl acetate, the organic layers combined, washed with brine, dried (MgSO₄) and evaporated to dryness. The residue was triturated with isohexane and the white solid filtered and dried (24.95 g). LC/MS Rt=3.45 min, [MH+] 357, 359.

The following compounds were prepared in a similar manner to 1-({5-chloro-2-[(phenylmethyl)oxy]phenyl}methyl)-5-methyl-1H-pyrazole-3-carboxylic acid from the appropriate intermediates.

| Structure | Name | Data |
|---|---|---|
| 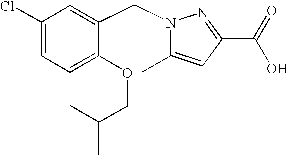 | 1-({5-Chloro-2-[(2- methylpropyl)oxy]phenyl}methyl)- 5-methyl-1H-pyrazole-3-carboxylic acid | LC/MS Rt = 3.02 min, [MH−] 321. |
| 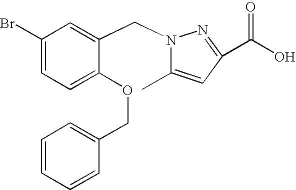 | 1-({5-Bromo-2- [(phenylmethyl)oxy]phenyl}methyl)- 5-methyl-1H-pyrazole-3- carboxylic acid | LC/MS Rt = 2.96 min [MH⁻] 401, 402 |
| 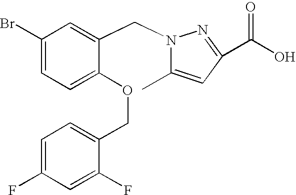 | 1-[(5-Bromo-2-{[(2,4- difluorophenyl)methyl]oxy}phenyl) methyl]-5-methyl-1H-pyrazole-3- carboxylic acid | LC/MS Rt = 3.14 min [MH⁻] 437, 438 |
| 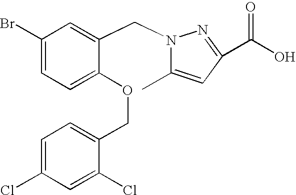 | 1-((5-Bromo-2-{[(2,4- dichlorophenyl)methyl]oxy}phenyl) methyl]-5-methyl-1H-pyrazole-3- carboxylic acid | LC/MS Rt = 3.47 min [MH⁻] 467, 469, 471 |
| 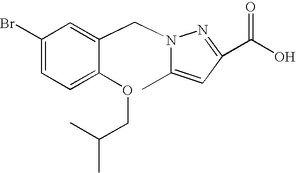 | 1-({5-Bromo-2-[(2- methylpropyl)oxy]phenyl}methyl)- 5-methyl-1H-pyrazole-3-carboxylic acid | LC/MS Rt = 3.09 min [MH⁻] 367, 368 |
| 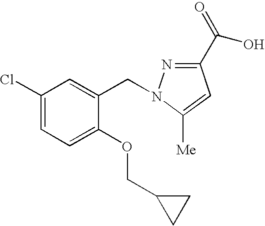 | 1-({5-Chloro-2- [(cyclopropylmethyl)oxy]phenyl} methyl)-5-methyl-1H-pyrazole-3- carboxylic acid | LC/MS Rt = 2.98 min, [MH]⁻ 319, 321 |
| 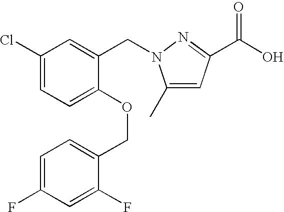 | 1-[(5-Chloro-2-{[(2,4- difluorophenyl)methyl]oxy}phenyl) methyl]-5-methyl-1H-pyrazole-3- carboxylic acid | |

### Example 1

### 1,1-Dimethylethyl[({5-chloro-2-[(phenylmethyl)oxy]phenyl}methyl)-5-methyl-1H-pyrazol-3-yl]carbamate

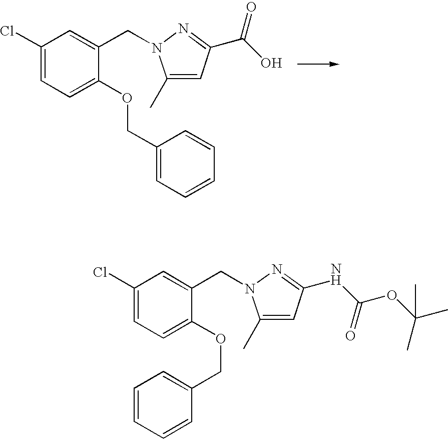

1-({5-Chloro-2-[(phenylmethyl)oxy]phenyl}methyl)-5-methyl-1H-pyrazole-3-carboxylic acid (19.62 g, 55 mmol) was dissolved in tert-butanol (200 ml). Triethylamine (8.47 ml, 60.5 mmol) and diphenylphosphoryl azide (13.05 ml, 60.5 mmol) were added to the solution. The mixture was heated to reflux under argon for 6 hours, then stirred at room temperature for 16 hours. The reaction mixture was evaporated and the residue purified by flash chromatography (15% ethyl acetate:hexane) to yield the title compound as a white solid (14.02 g). LC/MS Rt=3.97 min, [MH+] 428, 430.

The following examples were prepared in a similar manner to 1,1-dimethylethyl[1-({5-chloro-2-[(phenylmethyl)oxy]phenyl}methyl)-5-methyl-1H-pyrazol-3-yl]carbamate from the appropriate intermediates.

| Example | Structure | Name | Data |
|---|---|---|---|
| 2 | 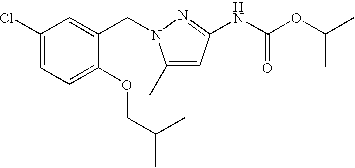 | 1-Methylethyl[1-({5- chloro-2-[(2- methylpropyl)oxy] phenyl}methyl)-5- methyl-1H-pyrazol- 3-yl]carbamate | LC/MS Rt = 3.70 min [MH⁺] 380, 382. |
| 3 | 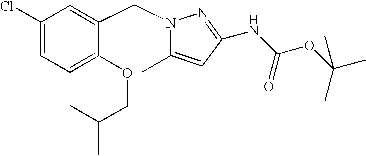 | 1,1-Dimethylethyl [1-({5-chloro-2-[(2- methylpropyl)oxy] phenyl}methyl)-5- methyl-1H-pyrazol- 3-yl]carbamate | ¹H NMR (CDCl₃) δ: 1.05 (6H, d), 1.50 (9H, s), 2.10 (1H, m), 2.20 (3H, s), 3.70 (2H, d), 5.10 (2H, s), 6.35 (1H, br s), 6.60 (1H, d), 6.75 (1H, d), 7.10 (1H, br s), 7.15 (1H, dd). |
| 4 | 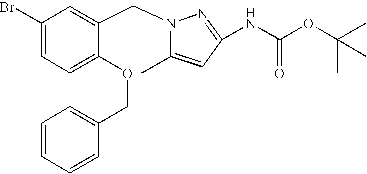 | 1,1-Dimethylethyl [1-({5-bromo-2- [(phenylmethyl)oxy] phenyl}methyl)-5- methyl-1H-pyrazol- 3-yl]carbamate | LC/MS Rt = 3.76 min [MH⁺] 472, 474 |
| 5 | 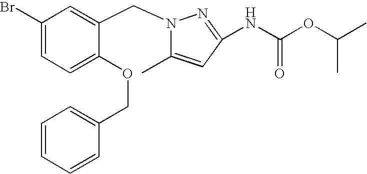 | 1-Methylethyl [1-({5- bromo-2- [(phenylmethyl)oxy] phenyl}methyl)-5- methyl-1H-pyrazol- 3-yl]carbamate | LC/MS Rt = 3.61 min [MH⁺] 458, 460 |
| 6 | 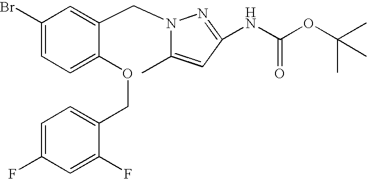 | 1,1-Dimethylethyl {1-[(5-bromo-2- {[(2,4- difluorophenyl)methyl]oxy}phenyl)methyl]- 5-methyl-1H- pyrazol-3- yl}carbamate | LC/MS Rt = 3.78 min [MH⁺] 508, 510 |
| 7 | 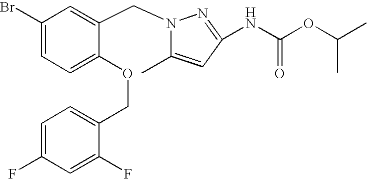 | 1-Methylethyl {1-[(5- bromo-2-{[(2,4- difluorophenyl)methyl] oxy}phenyl)methyl]- 5-methyl-1H- pyrazol-3- yl}carbamate | LC/MS Rt = 3.64 min [MH⁺] 494, 496 |
| 8 | 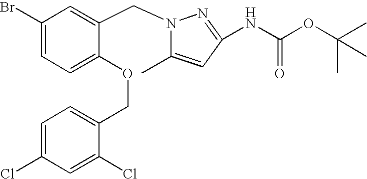 | 1,1-Dimethylethyl {1-[(5-bromo-2- {[(2,4- dichlorophenyl)methyl] oxy}phenyl)methyl]- 5-methyl-1H- pyrazol-3- yl}carbamate | LC/MS Rt = 4.00 [MH⁺] 542, 544 |
| 9 | 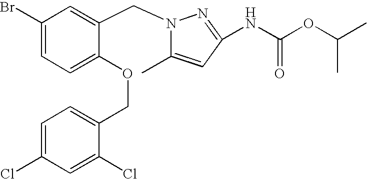 | 1-Methylethyl {1-[(5- bromo-2-{[(2,4- dichlorophenyl)methyl] oxy}phenyl)methyl]- 5-methyl-1H- pyrazol-3- yl]carbamate | LC/MS Rt = 3.80 [MH⁺] 528, 530 |
| 10 | 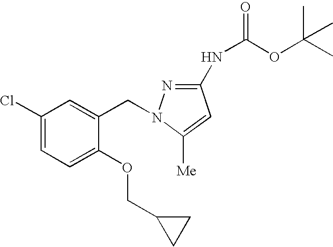 | 1,1-Dimethylethyl [1-({5-chloro-2- [(cyclopropylmethyl) oxy]phenyl}methyl)- 5-methyl-1H- pyrazol-3- yl]carbamate | LC/MS Rt = 3.53, [MH⁺] 392, 394 |

### Standard Method 1

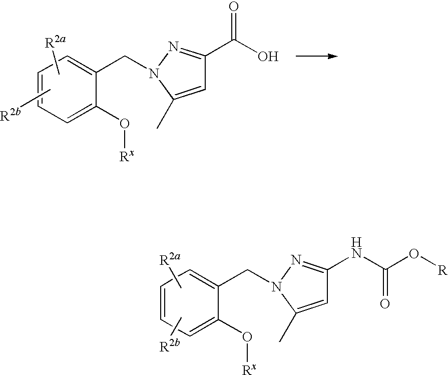

Diphenylphosphoryl azide (1 equivalent) was added to a solution of the relevant 5-methyl-1H-pyrazole-3-carboxylic acid (1 mmol) in toluene or 1,4-dioxan (0.5M) containing triethylamine (3 equivalents) then heated at 80° C. for 30 minutes. The alcohol (1-10 equivalents) was added and heating continued for 4-23.5 hours. Upon cooling to room temperature, the mixture was diluted with ethyl acetate and washed sequentially with 2M hydrochloric acid and saturated sodium bicarbonate solution, dried (Na₂SO₄), filtered and concentrated. The residue was purified by flash chromatography with hexane containing ethyl acetate (20-50%) to give the title compound. This was further purified by MDAP if necessary.

The following examples were prepared from the appropriate starting materials using similar methods to that described in Standard Method 1:

| Ex- | | | |
|---|---|---|---|
| ample | Structure | Name | Data |
| 10 | 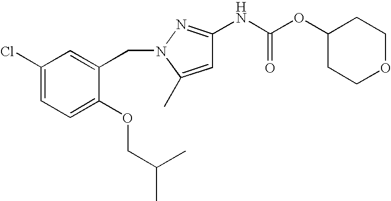 | Tetrahydro-2H-pyran-4-yl [1- ({5-chloro-2-[(2- methylpropyl)oxy]phenyl}meth yl)-5-methyl-1H-pyrazol-3- yl]carbamate | LC/MS Rt = 3.38 min [MH⁺] 422, 424 |
| 11 | 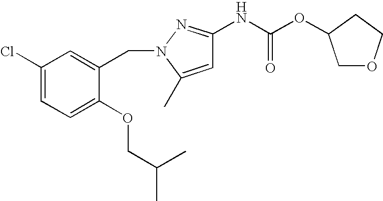 | Tetrahydro-3-furan [1-({5- chloro-2-[(2- methylpropyl)oxy]phenyl}meth yl)-5-methyl-1H-pyrazol-3- yl]carbamate | LC/MS Rt = 3.27 min [MH⁺] 408, 410 |
| 12 | 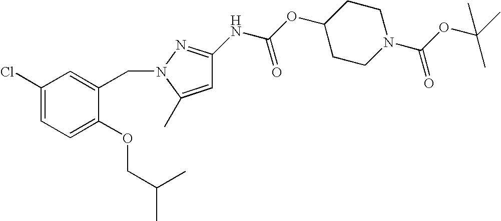 | 1,1-Dimethylethyl 4-[({[1-({5- chloro-2-[(2- methylpropyl)oxy]phenyl}meth yl)-5-methyl-1H-pyrazol-3- yl]amino}carbonyl)oxy]-1- piperidinecarboxylate | LC/MS Rt = 3.85 min [MH⁺] 521, 523 |
| 13 | 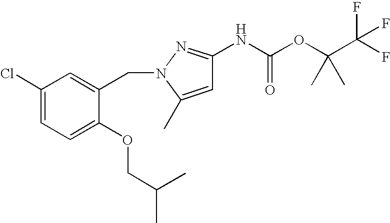 | 2,2,2-Trifluoro-1,1- dimethylethyl [1-({5-chloro-2- [(2- methylpropyl)oxy]phenyl}meth yl)-5-methyl-1H-pyraozl-3- yl]carbamate | LC/MS Rt = 3.96 min [MH⁺] 448, 450 |
| 14 | 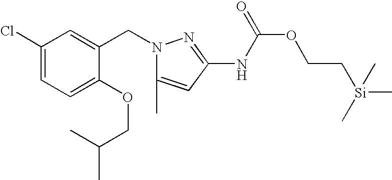 | 2-(Trimethylsilyl)ethyl [1-({5- chloro-2-[(2- methylpropyl)oxy]phenyl}meth yl)-5-methyl-1H-pyrazol-3- yl]carbamate | LC/MS Rt = 3.97 min [MH+] 438, 440. |
| 15 | 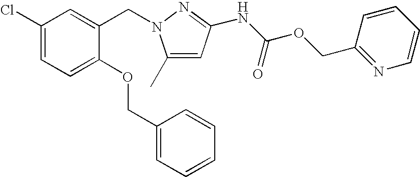 | 2-Pyridinylmethyl [1-({5- chloro-2- [(phenylmethyl)oxy]phenyl}meth- yl)-5-methyl-1H-pyrazol-3- yl]carbamate | LC/MS Rt = 3.51 min, [MH⁺] 463, 465. |
| 16 | 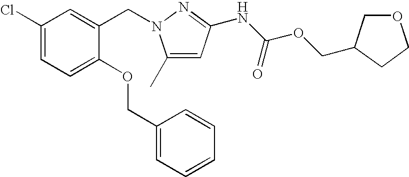 | Tetrahydro-3-furanylmethyl [1- ({5-chloro-2- [(phenylmethyl)oxy]phenyl}meth- yl)-5-methyl-1H-pyraozl-3- yl]carbamate | LC/MS Rt = 3.45 min, [MH⁺] 456, 458. |
| 17 | 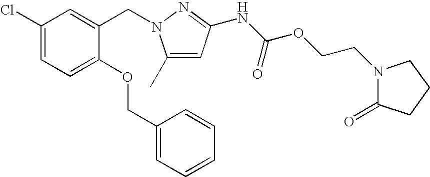 | 2-(2-Oxo-1-pyrrolidinyl)ethyl [1-({5-chloro-2- [(phenylmethyl)oxy]phenyl}meth- yl)-5-methyl-1H-pyrazol-3- yl]carbamate | LC/MS Rt = 3.15 min, [MH⁺] 483, 485. |
| 18 | 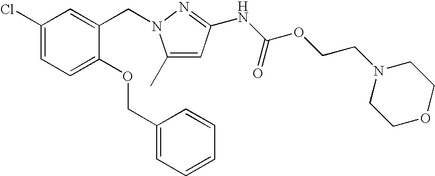 | 2-(4-Morpholinyl)ethyl [1-({5- chloro-2- [(phenylmethyl)oxy]phenyl}meth- yl)-5-methyl-1H-pyrazol-3- yl]carbamate | LC/MS Rt = 2.63 min, [MH⁺] 485, 487. |
| 19 | 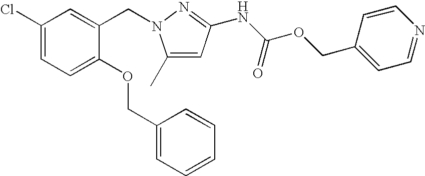 | 4-Pyridinylmethyl [1-({5- chloro-2- [(phenylmethyl)oxy]phenyl}meth- yl)-5-methyl-1H-pyrazol-3- yl]carbamate | LC/MS Rt = 3.26 min, [MH⁺] 463, 465. |
| 20 | 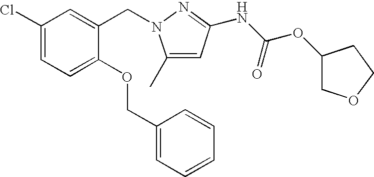 | (3S)-Tetrahydro-3-furanyl [1- ({5-chloro-2- [(phenylmethyl)oxy]phenyl}meth- yl)-5-methyl-1H-pyrazol-3- yl]carbamate | LC/MS Rt = 3.39 min, [MH⁺] 442, 444. |
| 21 | 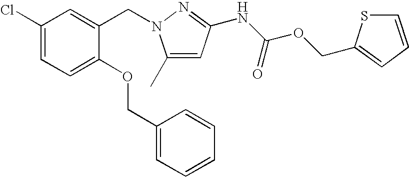 | 2-Thienylmethyl [1-({5-chloro- 2- [(phenylmethyl)oxy]phenyl}meth- yl)-5-methyl-1H-pyrazol-3- yl]carbamate | LC/MS Rt = 3.81 min, [MH⁺] 468, 470. |
| 22 | 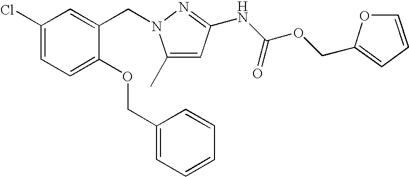 | 2-Furanylmethyl [1-({5-chloro- 2- [(phenylmethyl)oxy]phenyl}meth- yl)-5-methyl-1H-pyrazol-3- yl]carbamate | LC/MS Rt = 3.37 min, [MH⁺] 452, 454. |
| 23 | 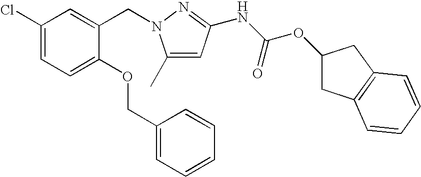 | 2,3-Dihydro-1H-inden-2-yl [1- ({5-chloro-2- [(phenylmethyl)oxy]phenyl}meth- yl)-5-methyl-1H-pyrazol-3- yl]carbamate | LC/MS Rt = 3.54 min, [MH⁺] 488, 490. |
| 24 | 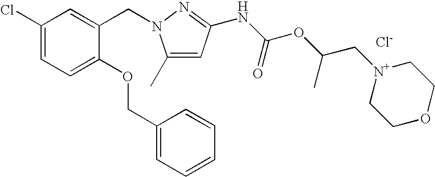 | 4-{2-[({[1-({5-Chloro-2- [(phenylmethyl)oxy]phenyl}meth- yl)-5-methyl-1H-pyrazol-3- yl]amino}carbonyl)oxy]propyl} morpholin-4-ium chloride | LC/MS Rt = 2.34 min, [MH⁺] 499, 501. |
| 25 | 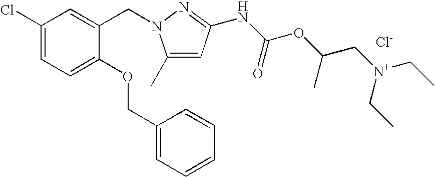 | 2-[({[1-({5-Chloro-2- [(phenylmethyl)oxy]phenyl}meth- yl)-5-methyl-1H-pyrazol-3- yl]amino}carbonyl)oxy]-N,N- diethyl-1-propanaminium chloride | LC/MS Rt = 2.38 min, [MH⁺] 485, 487. |
| 26 | 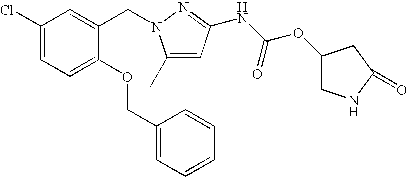 | (3R)-5-Oxo-3-pyrrolidinyl [1- ({5-chloro-2- [(phenylmethyl)oxy]phenyl}meth- yl)-5-methyl-1H-pyrazol-3- yl]carbamate | LC/MC Rt = 2.87 min, [MH⁺] 455, 457. |
| 27 | 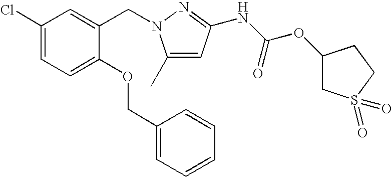 | 1,1-Dioxidotetrahydro-3- thienyl [1-({5-chloro-2- [(phenylmethyl)oxy]phenyl}meth- yl)-5-methyl-1H-pyrazol-3- yl]carbamate | LC/MS Rt = 3.17 min, [MH⁺] 490, 492. |
| 28 | 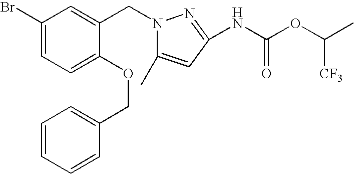 | 2,2,2-Trifluoro-1-methylethyl [1-({5-bromo-2- (phenylmethyl)oxy]phenyl}meth- yl)-5-methyl-1H-pyrazol-3- yl]carbamate | LC/MS Rt = 3.60 [MH⁺ ] 512, 514 |
| 29 | 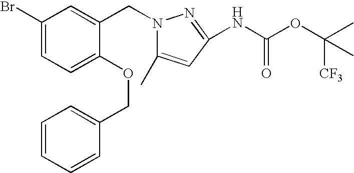 | 2,2,2-Trifluoro-1,1- dimethylethyl [1-({5-bromo-2- [(phenylmethyl)oxy]phenyl}meth- yl)-5-methyl-1H-pyrazol-3- yl]carbamate | LC/MS Rt = 3.73 min [MH⁺] 526, 528 |
| 30 | 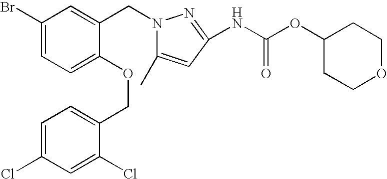 | Tetrahydro-2H-pyran-4-yl {1- [(5-bromo-2-{[(2,4- dichlorophenyl)methyl]oxy}phe- nyl)methyl]-5-methyl-1H- pyrazol-3-yl}carbamate | LC/MS Rt = 3.76 [MH⁺] 568, 570, 572 |
| 31 | 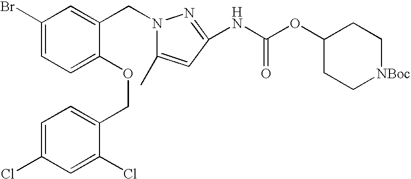 | 1,1-Dimethylethyl 4-{[({1-[(5- bromo-2-{[(2,4- dichlorophenyl)methyl]oxy}phe- nyl)methyl]-5-methyl-1H- pyrazol-3- yl}amino)carbonyl]oxy}-1- piperidinecarboxylate | LC/MS Rt = 4.09 min [MH⁺] 667, 669, 671 |
| 32 | 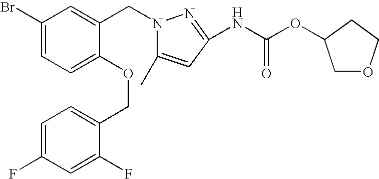 | Tetrahydro-3-furan {1-[(5- bromo-2-{[(2,4- difluorophenyl)methyl]oxy}phe- nyl)methyl]-5-methyl-1H- pyrazol-3-yl}carbamate | LC/MS Rt = 3.32 min [MH⁺] 522, 524 |
| 33 | 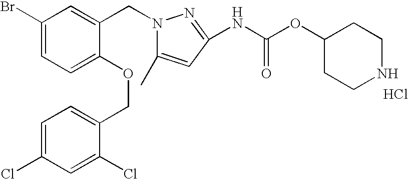 | 4-Piperidinyl {1-[(5-bromo-2- {[(2,4- dichlorophenyl)methyl]oxy}phe- nyl)methyl]-5-methyl-1H- pyraozl-3-yl}carbamate hydrochloride | |

### Example 34

### 1-Methyl-1-phenylethyl[1-({5-chloro-2-[(phenylmethyl)oxy]phenyl}methyl)-5-methyl-1H-pyrazol-3-yl]carbamate

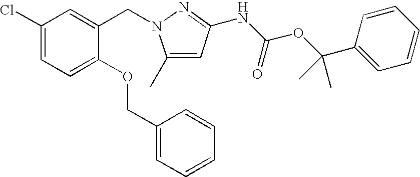

1-({5-Chloro-2-[(phenylmethyl)oxy]phenyl}methyl)-5-methyl-1H-pyrazole-3-carboxylic acid (357 mg, 1 mmol) was dissolved in toluene (4 ml) and triethylamine (240 μl 1.1 mmol) added, followed by diphenylphosphoryl azide (150 μl, 1.1 mmol). The mixture was stirred at room temperature for 2 h then heated at reflux for 1 h. After cooling, the reaction was washed with 5% ammonium chloride solution and water, dried (MgSO₄) and evaporated. The residue was dissolved in toluene (8 ml). 2 ml of this solution (assumed 0.25 mmol) was treated with 2-phenyl-2-propanol (140 μl, 1 mmol) and the solution stirred at 100° C. for 2 h. The solvent was evaporated and the residue purified by MDAP (13 mg).

¹H NMR (MeOD) δ: 1.80 (6H, s), 2.07 (3H, s), 5.12 (4H, s), 6.07 (1H, s), 6.67 (1H, d) 7.02 (1H, d), 7.21 (2H, m), 7.30-7.44 (9H, m).

### Example 35

### 4-Piperidinyl[({5-chloro-2-[(2-methylpropyl)oxy]phenyl}methyl)-5-methyl-1H-pyrazol-3-yl]carbamate hydrochloride

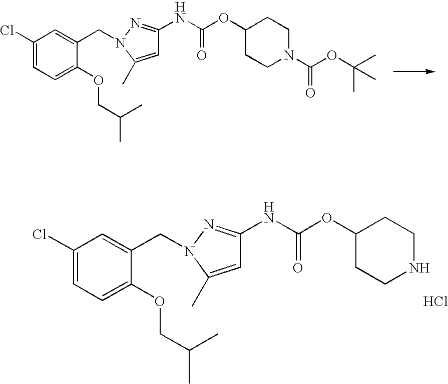

A 4M solution of HCl in dioxane (1.5 mL) was added to 1,1-dimethylethyl 4-[({[1-({5-chloro-2-[(2-methylpropyl)oxy]phenyl}methyl)-5-methyl-1H-pyrazol-3-yl]amino}carbonyl)oxy]-1-piperidinecarboxylate (0.156 g, 0.30 mmol) and stirred at room temperature for 3 hours. Evaporation of the solvent gave the title compound. LC/MS Rt=2.37 min [MH+] 421.

### 1-({5-Chloro-2-[(phenylmethyl)oxy]phenyl}methyl)-5-methyl-1,2-dihydro-3H-pyrazol-3-one

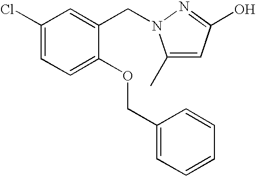

To a solution of 5-methyl-3-pyrazolidinone (3.16 g, 31.6 mmol) in methanol (30 ml) at 0° C. under argon, was added sodium methoxide (1.71 g, 31.6 mmol). The solution was stirred at 0° C. for 10 minutes, then 5-chloro-2-[(phenylmethyl)oxy]benzaldehyde (7.8 g, 31.6 mmol) was suspended in methanol (100 ml) and added to the reaction mixture. The reaction was stirred at room temperature for 1 hour. Sodium methoxide (1.71 g, 31.6 mmol) was then added to the reaction and this was heated to reflux overnight. The mixture was evaporated to a solid. Ethyl acetate and 2M hydrochloric acid were added to the residue, and the resulting yellow solid was collected by filtration and triturated with diethyl ether to give a cream powder (7.71 g). LC/MS Rt=2.94 min, [MH+] 329.

### 1-({5-Chloro-2-[(2-methylpropyl)oxy]phenyl}methyl)-5-methyl-1,2-dihydro-3H-pyrazol-3-one

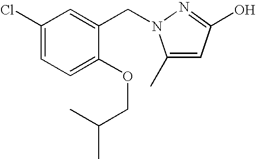

The title compound was prepared in a similar manner to 1-({5-chloro-2-[(phenylmethyl)oxy]phenyl}methyl)-5-methyl-1,2-dihydro-3H-pyrazol-3-one, from 5-methyl-3-pyrazolidinone and 5-chloro-2-[(2-methylpropyl)oxy]benzaldehyde.

LC/MS Rt=3.14 min, [MH+] 295.

### Example 36

### 1-({5-Chloro-2-[(phenylmethyl)oxy]phenyl}methyl)-5-methyl-1H-pyrazol-3-yl(1-methylethyl)carbamate

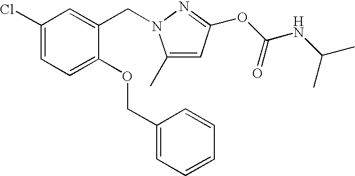

To a solution of 1-({5-chloro-2-[(phenylmethyl)oxy]phenyl}methyl)-5-methyl-1,2-dihydro-3H-pyrazol-3-one (0.05 g, 0.152 mmol) in dichloromethane (0.5 ml) and N,N-dimethylformamide (0.5 ml) was added triethylamine (0.023 ml, 0.167 mmol) followed by 2-isocyanatopropane (0.014 mg, 0.167 mmol). The mixture was stirred at room temperature under argon for 24 hours. The reaction mixture was partitioned between diethyl ether and water. The aqueous layer was re-extracted with diethyl ether, the organic layers combined and washed with water, dried (Na₂SO₄) filtered and evaporated to dryness. The residue was purified using MDAP to give the title compound (0.015 g). LC/MS Rt=3.42 min, [MH⁺] 414.

The following examples were prepared in a similar manner to 1-({5-chloro-2-[(phenylmethyl)oxy]phenyl}methyl)-5-methyl-1H-pyrazol-3-yl (1-methylethyl)carbamate, from either 1-({5-chloro-2-[(phenylmethyl)oxy]phenyl}methyl)-5-methyl-1,2-dihydro-3H-pyrazol-3-one or 1-({5-chloro-2-[(2-methylpropyl)oxy]phenyl}methyl)-5-methyl-1,2-dihydro-3H-pyrazol-3-one.

| Example | | Name | Data |
|---|---|---|---|
| 37 | 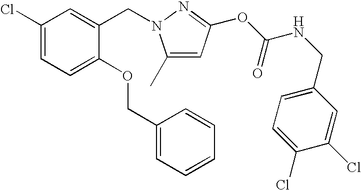 | 1-({5-Chloro-2- [(phenylmethyl)oxy]phenyl}meth- yl)-5-methyl-1H-pyrazol- 3-yl [(3,4- dichlorophenyl)methyl]carba mate | LC/MS Rt = 3.85 min, [MH⁺] 530, 532. |
| 38 | 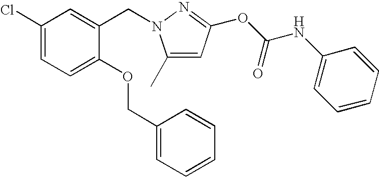 | 1-({5-Chloro-2- [(phenylmethyl)oxy]phenyl}meth- yl)-5-methyl-1H-pyrazol- 3-yl phenylcarbamate | LC/MS Rt = 3.64 min, [MH⁺] 448 |
| 39 | 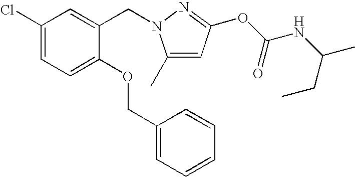 | 1-({5-Chloro-2- [(phenylmethyl)oxy]phenyl}meth- yl)-5-methyl-1H-pyrazol- 3-yl (1- methylpropyl)carbamate | LC/MS Rt = 3.57 min, [MH⁺] 428 |
| 40 | 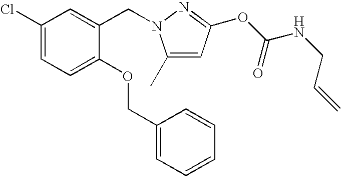 | 1-({5-Chloro-2- [(phenylmethyl)oxy]phenyl}meth- yl)-5-methyl-1H-pyraozl- 3-yl 2-propen-1-ylcarbamate | LC/MS Rt = 3.39 min, [MH⁺] 412 |
| 41 | 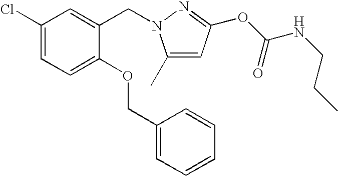 | 1-({5-Chloro-2- [(phenylmethyl)oxy]phenyl}meth- yl)-5-methyl-1H-pyrazol- 3-yl propylcarbamate | LC/MS Rt = 3.46 min, [MH⁺] 414 |
| 42 | 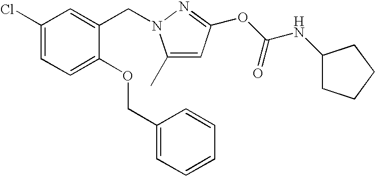 | 1-({5-Chloro-2- [(phenylmethyl)oxy]phenyl}meth- yl)-5-methyl-1H-pyrazol- 3-yl cyclopentylcarbamate | LC/MS Rt = 3.62 min, [MH⁺] 440 |
| 43 | 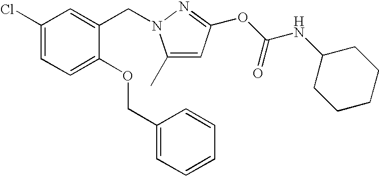 | 1-({5-Chloro-2- [(phenylmethyl)oxy]phenyl}meth- yl)-5-methyl-1H-pyrazol- 3-yl cyclohexylcarbamate | LC/MS Rt = 3.74 min, [MH⁺] 454. |
| 44 | 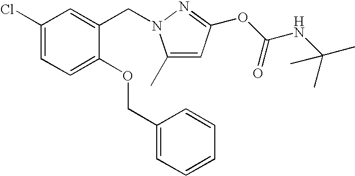 | 1-({5-Chloro-2- [(phenylmethyl)oxy]phenyl}meth- yl)-5-methyl-1H-pyrazol- 3-yl (1,1- dimethylethyl)carbamate | LC/MS Rt = 3.55 min, [MNa⁺] 450, 452. |
| 45 | 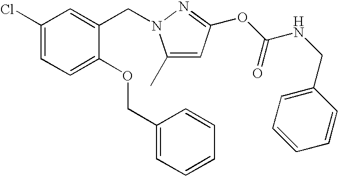 | 1-({5-Chloro-2- [(phenylmethyl)oxy]phenyl}meth- yl)-5-methyl-1H-pyrazol- 3-yl (phenylmethyl)carbamate | LC/MS Rt = 3.51 min, [MNa⁺] 484, 486. |
| 46 | 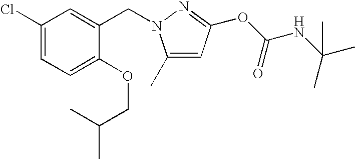 | 1-({5-Chloro-2-[(2- methylpropyl)oxy]phenyl}meth- yl)-5-methyl-1H-pyrazol-3-yl (1,1- dimethylethyl)carbamate | LC/MS Rt = 3.67 min, [MH⁺] 394 |
| 47 | 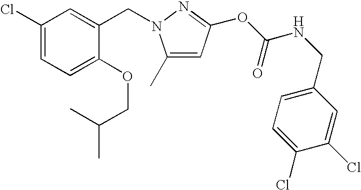 | 1-({5-Chloro-2-[(2- methylpropyl)oxy]phenyl}meth- yl)-5-methyl-1H-pyrazol-3-yl [(3,4- dichlorophenyl)methyl]carba mate | LC/MS Rt = 3.73 min, [MH⁺] 498. |
| 48 | 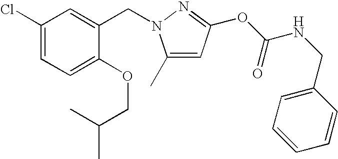 | 1-({5-Chloro-2-[(2- methylpropyl)oxy]phenyl}meth- yl)-5-methyl-1H-pyrazol-3-yl (phenylmethyl)carbamate | LC/MS Rt = 3.63 min, [MH⁺] 428 |
| 49 | 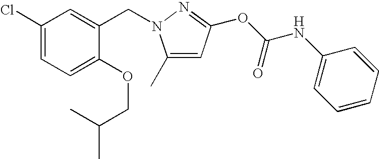 | 1-({5-Chloro-2-[(2- methylpropyl)oxy]phenyl}meth- yl)-5-methyl-1H-pyrazol-3-yl phenylcarbamate | LC/MS Rt = 3.60 min, [MH⁺] 414. |
| 50 | 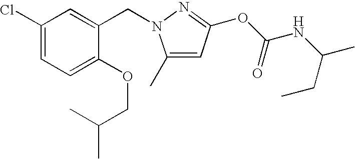 | 1-({5-Chloro-2-[(2- methylpropyl)oxy]phenyl}meth- yl)-5-methyl-1H-pyrazol-3-yl (1-methylpropyl)carbamate | LC/MS Rt = 3.62 min, [MH⁺] 394 |
| 51 | 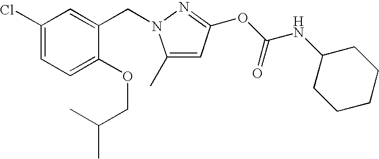 | 1-({5-Chloro-2-[(2- methylpropyl)oxy]phenyl}meth- yl)-5-methyl-1H-pyrazol-3-yl cyclohexylcarbamate | LC/MS Rt = 3.75 min, [MH⁺] 420. |
| 52 | 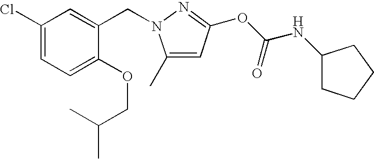 | 1-({5-Chloro-2-[(2- methylpropyl)oxy]phenyl}meth- yl)-5-methyl-1H-pyrazol-3-yl cyclopentylcarbamate | LC/MS Rt = 3.60 min, [MH⁺] 406 |
| 53 | 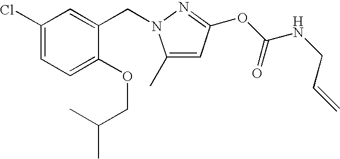 | 1-({5-Chloro-2-[(2- methylpropyl)oxy]phenyl}meth- yl)-5-methyl-1H-pyrazol-3-yl 2-propen-1-ylcarbamate | LC/MS Rt = 3.42 min, [MH⁺] 378. |
| 54 | 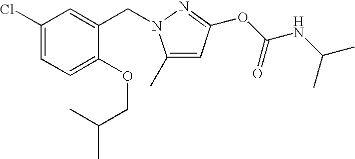 | 1-({5-Chloro-2-[(2- methylpropyl)oxy]phenyl}meth- yl)-5-methyl-1H-pyrazol-3-yl (1-methylethyl)carbamate | LC/MS Rt = 3.55 min, [MH⁺] 380 |

### Example 55

### 1-({5-Chloro-2-[(phenylmethyl)oxy]phenyl}methyl)-5-methyl-1H-pyrazol-3-yl{[4-(4-methyl-1-piperazinyl)phenyl]methyl}carbamate

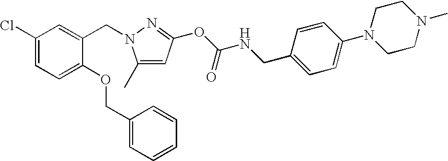

To a solution of 1-({5-chloro-2-[(phenylmethyl)oxy]phenyl}methyl)-5-methyl-1,2-dihydro-3H-pyrazol-3-one (0.10 g, 0.304 mmol) in dichloromethane (5 ml) under argon at −30° C., was added triphosgene (0.032 g, 0.107 mmol) and N,N-diisopropylethylamine (0.053 ml, 0.304 mmol) dropwise. The mixture was stirred at 0° C. for 2 hours, room temperature for 3 hours then refluxed for 2 hours. The reaction was then cooled to −15° C. and {[4-(4-methyl-1-piperazinyl)phenyl]methyl}amine (0.093 g, 0.487 mmol) was added. This was allowed to warm to room temperature and stirring continued overnight. The reaction was washed with 2M hydrochloric acid and saturated sodium bicarbonate solution, dried (Na₂SO₄), filtered and evaporated to dryness. The residue was purified by flash chromatography using 0-40% methanol in ethyl acetate, to yield the title compound as a beige foam (0.051 g). LC/MS Rt=2.51 min, [MH⁺]1560.

The following examples were prepared in a similar manner to 1-({5-chloro-2-[(phenylmethyl)oxy]phenyl}methyl)-5-methyl-1H-pyrazol-3-yl {[4-(4-methyl-1-piperazinyl)phenyl]methyl}carbamate, from 1-({5-chloro-2-[(phenylmethyl)oxy]phenyl}methyl)-5-methyl-1,2-dihydro-3H-pyrazol-3-one.

| Example | | Name | Data |
|---|---|---|---|
| 56 | 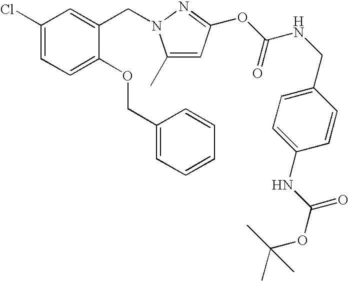 | 1-({5-Chloro-2- [(phenylmethyl)oxy]phenyl} methyl)-5-methyl-1H- pyrazol-3-yl {[4-({[(1,1- dimethylethyl)oxy]carbon yl}amino)phenyl]methyl}carba- mate | LC/MS Rt = 3.69 min, [MH⁺] 577. |
| 57 | 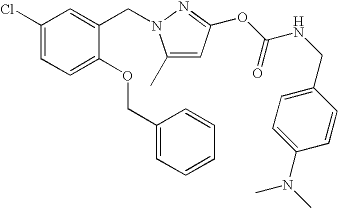 | 1-({5-Chloro-2- [(phenylmethyl)oxy]phenyl} methyl)-5-methyl-1H- pyrazol-3-yl {[4- (dimethylamino)phenyl]meth- yl}carbamate | LC/MS Rt = 3.20 min, [MH⁺] 505 |
| 58 | 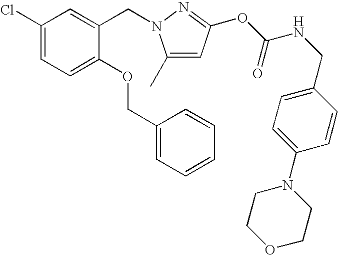 | 1-({5-Chloro-2- [(phenylmethyl)oxy]phenyl} methyl)-5-methyl-1H- pyrazol-3-yl {[4-(4- morpholinyl)phenyl]methyl} carbamate | LC/MS Rt = 3.45 min, [MH⁺] 547. |
| 59 | 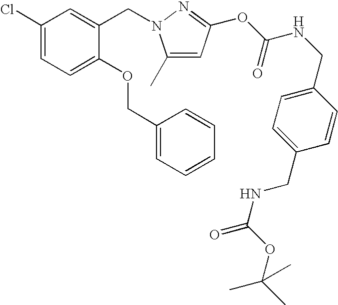 | 1-({5-Chloro-2- [(phenylmethyl)oxy]phenyl} methyl)-5-methyl-1H- pyrazol-3-yl 1,1- dimethylethyl (benzene- 1,4- dildimethanediyl)biscarb amate | LC/MS Rt = 3.65 min, [MH⁺] 591. |
| 60 | 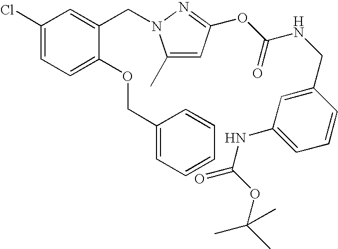 | 1-({5-Chloro-2- [(phenylmethyl)oxy]phenyl} methyl)-5-methyl-1H- pyrazol-3-yl {[3-({[(1,1- dimethylethyl)oxy]carbon yl}amino)phenyl]methyl}carba- mate | LC/MS Rt = 3.70 min, [MH⁺] 577. |
| 61 | 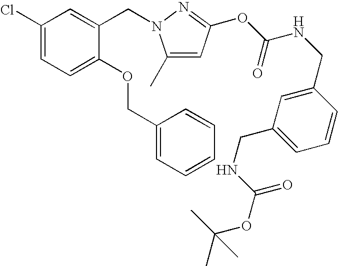 | 1-({5-Chloro-2- [(phenylmethyl)oxy]phenyl} methyl)-5-methyl-1H- pyrazol-3-yl 1,1- dimethylethyl (benzene- 1,3- diyldimethanediyl)biscarb amate | LC/MS Rt = 3.66 min, [MH⁺] 591. |

### Example 62

### 1-({5-Chloro-2-[(2-methylpropyl)oxy]phenyl}methyl)-5-methyl-1H-Pyrazol-3-yl{[4-(4-morpholinyl)phenyl}methyl]carbamate

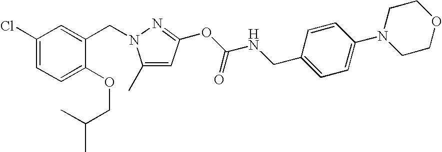

To a solution of 1-({5-chloro-2-[(2-methylpropyl)oxy]phenyl}methyl)-5-methyl-1,2-dihydro-3H-pyrazol-3-one (0.10 g, 0.339 mmol) in dichloromethane (5 ml) under argon at −30° C., was added triphosgene (0.101 g, 0.339 mmol) and N,N-diisopropylethylamine (0.053 ml, 0.305 mmol) dropwise. The mixture was stirred at 0° C. for 40 minutes. The reaction was then cooled to −5° C. and {[4-(4 morpholinyl)phenyl]methyl}amine (0.234 g, 1.22 mmol) was added. The reaction was allowed to warm to room temperature and stirring continued for 1 hour. The reaction was washed with 2M hydrochloric acid and saturated sodium bicarbonate solution, dried (Na₂SO₄), filtered and evaporated to dryness. The residue was purified using MDAP (0.018 g, 10%). LC/MS Rt=3.58 min, [MH+] 513, 515.

The following examples were prepared in a similar manner to 1-({5-chloro-2-[(2-methylpropyl)oxy]phenyl}methyl)-5-methyl-1H-pyrazol-3-yl {[4-(4-morpholinyl)phenyl]methyl}carbamate from 1-({5-chloro-2-[(2-methylpropyl)oxy]phenyl}methyl)-5-methyl-1,2-dihydro-3H-pyrazol-3-one and other appropriate starting materials.

| Example | | Name | Data |
|---|---|---|---|
| 63 | 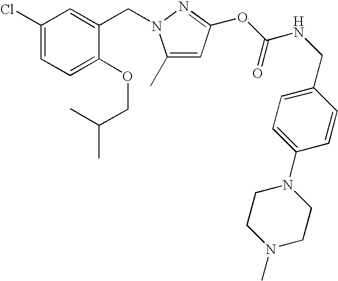 | 1-({5-Chloro-2-[(2- methylpropyl)oxy]phenyl}meth- yl)-5-methyl-1H-pyrazol- 3-yl {[4-(4-methyl-1- piperazinyl)phenyl]methyl}car- bamate | LC/MS Rt = 2.50 min, [MH⁺] 526. |
| 64 | 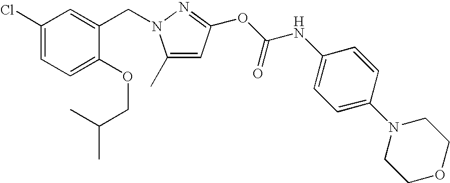 | 1-({5-Chloro-2-[(2- methylpropyl)oxy]phenyl}meth- yl)-5-methyl-1H-pyrazol- 3-yl [4-(4- morpholinyl)phenyl]carbamate | LC/MS Rt = 3.53 min, [MH⁺] 499. |

### 1-({5-Chloro-2-[(phenylmethyl)oxy]phenyl}methyl)-5-methyl-1H-pyrazol-3-amine

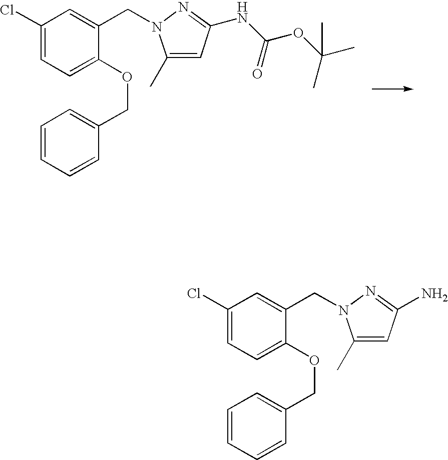

To a solution of 1,1-dimethylethyl[1-({5-chloro-2-[(phenylmethyl)oxy]phenyl}methyl)-5-methyl-1H-pyrazol-3-yl]carbamate (14.02 g, 32.78 mmol) in 1,4-dioxan (20 ml) was added 4M hydrogen chloride in 1,4-dioxan (20 ml). The reaction mixture was stirred at room temperature for 3 hours. The mixture was evaporated and the residue partitioned between ethyl acetate and 1M sodium hydroxide solution. The organic layer was washed with 1M sodium hydroxide solution, water and brine, dried (MgSO₄) and evaporated. The oil was triturated with diethyl ether:isohexane (1:3). The resulting white solid was collected by filtration (9.82 g). LC/MS Rt=3.22 min, [MH+] 328, 330.

### 1-({5-Chloro-2-[(cyclopropylmethyl)oxy]phenyl}methyl)-5-methyl-1H-pyrazol-3-amine

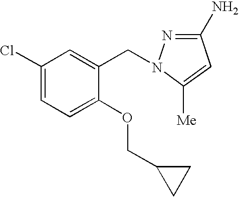

The title compound was prepared in a similar manner to 1-({5-chloro-2-[(phenylmethyl)oxy]phenyl}methyl)-5-methyl-1H-pyrazol-3-amine from the appropriate intermediates.

¹HNMR (CDCl₃) δ: 0.3-0.4 (2H, m), 0.61-0.65 (2H, m), 1.22-1.29 (1H, m), 2.170 (3H, s), 3.81 (2H, d), 5.07 (2H, s), 5.47 (1H, s), 6.78 (1H, d), 6.72 (1H, d), 7.13 (1H, dd).

### 1-({5-Chloro-2-[(2-methylpropyl)oxy]phenyl}methyl)-5-methyl-1H-pyrazol-3-amine

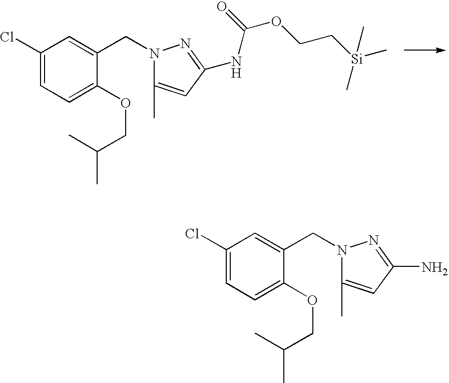

2-(Trimethylsilyl)ethyl[1-({5-chloro-2-[(2-methylpropyl)oxy]phenyl}methyl)-5-methyl-1H-pyrazol-3-yl]carbamate (3.58 g, 8.17 mmol) and 1M tetrabutylammonium fluoride in tetrahydrofuran (16.4 ml, 16.4 mmol) were stirred in tetrahydrofuran (16 ml). The mixture was stirred for 1 hour at 50° C. then evaporated to a yellow oil which was partitioned between water and diethyl ether. The organic layer was separated, dried over sodium sulphate and evaporated to yield the title compound (2.40 g).

LC/MS Rt=2.70 min [MH+] 294, 296.

### Example 65

### Methyl[1-({5-chloro-2-[(phenylmethyl)oxy]phenyl}methyl)-5-methyl-1H-pyrazol-3-yl]carbamate

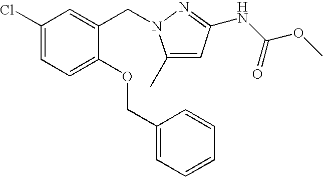

1-({5-Chloro-2-[(phenylmethyl)oxy]phenyl}methyl)-5-methyl-1H-pyrazol-3-amine (82 mg, 0.25 mmol) was dissolved in dichloromethane (2 ml) and pyridine (25 μl, 0.3 mmol) added, followed by methyl chloroformate (21 μl, 0.275 mmol). The mixture was stirred at room temperature for 2 h. Ethyl acetate was added and the solution washed with 1M hydrochloric acid, 5% sodium bicarbonate solution and brine, dried (MgSO₄) and evaporated. The residue was triturated with diethyl ether to give the title compound. (42 mg).

LC/MS Rt=3.51 min, [MH⁺] 386, 388.

The following examples were prepared in a similar manner to methyl[1-({5-chloro-2-[(phenylmethyl)oxy]phenyl}methyl)-5-methyl-1H-pyrazol-3-yl]carbamate from the appropriate intermediates.

| Example | | Name | Data |
|---|---|---|---|
| 66 | 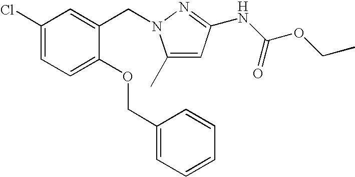 | Ethyl [1-({5-chloro-2- [(phenylmethyl)oxy]phe nyl}methyl)-5-methyl- 1H-pyrazol-3- yl]carbamate | LC/MS Rt = 3.68 min, [MH⁺] 400, 402. |
| 67 | 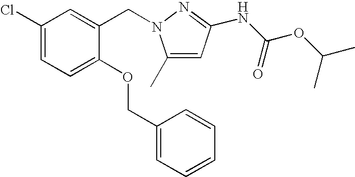 | 1-Methylethyl [1-({5- chloro-2- [(phenylmethyl)oxy]phe nyl}methyl)-5-methyl- 1H-pyrazol-3- yl]carbamate | LC/MS Rt = 3.80 min, [MH⁺] 414, 416. |
| 68 | 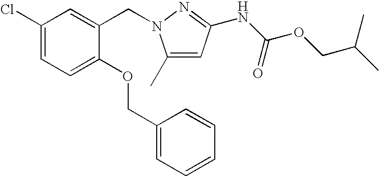 | 2-Methylpropyl [1-({5- chloro-2- [(phenylmethyl)oxy]phe nyl}methyl)-5-methyl- 1H-pyrazol-3- yl]carbamate | LC/MS Rt = 3.96 min, [MH⁺] 428, 430. |
| 69 | 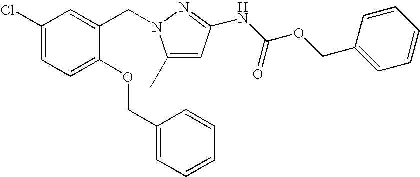 | Phenylmethyl [1-({5- chloro-2- [(phenylmethyl)oxy]phe nyl}methyl)-5-methyl- 1H-pyrazol-3- yl]carbamate | LC/MS Rt = 3.98 min, [MH⁺] 462, 464. |
| 70 | 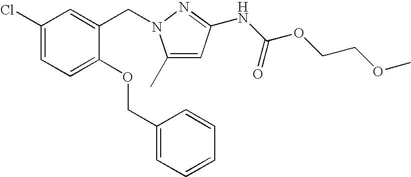 | 2-(Methyloxy)ethyl [1- ({5-chloro-2- [(phenylmethyl)oxy]phe nyl}methyl)-5-methyl- 1H-pyrazol-3- yl]carbamate | LC/MS Rt = 2.95 min, [MH⁺] 430, 432. |

### Example 71

### Phenyl[1-({5-chloro-2-[(phenylmethyl)oxy]phenyl}methyl)-5-methyl-1H-pyrazol-3-yl]carbamate

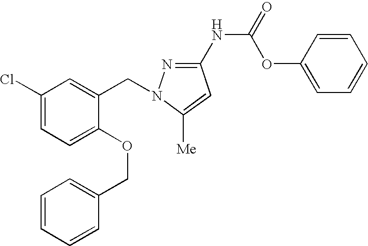

Phenyl chloroformate (43 mg, 0.275 mmol) was added to a solution of pyridine (24 mg, 0.3 mmol) and 1-({5-chloro-2-[(phenylmethyl)oxy]phenyl}methyl)-5-methyl-1H-pyrazol-3-amine (82 mg, 0.25 mmol) in dichloromethane (3 ml) and left at room temperature for 1 hour. The solution was diluted with ether, washed with saturated sodium bicarbonate, dried (magnesium sulphate), evaporated and triturated with ether/hexane to give the title compound as a white solid (71 mg). LC/MS Rt 3.90, [MH]⁺ 448.3, 450.4.

### Example 72

### 2-(Trimethylsilyl)ethyl[1-({5-chloro-2-[(phenylmethyl)oxy]phenyl}methyl)-5-methyl-1H-pyrazol-3-yl]carbamate

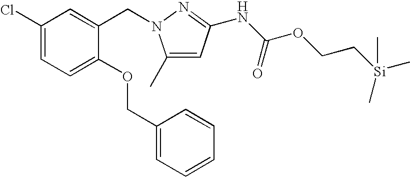

1-({5-Chloro-2-[(phenylmethyl)oxy]phenyl}methyl)-5-methyl-1H-pyrazol-3-amine (82 mg, 0.25 mmol) was dissolved in dichloromethane (2 ml) and triethylamine (60 μl, 0.43 mmol) added, followed by 1-[({[2-(trimethylsilyl)ethyl]oxy}carbonyl)oxy]-2,5-pyrrolidinedione (71 mg, 0.3 mmol). The mixture was stirred at room temperature for 76 h. The solvent was evaporated and the residue purified by flash chromatography, eluting with 3:1 isohexane:ethyl acetate. The product was triturated with isohexane and diethyl ether to give the title compound as a white powder. (61 mg). LC/MS Rt=4.28 min, [MH³⁰ ] 472, 474.

### Example 73

### 2-(Methylsulfonyl)ethyl[1-({5-chloro-2-[(phenylmethyl)oxy]phenyl}methyl)-5-methyl-1H-pyrazol-3-yl]carbamate

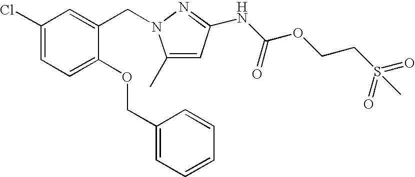

1-({5-Chloro-2-[(phenylmethyl)oxy]phenyl}methyl)-5-methyl-1H-pyrazol-3-amine (82 mg, 0.25 mmol) was dissolved in dichloromethane (2 ml) and triethylamine (60 μl, 0.43 mmol) added, followed by 2-(methylsulfonyl)ethyl 2,5-dioxo-1-pyrrolidinecarboxylate (73 mg, 0.3 mmol). The mixture was stirred at room temperature for 76 h then diluted with diethyl ether and washed with 1M hydrochloric acid, 5% sodium bicarbonate solution and water, dried (MgSO₄) and evaporated. The residue was purified by flash chromatography, eluting with 3:1 ethyl acetate:isohexane then by MDAP to give the title compound (16 mg).

LC/MS Rt=3.32 min, [MH⁺] 478, 480.

### Example 74

### Ethyl 2-[({[1-({5-chloro-2-[(phenylmethyl)oxy]phenyl}methyl)-5-methyl-1H-pyrazol-3-yl]amino}carbonyl)oxy]-2-methylpropanoate

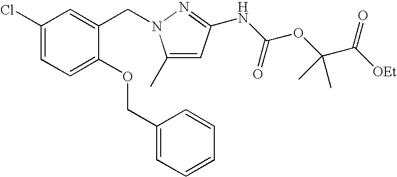

20% Phosgene in toluene solution (2 ml, 0.4 mmol) was cooled to −15° C. under argon and ethyl 2-hydroxyisobutyrate (274 μl, 2 mmol) in dichloromethane (1 ml) added, followed by dropwise addition of pyridine (182 μl, 2.25 mmol) in dichloromethane (1 ml). The mixture was stirred and allowed to warm to room temperature then stirred for 16 h. Ice-water was added and the organic layer washed with 5% sodium bicarbonate solution, dried (MgSO₄) and evaporated. The residue was dissolved in dichloromethane (1 ml) and added to a solution of 1-({5-chloro-2-[(phenylmethyl)oxy]phenyl}methyl)-5-methyl-1H-pyrazol-3-amine (82 mg, 0.25 mmol) and pyridine (50 μl, 0.6 mmol) in dichloromethane (1 ml). The reaction was stirred at room temperature for 2 h then diluted with ethyl acetate. The solution was washed with 2M hydrochloric acid, 5% sodium bicarbonate and water, dried (MgSO₄) and evaporated. The residue was purified by flash chromatography, eluting with 10-20% ethyl acetate/40-60° C. petroleum spirit to give the title compound as a white foam (21 mg). ¹H NMR (CDCl₃) δ: 1.27 (3H, t), 1.60 (6H, s), 2.11 (3H, s), 4.22 (2H, q), 5.07 (2H, s), 5.12 (2H, s), 6.31 (1H, bs), 6.66 (1H, d) 6.85 (1H, d), 7.10 (1H, bs), 7.16 (1H, dd), 7.35-7.42 (5H, m).

### Example 75

### 1,1-Dimethylethyl[({5-chloro-2-[(phenylmethyl)oxy]phenyl}methyl)-5-methyl-1H-pyrazol-3-yl]methylcarbamate

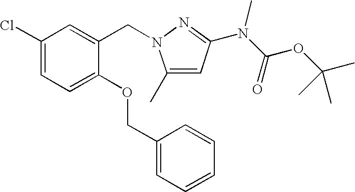

1,1-Dimethylethyl[1-({5-chloro-2-[(phenylmethyl)oxy]phenyl}methyl)-5-methyl-1H-pyrazol-3-yl]carbamate (104 mg, 0.25 mmol) was dissolved in dimethylformamide under argon with stirring. 60% sodium hydride dispersion in mineral oil (12 mg, 0.3 mmol) was added and the mixture stirred for 15 minutes. Methyl iodide (19 μl, 0.3 mmol) was added and the mixture stirred at room temperature for 2 h. Diethyl ether was added and the solution washed with water and brine (×3), dried (MgSO₄) and evaporated to a gum which solidified on trituration with diethyl ether. Hexane was added and the cream solid filtered and dried in vacuo (57 mg). LC/MS Rt=3.98 min, [MH⁺] 442, 444.

### Example 76

### N-[1-({5-Chloro-2-[(2-methylpropyl)oxy]phenyl}methyl)-5-methyl-1H-pyrazol-3-yl]-3-methylbutanamide

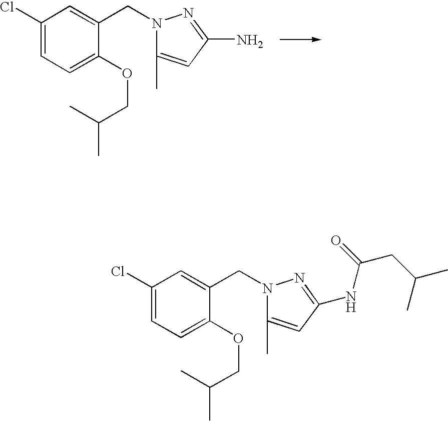

1-({5-Chloro-2-[(2-methylpropyl)oxy]phenyl}methyl)-5-methyl-1H-pyrazol-3-amine (0.1 g, 0.34 mmol) was dissolved in dichloromethane (1 ml) and stirred under argon. The solution was treated with triethylamine (0.14 ml, 1 mmol) and 3-methylbutanoyl chloride (0.064 ml, 0.51 mmol). Dichloromethane (3 ml) was added and the reaction stirred for 3 hours and then evaporated to an oil which was flash chromatographed with ethyl acetate/hexane (2/1) to yield the title compound (0.046 g). LC/MS Rt=2.14 min [MH+] 378, 380.

The following examples were prepared in a similar manner to N-[1-({5-chloro-2-[(2-methylpropyl)oxy]phenyl}methyl)-5-methyl-1H-pyrazol-3-yl]-3-methylbutanamide from the appropriate intermediates.

| Example | | Name | Data |
|---|---|---|---|
| 77 | 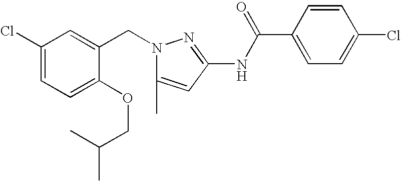 | 4-Chloro-N-[1-({5-chloro- 2-[(2- methylpropyl)oxy]phenyl} methyl)-5-methyl-1H- pyrazol-3-yl]benzamide | LC/MS Rt = 3.63 min [MH+] 432, 434 |
| 78 | 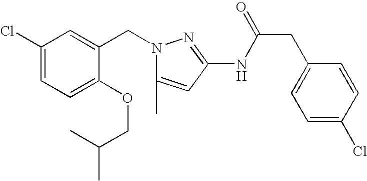 | N-[1-({5-Chloro-2-[(2- methylpropyl)oxy]phenyl} methyl)-5-methyl-1H- pyrazol-3-yl]-2-(4- chlorophenyl)acetamide | LC/MS Rt = 3.56 min [MH+] 446, 448 |
| 79 | 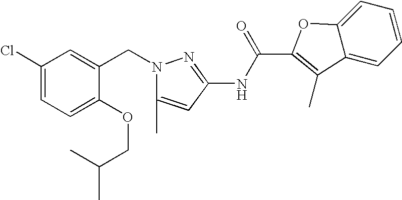 | N-[1-({5-Chloro-2-[(2- methylpropyl)oxy]phenyl} methyl)-5-methyl-1H- pyrazol-3-yl]-3-methyl-1- benzofuran-2- carboxamide | LC/MS Rt = 3.90 min [MH+] not observed |
| 80 | 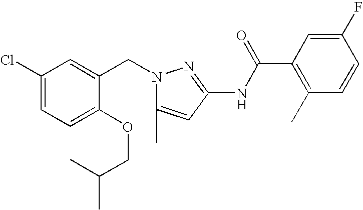 | N-[1-({5-Chloro-2-[(2- methylpropyl)oxy]phenyl} methyl)-5-methyl-1H- pyrazol-3-yl]-5-fluoro-2- methylbenzamide | LC/MS Rt = 3.54 min [MH+] 430, 432 |
| 81 | 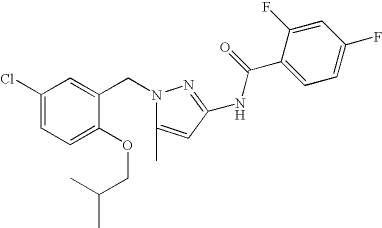 | N-[1-({5-Chloro-2-[(2- methylpropyl)oxy]phenyl} methyl)-5-methyl-1H- pyrazol-3-yl]-2,4- difluorobenzamide | LC/MS Rt = 3.57 min [MH+] 434, 436 |
| 82 | 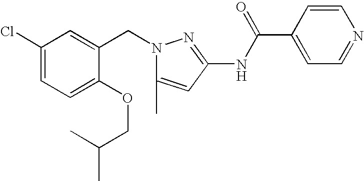 | N-[1-({5-Chloro-2-[(2- methylpropyl)oxy]phenyl} methyl)-5-methyl-1H- pyrazol-3-yl]-4- pyridinecarboxamide | LC/MS Rt = 3.57 min [MH+] 399, 401 |
| 83 | 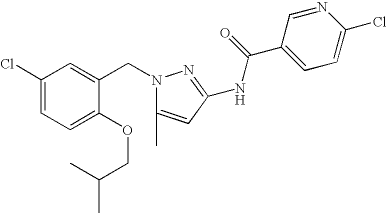 | 5-Chloro-N-[1-({5-chloro- 2-[(2- methylpropyl)oxy]phenyl} methyl)-5-methyl-1H- pyrazol-3-yl]-3- pyridinecarboxamide | LC/MS Rt = 3.46 min [MH+] 433, 435 |
| 84 | 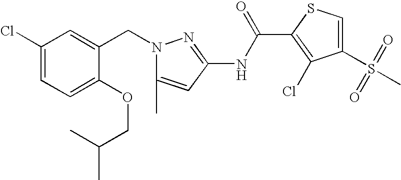 | 3-Chloro-N-[1-({5-chloro- 2-[(2- methylpropyl)oxy]phenyl} methyl)-5-methyl-1H- pyrazol-3-yl]-4- (methylsulfonyl)-2- thiophenecarboxamide | LC/MS Rt = 3.41 min [MH+] 516, 518 |
| 85 | 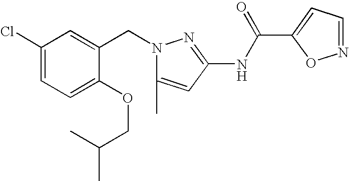 | N-[1-({5-Chloro-2-[(2- methylpropyl)oxy]phenyl} methyl)-5-methyl-1H- pyrazol-3-yl]-5- isoxazolecarboxamide | LC/MS Rt = 3.29 min [MH+] 389, 391 |
| 86 | 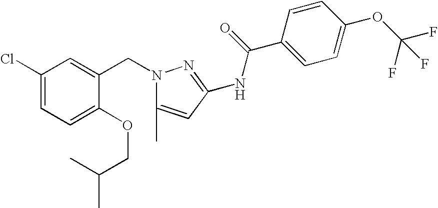 | N-[1-({5-Chloro-2-[(2- methylpropyl)oxy]phenyl} methyl)-5-methyl-1H- pyrazol-3-yl]-4- [(trifluoromethyl)oxy]benz amide | LC/MS Rt = 3.65 min [MH+] 482, 484 |
| 87 | 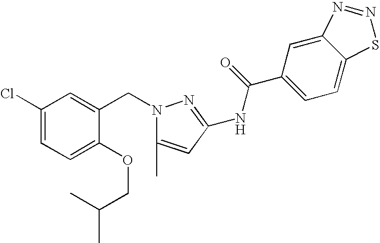 | N-[1-({5-Chloro-2-[(2- methylpropyl)oxy]phenyl} methyl)-5-methyl-1H- pyrazol-3-yl]-1,2,3- benzothiadiazole-5- carboxamide | LC/MS Rt = 3.51 min [MH+] 456, 458 |
| 88 | 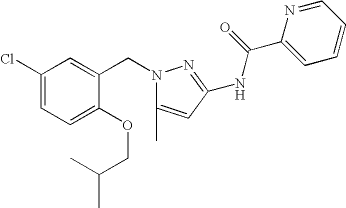 | N-[1-({5-Chloro-2-[(2- methylpropyl)oxy]phenyl} methyl)-5-methyl-1H- pyrazol-3-yl]-2- pyridinecarboxamide | LC/MS Rt = 3.59 min [MH+] 399, 401 |
| 89 | 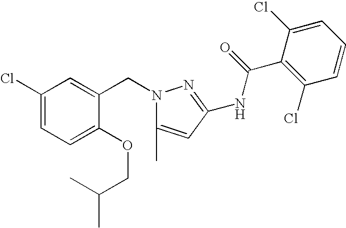 | 2,6-Dichloro-N-[1-({5- chloro-2-[(2- methylpropyl)oxy]phenyl} methyl)-5-methyl-1H- pyrazol-3-yl]benzamide | LC/MS Rt = 3.49 min [MH+] 466, 468, 470 |
| 90 | 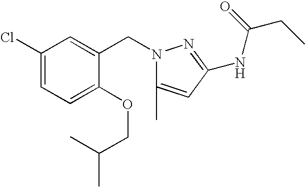 | N-[1-({5-Chloro-2-[(2- methylpropyl)oxy]phenyl} methyl)-5-methyl-1H- pyrazol-3-yl]propanamide | LC/MS Rt = 3.11 min [MH+] 350, 352 |
| 91 | 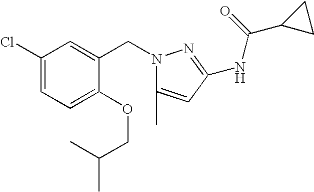 | N-[1-({5-chloro-2-[(2- methylpropyl)oxy]phenyl} methyl)-5-methyl-1H- pyrazol-3- yl]cyclopropanecarboxamide | LC/MS Rt = 3.15 min [MH+] 362, 364 |
| 92 | 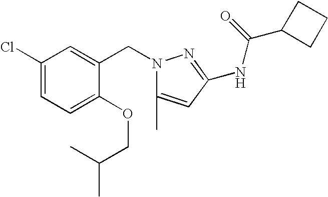 | N-[1-({5-Chloro-2-[(2- methylpropyl)oxy]phenyl} methyl)-5-methyl-1H- pyrazol-3- yl]cyclobutanecarboxamide | LC/MS Rt = 3.25 min [MH+] 376, 378 |
| 93 | 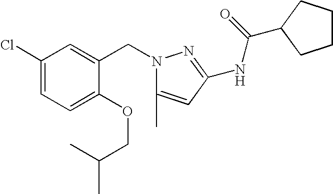 | N-[1-({5-Chloro-2-[(2- methylpropyl)oxy]phenyl} methyl)-5-methyl-1H- pyrazol-3- yl]cyclopentanecarboxamide | LC/MS Rt = 3.34 min [MH+] 390, 392 |
| 94 | 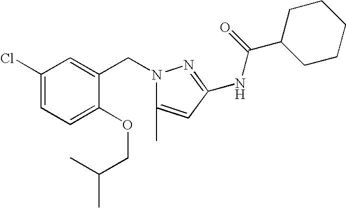 | N-[1-({5-Chloro-2-[(2- methylpropyl)oxy]phenyl} methyl)-5-methyl-1H- pyrazol-3- yl]cyclohexanecarboxamide | LC/MS Rt = 3.42 min [MH+] 404, 406 |

### Example 95

### N-[1-({5-Chloro-2-[(2-methylpropyl)oxy]phenyl}methyl)-5-methyl-1H-pyrazol-3-yl]-2,2,2-trifluoroacetamide

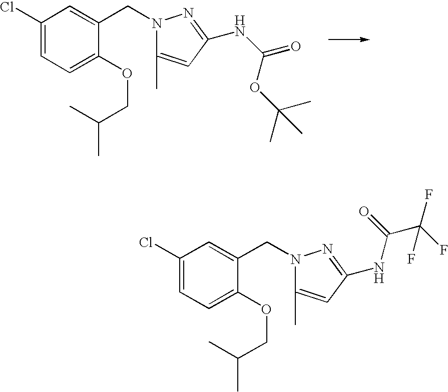

1,1-Dimethylethyl[1-({5-chloro-2-[(2-methylpropyl)oxy]phenyl}methyl)-5-methyl-1H-pyrazol-3-yl]carbamate (17.81 g, 45 mmol) and trifluoroacetic acid (25 ml) were stirred in dichloromethane (50 ml) for 7 hours and evaporated to a brown oil which was flash chromatographed with ethyl acetate/hexane (1/2) to yield the title compound (8.982 g).

LC/MS Rt=3.32 min [MH+] 388, 390

### Example 96

### N-[1-({5-Chloro-2-[(2-methylpropyl)oxy]phenyl}methyl)-5-methyl-1H-pyrazol-3-yl]-3-quinolinecarboxamide

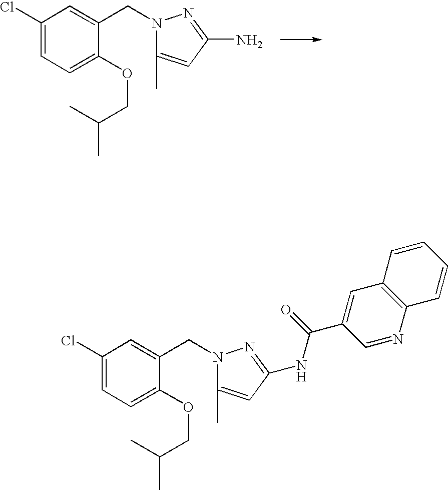

3-Quinolinecarboxylic acid (0.071 g, 0.41 mmol), N-(3-dimethylaminopropyl)-N′-ethylcarbodiimide hydrochloride (0.078 g, 0.41 mmol) and 1-hydroxy-7-azabenzotriazole (0.55 g, 0.41 mmol) were stirred in dichloromethane (5 ml) for 1 hour. 1-({5-Chloro-2-[(2-methylpropyl)oxy]phenyl}methyl)-5-methyl-1H-pyrazol-3-amine (0.1 g, 0.34 mmol) was added and the mixture stirred for 1 hour, diluted with ethyl acetate and acidified with 2M hydrochloric acid. The organic layer was separated, evaporated to a yellow solid and flash chromatographed on reverse phase silica gel eluting with 0-100% acetonitrile in water to give the title compound (0.045 g). LC/MS Rt=3.33 min [MH+] 449, 451.

### Example 97

### 1,1-Dimethylethyl 7-({[1-({5-chloro-2-[(2-methylpropyl)oxy]phenyl}methyl)-5-methyl-1H-pyrazol-3-yl]amino}carbonyl)-1,2,4,5-tetrahydro-3H-3-benzazepine-3-carboxylate

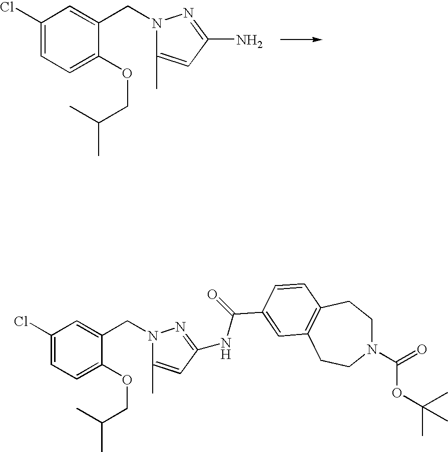

The title compound was prepared in a similar manner to N-[1-({5-chloro-2-[(2-methylpropyl)oxy]phenyl}methyl)-5-methyl-1H-pyrazol-3-yl]-3-quinolinecarboxamide but using 3-{[(1,1-dimethylethyl)oxy]carbonyl}-2,3,4,5-tetrahydro-1H-3-benzazepine-7-carboxylic acid instead of 3-quinolinecarboxylic acid. LC/MS Rt=4.05 min [MH+] 567, 569.

### Example 98

### N-[1-({5-Chloro-2-[(2-methylpropyl)oxy]phenyl}methyl)-5-methyl-1H-pyrazol-3-yl]-2,3,4,5-tetrahydro-1H-3-benzazepine-7-carboxamide hydrochloride

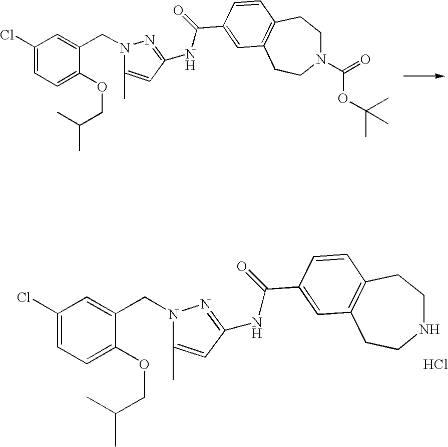

1,1-Dimethylethyl 7-({[1-({5-chloro-2-[(2-methylpropyl)oxy]phenyl}methyl)-5-methyl-1H-pyrazol-3-yl]amino}carbonyl)-1,2,4,5-tetrahydro-3H-3-benzazepine-3-carboxylate (1.384 g, 2.44 mmol) was stirred in 4M hydrogen chloride in dioxane (25 ml) for 45mins. The solution was evaporated to yield the title compound (1.367 g, assumed 100%).

LC/MS Rt=2.35 min [MH+] 467, 469

### Example 99

### N-[1-({5-Chloro-2-[(2-methylpropyl)oxy]phenyl}methyl)-5-methyl-1H-pyrazol-3-yl]-3-ethyl-2,3,4,5-tetrahydro-1H-3-benzazepine-7-carboxamide hydrochloride

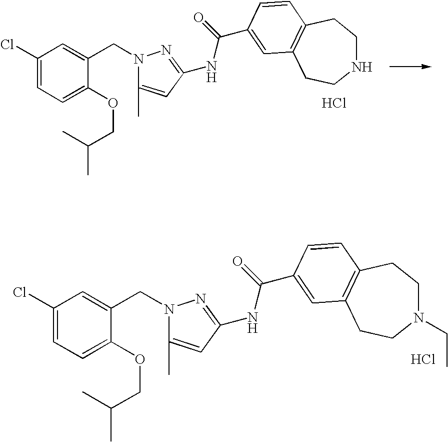

N-[1-({5-Chloro-2-[(2-methylpropyl)oxy]phenyl}methyl)-5-methyl-1H-pyrazol-3-yl]-2,3,4,5-tetrahydro-1H-3-benzazepine-7-carboxamide hydrochloride (0.058 g, 0.115 mmol), acetic acid (0.007 ml, 0.115 mmol), acetaldehyde (0.1 ml, 1.78 mmol) and sodium triacetoxyborohydride (0.073 g, 0.345 mmol) were stirred in dichloromethane (3ml) for 1 hour. The mixture was evaporated to a solid which was redissolved in methanol, purified on an SCX cartridge, evaporated to a solid and purified by MDAP. The product was treated with 1M hydrogen chloride in diethyl ether (1 ml) to yield the title compound (0.028 g). LC/MS Rt=2.48 min [MH+] 495, 497.

The following were prepared in a similar manner to N-[1-({5-chloro-2-[(2-methylpropyl)oxy]phenyl}methyl)-5-methyl-1H-pyrazol-3-yl]-3-ethyl-2,3,4,5-tetrahydro-1H-3-benzazepine-7-carboxamide hydrochloride using the relevant reagents

| Example | | Name | Data |
|---|---|---|---|
| 100 | 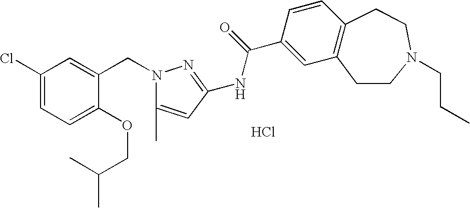 | N-[1-({5-Chloro-2-[(2- methylpropyl)oxy]phenyl}methyl)- 5-methyl-1H-pyrazol-3-yl]-3- propyl-2,3,4,5-tetrahydro-1H-3- benzazepine-7-carboxamide hydrochloride | LC/MS [MH+] 509, 511, Rt = 2.50 min |
| 101 | 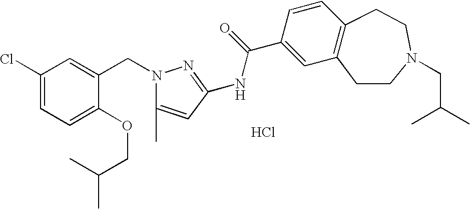 | N-[1-({5-Chloro-2-[(2- methylpropyl)oxy]phenyl}methyl)- 5-methyl-1H-pyrazol-3-yl]-3-(2- methylpropyl)-2,3,4,5-tetrahydro- 1H-3-benzazepine-7- carboxamide hydrochloride | LC/MS [MH+] 523, 525 Rt = 2.69 min |
| 102 | 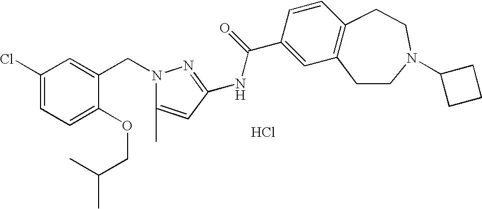 | N-[1-({5-Chloro-2-[(2- methylpropyl)oxy]phenyl}methyl)- 5-methyl-1H-pyrazol-3-yl]-3- cyclobutyl-2,3,4,5-tetrahydro-1H- 3-benzazepine-7-carboxamide hydrochloride | LC/MS [MH+] 521, 523, Rt = 2.57 min |

### Example 103

### N-[1-({5-Chloro-2-[(2-methylpropyl)oxy]phenyl}methyl)-5-methyl-1H-pyrazol-3-yl]acetamide

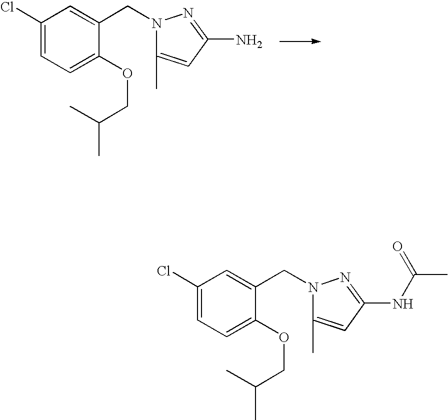

1-({5-Chloro-2-[(2-methylpropyl)oxy]phenyl}methyl)-5-methyl-1H-pyrazol-3-amine (0.1 g, 0.34 mmol) was stirred in glacial acetic acid (5 ml) and 33% hydrogen bromide in acetic acid (5 ml) added. The mixture was stirred for 1 hour and then evaporated to a brown gum which was purified by MDAP. The product was recrystallised from isopropanol (0.005 g). LC/MS Rt=3.11 min [MH+] 336, 338.

### Example 104

### N-[1-({5-Chloro-2-[(phenylmethyl)oxy]phenyl}methyl)-5-methyl-1H-pyrazol-3-yl]acetamide

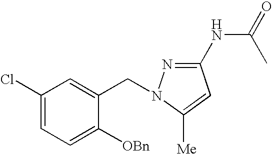

Acetyl chloride (28 mg, 0.36 mmol) was added to a solution of 1-({5-chloro-2-[(phenylmethyl)oxy]phenyl}methyl)-5-methyl-1H-pyrazol-3-amine (109 mg, 0.33 mmol) and triethylamine (40 mg, 0.4 mmol) in dichloromethane (3 ml) and left at room temperature for 1 hour. The resulting mixture was diluted with ether/water and the organic phase washed with 2M hydrochloric acid and saturated sodium bicarbonate then dried (magnesium sulphate), evaporated and the residue triturated with ether to give the title compound as a white solid (89 mg). LC/MS Rt 3.27, [MH]⁺ 370.6, 372.6.

The following compounds were prepared by a similar procedure from the appropriate starting materials:

| Example | | Name | Data |
|---|---|---|---|
| 105 | 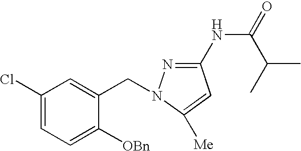 | N-[1-({5-Chloro-2- [(phenylmethyl)oxy]phenyl} methyl)-5-methyl-1H- pyrazol-3-yl]-2- methylpropanamide | LC/MS Rt = 3.55 [MH]⁺ 398.6, 400.5 |
| 106 | 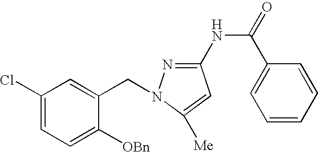 | N-[1-({5-Chloro-2- [(phenylmethyl)oxy]phenyl} methyl)-5-methyl-1H- pyrazol-3-yl]benzamide | LC/MS Rt = 3.80 [MH]⁺ 432.6, 434.6 |
| 107 | 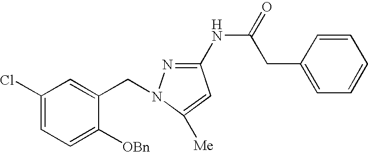 | N-[1-({5-Chloro-2- [(phenylmethyl)oxy]phenyl} methyl)-5-methyl-1H- pyrazol-3-yl]-2- phenylacetamide | LCMS Rt = 3.76 [MH]⁺ 446.6, 448.6 |
| 108 | 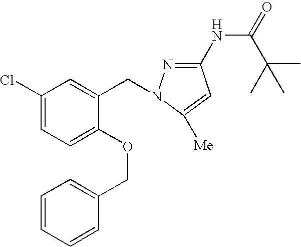 | N-[1-({5-Chloro-2- [(phenylmethyl)oxy]phenyl} methyl)-5-methyl-1H- pyrazol-3-yl]-2,2- dimethylpropanamide | LC/MS Rt = 3.74 [MH]⁺ 412.5, 414.4 |
| 109 | 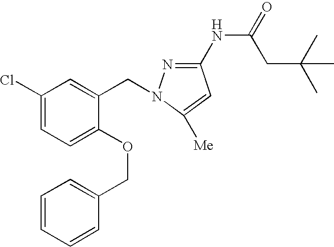 | N-[1-({5-Chloro-2- [(phenylmethyl)oxy]phenyl} methyl)-5-methyl-1H- pyrazol-3-yl]-3,3- dimethylbutanamide | LC/MS Rt = 3.83 [MH]⁺ 426.4, 428.5 |
| 110 | 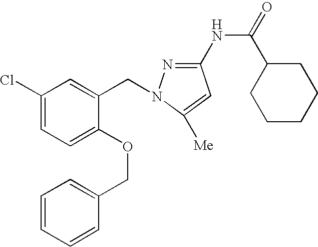 | N-[1-({5-Chloro-2- [(phenylmethyl)oxy]phenyl} methyl)-5-methyl-1H- pyrazol-3- yl]cyclohexanecarboxamide | LC/MS Rt = 3.88 [MH]⁺ 438.4, 440.4 |
| 111 | 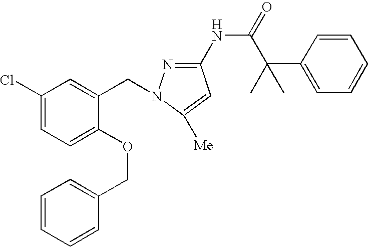 | N-[1-({5-Chloro-2- [(phenylmethyl)oxy]phenyl} methyl)-5-methyl-1H- pyrazol-3-yl]-2-methyl-2- phenylpropanamide | LC/MS Rt = 4.0 [MH]⁺ 474.4, 476.5 |
| 112 | 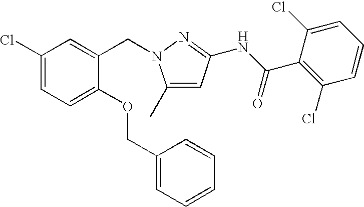 | 2,6-Dichloro-N-[1-({5- chloro-2- [(phenylmethyl)oxy]phenyl} methyl)-5-methyl-1H- pyrazol-3-yl]benzamide | LC/MS Rt = 3.92 min, [MH⁺] 500, 504. |

### N-{1-[(5-Chloro-2-hydroxyphenyl)methyl]-5-methyl-1H-pyrazol-3-yl}-2-phenylacetamide

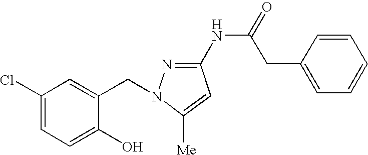

48% Hydrogen bromide in acetic acid (30 ml) was added to a solution of N-[1-({5-chloro-2-[(phenylmethyl)oxy]phenyl}methyl)-5-methyl-1H-pyrazol-3-yl]-2-phenylacetamide in acetic acid (10 ml) and left at room temperature for 2 hours. The resulting solution was diluted with ether/water and carefully basified with potassium carbonate. The organic phase was dried (magnesium sulphate), evaporated and purified by flash chromatography eluting with ethyl acetate/hexane (1:1). The product was dissolved in ethanol (20 ml) and 2M sodium hydroxide (5 ml), left at room temperature for 1 hour and evaporated to dryness. The residue was dissolved in ether/water, acidified with 2M hydrochloric acid and the organic phase dried (magnesium sulphate), evaporated and triturated with ether/hexane (1:3) to give the title compound as white solid (1.12 g). LC/MS Rt 3.20, [MH]⁺ 356.4, 358.4.

### N-{1-[(5-Chloro-2-hydroxyphenyl)methyl]-5-methyl-1H-pyrazol-3-yl}-3,3-dimethylbutanamide

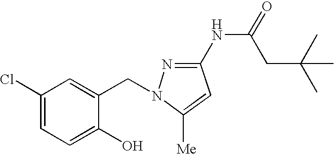

The title compound was prepared by the same method as for N-{1-[(5-chloro-2-hydroxyphenyl)methyl]-5-methyl-1H-pyrazol-3-yl}-2-phenylacetamide but using N-[1-({5-chloro-2-[(phenylmethyl)oxy]phenyl}methyl)-5-methyl-1H-pyrazol-3-yl]-3,3-dimethylbutanamide instead of N-[1-({5-chloro-2-[(phenylmethyl)oxy]phenyl}methyl)-5-methyl-1H-pyrazol-3-yl]-2-phenylacetamide.

### N-{1-[(5-Chloro-2-hydroxyphenyl)methyl]-5-methyl-1H-pyrazol-3-yl}-2-(2-pyridinyl)acetamide

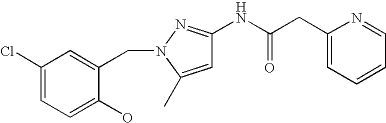

The title compound was prepared by the same method as for N-{1-[(5-chloro-2-hydroxyphenyl)methyl]-5-methyl-1H-pyrazol-3-yl}-2-phenylacetamide but using N-[1-({5-chloro-2-[(phenyl methyl)oxy]phenyl}methyl)-5-methyl-1H-pyrazol-3-yl]-2-(2-pyridinyl)acetamide instead of N-[1-({5-chloro-2-[(phenylmethyl)oxy]phenyl}methyl)-5-methyl-1H-pyrazol-3-yl]-2-phenylacetamide. LC/MS: Rt=2.66 min. [MH⁺] 357.

### Example 113

### N-[1-({5-Chloro-2-[(2-methylpropyl)oxy]phenyl}methyl)-5-methyl-1H-pyrazol-3-yl]-2-phenylacetamide

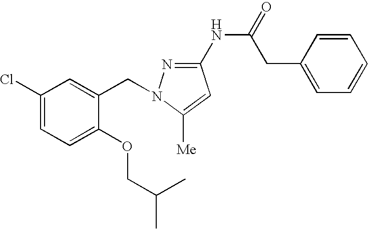

A mixture of N-{1-[(5-chloro-2-hydroxyphenyl)methyl]-5-methyl-1H-pyrazol-3-yl}-2-phenylacetamide (88 mg, 0.25 mmol), potassium carbonate (138 mg, 1 mmol) and isobutyl bromide (69 mg, 0.5 mmol) in DMF (3 ml) was stirred and heated at 70° C. for 16 hours. The mixture was cooled, diluted with ether/water and the organic phase washed with water (×3), dried (magnesium sulphate), evaporated, triturated with ether/hexane (1:2) and filtered off to give the title compound as a white solid (64 mg).

LC/MS Rt 3.85, [MH]⁺ 412.5, 414.5

The following examples were prepared in a similar manner to N-[1-({5-chloro-2-[(2-methylpropyl)oxy]phenyl}methyl)-5-methyl-1H-pyrazol-3-yl]-2-phenylacetamide from the appropriate intermediates.

| Example | | Name | Data |
|---|---|---|---|
| 114 | 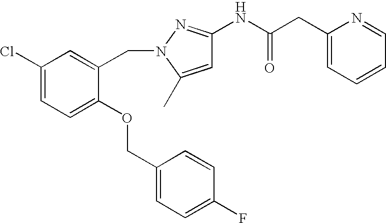 | N-{1-[(5-Chloro-2-{[(4- fluorophenyl)methyl]oxy}phen yl)methyl]-5-methyl-1H- pyrazol-3-yl}-2-(2- pyridinyl)acetamide | LC/MS: Rt = 3.43 min. [MH⁺] 465 |
| 115 | 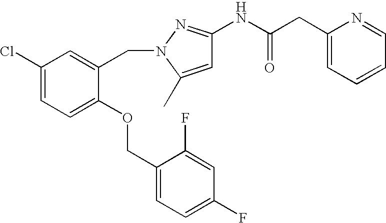 | N-{1-[(5-Chloro-2-{[(2,4- difluorophenyl)methyl]oxy}phe- nyl)methyl]-5-methyl-1H- pyrazol-3-yl}-2-(2- pyridinyl)acetamide | LC/MS: Rt = 3.46 min. [MH⁺] 483. |
| 116 | 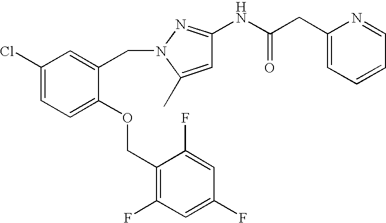 | N-{1-[(5-Chloro-2-{[(2,4,6- trifluorophenyl)methyl]oxy}phe- nyl)methyl]-5-methyl-1H- pyrazol-3-yl}-2-(2- pyridinyl)acetamide | LC/MS: Rt = 3.45 min. [MH⁺] 501. |
| 117 | 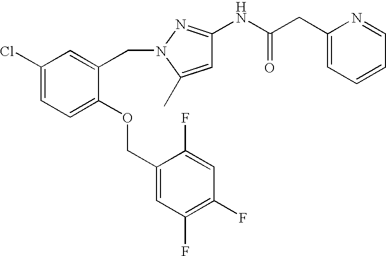 | N-{1-[(5-Chloro-2-{[(2,4,5- trifluorophenyl)methyl]oxy}phe- nyl)methyl]-5-methyl-1H- pyrazol-3-yl}-2-(2- pyridinyl)acetamide | LC/MS: Rt = 3.44 min. [MH⁺] 501. |
| 118 | 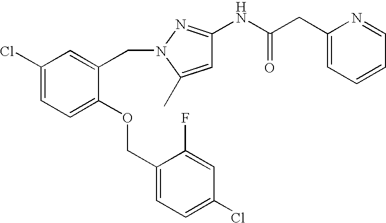 | N-{1-[(5-Chloro-2-{[(4-chloro- 2-fluorophenyl)methyl]oxy}phen yl)methyl]-5-methyl-1H- pyrazol-3-yl}-2-(2- pyridinyl)acetamide | LC/MS: Rt = 3.63 min. [MH⁺] 499. |
| 119 | 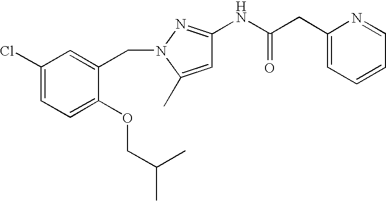 | N-[1-({5-Chloro-2-[(2- methylpropyl)oxy]phenyl}meth- yl)-5-methyl-1H-pyrazol-3- yl]-2-(2-pyridinyl)acetamide | LC/MS: Rt = 3.45 min. [MH⁺] 413. |

### Example 120

### N-{1-[(5-Chloro-2-{[(2-fluorophenyl)methyl]oxy}phenyl)methyl]-5-methyl-1H-pyrazol-3-yl}-2-phenylacetamide

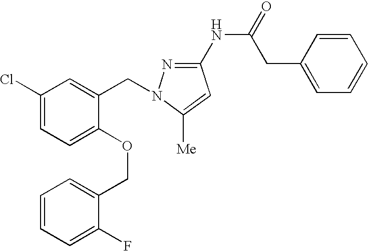

A mixture of N-{1-[(5-chloro-2-hydroxyphenyl)methyl]-5-methyl-1H-pyrazol-3-yl}-2-phenylacetamide (81 mg, 0.23 mmol), potassium carbonate (138 mg, 1 mmol) and 2-fluorobenzyl bromide (57 mg, 0.3 mmol) in acetone (5 ml) was stirred and refluxed for 4 hours. The mixture was cooled, filtered, evaporated and triturated with ether to give the title compound as white solid (69 mg). LC/MS Rt 3.80, [MH]⁺ 464.4, 466.4.

The following compounds were prepared by a similar procedure to that described in Example 120 using the appropriate intermediates:

| Example | Structure | Name | Data |
|---|---|---|---|
| 121 | 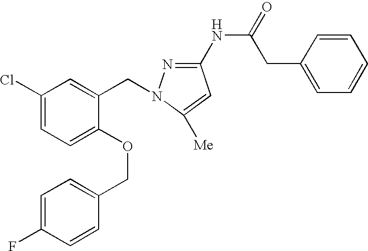 | N-{1-[(5-Chloro-2-{[(4- fluorophenyl)methyl]oxy}phe nyl)methyl]-5-methyl-1H- pyrazol-3-yl}-2- phenylacetamide | LC/MS Rt = 3.79 [MH]⁺ 464.4, 466.4 |
| 122 | 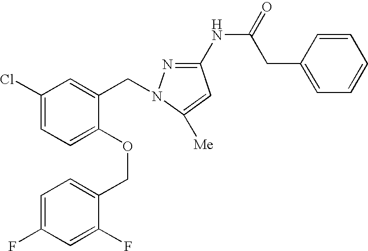 | N-{1-[(5-Chloro-2-{[(2,4- difluorophenyl)methyl]oxy}phe- nyl)methyl]-5-methyl-1H- pyrazol-3-yl}-2- phenylacetamide | LC/MS Rt = 3.83 [MH]⁺ 482.4, 484.4 |
| 123 | 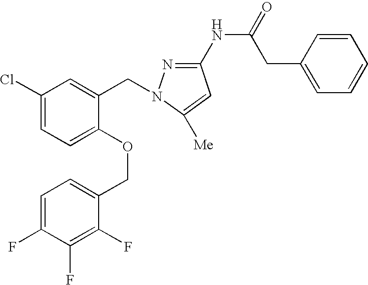 | N-{1-[(5-Chloro-2-{[(2,3,4- trifluorophenyl)methyl]oxy}phe- nyl)methyl]-5-methyl-1H- pyrazol-3-yl}-2- phenylacetamide | LC/MS Rt = 3.88 [MH]⁺ 500.4, 502.4 |
| 124 | 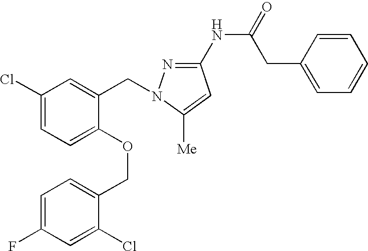 | N-{1-[(5-Chloro-2-{[(2-chloro- 4- fluorophenyl)methyl]oxy}phe nyl)methyl]-5-methyl-1H- pyrazol-3-yl}-2- phenylacetamide | LC/MS Rt = 3.98 [MH]⁺ 498.4, 501.4 |
| 125 | 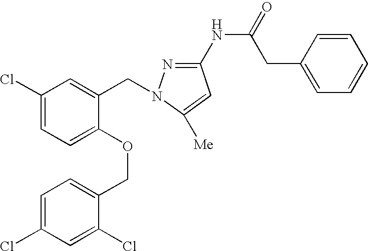 | N-{1-[(5-Chloro-2-{[(2,4- dichlorophenyl)methyl]oxy}phe- nyl)methyl]-5-methyl-1H- pyrazol-3-yl}-2- phenylacetamide | LC/MS Rt = 4.15 [MH]⁺ 514.3 518.3 |
| 126 | 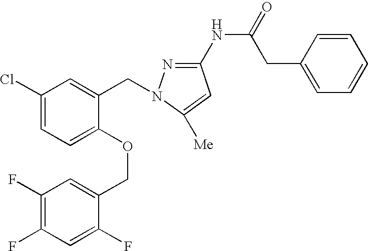 | N-{1-[(5-Chloro-2-{[(2,4,5- trifluorophenyl)methyl]oxy}phe- nyl)methyl]-5-methyl-1H- pyrazol-3-yl}-2- phenylacetamide | LC/MS Rt = 3.85 [MH]⁺ 500.4 502.4 |
| 127 | 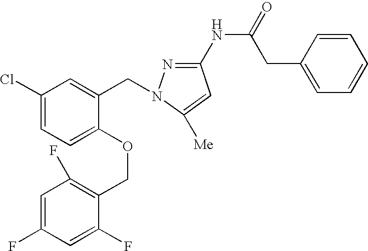 | N-{1-[(5-Chloro-2-{[(2,4,6- trifluorophenyl)methyl]oxy}phe- nyl)methyl]-5-methyl-1H- pyrazol-3-yl}-2- phenylacetamide | LC/MS Rt = 3.82 [MH]⁺ 500.4 502.4 |
| 128 | 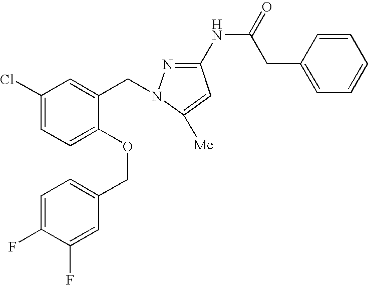 | N-{1-[(5-Chloro-2-{[(3,4- difluorophenyl)methyl]oxy}phe- nyl)methyl]-5-methyl-1H- pyrazol-3-yl}-2- phenylacetamide | LC/MS Rt = 3.82 [MH]⁺ 482.4 484.4 |
| 129 | 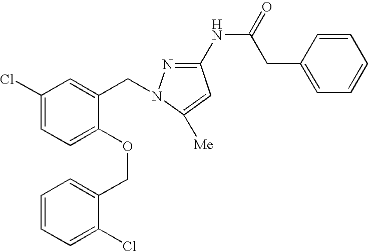 | N-{1-[(5-Chloro-2-{[(2- chlorophenyl)methyl]oxy}phe- nyl)methyl]-5-methyl-1H- pyrazol-3-yl}-2- phenylacetamide | LC/MS Rt = 3.96 [MH]⁺ 480.4 483.4 |
| 130 | 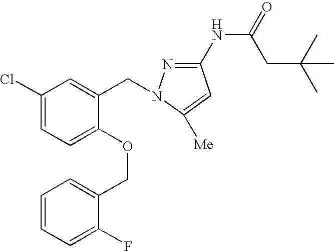 | N-{1-[(5-Chloro-2-{[(2- fluorophenyl)methyl]oxy}phe nyl)methyl]-5-methyl-1H- pyrazol-3-yl}-3,3- dimethylbutanamide | LC/MS Rt = 3.88 [MH]⁺ 444.4, 446.4 |
| 131 | 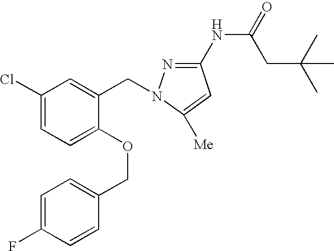 | N-{1-[(5-Chloro-2-{[(4- fluorophenyl)methyl]oxy}phe nyl)methyl]-5-methyl-1H- pyrazol-3-yl}-3,3- dimethylbutanamide | LC/MS Rt = 3.87 [MH]⁺ 444.4, 446.4 |
| 132 | 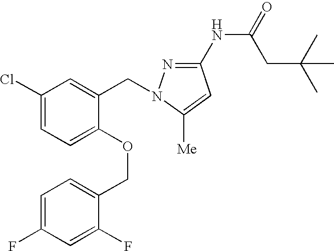 | N-{1-[(5-Chloro-2-{[(2,4- difluorophenyl)methyl]oxy}phe- nyl)methyl]-5-methyl-1H- pyrazol-3-yl}-3,3- dimethylbutanamide | LC/MS Rt = 3.91 [MH]⁺ 462.4, 464.4 |
| 133 | 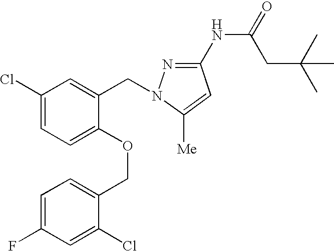 | N-{1-[(5-Chloro-2-{[(2-chloro- 4- fluorophenyl)methyl]oxy}phe nyl)methyl]-5-methyl-1H- pyrazol-3-yl}-3,3- dimethylbutanamide | LC/MS Rt = 4.06 [MH]⁺ 478.3, 481.4 |
| 134 | 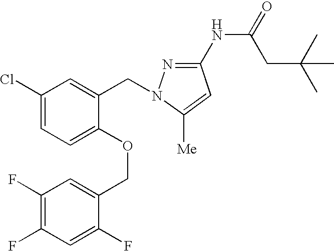 | N-{1-[(5-Chloro-2-{[(2,4,5- trifluorophenyl)methyl]oxy}phe- nyl)methyl]-5-methyl-1H- pyraozl-3-yl}-3,3- dimethylbutanamide | LC/MS Rt = 3.93 [MH]⁺ 480.4, 482.4 |
| 135 | 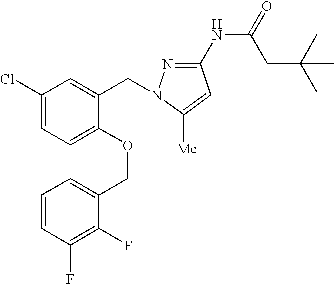 | N-{1-[(5-Chloro-2-{[(2,3- difluorophenyl)methyl]oxy}phe- nyl)methyl]-5-methyl-1H- pyrazol-3-yl}-3,3- dimethylbutanamide | LC/MS Rt = 3.91 [MH]⁺ 462.4, 464.4 |
| 136 | 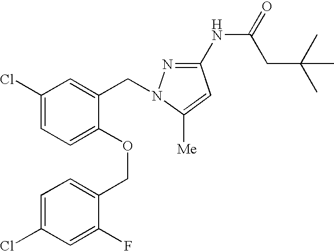 | N-{1-[(5-Chloro-2-{[(4-chloro- 2- fluorophenyl)methyl]oxy}phe nyl)methyl]-5-methyl-1H- pyrazol-3-yl}-3,3- dimethylbutanamide | LC/MS Rt = 4.07 [MH]⁺ 478.3, 481.4 |
| 137 | 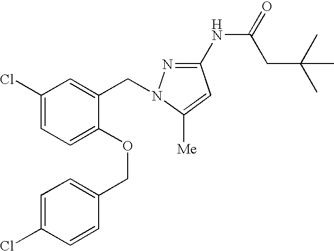 | N-{1-[(5-Chloro-2-{[(4- chlorophenyl)methyl]oxy}phe nyl)methyl]-5-methyl-1H- pyraozl-3-yl}-3,3- dimethylbutanamide | LC/MS Rt = 4.03 [MH]⁺ 460.4, 463.4 |
| 138 | 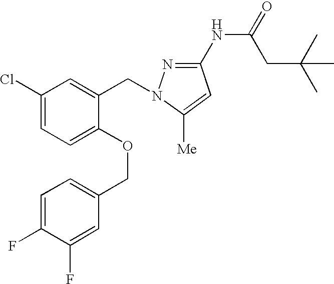 | N-{1-[(5-Chloro-2-{[(3,4- difluorophenyl)methyl]oxy}phe- nyl)methyl]-5-methyl-1H- pyrazol-3-yl}-3,3- dimethylbutanamide | LC/MS Rt = 3.91 [MH]⁺ 462.4, 464.4 |

### Example 139

### N-[1-({5-Chloro-2-[(phenylmethyl)oxy]phenyl}methyl)-5-methyl-1H-pyrazol-3-yl]-2-pyridinecarboxamide

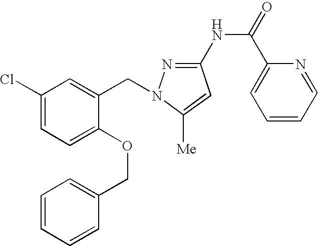

A mixture of 1-({5-chloro-2-[(phenylmethyl)oxy]phenyl}methyl)-5-methyl-1H-pyrazol-3-amine (164 mg, 0.5 mmol), picolinic acid (62 mg, 0.5 mmol), 1-hydroxybenzotriazole (92 mg, 0.6 mmol) and N-(3-dimethylaminopropyl)-N′ethylcarbodiimide (115 mg, 0.6 mmol) in dichloromethane (4 ml) was stirred at room temperature for 2 hours. The resulting solution was diluted with ether, washed with saturated sodium bicarbonate, dried (magnesium sulphate), evaporated and purified by flash chromatography eluting with ethyl acetate/hexane (1:3) to give the title compound as a white solid (130 mg).

LC/MS Rt 3.90, [MH]⁺ 433.4, 435.4.

The following compounds were prepared from appropriate intermediates by a similar procedure to that described in Example 139:

| Example | Structure | Name | LC/MS |
|---|---|---|---|
| 140 | 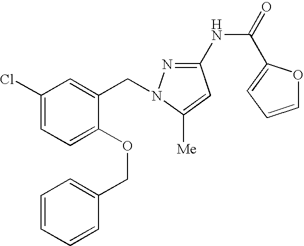 | N-[1-({5-Chloro-2- [(phenylmethyl)oxy]phenyl}meth- yl)-5-methyl-1H-pyrazol- 3-yl]-2-furancarboxamide | LC/MS Rt = 3.64 [MH]⁺ 422.4, 424.3 |
| 141 | 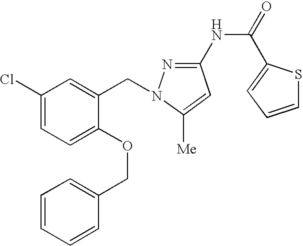 | N-[1-({5-Chloro-2- [(phenylmethyl)oxy]phenyl}meth- yl)-5-methyl-1H-pyrazol- 3-yl]-2- thiophenecarboxamide | LC/MS Rt = 3.83 [MH]⁺ 438.3, 440.3 |
| 142 | 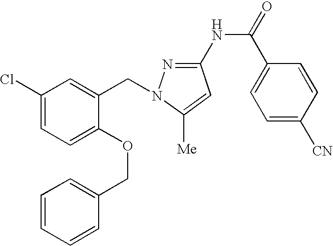 | N-[1-({5-Chloro-2- [(phenylmethyl)oxy]phenyl}meth- yl)-5-methyl-1H-pyrazol- 3-yl]-4-cyanobenzamide | LC/MS Rt = 3.79 [MH]⁺ 457.4, 459.4 |
| 143 | 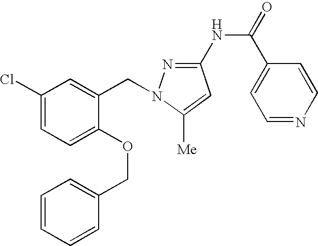 | N-[1-({5-Chloro-2- [(phenylmethyl)oxy]phenyl}meth- yl)-5-methyl-1H-pyrazol- 3-yl]-4-pyridinecarobxamide | LC/MS Rt = 3.51 [MH]⁺ 433.4, 435.4 |
| 144 | 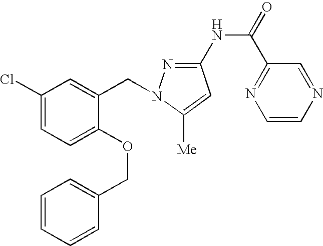 | N-[1-({5-Chloro-2- [(phenylmethyl)oxy]phenyl}meth- yl)-5-methyl-1H-pyrazol- 3-yl]-2-pyrazinecarboxamide | LC/MS Rt = 3.63 [MH]⁺ 434.4, 436.4 |
| 145 | 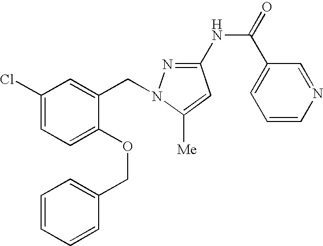 | N-[1-({5-Chloro-2- [(phenylmethyl)oxy]phenyl}meth- yl)-5-methyl-1H-pyrazol- 3-yl]-3-pyridinecarboxamide | LC/MS Rt = 3.48 [MH]⁺ 433.4, 435.4 |
| 146 | 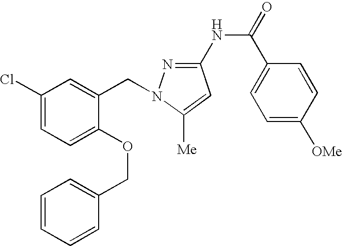 | N-[1-({5-Chloro-2- [(phenylmethyl)oxy]phenyl}meth- yl)-5-methyl-1H-pyrazol- 3-yl]-4-(methyloxy)- benzamide | LC/MS Rt = 3.81 [MH]⁺ 462.4, 464.4 |
| 147 | 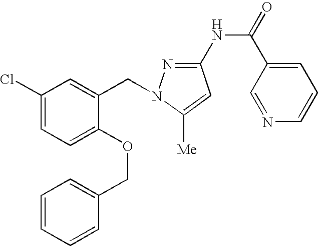 | N-[1-({5-Chloro-2- [(phenylmethyl)oxy]phenyl}meth- yl)-5-methyl-1H-pyrazol- 3-yl]-2-(2-pyridinyl)- acetamide | LC/MS Rt = 3.40 [MH]⁺ 447.4, 449.4 |
| 148 | 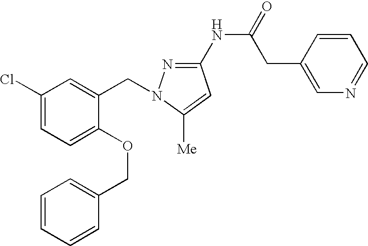 | N-[1-({5-Chloro-2- [(phenylmethyl)oxy]phenyl}meth- yl)-5-methyl-1H-pyrazol- 3-yl]-2-(3-pyridinyl)- acetamide | LC/MS Rt = 3.30 [MH]⁺ 447.4, 449.4 |
| 149 | 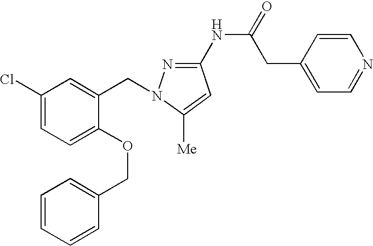 | N-[1-({5-Chloro-2- [(phenylmethyl)oxy]phenyl}meth- yl)-5-methyl-1H-pyrazol- 3-yl]-2-(4-pyridinyl)-acetamide | LC/MS Rt = 3.26 [MH]⁺ 447.4, 449.4 |
| 150 | 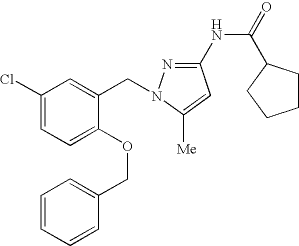 | N-[1-({5-Chloro-2- [(phenylmethyl)oxy]phenyl}meth- yl)-5-methyl-1H-pyrazol- 3-yl]cyclopentane- carboxamide | LC/MS Rt = 3.78 [MH]⁺ 424.4, 426.4 |
| 151 | 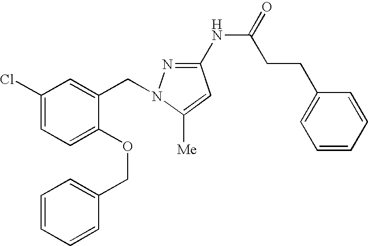 | N-[1-({5-Chloro-2- [(phenylmethyl)oxy]phenyl}meth- yl)-5-methyl-1H-pyrazol- 3-yl]-3-phenylpropanamide | LC/MS Rt = 3.85 [MH]⁺ 460.4, 462.4 |
| 152 | 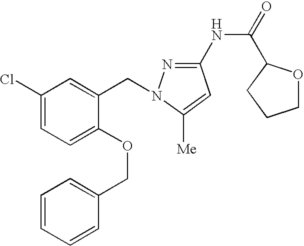 | N-[1-({5-Chloro-2- [(phenylmethyl)oxy]phenyl}meth- yl)-5-methyl-1H-pyraozl- 3-yl]tetrahydro-2- furancarboxamide | LC/MS Rt = 3.51 [MH]⁺ 426.4, 428.4 |
| 153 | 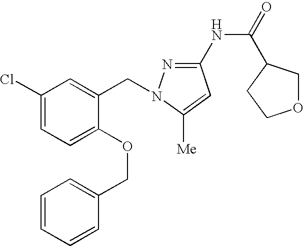 | N-[1-({5-Chloro-2- [(phenylmethyl)oxy]phenyl}meth- yl)-5-methyl-1H-pyrazol- 3-yl]tetrahydro-3- furancarboxamide | LC/MS Rt = 3.33 [MH]⁺ 426.4, 428.4 |
| 154 | 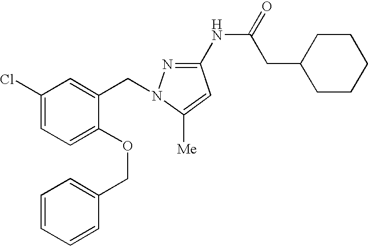 | N-[1-({5-Chloro-2- [(phenylmethyl)oxy]phenyl}meth- yl)-5-methyl-1H-pyrazol- 3-yl]-2-cyclohexylacetamide | LC/MS Rt = 4.03 [MH]⁺ 452.4, 454.4 |
| 155 | 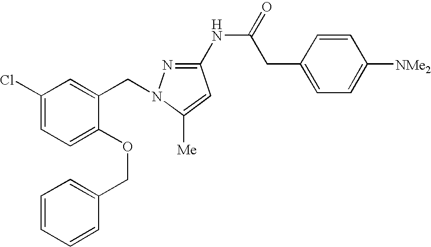 | N-[1-({5-Chloro-2- [(phenylmethyl)oxy]phenyl}meth- yl)-5-methyl-1H-pyrazol- 3-yl]-2-[4- (dimethylamino)phenyl]- acetamide | LC/MS Rt = 3.82 [MH]⁺ 489.4, 491.4 |
| 156 | 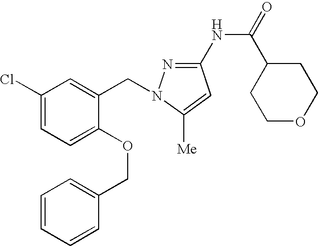 | N-[1-({5-Chloro-2- [(phenylmethyl)oxy]phenyl}meth- yl)-5-methyl-1H-pyrazol- 3-yl]tetrahydro-2H-pyran-4- carboxamide | LC/MS Rt = 3.35 [MH]⁺ 440.4, 442.4 |
| 157 | 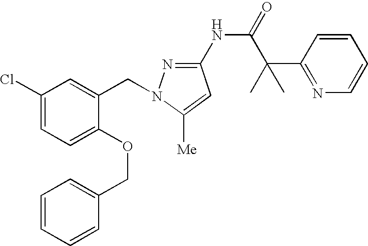 | N-[1-({5-Chloro-2- [(phenylmethyl)oxy]phenyl}meth- yl)-5-methyl-1H-pyrazol- 3-yl]-2-methyl-2-(2-pyridinyl)- propanamide | LC/MC Rt = 3.76 [MH]⁺ 475.4, 477.4 |
| 158 | 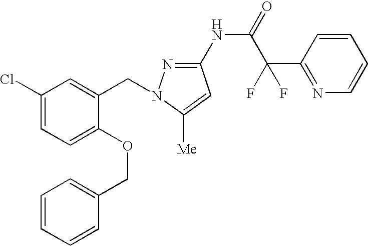 | N-[1-({5-Chloro-2- [(phenylmethyl)oxy]phenyl}meth- yl)-5-methyl-1H-pyrazol- 3-yl]-2,2-difluoro-2-(2- pyridinyl)acetamide | LC/MS Rt = 3.74 [MH]⁺ 483.4, 485.3 |
| 159 | 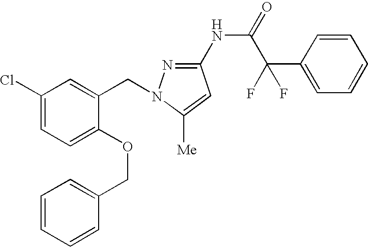 | N-[1-({5-Chloro-2- [(phenylmethyl)oxy]phenyl}meth- yl)-5-methyl-1H-pyrazol- 3-yl]-2,2-difluoro-2- phenylacetamide | LC/MS Rt = 4.01 [MH]⁺ 482.3, 484.4 |
| 160 | 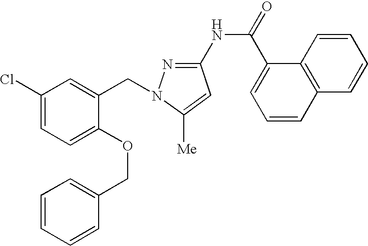 | N-[1-({5-Chloro-2- [(phenylmethyl)oxy]phenyl}meth- yl)-5-methyl-1H-pyrazol- 3-yl]-1-naphthalene- carboxamide | LC/MS Rt = 4.03 [MH]⁺ 482.4, 484.4 |
| 161 | 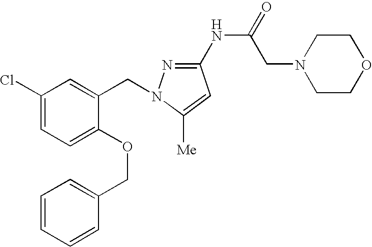 | N-[1-({5-Chloro-2- [(phenylmethyl)oxy]phenyl}meth- yl)-5-methyl-1H-pyrazol- 3-yl]-2-(4-morpholinyl)- acetamide | LC/MS Rt = 3.21 [MH]⁺ 455.4, 457.4 |
| | 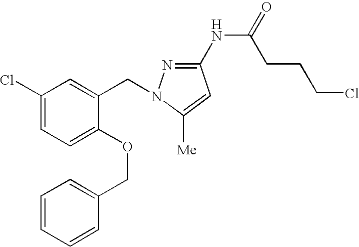 | 4-Chloro-N-[1-({5-chloro-2- [(phenylmethyl)oxy]phenyl}meth- yl)-5-methyl-1H-pyrazol- 3-yl]butanamide | LC/MS Rt = 3.67 [MH]⁺ 432.4, 435.4 |
| 162 | 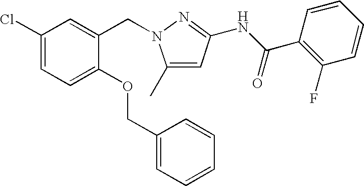 | N-[1-({5-Chloro-2- [(phenylmethyl)oxy]phenyl}meth- yl)-5-methyl-1H-pyrazol- 3-yl]-2-fluorobenzamide | LC/MS Rt = 3.89 min, [MH⁺] 450, 452. |
| 163 | 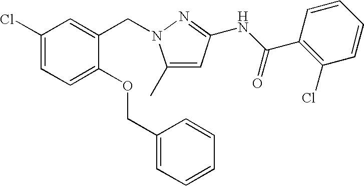 | 2-Chloro-N-[1-({5-chloro-2- [(phenylmethyl)oxy]phenyl}meth- yl)-5-methyl-1H-pyrazol- 3-yl]benzamide | LC/MS Rt = 3.85 min, [MH⁺] 466, 469. |
| 164 | 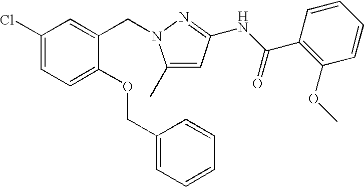 | N-[1-({5-Chloro-2- [(phenylmethyl)oxy]phenyl}meth- yl)-5-methyl-1H-pyrazol- 3-yl]-2-(methyloxy) benzamide | LC/MS Rt = 3.93 min, [MH⁺] 462, 464. |
| 165 | 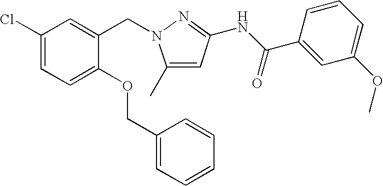 | N-[1-({5-Chloro-2- [(phenylmethyl)oxy]phenyl}meth- yl)-5-methyl-1H-pyrazol- 3-yl]-3-(methyloxy) benzamide | LC/MS Rt = 3.87 min, [MH⁺] 462, 464. |
| 166 | 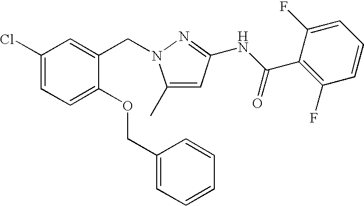 | N-[1-({5-Chloro-2- [(phenylmethyl)oxy]phenyl}meth- yl)-5-methyl-1H-pyrazol- 3-yl]-2,6-difluorobenzamide | LC/MS Rt = 3.80 min, [MH⁺] 468, 470. |
| 167 | 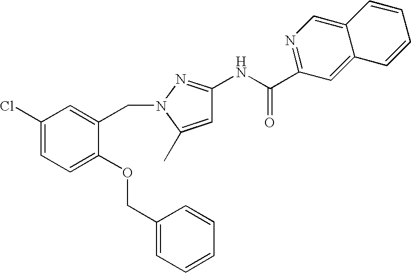 | N-[1-({5-Chloro-2- [(phenylmethyl)oxy]phenyl}meth- yl)-5-methyl-1H-pyrazol- 3-yl]-3- isoquinolinecarboxamide | LC/MS: Rt = 3.46 min. [MH⁺] 483. |
| 168 | 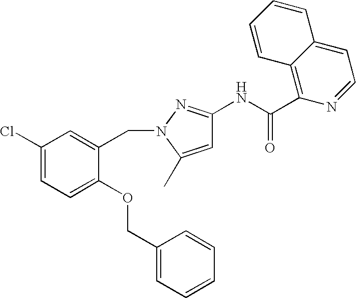 | N-[1-({5-Chloro-2- [(phenylmethyl)oxy]phenyl}meth- yl)-5-methyl-1H-pyrazol- 3-yl]-1- isoquinolinecarboxamide | LC/MS: Rt = 3.51 min. [MH⁺] 483. |
| 169 | 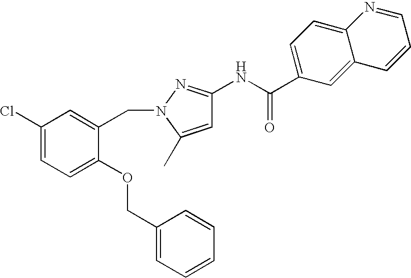 | N-[1-({5-Chloro-2- [(phenylmethyl)oxy]phenyl}meth- yl)-5-methyl-1H-pyrazol- 3-yl]-6-quinolinecarboxamide | LC/MS: Rt = 2.95 min. [MH⁺] 483. |
| 170 | 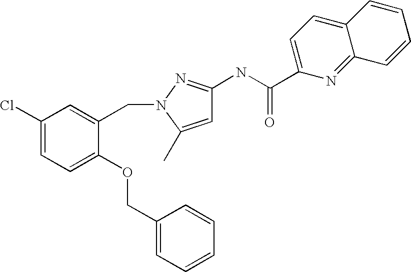 | N-[1-({5-Chloro-2- [(phenylmethyl)oxy]phenyl}meth- yl)-5-methyl-1H-pyrazol- 3-yl]-2-quinolinecarboxamide | LC/MS: Rt = 3.95 min. [MH⁺] 483. |
| 171 | 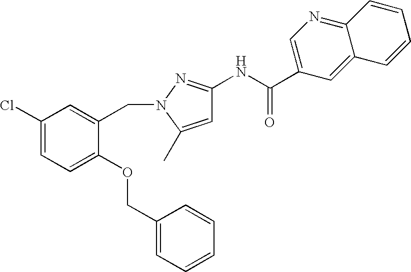 | N-[1-({5-Chloro-2- [(phenylmethyl)oxy]phenyl}meth- yl)-5-methyl-1H-pyrazol- 3-yl]-3-quinolinecarboxamide | LC/MS: Rt = 3.57 min. [MH⁻] 481. |
| 172 | 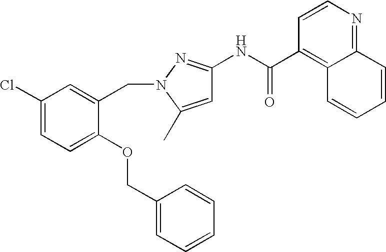 | N-[1-({5-Chloro-2- [(phenylmethyl)oxy]phenyl}meth- yl)-5-methyl-1H-pyrazol- 3-yl]-4-quinolinecarboxamide | LC/MS: Rt = 3.50 min. [MH⁻] 481. |
| 173 | 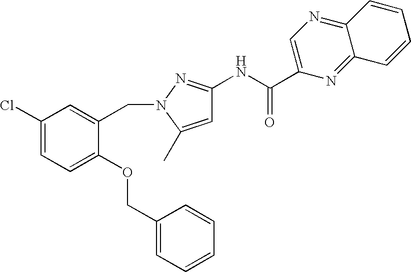 | N-[1-({5-Chloro-2- [(phenylmethyl)oxy]phenyl}meth- yl)-5-methyl-1H-pyrazol- 3-yl]-2- quinoxalinecarboxamide | LC/MS: Rt = 3.84 min. [MH⁻] 482. |
| 174 | 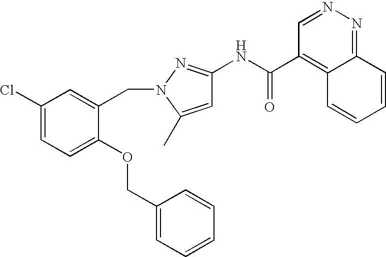 | N-[1-({5-chloro-2- [(phenylmethyl)oxy]phenyl}meth- yl)-5-methyl-1H-pyrazol- 3-yl]-4-cinnolinecarboxamide | LC/MS: Rt = 3.33 min. [MH⁻] 482. |
| 175 | 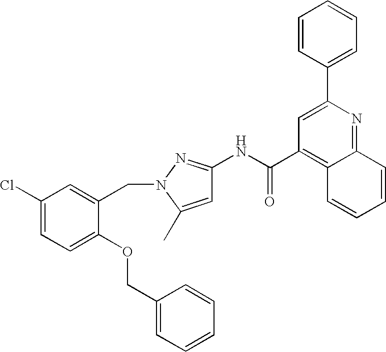 | N-[1-({5-Chloro-2- [(phenylmethyl)oxy]phenyl}meth- yl)-5-methyl-1H-pyrazol- 3-yl]-2-phenyl-4- quinolinecarboxamide | LC/MS: Rt = 3.66 min. [MH⁺] 559. |
| 176 | 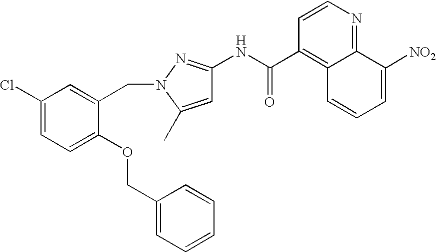 | N-[1-({5-Chloro-2- [(phenylmethyl)oxy]phenyl}meth- yl)-5-methyl-1H-pyrazol- 3-yl]-8-nitro-4- quinolinecarboxamide | LC/MS: Rt = 3.38 min. [MH⁺] 528. |
| 177 | 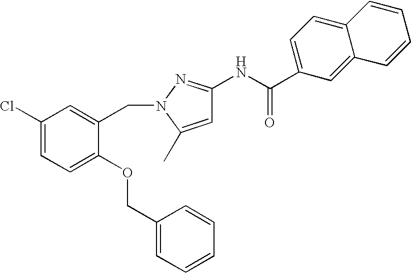 | N-[1-({5-Chloro-2- [(phenylmethyl)oxy]phenyl}meth- yl)-5-methyl-1H-pyrazol- 3-yl]-2- naphthalenecarboxamide | LC/MS: Rt = 3.72 min. [MH⁺] 482. |
| 178 | 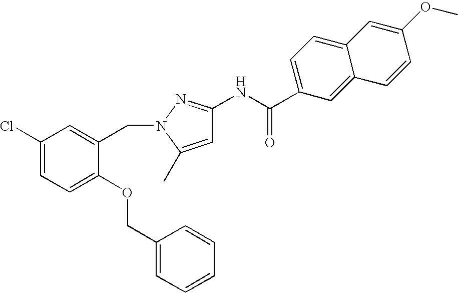 | N-[1-({5-Chloro-2- [(phenylmethyl)oxy]phenyl}meth- yl)-5-methyl-1H-pyrazol- 3-yl]-6-(methyloxy)-2- naphthalenecarboxamide | LC/MS: Rt = 3.71 min. [MH⁺] 512. |

### Example 179

### 1-[1-({5-Chloro-2-[(phenylmethyl)oxy]phenyl}methyl)-5-methyl-1H-pyrazol-3-yl]-2-pyrrolidinone

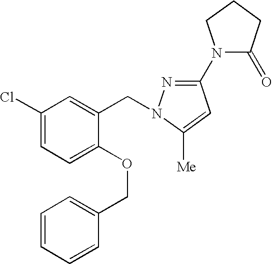

60% Sodium hydride (24 mg, 0.6 mmol) was added to a solution of 4-chloro-N-[1-({5-chloro-2-[(phenylmethyl)oxy]phenyl}methyl)-5-methyl-1H-pyrazol-3-yl]butanamide in tetrahydrofuran (5 ml) and heated at 60° C. for 30 minutes. The resulting mixture was diluted with ethyl acetate/water and the organic phase dried (magnesium sulphate), evaporated and the residue triturated with ether to give the title compound as a white solid (126 mg). LC/MS Rt 3.52, [MH]⁺ 396.1, 398.1

### [1-({5-Chloro-2-[(Phenylmethyl)oxy]phenyl}methyl)-5-methyl-1H-pyrazol-3-yl]formamide

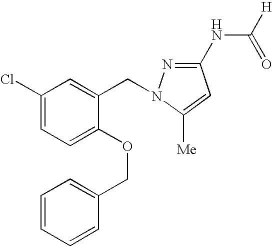

Formic acid (0.1 ml) was added to acetic anhydride (0.2 ml) and heated at 50° C. for 15 minutes then cooled to room temperature and added to 1-({5-chloro-2[(phenylmethyl)oxy]phenyl}methyl)-5-methyl-1H-pyrazol-3-amine with stirring. After 1 hour the solution was diluted with ether and washed with sodium bicarbonate solution. The organic phase was dried (magnesium sulphate), evaporated and purified by flash chromatography eluting with ethyl acetate/hexane (3:7) to give the title compound as a white solid (71 mg). LC/MS Rt 3.30, [MH]⁺ 356.4, 358.4.

### 1-({5-Chloro-2-[(phenylmethyl)oxy]phenyl}methyl)-N,5-dimethyl-1H-pyrazol-3-amine

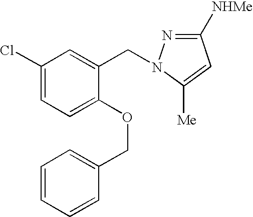

1M Lithium aluminium hydride solution in tetrahydrofuran (3 ml) was added to a stirred solution of [1-({5-chloro-2-[(phenylmethyl)oxy]phenyl}methyl)-5-methyl-1H-pyrazol-3-yl]formamide in tetrahydrofuran (20 ml) under nitrogen and the mixture stirred at room temperature for 16 hours then quenched with 2M sodium hydroxide and ether. The organic phase was dried (magnesium sulphate), evaporated and purified by flash chromatography eluting with ethyl acetate/hexane (3:7) to give the title compound as a pale coloured gum (175 mg). LC/MS Rt 3.43, [MH]⁺ 342.4, 344.4.

### Example 180

### N-[1-({5-Chloro-2-[(phenylmethyl)oxy]phenyl}methyl)-5-methyl-1H-pyrazol-3-yl]-N-methylbenzamide

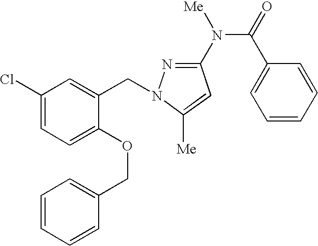

Benzoyl chloride (39 mg, 0.275 mmol) was added to a solution of 1-({5-chloro-2-[(phenylmethyl)oxy]phenyl}methyl)-N,5-dimethyl-1H-pyrazol-3-amine (85 mg, 0.25 mmol) and triethylamine (30 mg, 0.3 mmol) in dichloromethane (3 ml). After 1 hour at room temperature the solution was diluted with ether, washed with sodium bicarbonate solution, dried (magnesium sulphate), evaporated and purified by flash chromatography eluting with ethyl acetate/hexane (1:3) to give, after trituration with ether/hexane, the title compound as a white solid (51 mg). LC/MS Rt 3.79, [MH]⁺ 446.4, 448.3.

### Example 181

### 2-[1-({5-Chloro-2-[(phenylmethyl)oxy]phenyl}methyl)-5-methyl-1H-pyrazol-3-yl]-1H-isoindole-1,3(2H)-dione

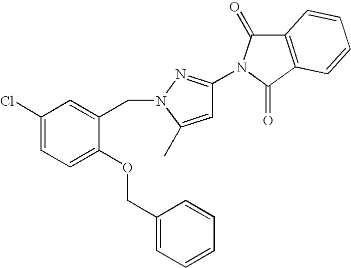

A mixture of 1-({5-chloro-2-[(phenylmethyl)oxy]phenyl}methyl)-5-methyl-1H-pyrazol-3-amine (164 mg, 0.5 mmol) and phthalic anhydride (74 mg, 0.5 mmol) in toluene (5 ml) was refluxed for 2 hours. The solvent was evaporated and the residue triturated with 20:1 hexane/ethyl acetate to give the title compound as a colourless solid (185 mg).

LC/MS: Rt=3.63 min. [MH⁺] 458.

### Example 182

### [2-({[1-({5-Chloro-2-[(2-methylpropyl)oxy]phenyl}methyl)-5-methyl-1H-pyrazol-3-yl]amino}carbonyl)phenyl]methyl benzoate

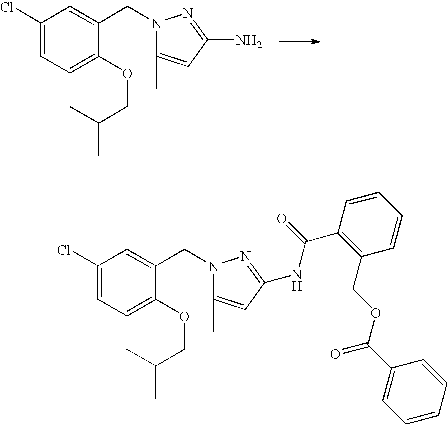

1-({5-Chloro-2-[(2-methylpropyl)oxy]phenyl}methyl)-5-methyl-1H-pyrazol-3-amine (1.5 g, 5.1 mmol) and 2-(benzoyloxymethyl)benzoyl chloride (2.1 g, 7.7 mmol) were stirred in dichloromethane (5 ml) and pyridine (5 ml) under argon for 2 hours. The solvent was evaporated and the residue diluted with ethyl acetate and water. The organic layer was separated, dried (Na₂SO₄) and evaporated and the crude product flash chromatographed with dichloromethane/methanol (9:1) to yield the title compound (2.28 g)

LC/MS [M+H] Rt=3.93 min [MH+] 532, 534.

### Example 183

### N-[1-({5-Chloro-2-[(2-methylpropyl)oxy]phenyl}methyl)-5-methyl-1H-pyrazol-3-yl]-2-(hydroxymethyl)benzamide

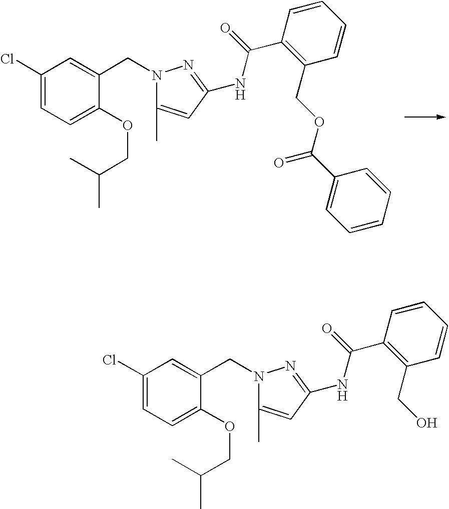

[2-({[1-({5-Chloro-2-[(2-methylpropyl)oxy]phenyl}methyl)-5-methyl-1H-pyrazol-3-yl]amino}carbonyl)phenyl]methyl benzoate (0.1 g, 0.19 mmol) was stirred in ethanol (5 ml) and 21% sodium ethoxide in ethanol (0.07 ml, 0.19 mmol) was added. The mixture was stirred at room temperature for 1 hour and evaporated. The yellow gum was flash chromatographed with ethyl acetate/hexane (3/1) to yield the title compound (0.048 g) LC/MS Rt=3.29 min [MH+] 428, 430.

### Example 184

### 2-[1-({5-Chloro-2-[(2-methylpropyl)oxy]phenyl}methyl)-5-methyl-1H-pyrazol-3-yl]-2,3-dihydro-1H-isoindol-1-one

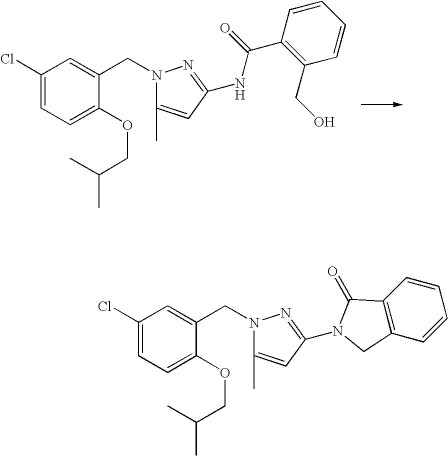

N-[1-({5-Chloro-2-[(2-methylpropyl)oxy]phenyl}methyl)-5-methyl-1H-pyrazol-3-yl]-2-(hydroxymethyl)benzamide (0.1 g, 0.23 mmol), triethylamine (0.065 ml, 0.46 mmol) and methanesulphonyl chloride (0.027 ml, 0.35 mmol) were stirred in dichloromethane (5 ml) for 90 minutes. The reaction was evaporated and the residue dissolved in DMF (5 ml) and potassium carbonate (0.048 g, 0.35 mmol) and pyrrolidine (0.019 ml, 0.23 mmol) added. The mixture was stirred and heated at 90° C. for 60 hours then evaporated to dryness and purified using flash chromatography eluting with ethyl acetate/hexane (1/2) to yield the title compound (0.045 g). LC/MS Rt=3.63 min [MH+] 410, 412.

### Example 185

### 5,6-Dichloro-N-[1-({5-chloro-2-[(2-methylpropyl)oxy]phenyl}methyl)-5-methyl-1H-pyrazol-3-yl]-3-pyridinecarboxamide

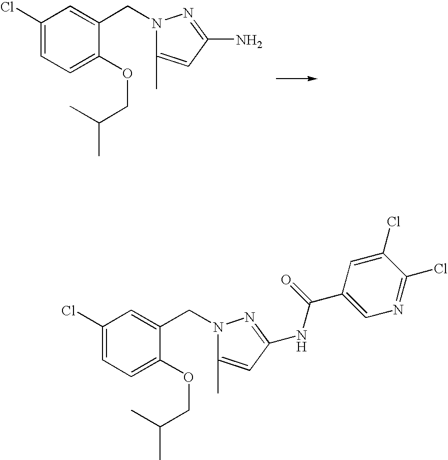

5,6-Dichloro-3-pyridinecarboxylic acid (1.5 g, 7.8 mmol) was suspended in toluene (15 ml) and thionyl chloride (4.5 ml, 39 mmol) added. The solution was refluxed under argon for 6 hours and then evaporated to a brown oil. The residue was stirred in dichloromethane (10 ml) and pyridine (10 ml) and 1-({5-chloro-2-[(2-methylpropyl)oxy]phenyl}methyl)-5-methyl-1H-pyrazol-3-amine (1.0 g, 3.4 mmol) added. The reaction was stirred for 1 hour then evaporated to a brown solid which was flash chromatographed with ethyl acetate/hexane (1/2). The product was stirred in ethanol (30 ml) and 2M sodium hydroxide (5 ml) for 30 minutes then evaporated. The yellow oil was dissolved in ethyl acetate, washed with 2M sodium hydroxide and the organic layer separated, dried over magnesium sulphate and evaporated to give the title compound (1.082 g).

LC/MS Rt=3.83 min [MH+] 466, 468 and 470

The following examples were prepared in a similar manner to 5,6-dichloro-N-[1-({5-chloro-2-[(2-methylpropyl)oxy]phenyl}methyl)-5-methyl-1H-pyrazol-3-yl]-3-pyridinecarboxamide from the appropriate intermediates.

| Example | | Name | Data |
|---|---|---|---|
| 186 | 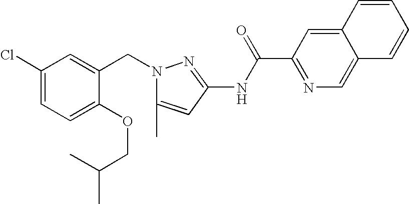 | N-[1-({5-Chloro-2-[(2- methylpropyl)oxy]phenyl}methyl)-5- methyl-1H-pyrazol-3-yl]-3- isoquinolinecarboxamide | LC/MS Rt = 4.00 min [MH+] 449, 451 |
| 187 | 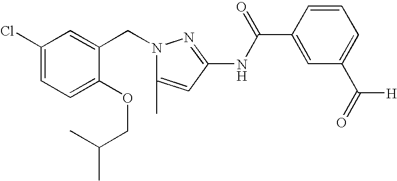 | N-[1-({5-Chloro-2-[(2- methylpropyl)oxy]phenyl}methyl)-5- methyl-1H-pyrazol-3-yl]-3- formylbenzamide | LC/MS Rt = 3.44 min [MH+] 426, 428 |
| 188 | 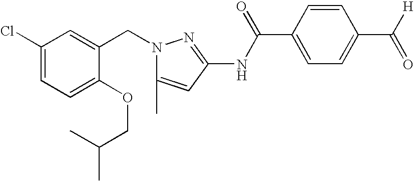 | N-[1-({5-Chloro-2-[(2- methylpropyl)oxy]phenyl}methyl)-5- methyl-1H-pyrazol-3-yl]-4- formylbenzamide | LC/MS Rt = 3.44 min [MH+] 426, 428 |

### Example 189

### N-[1-({5-Chloro-2-[(2-methylpropyl)oxy]phenyl}methyl)-5-methyl-1H-pyrazol-3-yl]-6-(4-morpholinyl)-3-pyridinecarboxamide

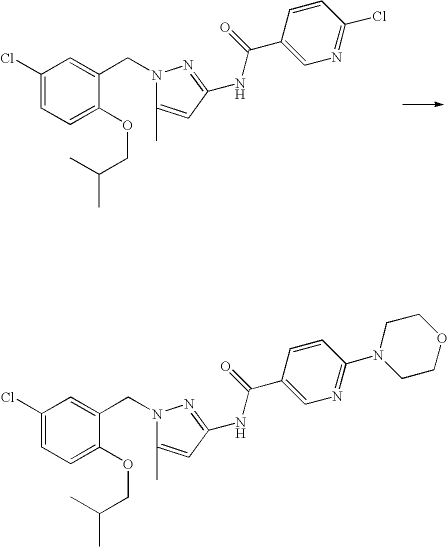

6-Chloro-N-[1-({5-chloro-2-[(2-methylpropyl)oxy]phenyl}methyl)-5-methyl-1H-pyrazol-3-yl]-3-pyridinecarboxamide (0.055 g, 0.13 mmol) and morpholine (0.066 ml, 0.78 mmol) were dissolved in ethanol (2 ml) and heated for 3 hours at 120° C. in a Smithcreatore microwave. The reaction mixture was evaporated to dryness and flash chromatographed with ethyl actetate/hexane (1/1) to give the title compound (0.049 g).

LC/MS Rt=3.33 min [MH+] 484, 486

### Example 190

### 5-Chloro-N-[1-({5-chloro-2-[(2-methylpropyl)oxy]phenyl}methyl)-5-methyl-1H-pyrazol-3-yl]-6-(4-morpholinyl)-3-pyridinecarboxamide

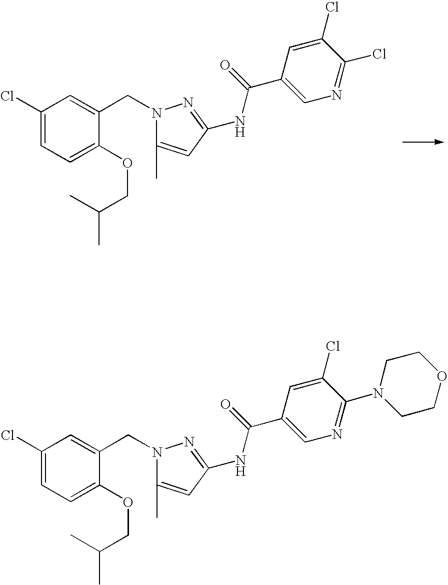

5,6-Dichloro-N-[1-({5-chloro-2-[(2-methylpropyl)oxy]phenyl}methyl)-5-methyl-1H-pyrazol-3-yl]-3-pyridinecarboxamide (0.1 g, 0.21 mmol) and morpholine (0.018 ml, 0.21 mmol) in dimethylformamide (2 ml) were heated at 250° C. in a Smithcreator®) microwave for 1 hour. The mixture was evaporated to a brown solid and purified by MDAP to yield the title compound (0.020 g). LC/MS Rt=3.66 min [MH+] 518, 512.

### Example 191

### 5-Chloro-N-[1-({5-chloro-2-[(2-methylpropyl)oxy]phenyl}methyl)-5-methyl-1H-pyrazol-3-yl]-6-(1-pyrrolidinyl)-3-pyridinecarboxamide

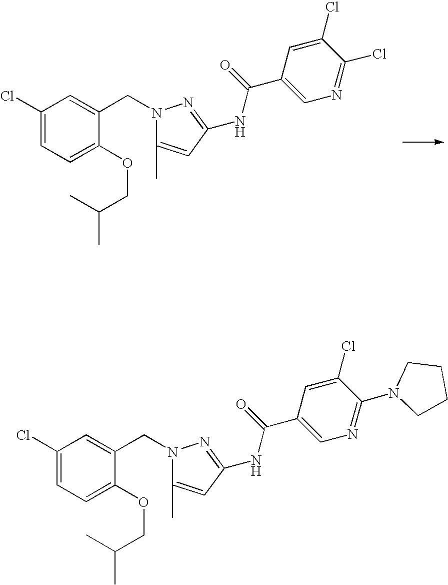

5,6-Dichloro-N-[1-({5-chloro-2-[(2-methylpropyl)oxy]phenyl}methyl)-5-methyl-1H-pyrazol-3-yl]-3-pyridinecarboxamide (0.1 g, 0.21 mmol) and pyrrolidine (0.019 ml, 0.23 mmol) in N-methylpyrrolidone (2 ml) were heated at 250° C. in a Smithcreator® microwave for 1 hour. The mixture was diluted with ethyl acetate and washed with water. The organic layer was separated and then evaporated to a brown solid and purified by MDAP to yield the title compound (0.044 g). LC/MS Rt=4.04 min [MH+] 502, 504.

### Example 192

### 5-Chloro-N-[1-({5-chloro-2-[(2-methylpropyl)oxy]phenyl}methyl)-5-methyl-1H-pyrazol-3-yl]-6-(4-methyl-1-piperazinyl)-3-pyridinecarboxamide hydrochloride

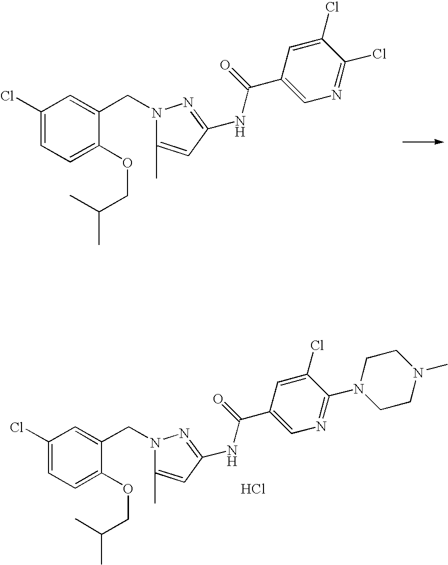

5,6-Dichloro-N-[1-({5-chloro-2-[(2-methylpropyl)oxy]phenyl}methyl)-5-methyl-1H-pyrazol-3-yl]-3-pyridinecarboxamide (0.2 g, 0.428 mmol), N-methylpiperazine (0.10 ml, 1.7 mmol) were heated in a Smithcreator® microwave in N-methylpyrrolidone (1.9 ml) at 250° C. for 10 min. The reaction mixture was purified on an SCX cartridge and by flash chromatography (Isolute® Flash NH₂ column) eluting with dichloromethane:methanol (19:1) and dried in vacuo to yield the title compound (0.180 g). LC/MS Rt=2.51 min [MH+] 531, 533.

### Example 193

### 5-Chloro-N-[1-({5-chloro-2-[(2-methylpropyl)oxy]-phenyl}methyl)-5-methyl-1H-pyrazol-3-yl]-6-oxo-1,6-dihydro-3-pyridinecarboxamide

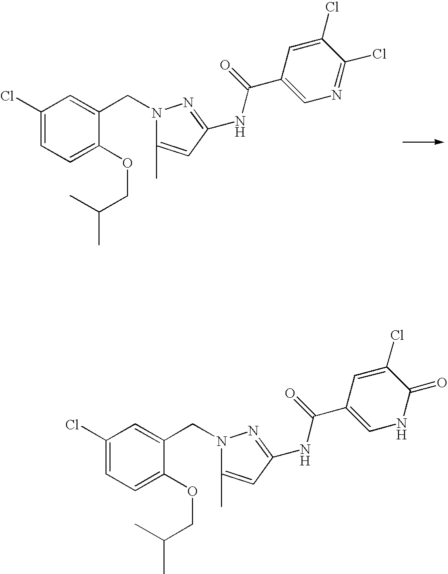

5,6-Dichloro-N-[1-({5-chloro-2-[(2-methylpropyl)oxy]phenyl}methyl)-5-methyl-1H-pyrazol-3-yl]-3-pyridinecarboxamide (0.1 g, 0.21 mmol), acetic acid (0.1 ml) and ethanol (0.1 ml) in N-methylpyrrolidone (2 ml) were heated to 250° C. in a Smithcreator® microwave for 3 hours. The mixture was diluted with ethyl acetate and washed with water. The organic layer was separated and evaporated to a brown solid. This was purified by MDAP and further purified by flash chromatography with ethyl acetate hexane (1/1) to yield the title compound (0.007 g). LC/MS Rt=3.14 min [MH+] 449, 451.

### Example 194

### N-[1-({5-Chloro-2-[(2-methylpropyl)oxy]phenyl}methyl)-5-methyl-1H-pyrazol-3-yl]-3-[(dimethylamino)methyl]benzamide hydrochloride

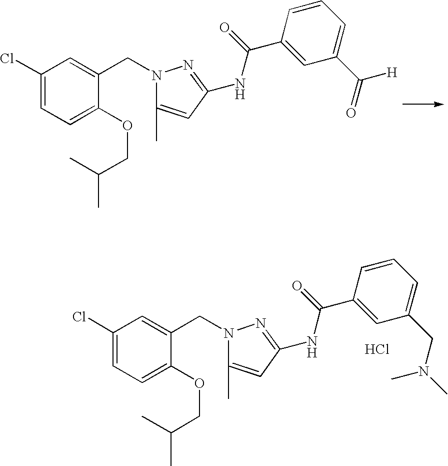

N-[1-({5-Chloro-2-[(2-methylpropyl)oxy]phenyl}methyl)-5-methyl-1H-pyrazol-3-yl]-3-formylbenzamide (0.058 g, 0.14 mmol), 40% dimethylamine in water (0.016 ml, 0.14 mmol) and acetic acid (0.08 ml, 1.4 mmol) were stirred together in dichloromethane (5 ml) and sodium triacetoxyborohydide (0.089 g, 0.42 mmol) was added cautiously. The mixture was stirred for 16 hours and acidified with 2M hydrochloric acid (5 ml). The reaction mixture was purified on an SCX ion exchange cartridge and by MDAP to yield the title compound (0.005 g). LC/MS Rt=2.39 min [MH+] 455, 457.

The following examples were synthesised in a similar manner to N-[1-({5-chloro-2-[(2-methylpropyl)oxy]phenyl}methyl)-5-methyl-1H-pyrazol-3-yl]-3-[(dimethylamino)methyl]benzamide hydrochloride from the appropriate intermediates:

| Example | | Compound name | Data |
|---|---|---|---|
| 195 | 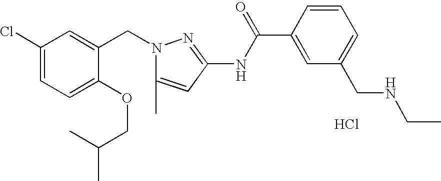 | N-[1-({5-Chloro-2-[(2- methylpropyl)oxy]phenyl}methyl)-5- methyl-1H-pyrazol-3-yl]-3- [(ethylamino)methyl]benzamide hydrochloride | LC/MS Rt = 2.39 min [MH+] 455, 457 |
| 196 | 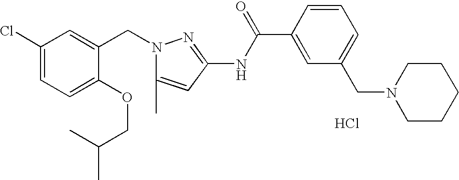 | N-[1-({5-Chloro-2-[(2- methylpropyl)oxy]phenyl}methyl)-5- methyl-1H-pyrazol-3-yl]-3-(1- piperidinylmethyl)benzamide hydrochloride | LC/MS Rt = 2.53 min [MH+] 495, 497 |
| 197 | 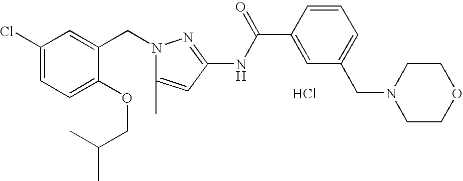 | N-[1-({5-Chloro-2-[(2- methylpropyl)oxy]phenyl}methyl)-5- methyl-1H-pyrazol-3-yl]-3-(4- morpholinylmethyl)benzamide hydrochloride | LC/MS Rt = 2.39 min [MH+] 497, 499 |
| 198 | 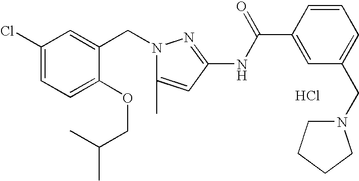 | N-[1-({5-Chloro-2-[(2- methylpropyl)oxy]phenyl}methyl)-5- methyl-1H-pyrazol-3-yl]-3-(1- pyrrolidinylmethyl)benzamide hydrochloride | LC/MS [MH+] 481, 483, Rt = 2.39 min |

### Example 199

### N-[1-({5-Chloro-2-[(2-methylpropyl)oxy]phenyl}methyl)-5-methyl-1H-pyrazol-3-yl]-4-[(dimethylamino)methyl]benzamide hydrochloride

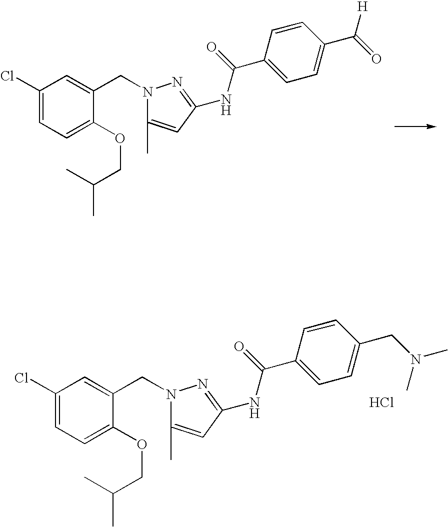

N-[1-({5-Chloro-2-[(2-methylpropyl)oxy]phenyl}methyl)-5-methyl-1H-pyrazol-3-yl]-4-formylbenzamide (0.1 g, 0.23 mmol), acetic acid (0.015 ml, 0.25 mmol), 40% dimethylamine in water (0.053 ml, 0.46 mmol) and sodium triacetoxyborohydride (0.149 g, 0.69 mmol) were stirred in dichloromethane (3.5 ml) for 3 hour and evaporated to dryness. The residue was redissolved in methanol and purified on an SCX ion exchange cartridge and further purified by MDAP. The residue was stirred in 1M hydrogen chloride in diethyl ether (1 ml) and the solid filtered to yield the title compound (0.045 g). LC/MS Rt=2.37 min [MH+] 455, 457.

The following examples were prepared in a similar manner to N-[1-({5-chloro-2-[(2-methylpropyl)oxy]phenyl}methyl)-5-methyl-1H-pyrazol-3-yl]-4-[(dimethylamino)methyl]benzamide hydrochloride from the appropriate intermediates:

| Example | | Compound name | Data |
|---|---|---|---|
| 200 | 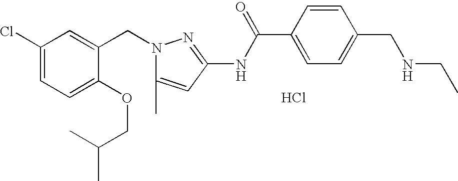 | N-[1-({5-Chloro-2-[(2- methylpropyl)oxy]phenyl}methyl)-5- methyl-1H-pyrazol-3-yl]-4- [(ethylamino)methyl]benzamide hydrochloride | LC/MS Rt = 2.37 min [MH+] 455, 457 |
| 201 | 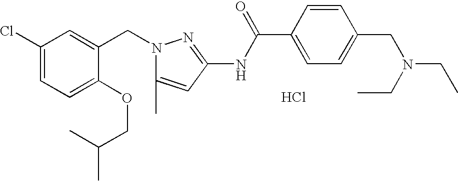 | N-[1-({5-Chloro-2-[(2- methylpropyl)oxy]phenyl}methyl)-5- methyl-1H-pyrazol-3-yl]-4- [(diethylamino)methyl]benzamide hydrochloride | LC/MS Rt = 2.45 min [MH+] 483, 485 |
| 202 | 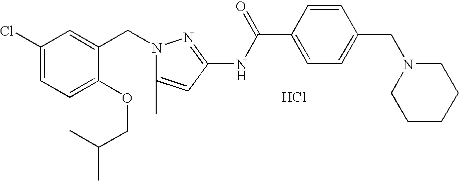 | N-[1-({5-Chloro-2-[(2- methylpropyl)oxy]phenyl}methyl)-5- methyl-1H-pyrazol-3-yl]-4-(1- piperidinylmethyl)benzamide hydrochloride | LC/MS Rt = 2.46 min [MH+] 495, 497 |
| 203 | 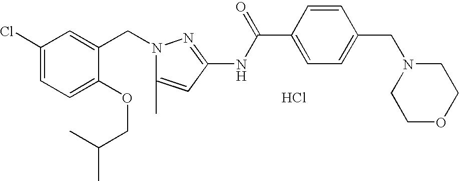 | N-[1-({5-Chloro-2-[(2- methylpropyl)oxy]phenyl}methyl)-5- methyl-1H-pyrazol-3-yl]-4-(4- morpholinylmethyl)benzamide hydrochloride | LC/MS Rt = 2.39 min [MH+] 497, 499 |
| 204 | 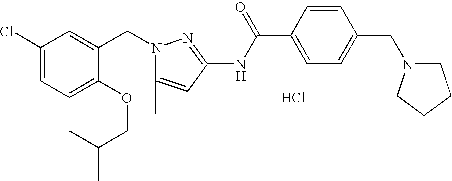 | N-[1-({5-Chloro-2-[(2- methylpropyl)oxy]phenyl}methyl)-5- methyl-1H-pyrazol-3-yl]-4-(1- pyrrolidinylmethyl)benzamide hydrochloride | LC/MS Rt = 2.44 min [MH+] 481, 483 |

### Example 205

### N-[1-({5-Chloro-2-[(2-methylpropyl)oxy]phenyl}methyl)-5-methyl-1H-pyrazol-3-yl]-4-[(3-oxo-1-piperazinyl)methyl]benzamide hydrochloride

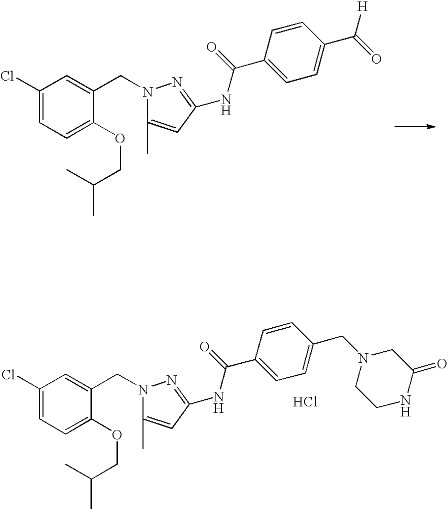

N-[1-({5-Chloro-2-[(2-methylpropyl)oxy]phenyl}methyl)-5-methyl-1H-pyrazol-3-yl]-4-formylbenzamide (0.1 g, 0.23 mmol), acetic acid (0.015 ml, 0.25 mmol), 2-oxopiperazine (0.046 g, 0.46 mmol) and sodium triacetoxyborohydride (0.69 mmol, 0.149 g) were stirred in dichloromethane (3.5 ml) for 3 hour and the mixture evaporated to dryness. The residue was flash chromatographed with 2M ammonia in methanol solution/dichloromethane (1/19). The product was purified by MDAP to yield the title compound (0.016 g) LC/MS Rt=2.63 min [MH+] 510, 512.

### 4-Ethenyl-2-fluorobenzoic acid

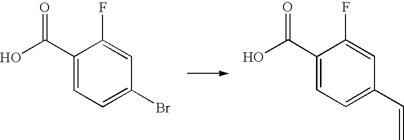

4-Bromo-2-fluorobenzoic acid (1.0 g, 4.5 mmol), magnesium sulphate (1.24 g, 10.4 mmol), 2,4,6-trivinylcyclotriboroxane pyridine complex (2.3 mmol, 0.55 g), tetrakis(triphenylphosphine)palladium(0) (58 mg, 0.05 mmol) and potassium carbonate (1.24 g, 9.0 mmol) were heated to reflux in water (18 ml) and 1,2-dimethoxyethane (66 ml) for 20 hours. The reaction was then diluted with ethyl acetate, and the organic phase separated, dried (MgSO₄), and evaporated to a solid. The solid was triturated with hexane to yield the title compound (0.673 g). LC/MS Rt=2.27 min [MH+] 167.

### Example 206

### N-[1-({5-Chloro-2-[(2-methylpropyl)oxy]phenyl}methyl)-5-methyl-1H-pyrazol-3-yl]-4-ethenyl-2-fluorobenzamide

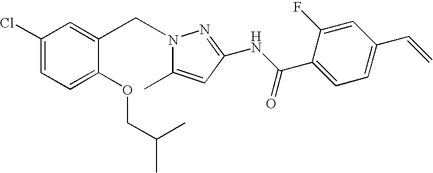

4-Ethenyl-2-fluorobenzoic acid (0.43 g, 2.5 mmol), N-(3-dimethylaminopropyl)-N′ethylcarbodiimide (0.497 g, 2.5 mmol) and 1-hydroxy-7-azabenzotriazole (0.353 g, 2.5 mmol) were stirred in dichloromethane (25 ml) for 90 min. 1-({5-Chloro-2-[(2-methylpropyl)oxy]phenyl}methyl)-5-methyl-1H-pyrazol-3-amine hydrochloride (0.57 g, 1.73 mmol) and triethylamine (0.361 ml, 2.5 mmol) were added and the mixture stirred for 3 hours. The reaction was diluted with ethyl acetate and washed with 2M hydrochloric acid, dried (MgSO₄), evaporated to a yellow oil and purified by flash chromatography, eluting with ethyl acetate:hexane (1:3) and dried in vacuo to yield the title compound (0.527 g). LC/MS Rt=3.86 min [MH+] 442 and 444

### Example 207

### N-[1-({5-Chloro-2-[(2-methylpropyl)oxy]phenyl}methyl)-5-methyl-1H-pyrazol-3-yl]-2-fluoro-4-formylbenzamide

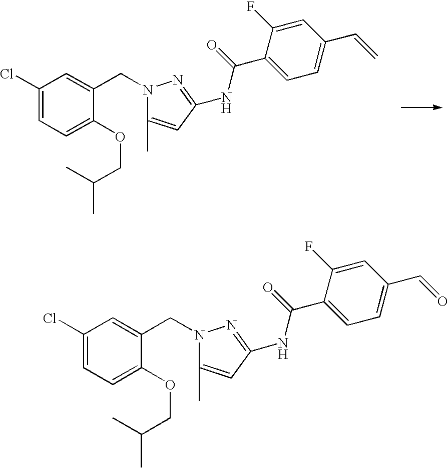

N-[1-({5-Chloro-2-[(2-methylpropyl)oxy]phenyl}methyl)-5-methyl-1H-pyrazol-3-yl]-4-ethenyl-2-fluorobenzamide (0.527 g, 1.2 mmol) was stirred in dichloromethane (25 ml) and cooled to −78° C. under argon. The solution was treated with ozone for 15 min then allowed to warm to room temperature. The mixture was stirred with polymer bound triphenylphosphine (3 mmol/g; 0.46 g, 1.38 mmol) for 1 hour. The reaction mixture was filtered and purified using flash chromatography eluting with ethyl acetate:hexane (1:3) and dried in vacuo to yield the title compound (0.322 g). LC/MS Rt=3.64 min, [MH+] 444, 446.

### N¹-[1-({5-Chloro-2-[(phenylmethyl)oxy]phenyl}methyl)-1H-pyrazol-3-yl]-N²-(2-hydroxyethyl)glycinamide

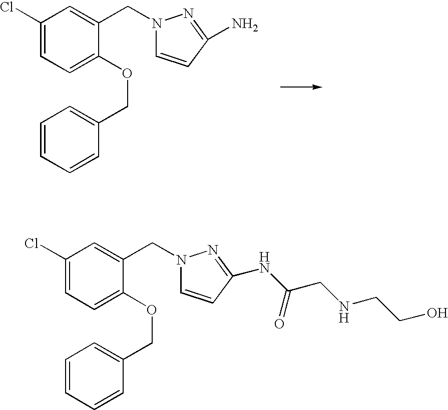

1-({5-Chloro-2-[(phenylmethyl)oxy]phenyl}methyl)-1H-pyrazol-3-amine (3 g, 9.57 mmol) was dissolved in isopropyl acetate (25 ml). This was treated with 20% potassium bicarbonate solution (25 ml) followed by chloroacetyl chloride (0.92 ml, 11.5 mmol). This was stirred at room temperature for 30 minutes under argon. The mixture was diluted with ethyl acetate and water and the layers separated. The aqueous phase was re-extracted with ethyl acetate, the extracts were dried (Na₂SO₄) and evaporated to a white solid. The solid was dissolved in isopropyl acetate (25 ml) and the solution treated with ethanolamine (2.65 ml, 47.8 mmol). The mixture was heated to 80° C. for 5 hours under argon, cooled, diluted with ethyl acetate and water and the layers separated. The aqueous phase was re-extracted with ethyl acetate, the extracts were dried (Na₂SO₄) and evaporated to a solid which was triturated with diethyl ether. The resulting white solid was collected by filtration (3.15 g, 79%). LC/MS Rt=2.18 min, [MH+] 415.

The following compounds were prepared in a similar manner to N′-[1-({5-chloro-2-[(phenylmethyl)oxy]phenyl}methyl)-1H-pyrazol-3-yl]-N²-(2-hydroxyethyl)glycinamide from the appropriate intermediates:

| | Compound name | Data |
|---|---|---|
| 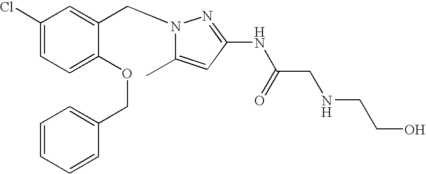 | N¹-[1-({5-Chloro-2- [(phenylmethyl)oxy]phenyl}methyl)-5- methyl-1H-pyrazol-3-yl]-N²-(2- hydroxyethyl)glycinamide | LC/MS Rt = 2.18 min, [MH+] 429. |
| 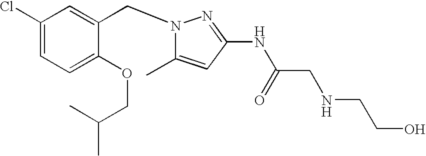 | N¹-[1-({5-Chloro-2-[(2- methylpropyl)oxy]phenyl}methy)-5- methyl-1H-pyrazol-3-yl]-N²-(2- hydroxyethyl)glycinamide | LC/MS Rt = 2.20 min, [MH+] 395. |
| 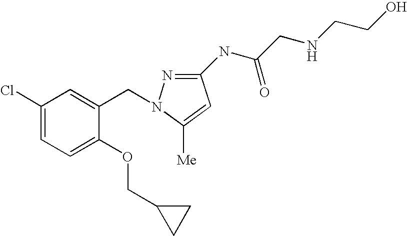 | N¹-[1-({5-Chloro-2- [(cyclopropylmethyl)oxy]phenyl}methyl)- 5-methyl-1H-pyrazol-3-yl]-N²-(2- hydroxyethyl)glycinamide | LC/MS Rt 1.92, [MH]⁺ 393 |

### 1-[1-({5-Chloro-2-[(phenylmethyl)oxy]phenyl}methyl)-1H-pyrazol-3-yl]-2-piperazinone

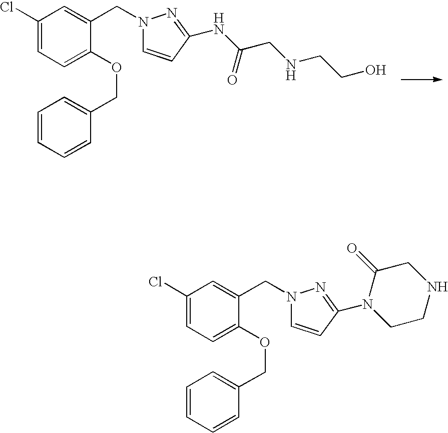

N¹-[1-({5-Chloro-2-[(phenylmethyl)oxy]phenyl}methyl)-1H-pyrazol-3-yl]-N²-(2-hydroxyethyl)glycinamide (3.15 g, 7.60 mmol) was dissolved in ethyl acetate (100 ml) and tetrahydrofuran (50 ml). To the solution was added triphenylphosphine (2.79 g, 10.6 mmol), followed by a solution of di-tert-butyldicarboxylate (2.45 g, 10.6 mmol) in ethyl acetate (25 ml). The mixture was stirred at room temperature for 3 hours. Further triphenylphosphine (1.39 g, 5.3 mmol) and di-tert-butyldicarboxylate (1.22 g, 5.3 mmol) was added to the reaction mixture and stirring continued for 1 hour. 4M Hydrogen chloride in 1,4-dioxane (19 ml, 76 mmol) was added to the reaction, but the hydrochloride salt did not precipitate out. This was therefore partitioned between ethyl acetate and 2M sodium hydroxide solution. The aqueous phase was re-extracted with ethyl acetate, the extracts were dried (Na₂SO₄) and evaporated to a yellow oil. This was purified using flash chromatography (0-25% methanol: ethyl acetate) to yield the title compound as a white foam. (2.45 g, 81%). LC/MS Rt=2.20 min, [MH+] 397.

The following compounds were prepared in a similar manner to 1-[1-({5-chloro-2-[(phenylmethyl)oxy]phenyl}methyl)-1H-pyrazol-3-yl]-2-piperazinone from the appropriate intermediates:

| | Compound name | Data |
|---|---|---|
| 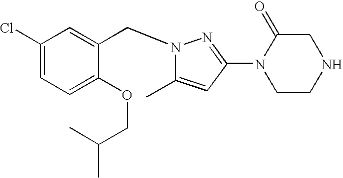 | 1-[1-({5-Chloro-2-[(2- methylpropyl)oxy]phenyl}methyl)-5- methyl-1H-pyrazol-3-yl]-2-piperazinone | LC/MS Rt = 2.26 min, [MH+] 377 |
| 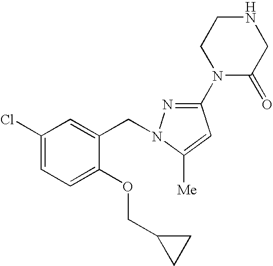 | 1-[1-({5-Chloro-2- [(cyclopropylmethyl)oxy]phenyl}methyl)- 5-methyl-1H-pyrazol-3-yl]-2- piperazinone | LC/MS Rt 2.09, [MH]⁺ 375, 377 |

### Example 208

### 1-[1-({5-Chloro-2-[(phenylmethyl)oxy]phenyl}methyl)-5-methyl-1H-pyrazol-3-yl]-4-(henylmethyl)-2-piperazinone

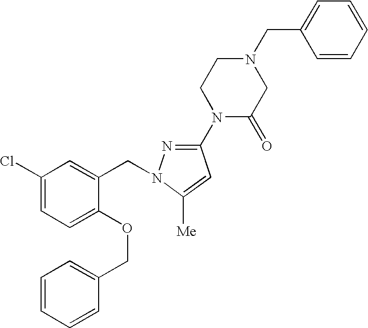

Benzyl bromide (57 mg, 0.33 mmol) was added to a solution of 1-[1-({5-chloro-2-[(phenylmethyl)oxy]phenyl}methyl)-5-methyl-1H-pyrazol-3-yl]-2-piperazinone (123 mg, 0.3 mmol) and diisopropylethylamine (52 mg, 0.4 mmol) in acetonitrile (3 ml). After 3 hours at room temperature the solution was diluted with ethyl acetate, washed with water and the organic phase dried (magnesium sulphate), evaporated and triturated with ether to give the title compound as a white solid (106 mg). LC/MS Rt 4.03, [MH]⁺ 501.4, 503.5.

### Example 209

### 1-[1-({5-Chloro-2-[(phenylmethyl)oxy]phenyl}methyl)-5-methyl-1H-pyrazol-3-yl]-4-methyl-2-piperazinone

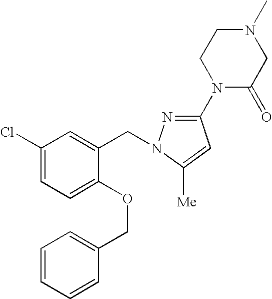

Iodomethane (43 mg, 0.33 mmol) was added to a solution of 1-[1-({5-chloro-2-[(phenylmethyl)oxy]phenyl}methyl)-5-methyl-1H-pyrazol-3-yl]-2-piperazinone (103 mg, 0.25 mmol) and diisopropylethylamine (52 mg, 0.4 mmol) in acetonitrile (2 ml). After 6 hours at room temperature the solution was diluted with ether, washed with water and the organic phase dried (magnesium sulphate), evaporated and triturated with ether/hexane to give the title compound as a white solid (26 mg). LC/MS Rt 3.12, [MH]⁺ 425.4, 427.4

### Example 210

### 4-[(4-Chlorophenyl)methyl]-1-[1-({5-chloro-2-[(phenylmethyl)oxy]phenyl}methyl)-5-methyl-1H-pyrazol-3-yl]-2-piperazinone hydrochloride

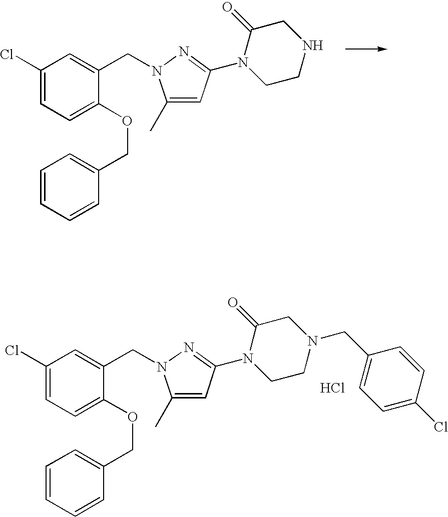

1-[1-({5-Chloro-2-[(phenylmethyl)oxy]phenyl}methyl)-1H-pyrazol-3-yl]-2-piperazinone (0.1 g, 0.24 mmol) was dissolved in acetonitrile (3 ml). To the solution was added 1-(bromomethyl)-4-chlorobenzene (0.055 g, 0.27 mmol), and N,N-diisopropylethylamine (0.056 ml, 0.032 mmol). The reaction mixture was stirred at room temperature for 5 hours. Water was added to the solution and the resulting white solid was collected by filtration and washed with water. The solid was dried under vacuum at 50° C. (75 mg). The solid (38 mg) was dissolved in dichloromethane (1 ml) and 1M hydrogen chloride in diethyl ether (0.50 ml, 0.50 mmol) was added. The residue was blown dry to yield a white solid (0.035 g).

LC/MS Rt=3.78 min, [MH+] 535

The following examples were prepared in a similar manner to 4-[(4-chlorophenyl)methyl]-1-[1-({5-chloro-2-[(phenylmethyl)oxy]phenyl}methyl)-5-methyl-1H-pyrazol-3-yl]-2-piperazinone hydrochloride from the appropriate intermediates.

| Example | Structure | Name | Data |
|---|---|---|---|
| 211 | 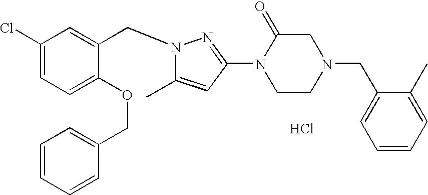 | 1-[1-({5-Chloro-2- [(phenylmethyl)oxy]phenyl}methyl)-5- methyl-1H-pyrazol-3- yl]-4-[(2-methylphenyl)methyl]- 2-piperazinone hydrochloride | LC/MS Rt = 3.76, [MH+] 515 |
| 212 | 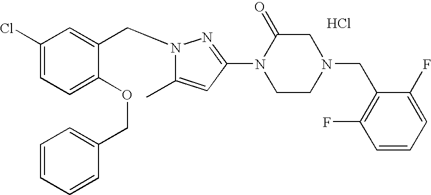 | 1-[1-({5-Chloro-2- [(phenylmethyl)oxy]phenyl}methyl)-5- methyl-1H-pyrazol-3- yl]-4-[(2-6-difluorophenyl)methyl]-2- piperazinone hydrochloride | LC/MS Rt = 3.67, [MH+] 537 |
| 213 | 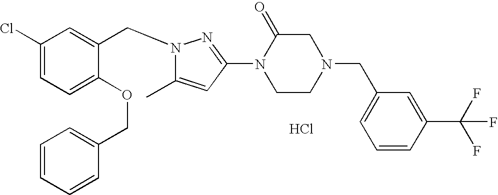 | 1-[1-({5-Chloro-2- [(phenylmethyl)oxy]phenyl}methyl)-5- methyl-1H-pyrazol-3- yl]-4-{[3- (trifluoromethyl)phenyl]methyl}-2- piperazinone hydrochloride | LC/MS Rt = 3.79, [MH+] 569 |
| 214 | 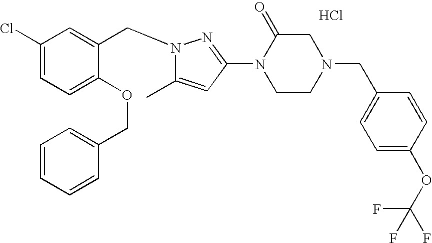 | 1-[1-({5-Chloro-2- [(phenylmethyl)oxy]phenyl}methyl)-5- methyl-1H-pyrazol-3- yl]-4-({4- [(trifluoromethyl)oxy]phenyl}methyl)- 2-piperazinone hydrochloride | LC/MS Rt = 3.80, [MH+] 585 |
| 215 | 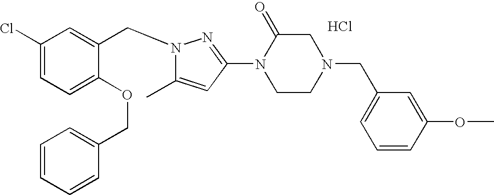 | 1-[1-({5-Chloro-2- [(phenylmethyl)oxy]phenyl}methyl)-5- methyl-1H-pyrazol-3- yl]-4-{[3- (methyloxy)phenyl]methyl}-2- piperazinone hydrochloride | LC/MS Rt = 3.44, [MH+] 531 |
| 216 | 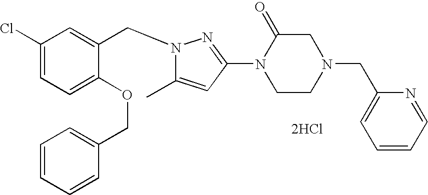 | 1-[1-({5-Chloro-2- [(phenylmethyl)oxy]phenyl}methyl)-5- methyl-1H-pyrazol-3- yl]-4-(2- pyridinylmethyl)-2- piperazinone hydrochloride | LC/MS Rt = 2.67, [MH+] 502 |
| 217 | 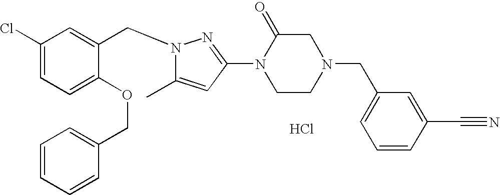 | 3-({4-[1-({5-Chloro-2- [(phenylmethyl)oxy]phenyl}methyl)-5- methyl-1H-pyrazol-3-yl]-3-oxo-1- piperazinyl}methyl)benzonitrile hydrochloride | LC/MS Rt = 3.54, [MH+] 526 |
| 218 | 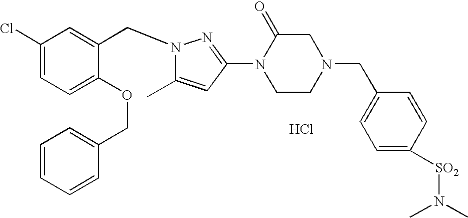 | 4-({4-[1-({5-Chloro-2- [(phenylmethyl)oxy]phenyl}methyl)-5- methyl-1H-pyrazol-3-yl]-3-oxo-1- piperazinyl}methyl)-N,N- dimethylbenzenesulfonamide hydrochloride | LC/MS Rt = 3.47, [MH+] 608 |
| 219 | 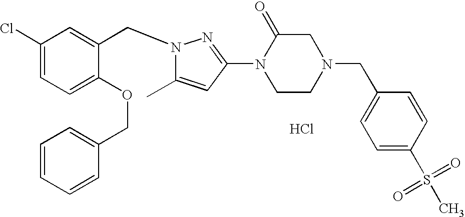 | 1-[1-({5-Chloro-2- [(phenylmethyl)oxy]phenyl}methyl)-5- methyl-1H-pyrazol-3- yl]-4-{[4- (methylsulfonyl)phenyl]methyl}-2- piperazinone hydrochloride | LC/MS Rt = 3.29, [MH+] 579 |
| 220 | 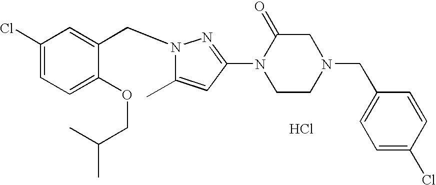 | 1-[1-({5-Chloro-2-[(2- methylpropyl)oxy]phenyl}methyl)-5- methyl-1H-pyrazol-3-yl]- 4-[(4-chlorophenyl)methyl]-2- piperazinone hydrochloride | LC/MS Rt = 3.92, [MH+] 501 |
| 221 | 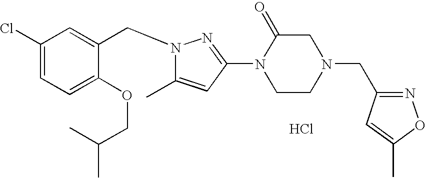 | 1-[1-({5-Chloro-2-[(2- methylpropyl)oxy]phenyl}methyl)-5- methyl-1H-pyrazol-3-yl]- 4-[(5-methyl-3 isoxazolyl)methyl]-2- piperazinone hydrochloride | LC/MS Rt = 3.46, [MH+] 472 |
| 222 | 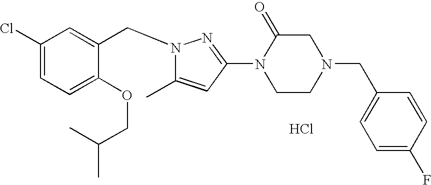 | 1-[1-({5-Chloro-2-[(2- methylpropyl)oxy]phenyl}methyl)-5- methyl-1H-pyrazol-3-yl]- 4-[(4-fluorophenyl)methyl]-2- piperazinone hydrochloride | LC/MS Rt = 3.67, [MH+] 485 |
| 223 | 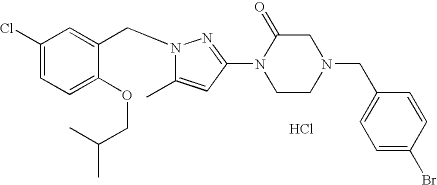 | 4-[(4-Bromophenyl)methyl]-1-[1-({5-Chloro-2-[(2- methylpropyl)oxy]phenyl}methyl)-5- methyl-1H-pyrazol-3-yl]-2- piperazinone hydrochloride | LC/MS Rt = 3.96, [MH+] 545, 547 |
| 224 | 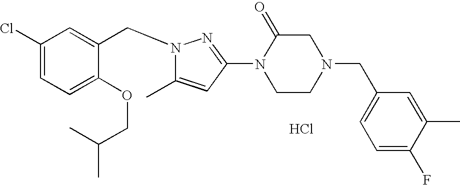 | 1-[1-({5-Chloro-2-[(2- methylpropyl)oxy]phenyl}methyl)-5- methyl-1H-pyrazol-3-yl]- 4-[(4-fluoro-3- methylphenyl)methyl]-2- piperazinone hydrochloride | LC/MS Rt = 3.80, [MH+] 499 |
| 225 | 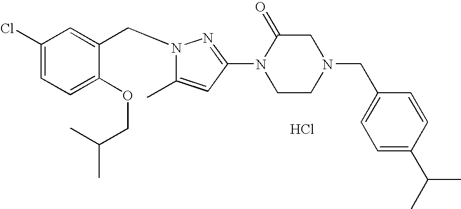 | 1-[1-({5-Chloro-2-[(2- methylpropyl)oxy]phenyl}methyl)-5- methyl-1H-pyrazol-3-yl]- 4-{[4-(1- methylethyl)phenyl]methyl}-2- piperazinone hydrochloride | LC/MS Rt = 4.00, [MH+] 509 |
| 226 | 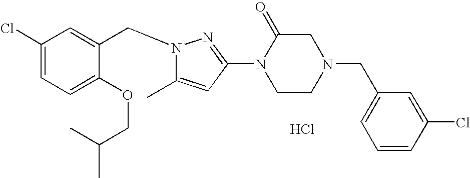 | 1-[1-({5-Chloro-2-[(2- methylpropyl)oxy]phenyl}methyl)-5- methyl-1H-pyrazol-3-yl]- 4-[(3-chlorophenyl)methyl]-2- piperazinone hydrochloride | LC/MS Rt = 3.93, [MH+] 501 |
| 227 | 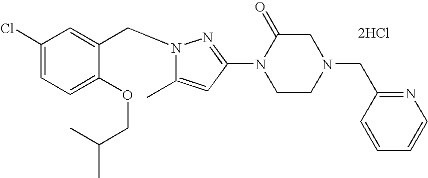 | 1-[1-({5-Chloro-2-[(2- methylpropyl)oxy]phenyl}methyl)-5- methyl-1H-pyrazol-3-yl]- 4-(2-pyridinylmethyl)-2- piperazinone dihydrochloride | LC/MS Rt = 2.76, [MH+] 468 |
| 228 | 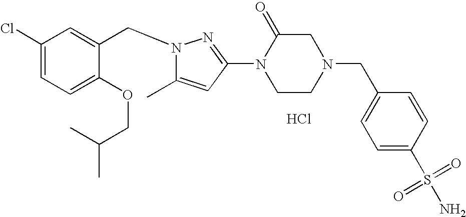 | 4-({4-[1-({5-Chloro-2-[(2- methylpropyl)oxy]phenyl}methyl)-5- methyl-1H-pyrazol-3-yl]- 3-oxo-1- piperazinyl}methyl)benzenesulfonamide hydrochloride | LC/MS Rt = 3.17, [MH+] 544 |
| 229 | 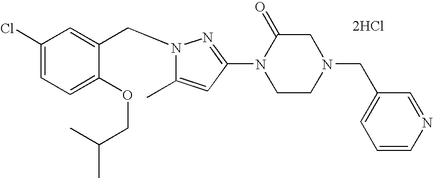 | 1-[1-({5-Chloro-2-[(2- methylpropyl)oxy]phenyl}methyl)-5- methyl-1H-pyrazol-3-yl]- 4-(3-pyridinylmethyl)-2- piperazinone dihydrochloride | LC/MS Rt = 2.86, [MH+] 468 |
| 230 | 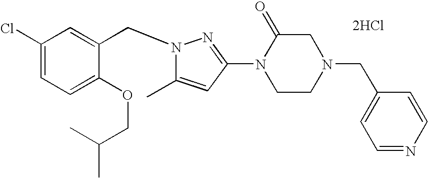 | 1-[1-({5-Chloro-2-[(2- methylpropyl)oxy]phenyl}methyl)-5- methyl-1H-pyrazol-3-yl]- 4-(4-pyridinylmethyl)-2- piperazinone dihydrochloride | LC/MS Rt = 2.73, [MH+] 468 |
| 231 | 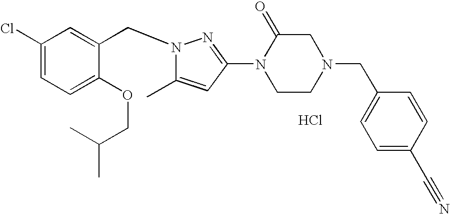 | 4-({4-[1-({5-Chloro-2-[(2- methylpropyl)oxy]phenyl}methyl)-5- methyl-1H-pyrazol-3-yl]- 3-oxo-1- piperazinyl}methyl)benzonitrile hydrochloride | LC/MS Rt = 3.62, [MH+] 492 |
| 232 | 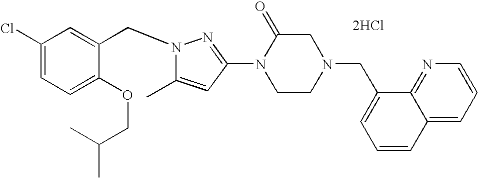 | 1-[1-({5-Chloro-2-[(2- methylpropyl)oxy]phenyl}methyl)-5- methyl-1H-pyrazol-3-yl]- 4-(8-quinolinylmethyl)-2- piperazinone dihydrochloride | LC/MS Rt = 3.80, [MH+] 518 |
| 233 | 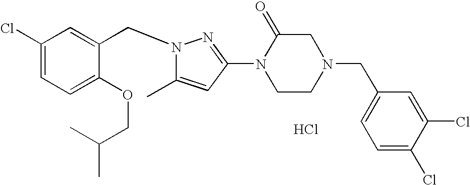 | 1-[1-({5-Chloro-2-[(2- methylpropyl)oxy]phenyl}methyl)-5- methyl-1H-pyrazol-3-yl]- 4-[(3,4- dichlorophenyl)methyl]-2- piperazinone hydrochloride | LC/MS Rt = 4.08, [MH+] no ion |
| 234 | 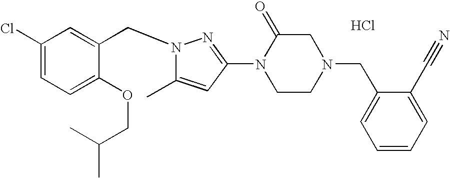 | 2-({4-[1-({5-Chloro-2-[(2- methylpropyl)oxy]phenyl}methyl)-5- methyl-1H-pyrazol-3-yl]- 3-oxo-1- piperazinyl}methyl)benzonitrile hydrochloride | LC/MS Rt = 3.65, [MH+] 492 |
| 235 | 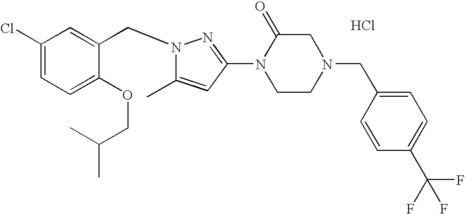 | 1-[1-({5-Chloro-2-[(2- methylpropyl)oxy]phenyl}methyl)-5- methyl-1H-pyrazol-3-yl]- 4-{[4- trifluoromethyl)phenyl]methyl}-2- piperazinone hydrochloride | LC/MS Rt = 3.86, [MH+] 535 |
| 236 | 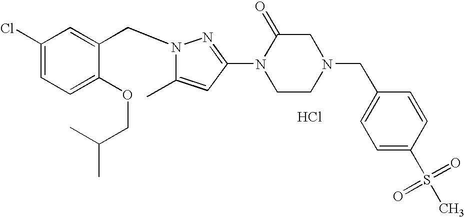 | 1-[1-({5-Chloro-2-[(2- methylpropyl)oxy]phenyl}methyl)-5- methyl-1H-pyrazol-3-yl]- 4-{[4- methylsulfonyl)phenyl]methyl}-2- piperazinone hydrochloride | LC/MS Rt = 3.37, [MH+] 545 |
| 237 | 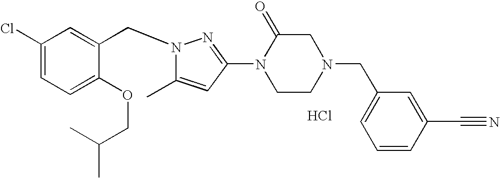 | 3-({4-[1-({5-Chloro-2-[(2- methylpropyl)oxy]phenyl}methyl)-5- methyl-1H-pyrazol-3-yl]- 3-oxo-1- piperazinyl}methyl)benzonitrile hydrochloride | LC/MS Rt = 3.61, [MH+] 492 |
| 238 | 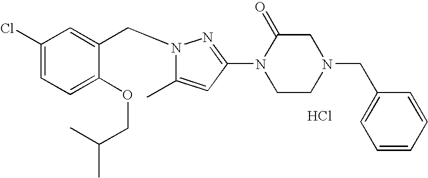 | 1-[1-({5-Chloro-2-[(2- methylpropyl)oxy]phenyl}methyl)-5- methyl-1H-pyrazol-3-yl]- 4-(phenylmethyl)-2- piperazinone hydrochloride | LC/MS Rt = 3.72, [MH+] 467 |

### Example 239

### 4-({4-[1-({5-Chloro-2-[(cyclopropylmethyl)oxy]phenyl}methyl)-5-methyl-1H-pyrazol-3-yl]-3-oxo-1-piperazinyl}methyl)-N,N-dimethylbenzene-sulfonamide

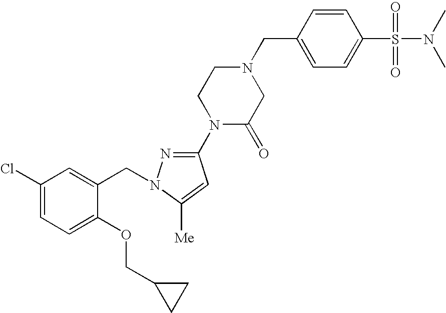

4-(Bromomethyl)-N,N-dimethylbenzenesulfonamide (65 mg, 0.23 mmol) was added to a solution of 1-[1-({5-chloro-2-[(cyclopropylmethyl)oxy]phenyl}methyl)-5-methyl-1H-pyrazol-3-yl]-2-piperazinone (80 mg, 0.21 mmol) and triethylamine (38 μl, 0.27 mmol) in acetonitrile (3 ml). After 3 hours at room temperature the solution was diluted with ethyl acetate, washed with water and the organic phase dried (magnesium sulphate) and evaporated. The residue was then purified by flash chromatography using 60% ethyl acetate in hexane to give the title compound. LC/MS Rt 3.46, [MH]⁺ 572, 574.

The following compounds were prepared in a similar manner to 4-({4-[1-({5-chloro-2-[(cyclopropylmethyl)oxy]phenyl}methyl)-5-methyl-1H-pyrazol-3-yl]-3-oxo-1-piperazinyl}methyl)-N,N-dimethylbenzenesulfonamide from the appropriate intermediates:

| Example | Structure | Compound name | Data |
|---|---|---|---|
| 240 | 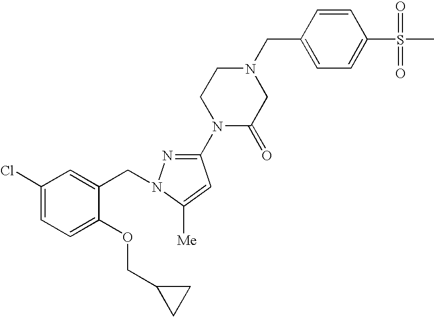 | 1-[1-({5-Chloro-2- [(cyclopropylmethyl)oxy]phenyl}methyl)- 5-methyl-1H-pyrazol-3-yl]-4-{[4- (methylsulfonyl)phenyl]methyl}-2- piperazinone | LC/MS Rt = 3.22, [MH]⁺ 543, 545 |
| 241 | 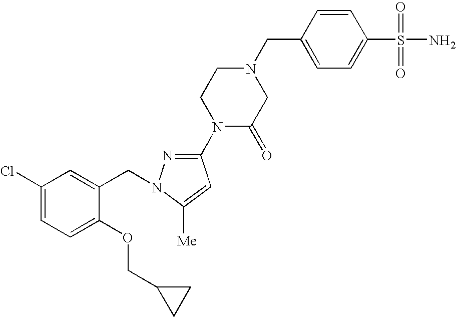 | 4-({4-[1-({5-Chloro-2- [(cyclopropylmethyl)oxy]phenyl}methyl)- 5-methyl-1H-pyrazol-3-yl]-3-oxo-1- piperazinyl}methyl)benzenesulfonamide | LC/MS Rt = 2.98, [MH]⁺ 544, 546 |
| 242 | 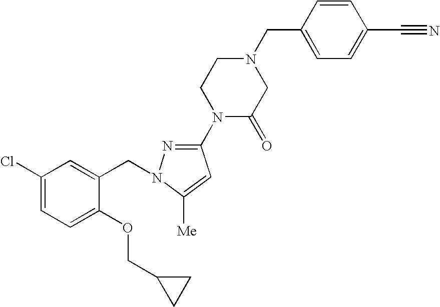 | 4-({4-[1-({5-Chloro-2- [(cyclopropylmethyl)oxy]phenyl}methyl)- 5-methyl-1H-pyrazol-3-yl]-3-oxo-1- piperazinyl}methyl)benzonitrile | LC/MS Rt = 3.56 [MH]⁺ 490, 492 |
| 243 | 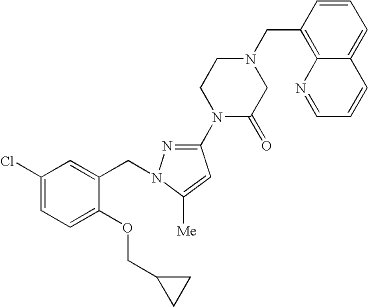 | 1-[1-({5-Chloro-2- [(cyclopropylmethyl)oxy]phenyl}methyl)- 5-methyl-1H-pyrazol-3-yl]-4-(8- (quinolinylmethyl)-2- piperazinone | LC/MS Rt = 2.62 [MH]⁺ 516, 518 |

### 2-[(1-Methylethyl)oxy]ethyl 4-methylbenzenesulfonate

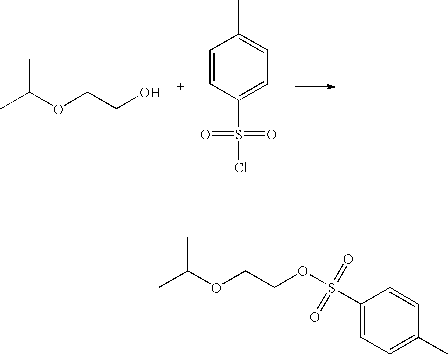

To a solution of 2-[(1-methylethyl)oxy]ethanol (2.0 g, 19.2 mmol) in dichloromethane (25 ml) was added pyridine (1.55 ml, 19.2 mmol) and 4-methylbenzenesulfonyl chloride (3.66 g, 19.2 mmol) under an argon atmosphere. The reaction was stirred at room temperature for 3 hours. The reaction was evaporated and purified using flash chromatography (0-50% ethyl acetate:hexane) to yield the title compound as a clear oil (2.98 g, 60%).

### Example 244

### 1-[1-({5-Chloro-2-[(phenylmethyl)oxy]phenyl}methyl)-5-methyl-1H-pyrazol-3-yl]-4-(2-methylpropyl)-2-piperazinone hydrochloride

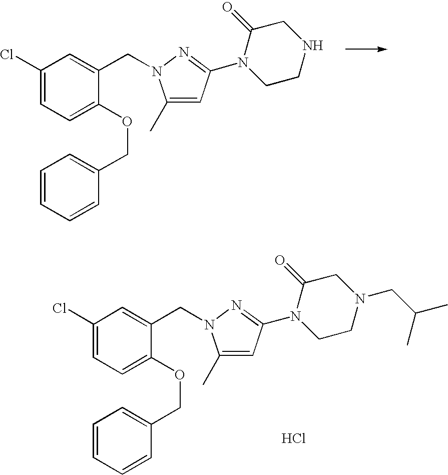

1-[1-({5-Chloro-2-[(phenylmethyl)oxy]phenyl}methyl)-1H-pyrazol-3-yl]-2-piperazinone (0.1 g, 0.24 mmol) was dissolved in N,N-dimethylformamide (2 ml). To the solution was added 1-bromo-2-methylpropane (0.079 ml, 0.73 mmol) and potassium carbonate (0.067 g, 0.49 mmol). The mixture was heated to 180° C. in a Smithcreator® microwave for 1 hour. Water and diethyl ether were added and the layers separated. The aqueous phase was re-extracted with diethyl ether, the extracts combined and washed with water then dried (Na₂SO₄) and evaporated to a yellow oil. This was purified using flash chromatography (0-15% ethyl acetate:hexane). The product was dissolved in dichloromethane (1 ml) and 1M hydrogen chloride in diethyl ether (0.50 ml, 0.50 mmol) was added. The residue was blown dry to yield a cream solid (0.022 g). LC/MS Rt=2.79, [MH+] 467.

The following examples were prepared in a similar manner to 1-[1-({5-chloro-2-[(phenyl methyl)oxy]phenyl}methyl)-5-methyl-1H-pyrazol-3-yl]-4-(2-methylpropyl)-2-piperazinone hydrochloride from the appropriate intermediates.

| Example | | Name | Data |
|---|---|---|---|
| 245 | 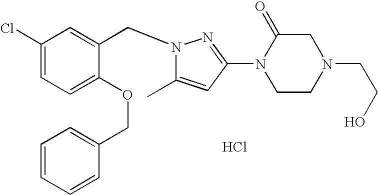 | 1-[1-({5-Chloro-2- [(phenylmethyl)oxy]phenyl}methyl)-5- methyl-1H-pyrazol-3-yl]-4-(2- hydroxyethyl)-2-piperazinone hydrochloride | LC/MS Rt = 2.34, [MH+] 455 |
| 246 | 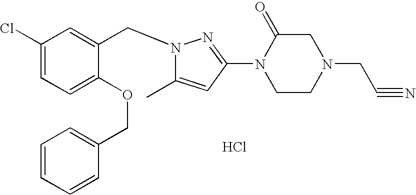 | {4-[1-({5-Chloro-2- [(phenylmethyl)oxy]phenyl}methyl)-5- methyl-1H-pyrazol-3-yl]-3-oxo-1- piperazinyl}acetonitrile hydrochloride | LC/MS Rt = 3.27, [MH+] 450 |
| 247 | 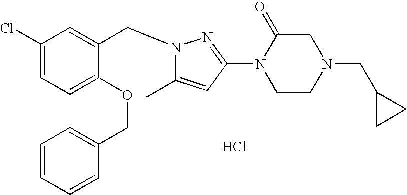 | 1-[1-({5-Chloro-2- [(phenylmethyl)oxy]phenyl}methyl)-5- methyl-1H-pyrazol-3-yl]-4- (cyclopropylmethyl)-2-piperazinone hydrochloride | LC/MS Rt = 2.47, [MH+] 465 |
| 248 | 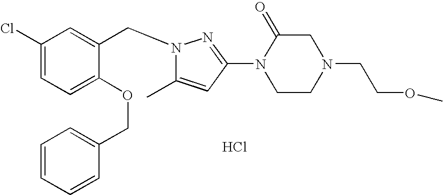 | 1-[1-({5-chloro-2- [(phenylmethyl)oxy]phenyl}methyl)-5- methyl-1H-pyrazol-3-yl]-4-[2- (methyloxy)ethyl]-2-piperazinone hydrochloride | LC/MS Rt = 2.59, [MH+] 469 |
| 249 | 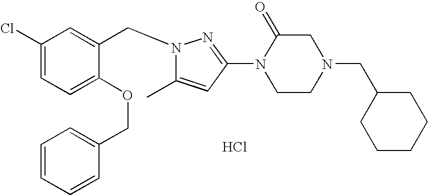 | 1-[1-({5-Chloro-2- [(phenylmethyl)oxy]phenyl}methyl)-5- methyl-1H-pyrazol-3-yl]-4- (cyclohexylmethyl)-2-piperazinone hydrochloride | LC/MS Rt = 3.14, [MH+] 507 |
| 250 | 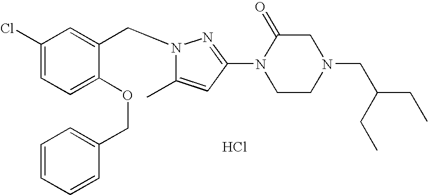 | 1-[1-({5-Chloro-2- [(phenylmethyl)oxy]phenyl}methyl)-5- methyl-1H-pyrazol-3-yl]-4- (2-ethylbutyl)-2-piperazinone hydrochloride | LC/MS Rt = 3.27, [MH+] 495 |
| 251 | 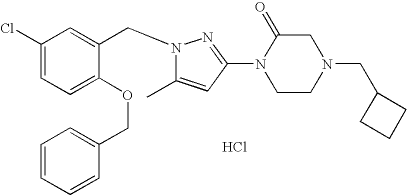 | 1-[1-({5-Chloro-2- [(phenylmethyl)oxy]phenyl}methyl)-5- methyl-1H-pyrazol-3-yl]-4- (cyclobutylmethyl)-2-piperazinone hydrochloride | LC/MS Rt = 2.69, [MH+] 479 |
| 252 | 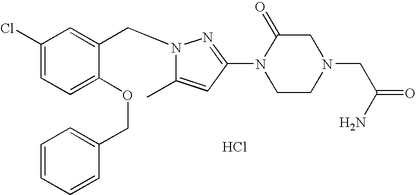 | 2-{4-[1-({5-Chloro-2- [(phenylmethyl)oxy]phenyl}methyl)-5- methyl-1H-pyrazol-3-yl]-3-oxo-1- piperazinyl}acetamide hydrochloride | LC/MS Rt = 2.85, [MH+] 468 |
| 253 | 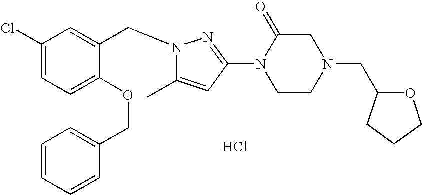 | 1-[1-({5-Chloro-2- [(phenylmethyl)oxy]phenyl}methyl)-5- methyl-1H-pyrazol-3-yl]-4- (tetrahydro-2-furanylmethyl)-2- piperazinone hydrochloride | LC/MS Rt = 3.54, [MH+] 495 |
| 254 | 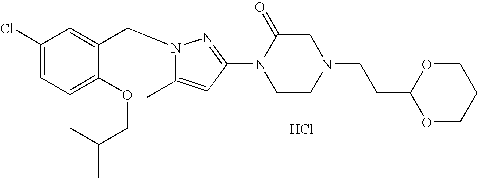 | 1-[1-({5-Chloro-2-[(2- methylpropyl)oxy]phenyl}methyl)-5- methyl-1H-pyrazol-3-yl]-4- [2-(1,3-dioxan-2-yl)ethyl]-2-piperazinone hydrochloride | LC/MS Rt = 2.62, [MH+] 491 |
| 255 | 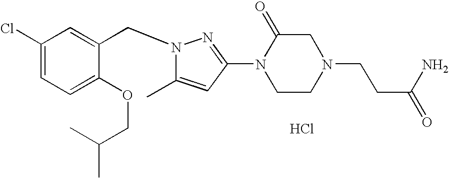 | 3-{4-[1-({5-Chloro-2-[(2- methylpropyl)oxy]phenyl}methyl)-5- methyl-1H-pyrazol-3-yl]-3-oxo-1- piperazinyl}propanamide hydrochloride | LC/MS Rt = 2.43, [MH+] 448 |
| 256 | 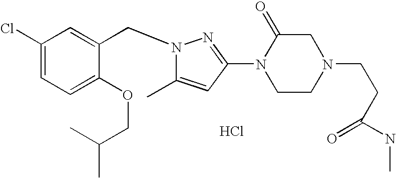 | 3-{4-[1-({5-Chloro-2-[(2- methylpropyl)oxy]phenyl}methyl)-5- methyl-1H-pyrazol-3-yl]-3-oxo-1- piperazinyl}-N-methylpropanamide hydrochloride | LC/MS Rt = 3.51, [MH+] 462 |
| 257 | 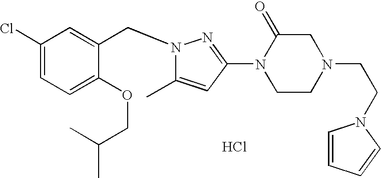 | 1-[1-({5-Chloro-2-[(2- methylpropyl)oxy]phenyl}methyl)-5- methyl-1H-pyrazol-3-yl]-4- [2-(1H-pyrrol-1-yl)ethyl]-2-piperazinone hydrochloride | LC/MS Rt = 3.50, [MH+] 470 |
| 258 | 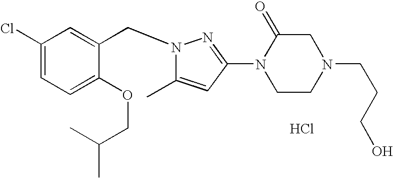 | 1-[1-({5-Chloro-2-[(2- methylpropyl)oxy]phenyl}methyl)-5- methyl-1H-pyrazol-3-yl]-4- (3-hydroxypropyl)-2-piperazinone hydrochloride | LC/MS Rt = 2.34, [MH+] 435 |
| 259 | 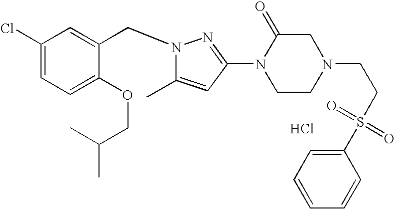 | 1-[1-({5-Chloro-2-[(2- methylpropyl)oxy]phenyl}methyl)-5- methyl-1H-pyrazol-3-yl]-4- [2-(phenylsulfonyl)ethyl]-2-piperazinone hydrochloride | LC/MS Rt = 3.43, [MH+] 545 |
| 260 | 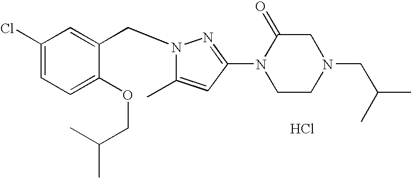 | 1-[1-({5-Chloro-2-[(2- methylpropyl)oxy]phenyl}methyl)-5- methyl-1H-pyrazol-3-yl]-4- (2-methylpropyl)-2-piperazinone hydrochloride | LC/MS Rt = 2.87, [MH+] 433 |
| 261 | 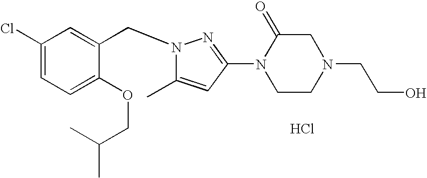 | 1-[1-({5-Chloro-2-[(2- methylpropyl)oxy]phenyl}methyl)-5- methyl-1H-pyrazol-3-yl]-4- (2-hydroxyethyl)-2-piperazinone hydrochloride | LC/MS Rt = 2.37, [MH+] 421 |
| 262 | 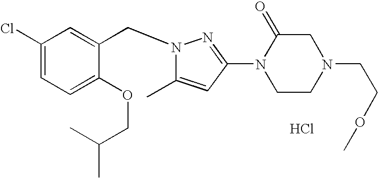 | 1-[1-({5-Chloro-2-[(2- methylpropyl)oxy]phenyl}methyl)-5- methyl-1H-pyrazol-3-yl]-4- [2-(methyloxy)ethyl]-2-piperazinone hydrochloride | LC/MS Rt = 2.60, [MH+] 435 |
| 263 | 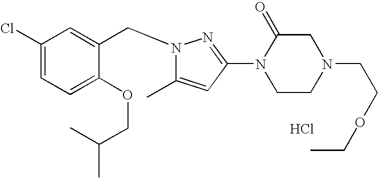 | 1-[1-({5-Chloro-2-[(2- methylpropyl)oxy]phenyl}methyl)-5- methyl-1H-pyrazol-3-yl]-4- [2-(ethyloxy)ethyl]-2-piperazinone hydrochloride | LC/MS Rt = 2.70, [MH+] 449 |
| 264 | 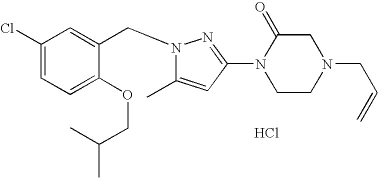 | 1-[1-({5-Chloro-2-[(2- methylpropyl)oxy]phenyl}methyl)-5- methyl-1H-pyrazol-3-yl]-4- (2-propen-1-yl)-2-piperazinone hydrochloride | LC/MS Rt = 2.84, [MH+] 417 |
| 265 | 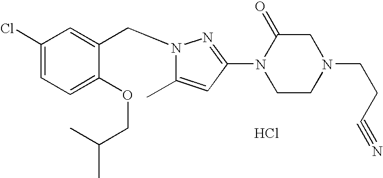 | 3-{4-[1-({5-Chloro-2-[(2- methylpropyl)oxy]phenyl}methyl)-5- methyl-1H-pyrazol-3-yl]-3-oxo-1- piperazinyl}propanenitrile hydrochloride | LC/MS Rt = 3.28, [MH+] 430 |
| 266 | 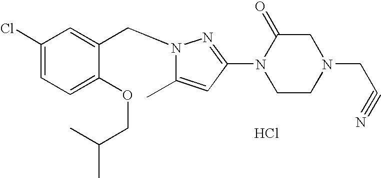 | {4-[1-({5-Chloro-2-[(2- methylpropyl)oxy]phenyl}methyl)-5- methyl-1H-pyrazol-3-yl]-3-oxo-1- piperazinyl}acetonitrile hydrochloride | LC/MS Rt = 3.33, [MH+] 416 |
| 267 | 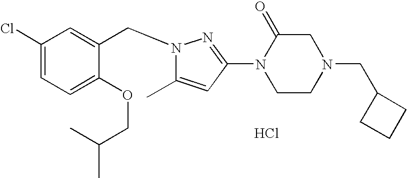 | 1-[1-({5-Chloro-2-[(2- methylpropyl)oxy]phenyl}methyl)-5- methyl-1H-pyrazol-3-yl]-4- (cyclobutylmethyl)-2-piperazinone hydrochloride | LC/MS Rt = 2.66, [MH+] 445 |
| 268 | 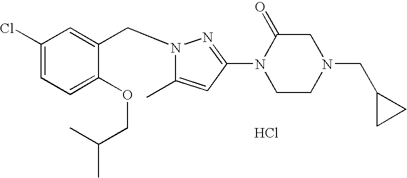 | 1-[1-({5-Chloro-2-[(2- methylpropyl)oxy]phenyl}methyl)-5- methyl-1H-pyrazol-3-yl]-4- (cyclopropylmethyl)-2-piperazinone hydrochloride | LC/MS Rt = 2.47, [MH+] 431 |
| 269 | 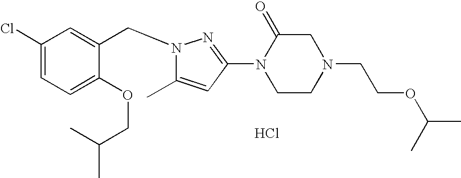 | 1-[1-({5-Chloro-2-[(2- methylpropyl)oxy]phenyl}methyl)-5- methyl-1H-pyrazol-3-yl]-4-{2-[(1- methylethyl)oxy]ethyl}-2-piperazinone hydrochloride | LC/MS Rt = 3.03, [MH+] 463 |
| 270 | 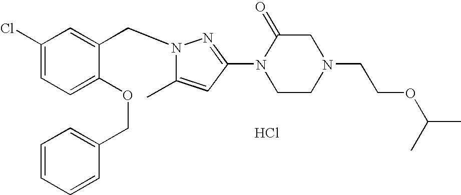 | 1-[1-({5-Chloro-2- [(phenylmethyl)oxy]phenyl}methyl)-5- methyl-1H-pyrazol-3-yl]-4-{2-[(1- methylethyl)oxy]ethyl}-2-piperazinone hydrochloride | LC/MS Rt = 2.94, [MH+] 497 |

### 1-{1-[(5-Chloro-2-hydroxyphenyl)methyl]-5-methyl-1H-pyrazol-3-yl}-4-(2-methylpropyl)-2-piperazinone

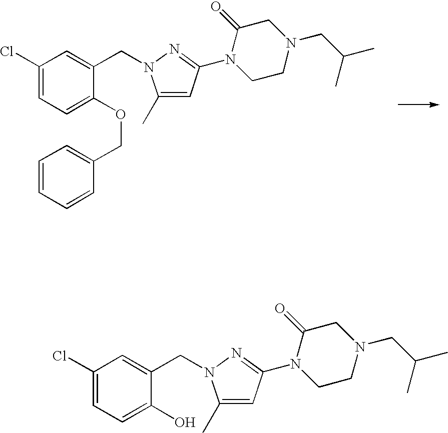

To a solution of 1-[1-({5-chloro-2-[(phenylmethyl)oxy]phenyl}methyl)-5-methyl-1H-pyrazol-3-yl]-4-(2-methylpropyl)-2-piperazinone (3.21 g, 6.88 mmol) in dichloromethane (200 ml) cooled to −78° C. under argon, was added 1M boron tribromide in dichloromethane (13.8 ml, 13.8 mmol) dropwise. The reaction mixture was allowed to warm to room temperature then quenched with water, diluted with dichloromethane and separated. The aqueous layer was re-extracted twice with dichloromethane. The combined organic extracts were dried (Na₂SO₄), concentrated and then purified using flash chromatography (0-70% ethyl acetate:hexane) to yield the title compound as a white solid (2.98 g).

LC/MS Rt=2.25, [MH+] 377

### 1-{1-[(5-Chloro-2-hydroxyphenyl)methyl]-5-methyl-1H-pyrazol-3-yl}-4-(2-pyridinylmethyl)-2-piperazinone

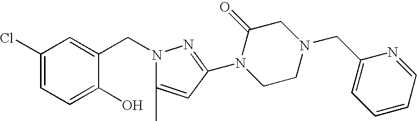

The title compound was prepared in a similar manner to 1-{1-[(5-chloro-2-hydroxyphenyl)methyl]-5-methyl-1H-pyrazol-3-yl}-4-(2-methylpropyl)-2-piperazinone from the appropriate intermediates. LC/MS Rt=2.20 min, [MH+] 412.

### 1-{1-[(5-Chloro-2-hydroxyphenyl)methyl]-5-methyl-1H-pyrazol-3-yl}-4-[(4-chlorophenyl)methyl]-2-piperazinone

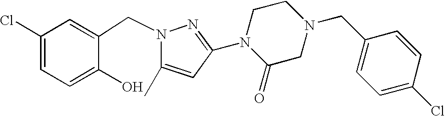

4-[(4-Chlorophenyl)methyl]-1-[1-({5-chloro-2-[(phenylmethyl)oxy]phenyl}methyl)-5-methyl-1H-pyrazol-3-yl]-2-piperazinone (844 mg, 1.6 mmol) was dissolved in 5 ml of hydrogen bromide in acetic acid (45% w/v) and stirred at room temperature during 18 hours under argon. The mixture was diluted with water and neutralized with sodium hydrogen carbonate saturated solution to pH 6-7. The aqueous phase was extracted with ethyl acetate, the extracts were dried (MgSO₄) and evaporated to give a yellow solid. This was purified by flash chromatography eluting with ethyl acetate/hexane to yield the title compound as a white solid (466 mg). LC/MS Rt=2.87, [MH+] 445.3 447.3.

### Example 271

### 1-[1-({5-Chloro-2-[(2-methyl-2-propen-1-yl)oxy]phenyl}methyl)-5-methyl-1H-pyrazol-3-yl]-4-(2-methylpropyl)-2-piperazinone hydrochloride

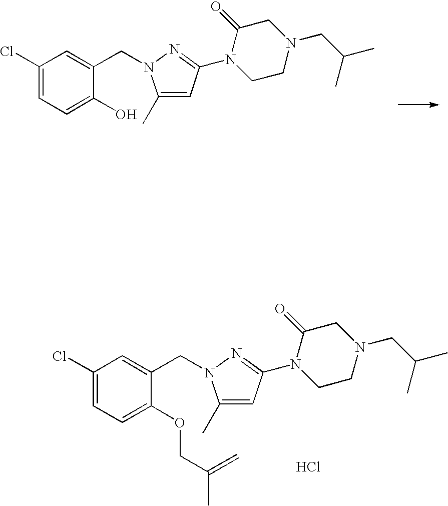

To a solution of 1-{1-[(5-chloro-2-hydroxyphenyl)methyl]-5-methyl-1H-pyrazol-3-yl}-4-(2-methylpropyl)-2-piperazinone (0.10 g, 0.27 mmol) in N,N-dimethylformamide (3 ml) was added 3-bromo-2-methyl-1-propene (0.039 g, 0.29 mmol) and potassium carbonate (0.092 g, 0.66 mmol). The mixture was heated to 90° C. under argon for 1 hour then partitioned between diethyl ether and water. The aqueous layer was re-extracted with diethyl ether. The combined extracts were washed with water, dried (Na₂SO₄), concentrated and then purified using flash chromatography (0-20% ethyl acetate:hexane) to yield a clear oil. The oil was dissolved in dichloromethane (1 ml) and 1M hydrogen chloride in diethyl ether (0.50 ml, 0.50 mmol) was added. The residue was blown dry to yield a white solid (0.10 g, 81%). LC/MS Rt=2.77, [MH+] 431.

The following examples were prepared in a similar manner to 1-[1-({5-chloro-2-[(2-methyl-2-propen-1-yl)oxy]phenyl}methyl)-5-methyl-1H-pyrazol-3-yl]-4-(2-methylpropyl)-2-piperazinone hydrochloride from the appropriate intermediates.

| Example | | Name | Data |
|---|---|---|---|
| 272 | 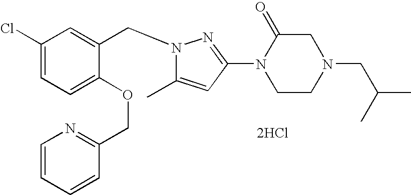 | 1-[1-({5-Chloro-2-[(2- pyridinylmethyl)oxy]phenyl} methyl)-5-methyl-1H-pyrazol-3-yl]-4-(2- methylpropyl)-2-piperazinone dihydrochloride | LC/MS Rt = 2.40, [MH+] 468 |
| 273 | 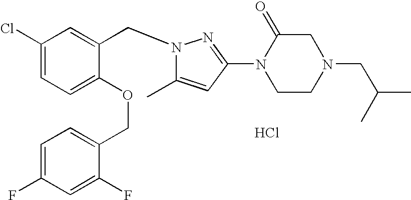 | 1-{1-[(5-Chloro-2-{[(2,4- difluorophenyl)methyl]oxy}phenyl)methyl]-5- methyl-1H-pyrazol-3-yl}-4-(2- methylpropyl)-2-piperazinone hydrochloride | LC/MS Rt = 3.03, [MH+] 503 |
| 274 | 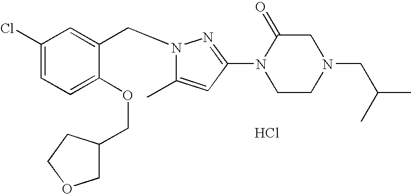 | 1-[1-({5-Chloro-2-[(tetrahydro-3- furanylmethyl)oxy]phenyl}methyl)-5- methyl-1H-pyrazol-3-yl]-4-(2- methylpropyl)-2-piperazinone hydrochloride | LC/MS Rt = 2.52, [MH+] 461 |
| 275 | 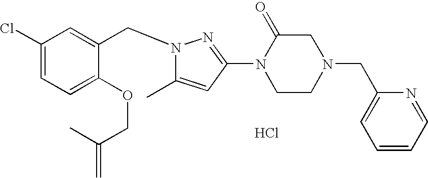 | 1-[1-({5-Chloro-2-[(2-methyl- 2-propen-1-yl)oxy]phenyl}methyl)-5- methyl-1H-pyrazol-3-yl]-4-(2- pyridinylmethyl)-2-piperazinone dihydrochloride | LC/MS Rt = 2.76, [MH+] 466 |
| 276 | 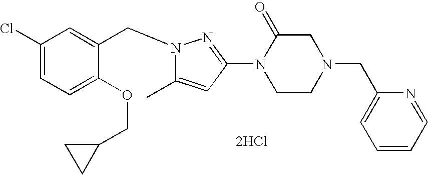 | 1-[1-({5-Chloro-2- [(cyclopropylmethyl)oxy]phenyl}methyl)-5- methyl-1H-pyrazol-3-yl]-4-(2- pyridinylmethyl)-2-piperazinone dihydrochloride | LC/MS Rt = 2.67, [MH+] 466 |
| 277 | 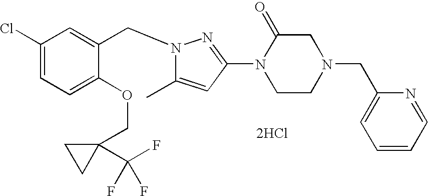 | 1-(1-{[5-Chloro-2-({[1- (trifluoromethyl)cyclopropyl] methyl}oxy)phenyl]methyl}- 5-methyl-1H-pyrazol-3-yl)-4-(2- pyridinylmethyl)-2-piperazinone dihydrochloride | LC/MS Rt = 2.78, [MH+] 534 |
| 278 | 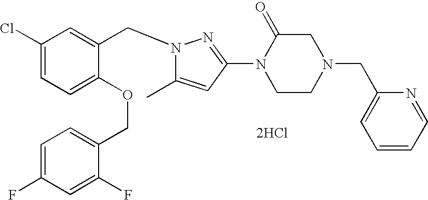 | 1-{1-[(5-Chloro-2-{[(2,4- difluorophenyl)methyl]oxy}phenyl)methyl]-5- methyl-1H-pyrazol-3-yl}-4-(2- pyridinylmethyl)-2-piperazinone dihydrochloride | LC/MS Rt = 2.83, [MH+] 538 |
| 279 | 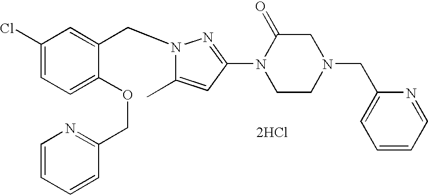 | 1-[1-({5-Chloro-2-[(2- pyridinylmethyl)oxy]phenyl} methyl)-5-methyl-1H-pyrazol-3-yl]-4-(2- pyridinylmethyl)-2-piperazinone dihydrochloride | LC/MS Rt = 2.38, [MH+] 503 |
| 280 | 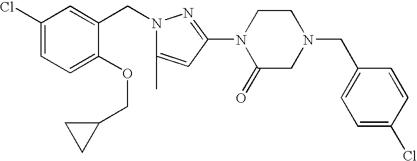 | 1-[1-({5-Chloro-2- [(cyclopropylmethyl)oxy]phenyl}methyl)-5- methyl-1H-pyrazol-3-yl]-4-[(4- chlorophenyl)methyl]-2-piperazinone | LC/MS Rt = 3.80, [MH]⁺ 499.2, 502.2 |
| 281 | 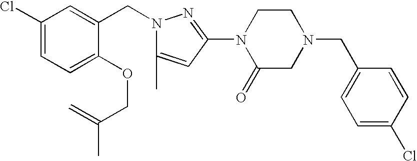 | 1-[1-({5-Chloro-2-[(2-methyl- 2-propen-1-yl)oxy]phenyl}methyl)-5- methyl-1H-pyrazol-3-yl]-4- [(4-chlorophenyl)methyl]-2-piperazinone | LC/MS Rt = 3.80 [MH]⁺ 499.2, 502.2 |
| 282 | 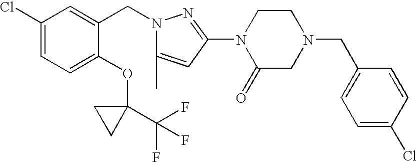 | 4-[(4-Chlorophenyl)methyl]- 1-(1-{[5-chloro-2-({[1- (trifluoromethyl)cyclopropyl] methyl}oxy)phenyl]methyl}- 5-methyl-1H-pyrazol-3-yl)-2- piperazinone | LC/MS Rt = 3.72 [MH]⁺ 567.2, 570.2 |
| 283 | 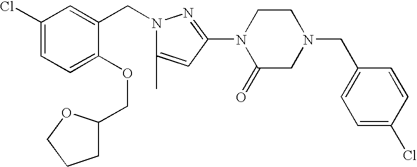 | 4-[(4-Chlorophenyl)methyl]- 1-[1-({5-chloro-2-[(tetrahydro-2- furanylmethyl)oxy]phenyl}methyl)-5- methyl-1H-pyrazol-3-yl]-2- piperazinone | LC/MS Rt = 3.29 [MH]⁺ 529.2, |
| 284 | 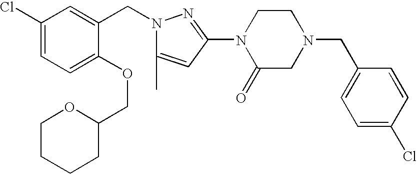 | 4-[(4-Chlorophenyl)methyl]- 1-[1-({5-chloro-2-[(tetrahydro-2H-pyran-2- ylmethyl)oxy]phenyl}methyl)-5-methyl- 1H-pyrazol-3-yl]-2-piperazinone | LC/MS Rt = 3.58 [MH]⁺ 543.4 |

### Example 285

### 1-[1-({5-Chloro-2-[(2-chloro-2-propen-1-yl)oxy]phenyl}methyl)-5-methyl-1H-pyrazol-3-yl]-4-(2-methylpropyl)-2-piperazinone hydrochloride

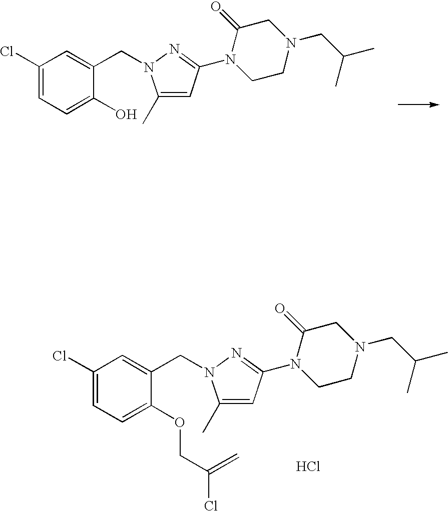

To a solution of 1-{1-[(5-chloro-2-hydroxyphenyl)methyl]-5-methyl-1H-pyrazol-3-yl}-4-(2-methylpropyl)-2-piperazinone (0.10 g, 0.27 mmol) in tetrahydrofuran (3 ml), was added triphenylphosphine (0.077 g, 0.29 mmol), diisopropylazodicarboxylate (0.059 g, 0.29 mmol) and 2-chloro-2-propen-1-ol (0.027 g, 0.29 mmol). The mixture was heated to 50° C. under argon overnight. The reaction was concentrated and then purified using flash chromatography (0-40% ethyl acetate:hexane) to yield a clear oil. This was dissolved in dichloromethane (1 ml) and 1M hydrogen chloride in diethyl ether (0.50 ml, 0.50 mmol) was added. The residue was blown dry to yield a white solid (0.058 g, 45%).

LC/MS Rt=2.76, [MH+] 451.

The following examples were prepared in a similar manner to 1-[1-({5-chloro-2-[(2-methyl-2-propen-1-yl)oxy]phenyl}methyl)-5-methyl-1H-pyrazol-3-yl]-4-(2-methylpropyl)-2-piperazinone hydrochloride from the appropriate intermediates.

| Example | Structure | Name | Data |
|---|---|---|---|
| 286 | 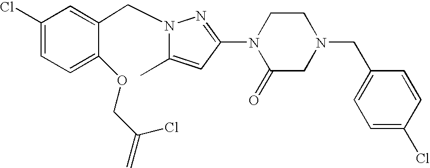 | 1-[1-({5-Chloro-2-[(2-chloro- 2-propen-1-yl)oxy]phenyl}methyl)-5- methyl-1H-pyrazol-3-yl]-4- [(4-chlorophenyl)methyl]-2- piperazinone | LC/MS Rt = 3.61, [MH]⁺ 521.1, 523.1 |
| 287 | 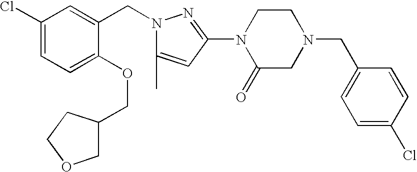 | 4-[(4-Chlorophenyl)methyl]- 1-[1-({5-chloro-2-[(tetrahydro-3- furanylmethyl)oxy]phenyl}methyl)-5- methyl-1H-pyrazol-3-yl]-2- piperazinone | LC/MS Rt = 3.25 [MH]⁺ 529.2, 531.2 |

### Example 288

### 4-[1-({5-Chloro-2-[(phenylmethyl)oxy]phenyl}methyl)-5-methyl-1H-pyrazol-3-yl]-1-(phenylmethyl)piperazin-1-ium chloride

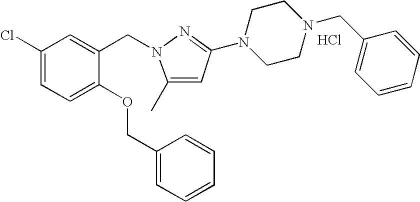

1-({5-Chloro-2-[(phenylmethyl)oxy]phenyl}methyl)-3-iodo-5-methyl-1H-pyrazole (166 mg, 0.379 mmol) and N-benzylpiperidine (138 μl, 0.797 mmol) were dissolved in toluene (3 ml) under argon. Sodium t-butoxide (52 mg, 0.53 mmol) and bis(tri-t-butylphosphine)palladium(0) (10 mg, 0.019 mmol) were added and the reaction stirred at 115° C. for 16 h. The mixture was filtered through a pad of Kieselguhr and the filtrate evaporated. The residue was twice purified by flash chromatography, initially with 1-5% methanol in dichloromethane and finally with 2% methanol in dichloromethane. The product was stirred in 1M hydrochloric acid in diethyl ether for 30 minutes and the resulting solid washed by decantation from diethyl ether and dried in vacuo (38 mg).

LC/MS Rt=2.46 min, [MH⁺] 487, 489.

### Example 289

### 4-[1-({5-Chloro-2-[(phenylmethyl)oxy]phenyl}methyl)-5-methyl-1H-pyrazol-3-yl]-1-(phenylmethyl)-2-piperazinone

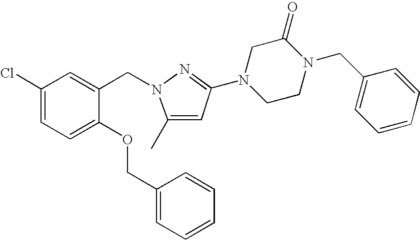

2-Chloro-N-(2-oxoethyl)-N-(phenylmethyl)acetamide (63 mg, 0.28 mmol) was dissolved in dichloromethane (1 ml) with stirring under argon. Powdered 4A molecular sieve (10 mg) was added, followed by 1-({5-chloro-2-[(phenylmethyl)oxy]phenyl}methyl)-5-methyl-1H-pyrazol-3-amine (110 mg, 0.336 mmol). After stirring for 1 minute, sodium triacetoxyborohydride (71 mg, 0.336 mmol) was added. The reaction was stirred at 0° C. for 5 minutes then allowed to warm to room temperature and stirred for 2 h. 2M Hydrochloric acid was added and the mixture stirred for 30 minutes. The organic layer was washed with 5% sodium bicarbonate solution and water, dried (MgSO₄) and evaporated. The residue was dissolved in toluene (2 ml) and triethylamine (47 μl, 0.336 mmol) added. The mixture was heated at 180° C. in a Smithcreator® microwave for 20 minutes. The cooled mixture was evaporated and the residue purified by MDAP to give the title compound (24 mg). LC/MS Rt=3.45 min, [MH⁺] 501, 503.

### Example 290

### 1-({5-Chloro-2-[(phenylmethyl)oxy]phenyl}methyl)-5-methyl-N-(1-methylethyl)-1H-pyrazole-3-carboxamide

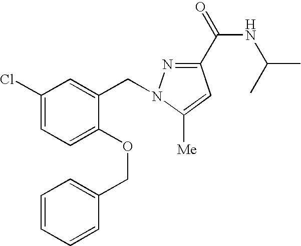

A mixture of 1-({5-chloro-2-[(phenylmethyl)oxy]phenyl}methyl)-5-methyl-1H-pyrazole-3-carboxylic acid (178 mg, 0.5 mmol), isopropylamine (35 mg, 0.6 mmol), 1-hydroxybenzotriazole (92 mg, 0.6 mmol) and N-(3-dimethylaminopropyl)-N′-ethylcarbodiimide (115 mg, 0.6 mmol) in dichloromethane (4 ml) was stirred at room temperature for 3 hours. The resulting solution was diluted with ether, washed with saturated sodium bicarbonate and 2M hydrochloric acid, dried (magnesium sulphate), evaporated and triturated with ether to give the title compound as a white solid (113 mg). LC/MS Rt 3.58, [MH]⁺ 398.6, 400.6.

The following examples were prepared in a similar manner to 1-({5-chloro-2-[(phenylmethyl)oxy]phenyl}methyl)-5-methyl-N-(1-methylethyl)-1H-pyrazole-3-carboxamide from the appropriate intermediates:

| Example | Structure | Name | Data |
|---|---|---|---|
| 291 | 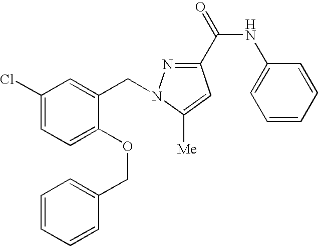 | 1-({5-Chloro-2- [(phenylmethyl)oxy]phenyl}methyl)-5- methyl-N-phenyl-1H-pyrazol-3- carboxamide | LC/MS Rt = 3.96 [MH]⁺ 432.6, 434.6 |
| 292 | 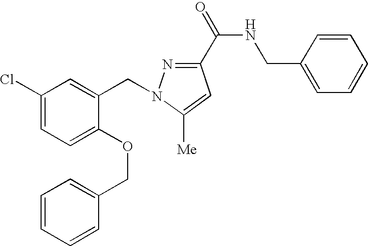 | 1-({5-Chloro-2- [(phenylmethyl)oxy]phenyl}methyl)-5- methyl-N-(phenylmethyl)-1H-pyrazol-3- carboxamide | LC/MS Rt = 3.80 [MH]⁺ 446.6, 448.6 |
| 293 | 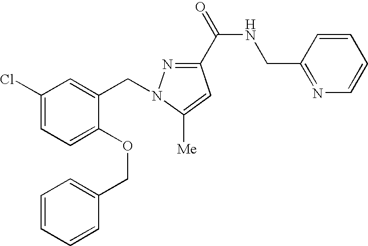 | 1-({5-Chloro-2- [(phenylmethyl)oxy]phenyl}methyl)-5- methyl-N-(2-pyridinylmethyl)-1H-pyrazol- 3-carboxamide | LC/MS Rt = 3.42 [MH]⁺ 447.5, 449.5 |
| 294 | 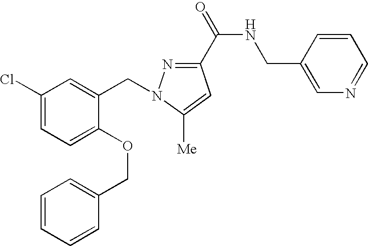 | 1-({5-Chloro-2- [(phenylmethyl)oxy]phenyl}methyl)-5- methyl-N-(3-pyridinylmethyl)-1H- pyrazole-3-carboxamide | LC/MS Rt = 3.27 [MH]⁺ 447.5, 449.5 |
| 295 | 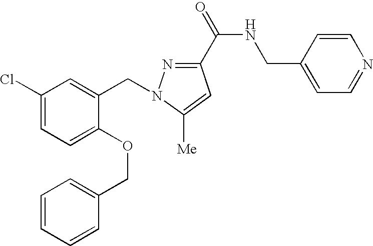 | 1-({5-Chloro-2- [(phenylmethyl)oxy]phenyl}methyl)-5- methyl-N-(4-pyridinylmethyl)-1H- pyrazole-3-carboxamide | LC/MS Rt = 3.20 [MH]⁺ 447.5, 449.5 |
| 296 | 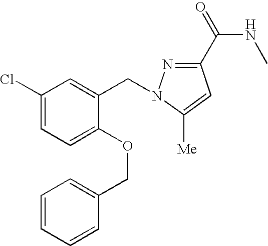 | 1-({5-Chloro-2- [(phenylmethyl)oxy]phenyl}methyl)-N,5- dimethyl-1H-pyrazole- 3-carboxamide | LC/MS Rt = 3.34 [MH]⁺ 370.4, 372.4 |

### Example 297

### 1-({5-Chloro-2-[(phenylmethyl)oxy]phenyl}methyl)-5-methyl-N-2-pyridinyl-1H-pyrazole-3-carboxamide

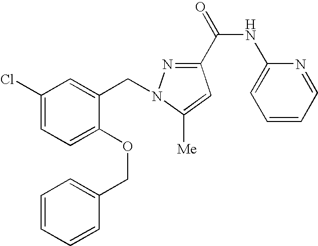

Oxalyl chloride (1.2 ml) was added to a solution of 1-({5-chloro-2-[(phenylmethyl)oxy]phenyl}methyl)-5-methyl-1H-pyrazole-3-carboxylic acid (427 mg, 1.2 mmol) and dimethylformamide (1 drop) in dichloromethane (15 ml) and left at room temperature for 1 hour. The resulting solution was evaporated to dryness, dissolved in dichloromethane (10 ml) and 5 ml of this solution was added to a solution of 2-aminopyridine (62 mg, 0.66 mmol) and triethylamine (101 mg, 1 mmol) in dichloromethane (5 ml). After 2 hours at room temperature the solution was diluted with ethyl acetate, washed with water, dried (magnesium sulphate), evaporated and purified by flash chromatography eluting with ethyl acetate/hexane (1:4) to give the title compound as a white solid (21 mg). LC/MS Rt=3.96 [MH]⁺ 433.4, 435.4.

The following examples were prepared in a similar manner to 1-({5-chloro-2-[(phenylmethyl)oxy]phenyl}methyl)-5-methyl-N-2-pyridinyl-1H-pyrazole-3-carboxamide from the appropriate intermediates:

| Example | | Name | Data |
|---|---|---|---|
| 298 | 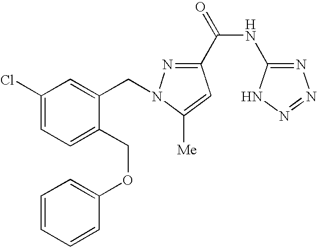 | 1-({5-Chloro-2- [(phenylmethyl)oxy]phenyl}meth- yl)-5-methyl-N-1H-tetrazol- 5-yl-1H-pyrazole-3- carboxamide | LC/MS Rt = 3.46 [MH]⁺ 424.4, 426.4 |
| 299 | 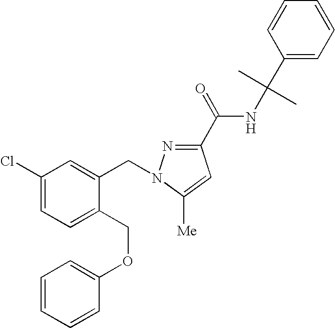 | 1-({5-Chloro-2- [(phenylmethyl)oxy]phenyl}meth- yl)-5-methyl-N-(1-methyl-1- phenylethyl)-1H-pyrazole-3- carboxamide | LC/MS Rt = 4.08 [MH]⁺ 474.4 |

### 1-({5-Chloro-2-[(phenylmethyl)oxy]phenyl}methyl)-5-methyl-1H-pyrazole-3-carbonyl chloride

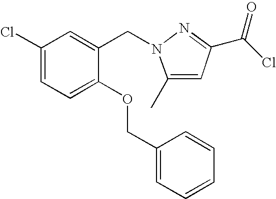

1-({5-Chloro-2-[(phenylmethyl)oxy]phenyl}methyl)-5-methyl-1H-pyrazole-3-carboxylic acid (2.14 g, 6 mmol) was dissolved in dichloromethane (50 ml) and oxalyl chloride (6 ml) added, followed by careful addition of dimethylformamide (2 drops). The mixture was stirred at room temperature for 1.5 h. The solvent was evaporated and the residue re-evaporated from toluene to leave an off-white solid (2.25 g).

1-({5-Chloro-2-[(2-methylpropyl)oxy]phenyl}methyl)-5-methyl-1H-pyrazole-3-carbonyl chloride

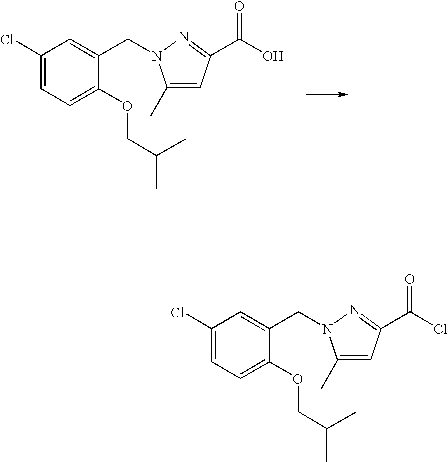

Thionyl chloride (0.22 mL, 3.13 mmol) was added to 1-({5-chloro-2-[(2-methylpropyl)oxy]phenyl}methyl)-5-methyl-1H-pyrazole-3-carboxylic acid (0.197 g, 0.61 mmol) in dichloromethane (1.2 ml). The mixture was heated at reflux for 19 hours then cooled to room temperature. Evaporation of the volatile components gave the title compound which was used without purification.

### 1-({5-Bromo-2-[(phenylmethyl)oxy]phenyl}methyl)-5-methyl-1H-pyrazole-3-carbonyl chloride

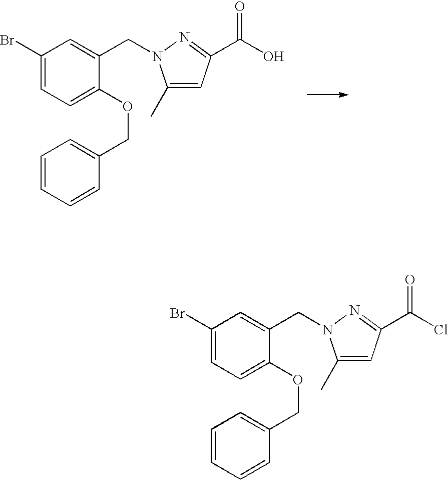

A solution of 1-({5-bromo-2-[(phenylmethyl)oxy]phenyl}methyl)-5-methyl-1H-pyrazole-3-carboxylic acid (1.50 g, 3.73 mmol) in dry dichloromethane (12 ml) was stirred at 0° C. under an atmosphere of argon. Thionyl chloride (1.4 ml, 18.7 mmol) was added dropwise to the solution. The solution was allowed to warm to room temperature and stirred for 2 hours. The solvent was removed under reduced pressure to give a white solid (1.26 g). LC/MS Rt=3.67 [M+Na⁺] 443, 445.

The following examples were prepared in a similar manner to 1-({5-bromo-2-[(phenylmethyl)oxy]phenyl}methyl)-5-methyl-1H-pyrazole-3-carbonyl chloride from the appropriate intermediates:

| Structure | Name | Data |
|---|---|---|
| 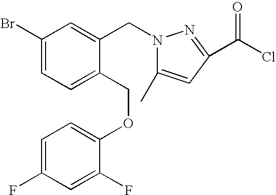 | 1-[(5-Bromo-2-{[(2,4- difluorophenyl)methyl]oxy} phenyl)methyl]-5-methyl- 1H-pyrazole-3-carbonyl chloride | LC/MS Rt = 3.68 [M + Na⁺] 479, 481 |
| 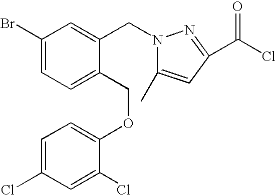 | 1-[(5-bromo-2-{[(2,4- dichlorophenyl)methyl]oxy} phenyl)methyl]-5-methyl- 1H-pyrazole-3-carbonyl chloride | LC/MS Rt = 4.05 [MH⁺] 487, 489 |
| 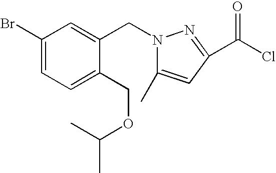 | 1-({5-bromo-2-[(2- methylpropyl)oxy]phenyl} methyl)-5-methyl-1H- pyrazole-3-carbonyl chloride | LC/MS Rt = 3.88 [MH⁺] 387, 389 |

### Example 300

### N-(2-Chlorophenyl)-1-({5-chloro-2-[(phenylmethyl)oxy]phenyl}methyl)-5-methyl-1H-pyrazole-3-carboxamide

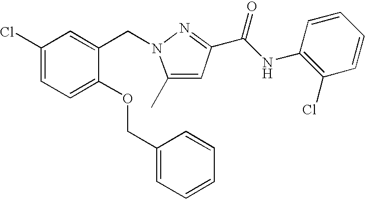

1-({5-Chloro-2-[(phenylmethyl)oxy]phenyl}methyl)-5-methyl-1H-pyrazole-3-carbonyl chloride (94 mg, 0.25 mmol) was dissolved in dichloromethane (2 ml) and triethylamine (58 μl, 0.42 mmol) and 2-chloroaniline (29 μl, 0.275 mmol) were added. The reaction was stirred at room temperature for 2 h then diluted with dichloromethane and washed with water, dried (MgSO₄) and evaporated. The residue was triturated with isohexane and diethyl ether and the solid collected by decantation and dried in vacuo (60 mg).

LC/MS Rt=4.42 min, [MH⁺] 466, 469.

The following examples were prepared in a similar manner to N-(2-chlorophenyl)-1-({5-chloro-2-[(phenylmethyl)oxy]phenyl}methyl)-5-methyl-1H-pyrazole-3-carboxamide from the appropriate intermediates.

| Example | Structure | Name | Data |
|---|---|---|---|
| 301 | 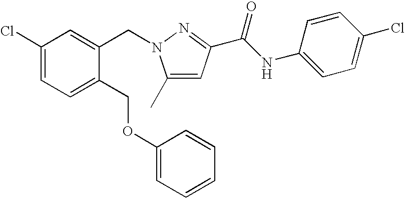 | N-(4-Chlorophenyl)-1-({5- chloro-2- [(phenylmethyl)oxy]phenyl} methyl)-5-methyl-1H- pyrazole-3-carboxamide | LC/MS Rt = 4.23 min, [MH⁺] 466, 469. |
| 302 | 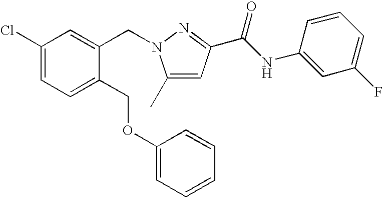 | 1-({5-Chloro-2- [(phenylmethyl)oxy]phenyl} methyl)-N-(3-fluorophenyl)- 5-methyl-1H-pyrazole-3- carboxamide | LC/MS Rt = 4.09 min, [MH⁺] 450, 452. |
| 303 | 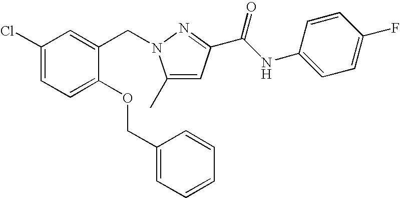 | 1-({5-Chloro-2- [(phenylmethyl)oxy]phenyl} methyl)-N-(4-fluorophenyl)- 5-methyl-1H-pyrazole-3- carboxamide | LC/MS Rt = 4.01 min, [MH⁺] 450, 452. |
| 304 | 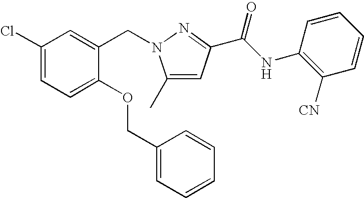 | 1-({5-Chloro-2- [(phenylmethyl)oxy]phenyl} methyl)-N-(2-cyanophenyl)- 5-methyl-1H-pyrazole-3- carboxamide | ¹H NMR (CDCl₃) δ: 2.21(3 H, s), 5.20(2 H, s), 5.37(2 H, s), 6.66(1 H, s), 6.80(1 H, d), 7.17(1 H, d), 7.33-7.44(7 H, m) 7.70(1 H, t), 7.79(1 H, d), 7.82(1 H, d), 10.05(1 H, s). |
| 305 | 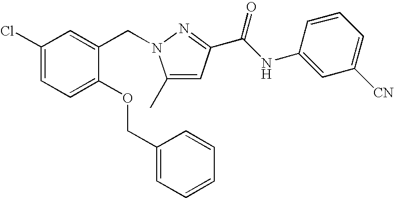 | 1-({5-Chloro-2- [(phenylmethyl)oxy]phenyl} methyl)-N-(3-cyanophenyl)- 5-methyl-1H-pyrazole-3- carboxamide | LC/MS Rt = 3.98 min, [MH⁺] 457, 459. |
| 306 | 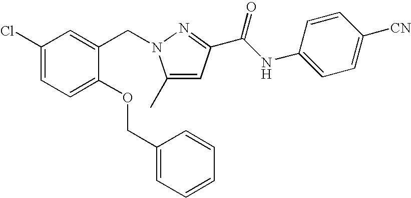 | 1-({5-Chloro-2- [(phenylmethyl)oxy]phenyl} methyl)-N-(4-cyanophenyl)- 5-methyl-1H-pyrazole-3- carboxamide | LC/MS Rt = 3.99 min, [MH⁺] 457, 459. |
| 307 | 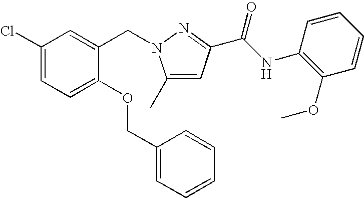 | 1-({5-Chloro-2- [(phenylmethyl)oxy]phenyl} methyl)-5-methyl-N-[2- (methyloxy)phenyl]-1H- pyrazole-3-carboxamide | LC/MS Rt = 4.18 min, [MH⁺] 462, 464. |
| 308 | 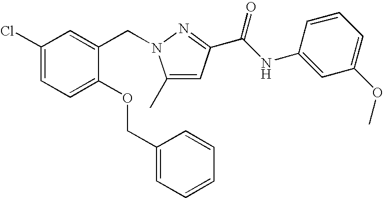 | 1-({5-Chloro-2- [(phenylmethyl)oxy]phenyl} methyl)-5-methyl-N-[3- (methyloxy)phenyl]-1H- pyrazole-3-carboxamide | LC/MS Rt = 4.00 min, [MH⁺] 462, 464. |
| 309 | 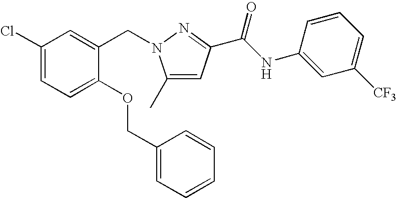 | 1-({5-Chloro-2- [(phenylmethyl)oxy]phenyl} methyl)-5-methyl-N-[3- (trifluoromethyl)phenyl]-1H- pyrazole-3-carboxamide | LC/MS Rt = 4.27 min, [MH⁺] 500, 502. |
| 310 | 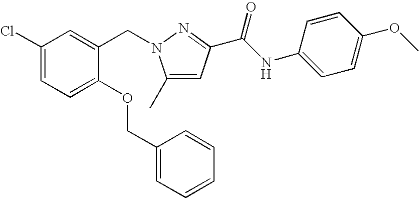 | 1-({5-Chloro-2- [(phenylmethyl)oxy]phenyl} methyl)-5-methyl-N-[4- (methyloxy)phenyl]-1H- pyrazole-3-carboxamide | LC/MS Rt = 3.91 min, [MH⁺] 462, 464. |
| 311 | 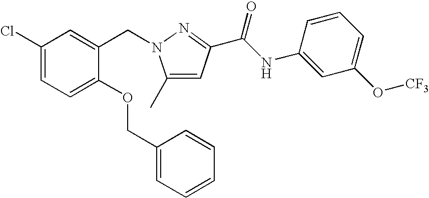 | 1-({5-Chloro-2- [(phenylmethyl)oxy]phenyl} methyl)-5-methyl-N-{3- [(trifluoromethyl)oxy]phenyl}- 1H-pyrazole-3- carboxamide | LC/MS Rt = 4.31 min, [MH⁺] 516, 518. |
| 312 | 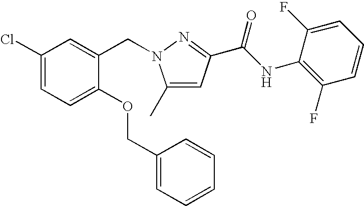 | 1-({5-Chloro-2- [(phenylmethyl)oxy]phenyl} methyl)-N-(2,6- difluorophenyl)-5-methyl- 1H-pyrazole-3- carboxamide | LC/MS Rt = 3.80 min, [MH⁺] 468, 470. |
| 313 | 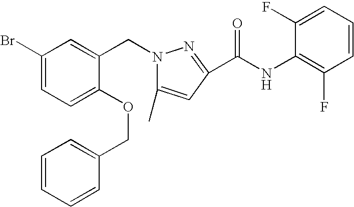 | 1-({5-Bromo-2- [(phenylmethyl)oxy]phenyl} methyl)-N-(2,6- difluorophenyl)-5-methyl- 1H-pyrazole-3- carobxamide | LC/MS Rt = 3.60 [MH⁺] 514, 516 |
| 314 | 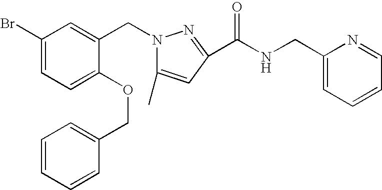 | 1-({5-Bromo-2- [(phenylmethyl)oxy]phenyl} methyl)-5-methyl-N-(2- pyridinylmethyl)-1H- pyrazole-3-carboxamide | LC/MS Rt = 3.09 [MH⁺] 493, 495 |
| 315 | 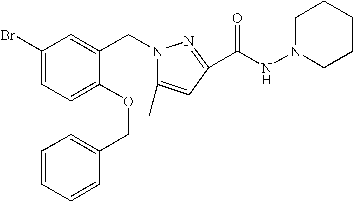 | 1-({5-Bromo-2- [(phenylmethyl)oxy]phenyl} methyl)-5-methyl-N-1- piperidinyl-1H-pyrazole-3- carboxamide | LC/MS Rt = 3.28 [MH⁺] 485, 487 |
| 316 | 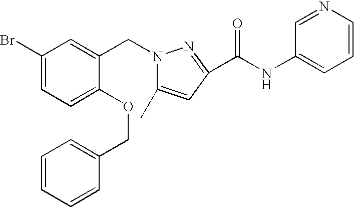 | 1-({5-Bromo-2- [(phenylmethyl)oxy]phenyl} methyl)-5-methyl-N-3- pyridinyl-1H-pyrazole-3- carboxamide | LC/MS Rt = 2.86 [MH⁺] 479, 481 |
| 317 | 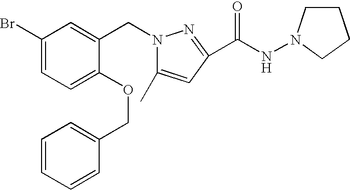 | 1-({5-Bromo-2- [(phenylmethyl)oxy]phenyl} methyl)-5-methyl-N-1- pyrrolidinyl-1H-pyrazole-3- carboxamide | LC/MS Rt = 2.96 [MH⁺] 469, 472 |
| 318 | 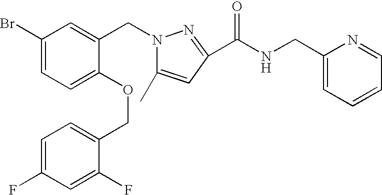 | 1-[(5-Bromo-2-{[(2,4- difluorophenyl)methyl]oxy} phenyl)methyl]-5-methyl-N- (2-pyridinylmethyl)-1H- pyrazole-3-carboxamide | LC/MS Rt = 2.93 [MH⁺] 527, 529 |
| 319 | 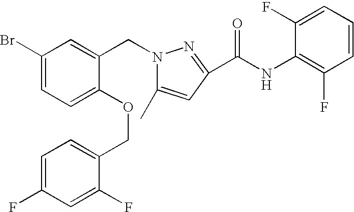 | 1-[(5-Bromo-2-{[(2,4- difluorophenyl)methyl]oxy} phenyl)methyl]-N-(2,6- difluorophenyl)-5-methyl- 1H-pyrazole-3- carboxamide | LC/MS Rt = 3.62 [MH⁺] 549, 551 |
| 320 | 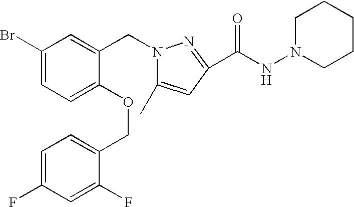 | 1-[(5-Bromo-2-{[(2,4- difluorophenyl)methyl]oxy} phenyl)methyl]-5-methyl-N- 1-piperidinyl-1H-pyrazole- 3-carboxamide | LC/MS Rt = 3.30 [MH⁺] 521, 523 |
| 321 | 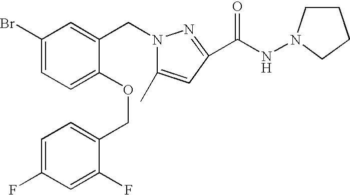 | 1-[(5-Bromo-2-{[(2,4- difluorophenyl)methyl]oxy} phenyl)methyl]-5-methyl-N- 1-pyrrolidinyl-1H-pyrazole- 3-carboxamide | LC/MS Rt = 3.00 [MH⁺] 505, 508 |
| 322 | 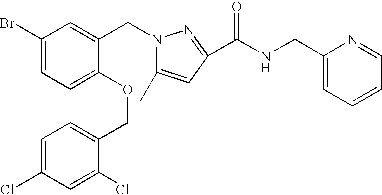 | 1-[(5-Bromo-2-{[(2,4- dichlorophenyl)methyl]oxy} phenyl)methyl]-5-methyl-N- (2-pyridinylmethyl)-1H- pyrazole-3-carboxamide | LC/MS Rt = 3.35 [MH⁺] 561, 563 |
| 323 | 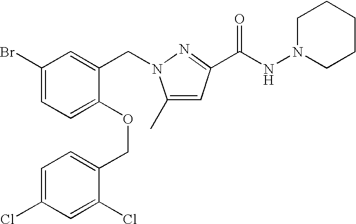 | 1-[(5-Bromo-2-{[(2,4- dichlorophenyl)methyl]oxy} phenyl)methyl]-5-methyl-N- 1-piperidinyl-1H-pyrazole- 3-carboxamide | LC/MS Rt = 3.70 [MH⁺] 553, 555 |
| 324 | 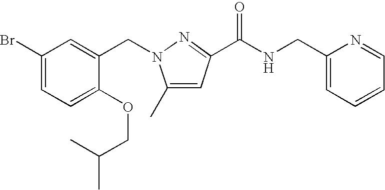 | 1-({5-Bromo-2-[(2- methylpropyl)oxy]phenyl}meth- yl)-5-methyl-N-(2- pyridinylmethyl)-1H- pyrazole-3-carboxamide | LC/MS Rt = 2.95 [MH⁺] 459, 461 |
| 325 | 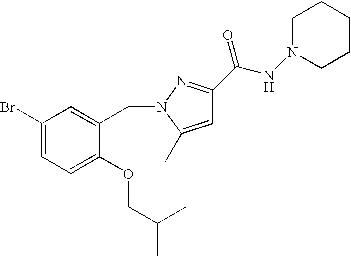 | 1-({5-Chloro-2-[(2- methylpropyl)oxy]phenyl}meth- yl)-5-methyl-N-1- piperidinyl-1H-pyrazole-3- carboxamide | LC/MS Rt = 3.37 min [MH⁺] 405, 407 |
| 326 | 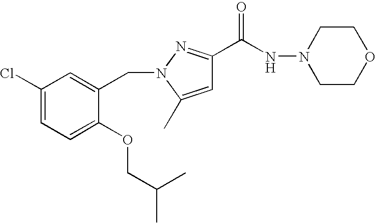 | 1-({5-Chloro-2-[(2- methylpropyl)oxy]phenyl}meth- yl)-5-methyl-N-4- morpholinyl-1H-pyrazole-3- carboxamide | LC/MS Rt = 2.99 min [MH⁺] 407, 409 |
| 327 | 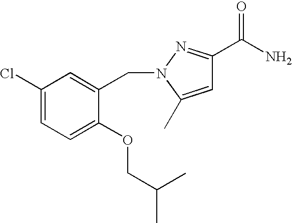 | 1-({5-Chloro-2-[(2- methylpropyl)oxy]phenyl}meth- yl)-5-methyl-1H- pyrazole-3-carboxamide | LC/MS Rt = 3.03 min [MH⁺] 322 |
| 328 | 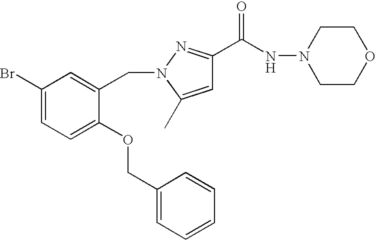 | 1-({5-Bromo-2- [(phenylmethyl)oxy]phenyl} methyl)-5-methyl-N-4- morpholinyl-1H-pyrazole-3- carboxamide | LC/MS Rt = 2.96 min [MH⁺] 485, 487 |
| 329 | 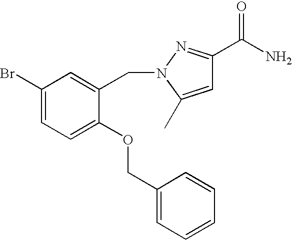 | 1-({5-Bromo-2- [(phenylmethyl)oxy]phenyl} methyl)-5-methyl-1H- pyrazole-3-carboxamide | LC/MC Rt = 2.99 min [MH⁺] 401, 403 |
| 330 | 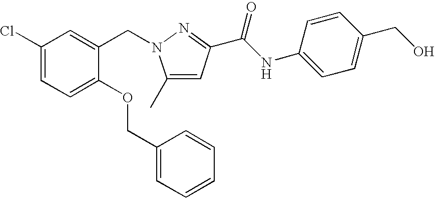 | 1-({5-Chloro-2- [(phenylmethyl)oxy]phenyl} methyl)-N-[4- (hydroxymethyl)phenyl]-5- methyl-1H-pyrazole-3- carboxamide | LC/MS Rt = 3.23 min [MH⁺] 462, 464 |
| 331 | 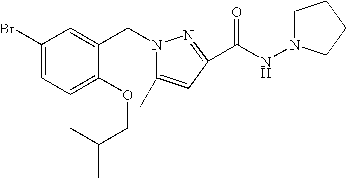 | 1-({5-Bromo-2-[(2- methylpropyl)oxy]phenyl}meth- yl)-5-methyl-N-1- pyrrolidinyl-1H-pyrazole-3- carboxamide | LC/MS Rt = 3.08 [MH⁺] 437, 438 |
| 332 | 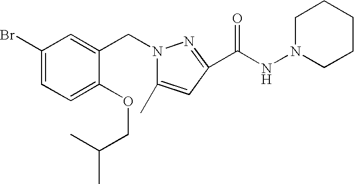 | 1-({5-Bromo-2-[(2- methylpropyl)oxy]phenyl}meth- yl)-5-methyl-N-1- piperidinyl-1H-pyrazole-3- carboxamide | LC/MS Rt = 3.38 [MH⁺] 451, 452 |
| 333 | 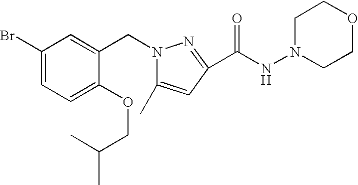 | 1-({5-Bromo-2-[(2- methylpropyl)oxy]phenyl}meth- yl)-5-methyl-N-4- morpholinyl-1H-pyrazole-3- carboxamide | LC/MS Rt = 3.04 [MH⁺] 453, 454 |

### 1-[(5-Chloro-2-hydroxyphenyl)methyl]-5-methyl-N-(2-pyridinylmethyl)-1H-pyrazole-3-carboxamide

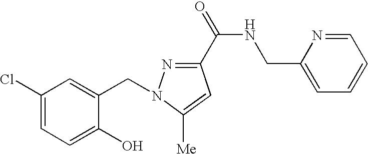

1-({5-Chloro-2-[(phenylmethyl)oxy]phenyl}methyl)-5-methyl-N-(2-pyridinylmethyl)-1H-pyrazole-3-carboxamide (2.05 g, 4.59 mmol) was dissolved in 48% hydrogen bromide in acetic acid (30 ml) and left at room temperature for 18 hours. The resulting solution was diluted with ether/water, carefully basified with potassium carbonate and filtered to remove precipitated solid. The organic phase was dried (magnesium sulphate), evaporated and the residue triturated with hexane and the solid filtered off. The combined solids were dissolved in ethanol (30 ml) and 2M sodium hydroxide (20 ml), left at room temperature for 15 minutes and evaporated to dryness. The residue was dissolved in ethyl acetate/water, acidified with acetic acid and the precipitated solid filtered off and dried. The organic phase from the filtrate was dried (magnesium sulphate), evaporated and triturated with ethyl acetate and filtered off. The two batches of solid were combined to give the title compound as white solid (1.5 g). LC/MS Rt 2.69, [MH]⁺ 357.4, 359.4.

### Example 334

### 1-[(5-Chloro-2-{[(2-fluorophenyl)methyl]oxy}phenyl)methyl]-5-methyl-N-(2-pyridinylmethyl)-1H-pyrazole-3-carboxamide

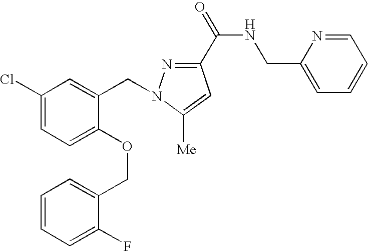

A mixture of 1-[(5-chloro-2-hydroxyphenyl)methyl]-5-methyl-N-(2-pyridinylmethyl)-1H-pyrazole-3-carboxamide (100 mg, 0.28 mmol), potassium carbonate (138 mg, 1 mmol) and 2-fluorobenzyl bromide (63 mg, 0.33 mmol) in acetone (5 ml) was stirred and refluxed for 3 hours then cooled, filtered, evaporated and triturated with ether to give the title compound as an off-white solid (110 mg). LC/MS Rt 3.46, [MH]⁺ 465.3, 467.4.

The following examples were prepared from the appropriate starting materials using a similar procedure to that described for Example 334:

| Example | Structure | Name | Data |
|---|---|---|---|
| 335 | 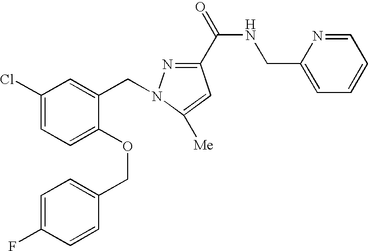 | 1-[(5-Chloro-2-{[(4- fluorophenyl)methyl]oxy}phe nyl)methyl]-5-methyl-N-(2- pyridinylmethyl)-1H- pyrazole-3-carboxamide | LC/MS Rt = 3.45 [MH]⁺ 465.3, 467.4 |
| 336 | 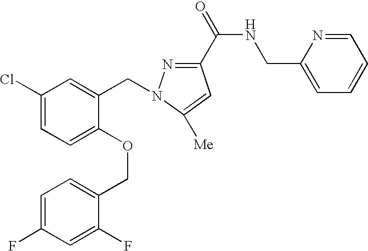 | 1-[(5-Chloro-2-{[(2,4- difluorophenyl)methyl]oxy}phen- nyl)methyl]-5-methyl-N-(2- pyridinylmethyl)-1H- pyrazole-3-carboxamide | LC/MS Rt = 3.49 [MH]⁺ 483.3, 485.3 |
| 337 | 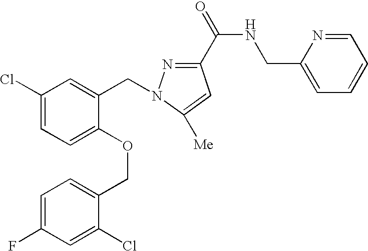 | 1-[(5-Chloro-2-{[(2-chloro-4- fluorophenyl)methyl]oxy}phe nyl)methyl]-5-methyl-N-(2- pyridinylmethyl)-1H- pyrazole-3-carboxamide | LC/MS Rt = 3.65 [MH]⁺ 499.3, 502.3 |
| 338 | 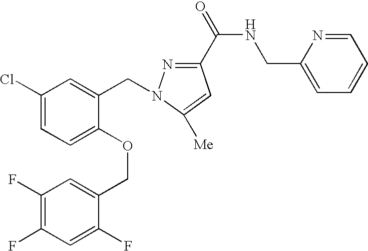 | 1-[(5-Chloro-2-{[(2,4,5- trifluorophenyl)methyl]oxy}phe- nyl)methyl]-5-methyl-N-(2- pyridinylmethyl)-1H- pyrazole-3-carboxamide | LC/MS Rt = 3.52 [MH]⁺ 501.3, 503.3 |
| 339 | 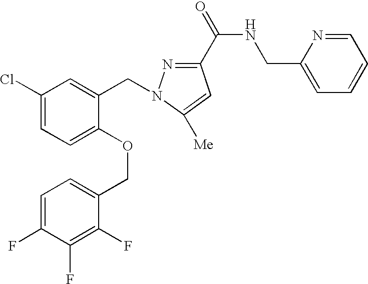 | 1-[(5-Chloro-2-{[(2,3,4- trifluoro- phenyl)methyl]oxy}phe- nyl)methyl]-5-methyl-N-(2- pyridinylmethyl)-1H- pyrazole-3-carboxamide | LC/MS Rt = 3.55 [MH]⁺ 501.3, 503.3 |
| 340 | 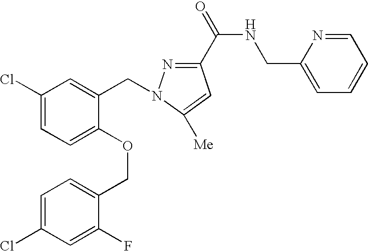 | 1-[(5-Chloro-2-{[(4-chloro-2- fluorophenyl)methyl]oxy}phe- nyl)methyl]-5-methyl-N-(2- pyridinylmethyl)-1H- pyrazole-3-carboxamide | LC/MS Rt = 3.66 [MH]⁺ 499.3, 502.3 |
| 341 | 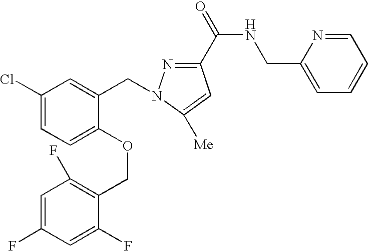 | 1-[(5-Chloro-2-{[(2,4,6- trifluorophenyl)methyl] oxy}phe- nyl)methyl]-5-methyl-N-(2- pyridinylmethyl)-1H- pyrazole-3-carboxamide | LC/MS Rt = 3.48 [MH]⁺ 501.3, 503.3 |
| 342 | 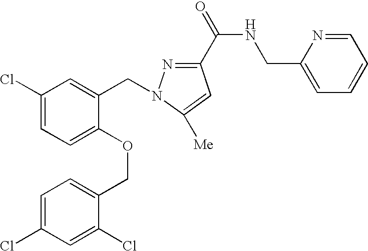 | 1-[(5-Chloro-2-{[(2,4- dichlorophenyl)methyl] oxy}phe- nyl)methyl]-5-methyl-N-(2- pyridinylmethyl)-1H- pyrazole-3-carboxamide | LC/MS Rt = 3.78 [MH]⁺ 515.3, 517.3 |
| 343 | 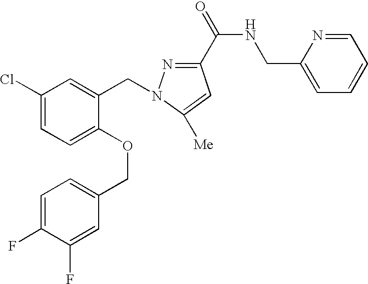 | 1-[(5-Chloro-2-{[(3,4- difluorophenyl)methyl] oxy}phe- nyl)methyl]-5-methyl-N-(2- pyridinylmethyl)-1H- pyrazole-3-carboxamide | LC/MS Rt = 3.49 [MH]⁺ 483.3, 485.3 |

### 1-({5-Chloro-2-[(phenylmethyl)oxy]phenyl}methyl)-N-(4-formylphenyl)-5-methyl-1H-pyrazole-3-carboxamide

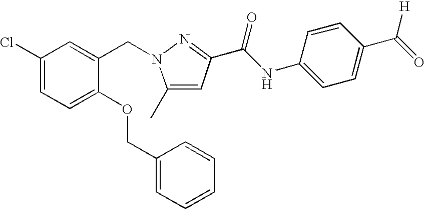

1-({5-Chloro-2-[(phenylmethyl)oxy]phenyl}methyl)-N-[4-(hydroxymethyl)phenyl]-5-methyl-1H-pyrazole-3-carboxamide (925 mg, 2 mmol) was dissolved in dichloromethane (20 ml) and Dess-Martin periodinane (910 mg, 2.2 mmol) added. The mixture was stirred at room temperature for 2 h. then washed with 5% sodium thiosulphate solution and water, dried (MgSO₄) and evaporated. The residue was triturated with diethyl ether and the cream solid filtered and dried (790 mg). LC/MS Rt=3.63 min [MH]⁺ 460, 462.

### Example 344

### 1-({5-Chloro-2-[(phenylmethyl)oxy]phenyl}methyl)-5-methyl-N-{4-[(methylamino)methyl]phenyl}-1H-pyrazole-3-carboxamide hydrochloride

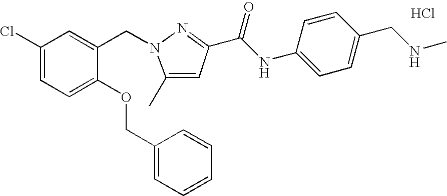

1-({5-Chloro-2-[(phenylmethyl)oxy]phenyl}methyl)-N-(4-formylphenyl)-5-methyl-1H-pyrazole-3-carboxamide (88 mg, 0.19 mmol) was dissolved in dichloromethane (2 ml) and 2M methylamine in tetrahydrofuran (190 μl, 0.38 mmol) and acetic acid (12.5 μl, 0.21 mmol) and sodium triacetoxyborohydride (124 mg, 0.57 mmol) added. The reaction was stirred at room temperature for 2 hours then diluted with dichloromethane and water. The organic phase was evaporated and the residue purified by flash chromatography eluting with 10-20% methanol in dichloromethane. The product was dissolved in dichloromethane (1 ml) and 1M hydrogen chloride in diethyl ether (1 ml) added. The resulting solid was collected by decantation and dried to give the title compound (32 mg).

¹H NMR (MeOD) δ: 2.18 (3H, s), 2.71 (3H, s), 4.16 (2H, s), 5.12 (2H, s), 5.35, (2H, s), 6.64 (1H, s), 6.86 (1H, s), 7.08 (1H, d), 7.25-7.37 (9H, m), 7.80 (2H, d).

The following examples were prepared from the appropriate starting materials using a similar procedure to that described for Example 344.

| Ex- | | | |
|---|---|---|---|
| ample | Structure | Name | Data |
| 345 | 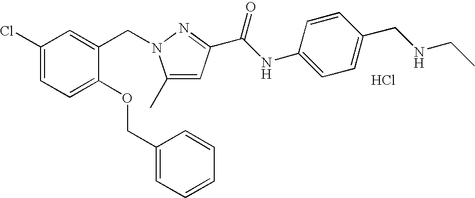 | 1-({5-Chloro-2- [(phenylmethyl)oxy]phenyl} methyl)-N-{4- [(ethylamino)methyl]phenyl}- 5-methyl-1H-pyrazole-3- carboxamide hydrochloride | ¹H NMR (MeOD) δ: 1.33 (3 H, t), 2.19(3 H, s), 3.10 (2 H, q), 4.16(2 H, s), 5.14(2 H, s), 5.37, (2 H, s), 6.64(1 H, s), 6.85(1 H, d), 7.08(1 H, d), 7.25- 7.37(9 H, m), 7.80(2 H, d). |
| 346 | 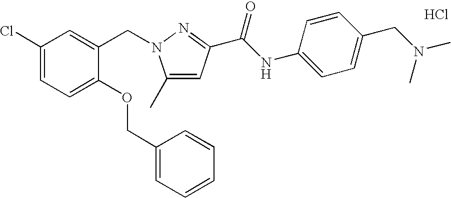 | 1-({5-Chloro-2- [(phenylmethyl)oxy]phenyl} methyl)-N-{4- [(dimethylamino)methyl]phe- nyl}-5-methyl-1H- pyrazole-3-carboxamide hydrochloride | LC/MS Rt = 2.45 min [MH]⁺ 489, 491. |
| 347 | 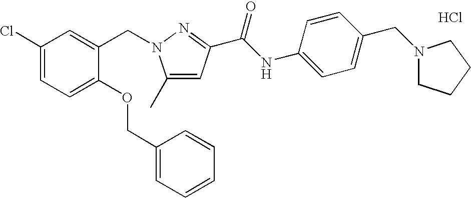 | 1-({5-Chloro-2- [(phenylmethyl)oxy]phenyl} methyl)-5-methyl-N-[4-(1- pyrrolidinylmethyl)phenyl]- 1H-pyrazole-3- carobxamide hydrochloride | LC/MS Rt = 2.45 min [MH]⁺ 515, 517. |
| 348 | 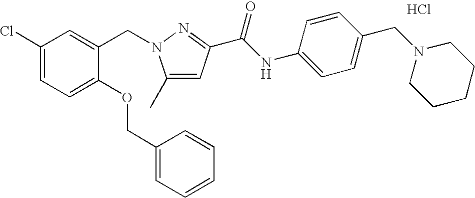 | 1-({5-Chloro-2- [(phenylmethyl)oxy]phenyl} methyl)-5-methyl-N-[4-(1- piperidinylmethyl)phenyl]- 1H-pyrazole-3- carboxamide hydrochloride | LC/MS Rt = 2.51 min [MH]⁺ 529, 531. |
| 349 | 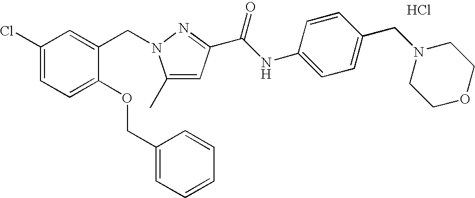 | 1-({5-Chloro-2- [(phenylmethyl)oxy]phenyl} methyl)-5-methyl-N-[4-(4- morpholinylmethyl)phenyl]- 1H-pyrazole-3- carboxamide hydrochloride | LC/MS Rt = 2.44 min [MH]⁺ 531, 533. |
| 350 | 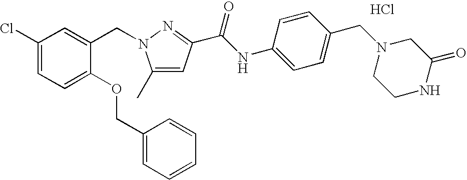 | 1-({5-Chloro-2- [(phenylmethyl)oxy]phenyl} methyl)-5-methyl-N-{4-[(2- oxo-1- piperazinyl)methyl]phenyl}- 1H-pyrazole-3- carboxamide hydrochloride | LC/MS Rt = 2.47 min [MH]⁺ 544, 546. |

### Example 351

### 1-({5-Chloro-2-[(phenylmethyl)oxy]phenyl}methyl)-5-methyl-N-[4-(1-piperazinylmethyl)phenyl]-1H-pyrazole-3-carboxamide hydrochloride

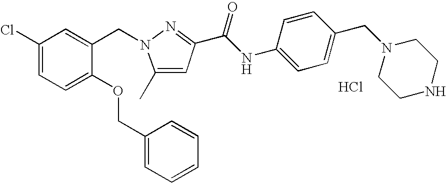

1-({5-Chloro-2-[(phenylmethyl)oxy]phenyl}methyl)-N-(4-formylphenyl)-5-methyl-1H-pyrazole-3-carboxamide (88 mg, 0.19 mmol) was dissolved in dichloromethane (2 ml) and 1,1-dimethylethyl 1-piperazinecarboxylate (71 mg, 0.38 mmol) and acetic acid (12.5 μl, 0.21 mmol) and sodium triacetoxyborohydride (124 mg, 0.57 mmol) added. The reaction was stirred at room temperature for 2 hours then diluted with dichloromethane and water. The organic phase was evaporated and the residue purified by flash chromatography eluting with 10-20% methanol in dichloromethane. The product was dissolved in dichloromethane (1 ml) and 1M hydrogen chloride in diethyl ether (1 ml) added. The resulting solid was collected by decantation and dried to give the title compound (91 mg). LC/MS Rt=2.25 min [MH]⁺ 530, 532.

### Example 352

### 1-({5-Chloro-2-[(phenylmethyl)oxy]phenyl}methyl)-5-methyl-1H-pyrazole-3-carboxamide

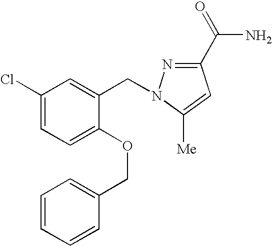

A mixture of 1-({5-chloro-2-[(phenylmethyl)oxy]phenyl}methyl)-5-methyl-1H-pyrazole-3-carboxylic acid (713 mg, 2 mmol), N-methylmorpholine (404 mg, 4 mmol), N-(3-dimethylaminopropyl)-N′-ethylcarbodiimide (460 mg, 2.4 mmol) and ammonium 1H-1,2,3-benzotriazol-1-olate (334 mg, 2.2 mmol) in dichloromethane (10 ml) was stirred at room temperature for 18 hours. The solution was washed with 2M hydrochloric acid and saturated sodium bicarbonate then dried (magnesium sulphate), evaporated and triturated with ether to give the title compound as a white solid (646 mg).

LC/MS Rt 3.20, [MH]⁺ 356.4, 358.4

### 1-({5-Chloro-2-[(2-methylpropyl)oxy]phenyl}methyl)-5-methyl-1H-pyrazole-3-carboxamide

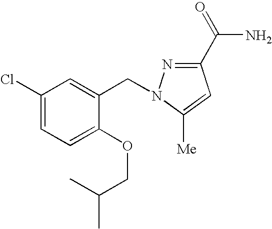

Oxalyl chloride (5 ml) was added to a solution of 1-({5-chloro-2-[(2-methylpropyl)oxy]phenyl}methyl)-5-methyl-1H-pyrazole-3-carboxylic acid (5 g, 15.5 mmol) and one drop of dimethylformamide in dichloromethane (60 ml) and left at room temperature for 2 hours. The solution was evaporated to dryness, dissolved in dichloromethane (50 ml) and concentrated aqueous ammonia (50 ml) added carefully with ice cooling and vigorous stirring. The organic phase was washed with water, dried (magnesium sulphate), evaporated and triturated with ether to give the title compound as a white solid (4.76 g) which was used without further purification.

### 1-[(5-Chloro-2-{[(2,4-difluorophenyl)methyl]oxy}phenyl)methyl]-5-methyl-1H-pyrazole-3-carboxamide

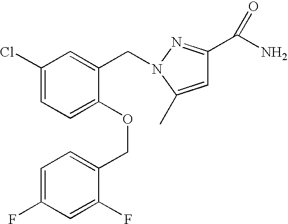

The title compound was prepared in a similar manner to 1-({5-chloro-2-[(2-methylpropyl)oxy]phenyl}methyl)-5-methyl-1H-pyrazole-3-carboxamide from the appropriate intermediates and used without further purification.

### 1-({5-Chloro-2-[(phenylmethyl)oxy]phenyl}methyl)-5-methyl-1H-pyrazole-3-carbonitrile

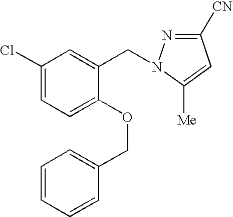

A mixture of 1-({5-chloro-2-[(phenylmethyl)oxy]phenyl}methyl)-5-methyl-1H-pyrazole-3-carboxamide (550 mg, 1.55 mmol) and phosphorus oxychloride (6 ml) was heated at 90° C. for 1 hour. The solution was cooled, evaporated, dissolved in ether/water and the organic phase washed with saturated sodium bicarbonate, dried (magnesium sulphate), evaporated and purified by flash chromatography eluting with ethyl acetate/hexane (1:4) to give the title compound as a white solid (410 mg). LC/MS Rt 3.78, [MH]⁺ 338.4.

### 1-[(5-Chloro-2-{[(2,4-difluorophenyl)methyl]oxy}phenyl)methyl]-5-methyl-1H-pyrazole-3-carbonitrile

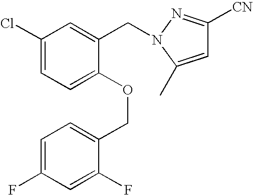

The title compound was prepared in a similar manner to 1-({5-chloro-2-[(phenylmethyl)oxy]phenyl}methyl)-5-methyl-1H-pyrazole-3-carbonitrile from the appropriate intermediates.

### 1-({5-Chloro-2-[(2-methylpropyl)oxy]phenyl}methyl)-5-methyl-1H-pyrazole-3-carbonitrile

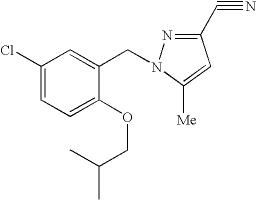

The title compound was prepared in a similar manner to 1-({5-chloro-2-[(phenylmethyl)oxy]phenyl}methyl)-5-methyl-1H-pyrazole-3-carbonitrile from the appropriate intermediates. LC/MS Rt 3.66, [MH]⁺ 304.3.

### Example 353

### 5-[1-({5-Chloro-2-[(phenylmethyl)oxy]phenyl}methyl)-5-methyl-1H-pyrazol-3-yl]-1H-tetrazole

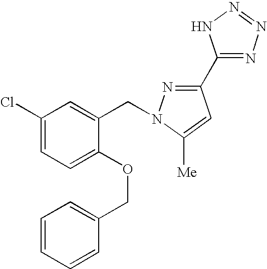

A mixture of ammonium chloride (147 mg, 2.73 mmol), sodium azide (178 mg, 2.73 mmol) and 1-({5-chloro-2-[(phenylmethyl)oxy]phenyl}methyl)-5-methyl-1H-pyrazole-3-carbonitrile (308 mg, 0.91 mmol) in dimethylformamide (6 ml) was stirred and heated at 100° C. under nitrogen for 60 hours. The suspension was cooled, diluted with ethyl acetate/water and the organic phase washed three times with water, dried (magnesium sulphate), evaporated and triturated with ether to give the title compound as a white solid (275 mg).

LC/MS Rt 3.60, [MH]⁺ 381.4, 383.4

### 1-({5-Chloro-2-[(phenylmethyl)oxy]phenyl}methyl)-N-[(1E)-1-(dimethylamino)ethylidene]-5-methyl-1H-pyrazole-3-carboxamide

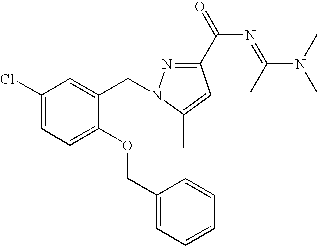

1-({5-Chloro-2-[(phenylmethyl)oxy]phenyl}methyl)-5-methyl-1H-pyrazole-3-carboxamide (500 mg, 1.41 mmol) was stirred in N,N-dimethylacetamide dimethyl acetal (2 ml) and heated to reflux under argon for 2 h. The mixture was diluted with ethyl acetate, washed with water, dried (MgSO₄) and evaporated. The residue was purified by flash chromatography, eluting with 1-2% methanol in dichloromethane to give the title compound as an orange oil (475 mg). LC/MS Rt 2.37 min, [MH]⁺ 425, 427.

### Example 354

### 3-[1-({5-Chloro-2-[(phenylmethyl)oxy]phenyl}methyl)-5-methyl-1H-pyrazol-3-yl]-5-methyl-1H-1,2,4-triazole

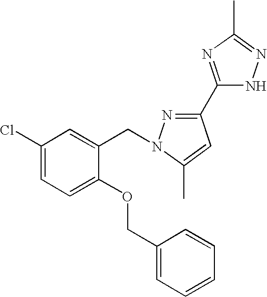

1-({5-Chloro-2-[(phenylmethyl)oxy]phenyl}methyl)-N-[(1E)-1-(dimethylamino)ethylidene]-5-methyl-1H-pyrazole-3-carboxamide (425 mg, 1 mmol) was dissolved in acetic acid (1 ml) and 1,4-dioxan (1 ml) and hydrazine hydrate (55 mg, 54 μl, 1.1 mmol) was added. The mixture was heated at 120° C. for 1 h. The solvent was evaporated and the residue re-evaporated from toluene, then triturated with 1:1 ethyl acetate to diethyl ether. The cream solid was filtered and dried (177 mg). LC/MS Rt 2.82 min, [MH]⁺ 394, 396.

### Example 355

### 1-({5-Chloro-2-[(phenylmethyl)oxy]phenyl}methyl)-5-methyl-N-(phenylsulfonyl)-1H-pyrazole-3-carboxamide

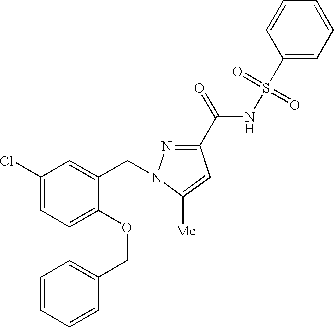

A mixture of 1-({5-chloro-2-[(phenylmethyl)oxy]phenyl}methyl)-5-methyl-1H-pyrazole-3-carboxylic acid (357 mg, 1 mmol), benzenesulphonamide (158 mg, 1 mmol), N-(3-dimethylaminopropyl)-N′-ethylcarbodiimide (212 mg, 1.1 mmol) and 4-dimethylaminopyridine (3 mg) in 1:1 dichloromethane/tetrahydrofuran was stirred at room temperature for 3 days. The solution was diluted with ether, washed with saturated sodium bicarbonate and the organic phase dried (magnesium sulphate), evaporated and purified by flash chromatography eluting with methanol/dichloromethane (1:49) to give, after trituration with ether, the title compound as a white solid (26 mg).

LC/MS Rt 3.78, [MH]⁺ 496.3, 498.3.

### Example 356

### 1-({5-Chloro-2-[(phenylmethyl)oxy]phenyl}methyl)-N-[(3,5-dimethyl-4-isoxazolyl)sulfonyl]-5-methyl-1H-pyrazole-3-carboxamide

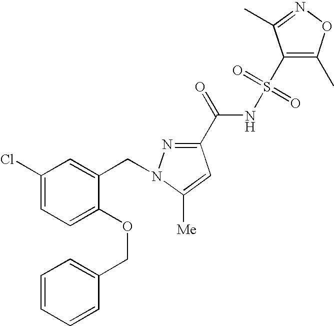

A mixture of 1-({5-chloro-2-[(phenylmethyl)oxy]phenyl}methyl)-5-methyl-1H-pyrazole-3-carboxylic acid (178 mg, 0.5 mmol), 3,5-dimethyl-4-isoxazolesulfonamide (88 mg, 0.5 mmol), N-(3-dimethylaminopropyl)-N′-ethylcarbodiimide (115 mg, 0.6 mmol) and 4-dimethylaminopyridine (3 mg) in 1:1 dichloromethane/tetrahydrofuran (4 ml) was stirred at room temperature for 3 days. The solution was diluted with ethyl acetate, washed with saturated sodium bicarbonate and the organic phase dried (magnesium sulphate), evaporated and purified by flash chromatography eluting with methanol/dichloromethane (1:49) then with ethyl acetate/hexane (2:1) to give, after trituration with ether, the title compound as a white solid (33 mg). LC/MS Rt 3.73, [MH]⁺ 515.3, 517.3

### Example 357

### 1-({5-Chloro-2-[(phenylmethyl)oxy]phenyl}methyl)-N-hydroxy-5-methyl-1H-pyrazole-3-carboxamide

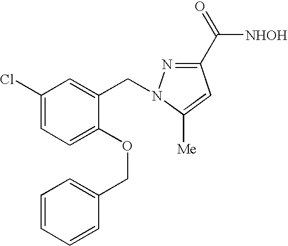

Ethyl chloroformate (142 mg, 1.3 mmol) was added to a solution of 1-({5-chloro-2-[(phenylmethyl)oxy]phenyl}methyl)-5-methyl-1H-pyrazole-3-carboxylic acid (357 mg, 1 mmol) and triethylamine (131 mg, 1.3 mmol) in tetrahydrofuran (5 ml) at 0° C., stirred for 15 minutes then filtered. A solution of hydroxylamine hydrochloride (1 g, 14.4 mmol) in methanol (10 ml) was added to a solution of potassium hydroxide (840 mg, 15 mmol) in methanol (4 ml) at 0° C. The resulting mixture was filtered and 1.5 ml of the filtrate was added to the solution of mixed anhydride prepared above. After 15 minutes the mixture was diluted with ether, washed with water and the organic phase dried (magnesium sulphate), evaporated and purified by flash chromatography eluting with ethyl acetate/hexane (3:2) to give, after trituration with ether, the title compound as a white solid (33 mg). LC/MS Rt 3.07, [MH]⁺ 372.4, 374.4.

### Example 358

### 2-[1-({5-Chloro-2-[(phenylmethyl)oxy]phenyl}methyl)-5-methyl-1H-pyrazol-3-yl]-1,3-benzoxazole

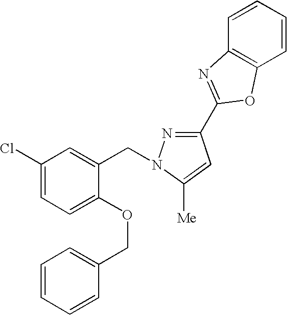

Oxalyl chloride (1 ml) was added to a solution of 1-({5-chloro-2-[(phenylmethyl)oxy]phenyl}methyl)-5-methyl-1H-pyrazole-3-carboxylic acid (357 mg, 1 mmol) and one drop of dimethylformamide in dichloromethane (10 ml). After 45 minutes at room temperature the solution was evaporated to dryness and the residue dissolved in pyridine (3 ml) and 2-aminophenol (120 mg, 1.1 mmol) added. The mixture was heated at 100° C. for 1 hour, cooled and diluted with ether/2M hydrochloric acid. The organic phase was dried (magnesium sulphate), evaporated and purified by flash chromatography eluting with ethyl acetate/hexane (1:3) to give an off-white solid (264 mg). The solid was added to a solution of 4-toluehesulphonic acid (380 mg, 2 mmol) and pyridine (158 mg, 2 mmol) in xylene (5 ml) and evaporated to dryness. The residue was heated at 300° C. for 30 minutes, cooled and purified by flash chromatography eluting with ethyl acetate/hexane (1:4) to give, after trituration with ether, the title compound as an off-white solid (9 mg).

LC/MS Rt 4.04, [MH]⁺ 430.4, 432.4.

### 1-({5-Chloro-2-[(phenylmethyl)oxy]phenyl}methyl)-5-methyl-1H-pyrazole-3-sulfonyl chloride

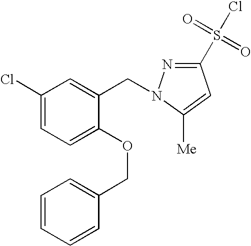

A solution of sodium nitrite (145 mg, 2.1 mmol) in water (1 ml) was added dropwise to a stirred solution of 1-({5-chloro-2-[(phenylmethyl)oxy]phenyl}methyl)-5-methyl-1H-pyrazol-3-amine (655 mg, 2 mmol) in 2:1:1 acetic acid/concentrated hydrochloric acid/water (10 ml) at −10° C. and stirred for 15 minutes then added to a stirred suspension of copper (I) chloride in acetic acid saturated with sulphur dioxide (8 ml) at −10° C. The mixture was allowed to warm to room temperature diluted with ether, washed with water and the organic phase dried (magnesium sulphate), evaporated and purified by flash chromatography eluting with ethyl acetate/hexane (1:4) to give the title compound as a solid (86 mg).

LC/MS Rt 3.95, [MH]⁺ 411.3

### 1-({5-Chloro-2-[(phenylmethyl)oxy]phenyl}methyl)-5-methyl-1H-pyrazole-3-sulfonamide

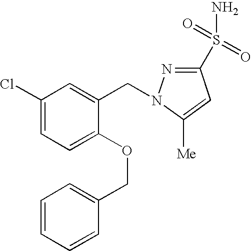

30% Aqueous ammonia solution (1 ml) was added to a solution of 1-({5-chloro-2-[(phenylmethyl)oxy]phenyl}methyl)-5-methyl-1H-pyrazole-3-sulfonyl chloride (200 mg, 0.49 mmol) in tetrahydrofuran (4 ml). After stirring for 30 minutes at room temperature the solution was diluted with ethyl acetate/water and the organic phase dried (magnesium sulphate), evaporated and triturated with ether to give the title compound as a white solid (175 mg). LC/MS Rt 3.22, [MH]⁺ 392.4, 394.3.

### Example 359

### N-{[1-({5-Chloro-2-[(phenylmethyl)oxy]phenyl}methyl)-5-methyl-1H-pyrazol-3-yl]sulfonyl}benzamide

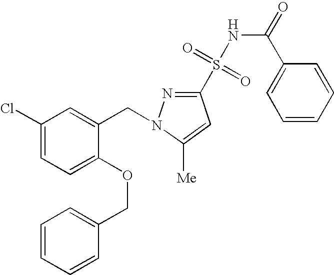

A mixture of 1-({5-chloro-2-[(phenylmethyl)oxy]phenyl}methyl)-5-methyl-1H-pyrazole-3-sulfonamide (157 mg, 0.4 mmol), benzoic acid (61 mg, 0.5 mmol), N-(3-dimethylaminopropyl)-N′-ethylcarbodiimide (115 mg, 0.6 mmol) and 4-dimethylaminopyridine (2 mg) in 1:1 dichloromethane/tetrahydrofuran (4 ml) was stirred at room temperature for 4 days. The solution was diluted with ethyl acetate/2M hydrochloric acid and the organic phase washed with saturated sodium bicarbonate, dried (magnesium sulphate), evaporated and purified by flash chromatography eluting with methanol/dichloromethane (1:49) then repeated with ethyl acetate/hexane (1:3) to give, after trituration with ether, the title compound as a white solid (14 mg).

LC/MS Rt 3.79, [MH]⁺ 496.3, 498.3.

### 1-({5-Chloro-2-[(phenylmethyl)oxy]phenyl}methyl)-N,5-dimethyl-N-(methyloxy)-1H-pyrazole-3-carboxamide

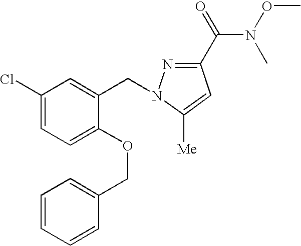

A mixture of 1-({5-chloro-2-[(phenylmethyl)oxy]phenyl}methyl)-5-methyl-1H-pyrazole-3-carboxylic acid (178 mg, 0.5 mmol), N,O-dimethylhydroxylamine hydrochloride (98 mg, 1 mmol), 1-hydroxybenzotriazole (92 mg, 0.6 mmol), N-(3-dimethylaminopropyl)-N′-ethylcarbodiimide (115 mg, 0.6 mmol) and triethylamine (111 mg, 1.1 mmol) in dichloromethane (4 ml) was stirred at room temperature for 16 hours. The resulting solution was diluted with ether, washed with saturated sodium bicarbonate and 2M hydrochloric acid, dried (magnesium sulphate), evaporated to give the title compound as a white solid (185 mg). LC/MS Rt 3.45, [MH]⁺ 400.4, 402.4.

### 1-({5-Chloro-2-[(2-methylpropyl)oxy]phenyl}methyl)-N,5-dimethyl-N-(methyloxy)-1H-pyrazole-3-carboxamide

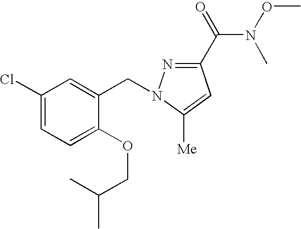

The title compound was prepared in a similar manner to 1-({5-chloro-2-[(phenylmethyl)oxy]phenyl}methyl)-N,5-dimethyl-N-(methyloxy)-1H-pyrazole-3-carboxamide starting from 1-({5-chloro-2-[(2-methylpropyl)oxy]phenyl}methyl)-5-methyl-1H-pyrazole-3-carboxylic acid. LC/MS Rt 3.32, [MH]⁺ 366.3, 368.3.

### 1-[1-({5-Chloro-2-[(phenylmethyl)oxy]phenyl}methyl)-5-methyl-1H-pyrazol-3-yl]ethanone

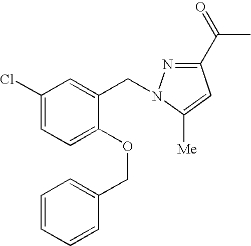

3M Methylmagnesium bromide in ether (0.3 ml) was added to a stirred solution of 1-({5-chloro-2-[(phenylmethyl)oxy]phenyl}methyl)-N,5-dimethyl-N-(methyloxy)-1H-pyrazole-3-carboxamide (180 mg, 0.45 mmol) in tetrahydrofuran (5 ml) under nitrogen. After 30 minutes at room temperature the solution was diluted with ether/2M hydrochloric acid. The organic phase was dried (magnesium sulphate), evaporated and triturated with ether to give the title compound as a white solid (112 mg). LC/MS Rt 3.69, [MH]⁺ 355.4, 357.5.

### 1-[1-({5-Chloro-2-[(2-methylpropyl)oxy]phenyl}methyl)-5-methyl-1H-pyrazol-3-yl]ethanone

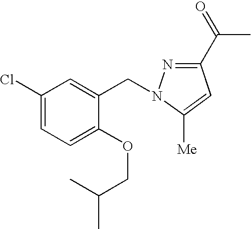

The title compound was prepared in a similar manner to 1-[1-({5-chloro-2-[(phenylmethyl)oxy]phenyl}methyl)-5-methyl-1H-pyrazol-3-yl]ethanone starting from 1-({5-chloro-2-[(2-methylpropyl)oxy]phenyl}methyl)-N,5-dimethyl-N-(methyloxy)-1H-pyrazole-3-carboxamide. LC/MS Rt 3.60, [MH]⁺ 321.3.

### 1-({5-Chloro-2-[(phenylmethyl)oxy]phenyl}methyl)-3-(4,5-dihydro-1H-imidazol-2-yl)-5-methyl-1H-pyrazole

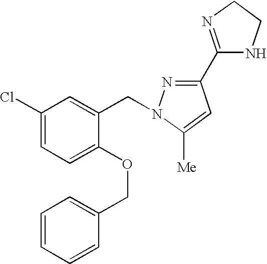

A mixture of 1-({5-chloro-2-[(phenylmethyl)oxy]phenyl}methyl)-5-methyl-1H-pyrazole-3-carbonitrile (169 mg, 0.5 mmol) and phosphorus pentasulphide (7 mg) in ethylenediamine (2 ml) was stirred and heated at 120° C. for 1 hour. After cooling the mixture was diluted with ethyl acetate/water and the organic phase washed three times with water, dried (magnesium sulphate), evaporated and triturated with ether to give the title compound as a white solid (119 mg). LC/MS Rt 2.61, [MH]⁺ 381.4, 383.4.

### 1-[(5-Chloro-2-{[(2,4-difluorophenyl)methyl]oxy}phenyl)methyl]-3-(4,5-dihydro-1H-imidazol-2-yl)-5-methyl-1H-pyrazole

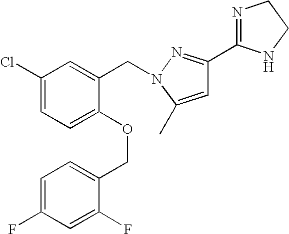

The title compound was prepared in a similar manner to 1-({5-chloro-2-[(phenylmethyl)oxy]phenyl}methyl)-3-(4,5-dihydro-1H-imidazol-2-yl)-5-methyl-1H-pyrazole from the appropriate intermediates.

### Example 360

### 1-({5-Chloro-2-[(phenylmethyl)oxy]phenyl}methyl)-3-(1H-imidazol-2-yl)-5-methyl-1H-pyrazole

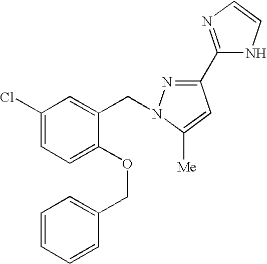

A solution of 1-({5-chloro-2-[(phenylmethyl)oxy]phenyl}methyl)-3-(4,5-dihydro-1H-imidazol-2-yl)-5-methyl-1H-pyrazole (130 mg, 0.34 mmol) in dimethyl sulphoxide (2 ml) was heated at 120° C. for 8 hours then at 150° C. for 8 hours. The mixture was cooled, diluted with ether/water and the organic phase washed twice with water, dried (magnesium sulphate), evaporated and purified by flash chromatography eluting with ethyl acetate/hexane (4:1) to give the title compound as a white solid (26 mg). LC/MS Rt 2.57, [MH]⁺ 379.4, 381.4.

### Example 361

### 1-[(5-Chloro-2-{[(2,4-difluorophenyl)methyl]oxy}phenyl)methyl]-3-(1H-imidazol-2-yl)-5-methyl-1H-pyrazole

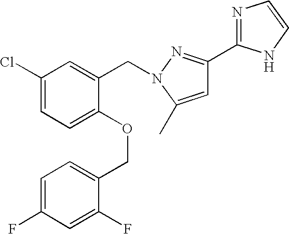

1-[(5-Chloro-2-{[(2,4-difluorophenyl)methyl]oxy}phenyl)methyl]-3-(4,5-dihydro-1H-imidazol-2-yl)-5-methyl-1H-pyrazole (0.065 g, 0.15 mmol) was dissolved in dichloromethane (15 ml). Dess-Martin periodinane (0.1 g, 0.23 mmol) was added portionwise to the mixture over 3 hrs. Stirring was continued for 2 hrs. The mixture was quenched with 5% sodium thiosulphate in saturated sodium bicarbonate solution, stirring was continued for 30 mins. The organic phase was separated, dried (MgSO₄), filtered and concentrated. The residue was purified by chromatography on silica gel with hexane containing ethyl acetate (50%) as eluent, to give the title compound as a white solid (0.034 g). LC/MS Rt 2.85 [MH⁺] 415, 416, 417.

### 2-Bromo-1-[1-({5-chloro-2-[(2-methylpropyl)oxy]phenyl}methyl)-5-methyl-1H-pyrazol-3-yl]ethanone

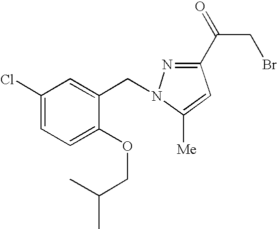

Phenyltrimethylammonium tribromide (377 mg, 1 mmol) was added to a solution of 1-[1-({5-chloro-2-[(2-methylpropyl)oxy]phenyl}methyl)-5-methyl-1H-pyrazol-3-yl]ethanone (321 mg, 1 mmol) in tetrahydrofuran (10 ml) and left at room temperature for 5 hours. The resulting suspension was filtered, evaporated and purified by flash chromatography eluting with ethyl acetate/hexane (1:9) and trituration with ether to give the title compound as a white solid (195 mg). LC/MS Rt 3.85, [MH]⁺ 401.1.

### 2-Bromo-1-[1-({5-chloro-2-[(phenylmethyl)oxy]phenyl}methyl)-5-methyl-1H-pyrazol-3-yl]ethanone

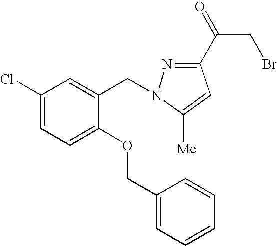

The title compound was prepared in a similar manner to 2-bromo-1-[1-({5-chloro-2-[(2-methylpropyl)oxy]phenyl}methyl)-5-methyl-1H-pyrazol-3-yl]ethanone starting from 1-[1-({5-chloro-2-[(phenylmethyl)oxy]phenyl}methyl)-5-methyl-1H-pyrazol-3-yl]ethanone.

LC/MS Rt 3.73, [MH]⁺ 435.

### Example 362

### 1-({5-Chloro-2-[(2-methylpropyl)oxy]phenyl}methyl)-3-(1H-imidazol-4-yl)-5-methyl-1H-pyrazole hydrochloride

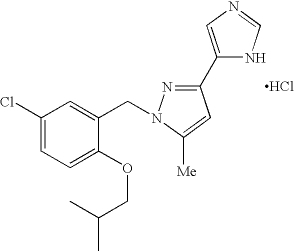

A solution of 2-bromo-1-[1-({5-chloro-2-[(2-methylpropyl)oxy]phenyl}methyl)-5-methyl-1H-pyrazol-3-yl]ethanone (200 mg, 0.5 mmol) in formamide (3 ml) was stirred and heated at 170° C. for 2 hours then cooled, diluted with ethyl acetate/water and made basic with 2M sodium hydroxide. The organic phase was dried (magnesium sulphate), evaporated and purified by flash chromatography eluting with methanol/dichloromethane (1:24). The product was dissolved in methanol, 1M hydrogen chloride in ether (1 ml) added and the solution evaporated to dryness. The residue was triturated with ether to give the title compound as a light brown solid (11 mg). LC/MS Rt 2.33, [MH]⁺ 345.3, 347.3.

### Example 363

### 1-({5-Chloro-2-[(phenylmethyl)oxy]phenyl}methyl)-3-(1H-imidazol-4-yl)-5-methyl-1H-pyrazole hydrochloride

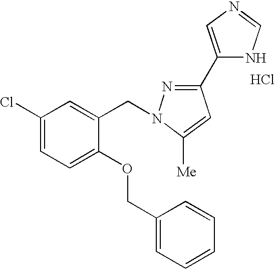

2-Bromo-1-[1-({5-chloro-2-[(phenylmethyl)oxy]phenyl}methyl)-5-methyl-1H-pyrazol-3-yl]ethanone (109 mg, 0.25 mmol) was suspended in formamide (2 ml) and heated at 150° C. in a Smithcreator® microwave for 10 mins. The orange suspension was partitioned between ethyl acetate and water and the organic layer washed twice with water, dried (MgSO₄) and evaporated. The residue was purified by MDAP and the product stirred in 1M hydrogen chloride in diethyl ether for 10 mins, then evaporated. Trituration of the residue with diethyl ether gave the title compound as a cream solid (5 mg).

LC/MS Rt 2.29, [MH]⁺ 379, 381.

### Example 364

### 2-[1-({5-Chloro-2-[(2-methylpropyl)oxy]phenyl}methyl)-5-methyl-1H-pyrazol-3-yl]imidazo[1,2-a]pyridine hydrochloride

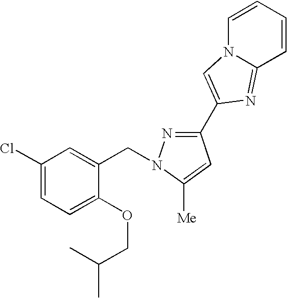

A mixture of 2-bromo-1-[1-({5-chloro-2-[(2-methylpropyl)oxy]phenyl}methyl)-5-methyl-1H-pyrazol-3-yl]ethanone (200 mg, 0.5 mmol) and 2-aminopyridine (94 mg, 1 mmol) in ethanol (4 ml) was stirred and refluxed for 4 hours. The solution was diluted with ether/water and the organic phase dried (magnesium sulphate), evaporated, triturated with ether and the solid filtered off. Dissolved in methanol, 1M hydrogen chloride in ether (1 ml) added and the solution evaporated to dryness. The residue was triturated with ether to give the title compound as a white solid (124 mg). LC/MS Rt 2.48, [MH]⁺ 395.3, 397.3.

The following compounds were prepared using a similar procedure to that described for Example 364 by treating 2-bromo-1-[1-({5-chloro-2-[(2-methylpropyl)oxy]phenyl}methyl)-5-methyl-1H-pyrazol-3-yl]ethanone with the appropriate aminoheterocycle.

| Example | Structure | Name | Data |
|---|---|---|---|
| 365 | 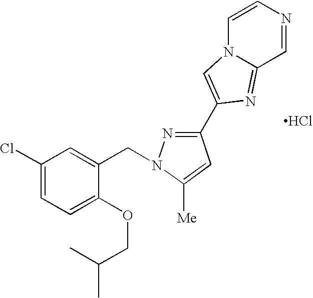 | 2-[1-({5-Chloro-2-[(2- methylpropyl)oxy]phenyl}meth yl)-5-methyl-1H-pyrazol-3- yl]imidazo[1,2-a]pyrazine hydrochloride | LC/MS Rt = 3.15 [MH]⁺ 396.3, 398.3 |
| 366 | 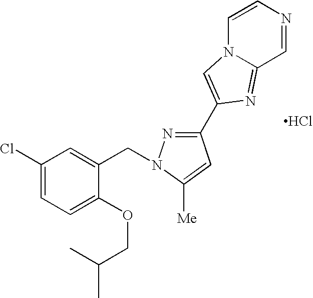 | 2-[1-({5-Chloro-2-[(2- methylpropyl)oxy]phenyl}meth yl)-5-methyl-1H-pyrazol-3- yl]imidazo[1,2-a]pyrimidine hydrochloride | LC/MS Rt = 2.80 [MH]⁺ 396.3, 398.3 |

### Example 367

### 2-[1-({5-Chloro-2-[(phenylmethyl)oxy]phenyl}methyl)-5-methyl-1H-pyrazol-3-yl]-1H-imidazo[4,5-b]pyridine hydrochloride

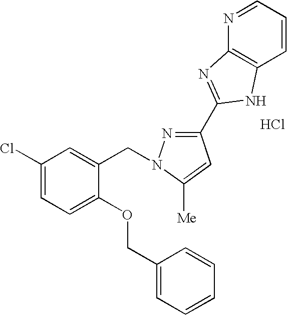

1-({5-Chloro-2-[(phenylmethyl)oxy]phenyl}methyl)-5-methyl-1H-pyrazole-3-carboxylic acid (179 mg, 0.5 mmol) and 2,3-pyridinediamine (55 mg, 0.5 mmol) were dissolved in phosphorus oxychloride (1 ml) and the mixture stirred and heated at 100° C. for 2 hours. The mixture was cooled and poured onto ice water and extracted with ethyl acetate which was washed with water, dried (MgSO₄) and evaporated. The residue was triturated with dichloromethane and the resulting solid stirred in 1M hydrogen chloride in diethyl ether for 10 minutes, filtered and dried to give the title compound as a cream powder (61 mg).

LC/MS Rt=2.83 min. [MH]⁺ 430, 431.

The following compounds were prepared by a similar procedure to that described for Example 367 by treating 2-bromo-1-[1-({5-chloro-2-[(2-methylpropyl)oxy]phenyl}methyl)-5-methyl-1H-pyrazol-3-yl]ethanone with the appropriate diaminoheterocycle.

| Example | Structure | Name | Data |
|---|---|---|---|
| 368 | 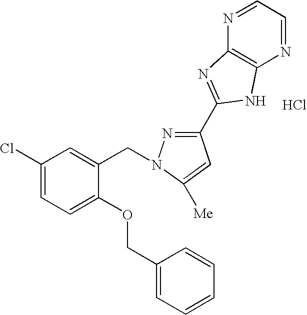 | 2-[1-({5-Chloro-2- [(phenylmethyl)oxy]phenyl} methyl)-5-methyl-1H- pyrazol-3-yl]-1H- imidazo[4,5-b]pyrazine hydrochloride | LC/MS Rt 3.02 min, [MH]⁺ 431, 432. |
| 369 | 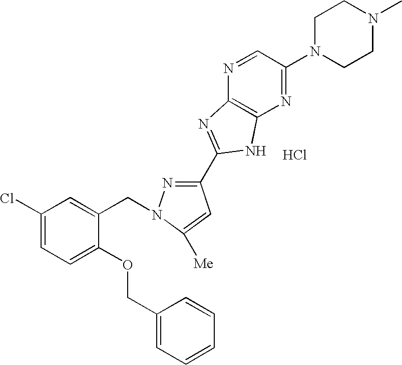 | 2-[1-({5-Chloro-2- [(phenylmethyl)oxy]phenyl} methyl)-5-methyl-1H- pyrazol-3-yl]-5-(4-methyl-1- piperazinyl)-1H- benzimidazole hydrochloride | LC/MS Rt 2.02 min, [MH]⁺ 527, 529. |

### Methyl 1-({5-chloro-2-[(2-methylpropyl)oxy]phenyl}methyl)-5-methyl-1H-pyrazole-3-carboximidoate hydrochloride

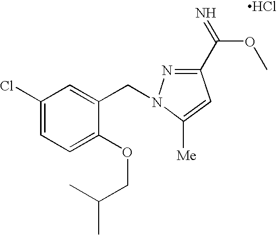

60% Sodium hydride (57 mg, 1.43 mmol) was added to a solution of 1-({5-chloro-2-[(2-methylpropyl)oxy]phenyl}methyl)-5-methyl-1H-pyrazole-3-carbonitrile (4.3 g, 14.2 mmol) in methanol (60 ml) and left at room temperature for 3 days. The solution was evaporated to dryness and the residue dissolved in ether/water and the organic phase dried (magnesium sulphate), filtered and 1M hydrogen chloride in ether (20 ml) added to the filtrate. The gum which separated crystallised on trituration and was filtered off to give the title compound as a white solid (3.6 g). LC/MS Rt 2.21, [MH]⁺ 336.3, 338.3.

### Ethyl 1-[(5-chloro-2-{[(2,4-difluorophenyl)methyl]oxy}phenyl)methyl]-5-methyl-1H-pyrazole-3-carboximidoate hydrochloride

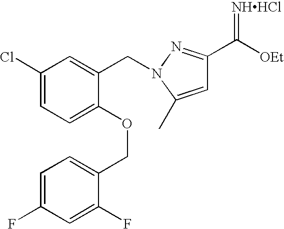

1-[(5-Chloro-2-{[(2,4-difluorophenyl)methyl]oxy}phenyl)methyl]-5-methyl-1H-pyrazole-3-carbonitrile (0.26 g, 0.69 mmol) was suspended in diethyl ether (25 ml) containing ethanol (6 ml) and cooled to 0° C. Hydrogen chloride was bubbled through the mixture to give a saturated solution and the mixture left to stir for 18 hrs. After this time a white solid had precipitated. The solid was filtered and dried to give the title compound. (0.132 g).

### Example 370

### 2-{1-[(5-chloro-2-{[(2,4-difluorophenyl)methyl]oxy}phenyl)methyl]-5-methyl-1H-pyrazol-3-yl}-4,5-dihydro-1,3-oxazole

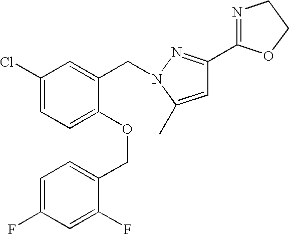

Ethyl 1-[(5-chloro-2-{[(2,4-difluorophenyl)methyl]oxy}phenyl)methyl]-5-methyl-1H-pyrazole-3-carboximidoate hydrochloride (0.132 g, 0.29 mmol) was suspended in ethanol (15 ml). Ethanolamine (0.017 g, 0.29 mmol) was added to the mixture which was heated to reflux for 96 hrs. The mixture was allowed to cool to room temperature then diluted with diethyl ether and water. The layers were separated and the organic phase was dried (MgSO₄), filtered and concentrated. The residue was purified by chromatography on silica gel with hexane containing ethyl acetate (50%) as eluent, to give the title compound (0.013 g)

LC/MS Rt 3.16 [MH⁺] 418, 419, 420

### Example 371

### 2-[1-({5-Chloro-2-[(2-methylpropyl)oxy]phenyl}methyl)-5-methyl-1H-pyrazol-3-yl]-1H-imidazo[4,5-b]pyrazine hydrochloride

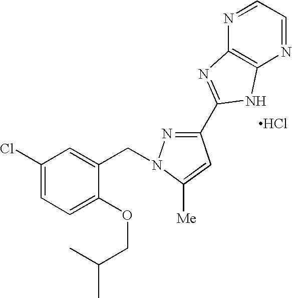

A mixture of 2,3-diaminopyrazine (55 mg, 0.5 mmol) and methyl 1-({5-chloro-2-[(2-methylpropyl)oxy]phenyl}methyl)-5-methyl-1H-pyrazole-3-carboximidoate hydrochloride (186 mg, 0.5 mmol) in ethanol (4 ml) was stirred and refluxed for 4 hours. The solid which separated was filtered, dissolved in hot methanol and 1M hydrogen chloride in ether (0.5 ml) added. The solution was evaporated to dryness and the residue triturated with ether to give the title compound as an off-white solid (17 mg).

LC/MS Rt 3.10, [MH]⁺ 397.2, 399.2

### Example 372

### 2-[1-({5-Chloro-2-[(2-methylpropyl)oxy]phenyl}methyl)-5-methyl-1H-pyrazol-3-yl]-1H-imidazo[4,5-b]pyridine hydrochloride

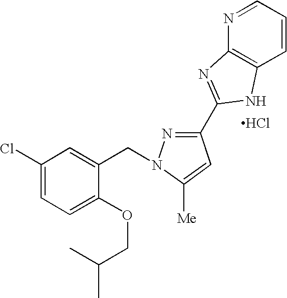

A mixture of 2,3-diaminopyridine (55 mg, 0.5 mmol) and methyl 1-({5-chloro-2-[(2-methylpropyl)oxy]phenyl}methyl)-5-methyl-1H-pyrazole-3-carboximidoate hydrochloride (186 mg, 0.5 mmol) in ethanol (4 ml) was stirred and refluxed for 6 hours. The solution was cooled, diluted with ether/water and the organic phase dried (magnesium sulphate), evaporated and purified by flash chromatography eluting with ethyl acetate. The product was dissolved in methanol, 1M hydrogen chloride in ether (0.5 ml) added and the solution evaporated to dryness. The residue was triturated with ether to give the title compound as an off-white solid (17 mg). LC/MS Rt 2.89, [MH]⁺ 396.2, 398.2.

### Example 373

### 2-[1-({5-Chloro-2-[(2-methylpropyl)oxy]phenyl}methyl)-5-methyl-1H-pyrazol-3-yl]-1H-imidazo[4,5-c]pyridine hydrochloride

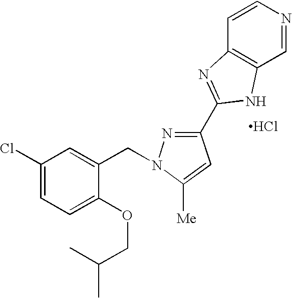

A mixture of 3,4-diaminopyridine (55 mg, 0.5 mmol) and methyl 1-({5-chloro-2-[(2-methylpropyl)oxy]phenyl}methyl)-5-methyl-1H-pyrazole-3-carboximidoate hydrochloride (186 mg, 0.5 mmol) in acetic acid (4 ml) was stirred and refluxed for 4 hours. The solution was cooled, evaporated, the residue dissolved in ethyl acetate/water, made basic with 2M sodium hydroxide and the organic phase dried (magnesium sulphate), evaporated and purified by flash chromatography eluting with methanol/ethyl acetate (1:49). The product was dissolved in methanol, 1M hydrogen chloride in ether (0.5 ml) added and the solution, evaporated to dryness. The residue was triturated with ether to give the title compound as an off-white solid (44 mg). LC/MS Rt 2.28, [MH]⁺ 396.2, 398.3.

### Example 374

### 8-[1-({5-Chloro-2-[(2-methylpropyl)oxy]phenyl}methyl)-5-methyl-1H-pyrazol-3-yl]-1H-purine hydrochloride

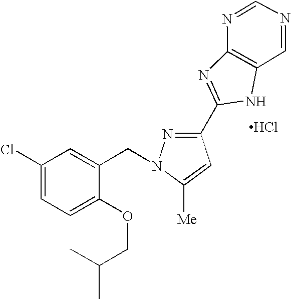

A mixture of 4,5-diaminopyrimidine (55 mg, 0.5 mmol) and methyl 1-({5-chloro-2-[(2-methylpropyl)oxy]phenyl}methyl)-5-methyl-1H-pyrazole-3-carboximidoate hydrochloride (186 mg, 0.5 mmol) in acetic acid (4 ml) was stirred and refluxed for 4 hours. The solution was cooled, evaporated, the residue dissolved in ethyl acetate/water, basified with 2M sodium hydroxide and the organic phase dried (magnesium sulphate), evaporated and purified by flash chromatography eluting with methanol/ethyl acetate (1:49). The product was dissolved in methanol, 1M hydrogen chloride in ether (0.5 ml) added and the solution, evaporated to dryness. The residue was triturated with ether to give the title compound as an off-white solid (13 mg). LC/MS Rt 2.94, [MH]⁺ 397.3, 399.2.

### Example 375

### 2-[1-({5-Chloro-2-[(2-methylpropyl)oxy]phenyl}methyl)-5-methyl-1H-pyrazol-3-yl]-5-(4-methyl-1-piperazinyl)-1H-benzimidazole trihydrochloride

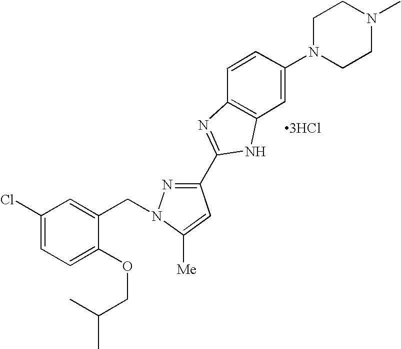

A mixture of 4-(4-methyl-1-piperazinyl)-1,2-benzenediamine (113 mg, 0.55 mmol) and methyl 1-({5-chloro-2-[(2-methylpropyl)oxy]phenyl}methyl)-5-methyl-1H-pyrazole-3-carboximidoate hydrochloride (186 mg, 0.5 mmol) in ethanol (4 ml) was stirred and refluxed for 3 hours. The solution was cooled, diluted with ether/water and the organic phase dried (magnesium sulphate), evaporated and purified by flash chromatography eluting with methanol/dichloromethane (8:92). The product was dissolved in methanol, 1M hydrogen chloride in ether (1 ml) added and the solution, evaporated to dryness. The residue was triturated with ether to give the title compound as an off-white solid (98 mg).

LC/MS Rt 1.98, [MH]⁺ 493.3.

The following compounds were prepared by a similar method to that described for 2-[1-({5-chloro-2-[(2-methylpropyl)oxy]phenyl}methyl)-5-methyl-1H-pyrazol-3-yl]-5-(4-methyl-1-piperazinyl)-1H-benzimidazole trihydrochloride.

| Example | | Name | Data |
|---|---|---|---|
| 376 | 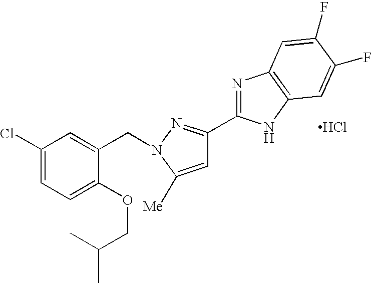 | 2-[1-({5-Chloro-2-[(2- methylpropyl)oxy]phenyl}meth yl)-5-methyl-1H-pyrazol-3-yl]- 5,6-difluoro-1H-benzimidazole | LC/MS Rt = 3.45 [MH]⁺ 431.2, 433.2 |
| | 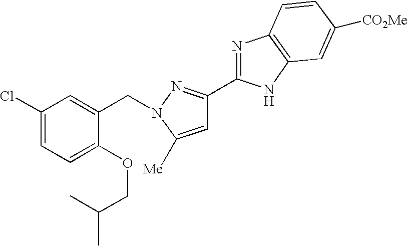 | Methyl 2-[1-({5-chloro-2-[(2- methylpropyl)oxy]phenyl}meth yl)-5-methyl-1H-pyrazol-3-yl]- 1H-benzimidazole-5- carboxylate | LC/MS Rt = 3.32 [MH]⁺ 453.2, 455.3 |

### Example 377

### {2-[1-({5-Chloro-2-[(2-methylpropyl)oxy]phenyl}methyl)-5-methyl-1H-pyrazol-3-yl]-1H-benzimidazol-5-yl}methanol hydrochloride

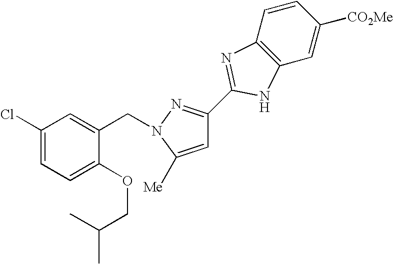

1M Lithium aluminium hydride in ether (3 ml, 3 mmol) was added to a stirred solution of methyl 2-[1-({5-chloro-2-[(2-methylpropyl)oxy]phenyl}methyl)-5-methyl-1H-pyrazol-3-yl]-1H-benzimidazole-5-carboxylate (670 mg, 1.48 mmol) in tetrahydrofuran (25 ml) under argon. After 1 hour the mixture was quenched with 2M sodium hydroxide and extracted with ethyl acetate. The organic phase was dried (magnesium sulphate) and evaporated to give a white solid after trituration with ether (580 mg). The product was dissolved in dichloromethane (5 ml), 1M hydrogen chloride in ether (2 ml) added and the solution evaporated to dryness. The residue was triturated with ether to give the title compound as a white solid. LC/MS: Rt=2.39, [MH]⁺ 425.3, 427.3.

### 2-[1-({5-Chloro-2-[(2-methylpropyl)oxy]phenyl}methyl)-5-methyl-1H-pyrazol-3-yl]-1H-benzimidazole-5-carbaldehyde

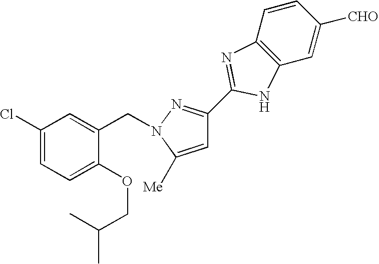

Dess-Martin periodinane (636 mg, 1.5 mmol) was added to a stirred solution of {2-[1-({5-chloro-2-[(2-methylpropyl)oxy]phenyl}methyl)-5-methyl-1H-pyrazol-3-yl]-1H-benzimidazol-5-yl}methanol (510 mg, 1.2 mmol) in dichloromethane (20 ml) under argon. After 1 hour the solution was washed with saturated sodium bicarbonate, dried (magnesium sulphate), evaporated and triturated with ether to give the title compound as a white solid (466 mg). LC/MS: Rt=3.24, [MH]⁺ 423.2, 425.2

### Example 378

### ({2-[1-({5-Chloro-2-[(2-methylpropyl)oxy]phenyl}methyl)-5-methyl-1H-pyrazol-3-yl]-1H-benzimidazol-5-yl}methyl)dimethylamine dihydrochloride

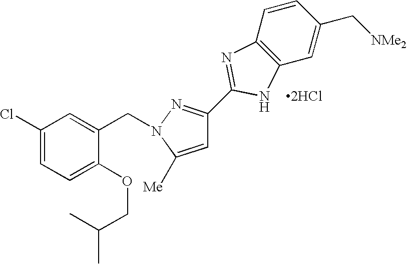

Sodium triacetoxyborohydride (85 mg, 0.4 mmol) was added to a solution of 2M dimethylamine in tetrahydrofuran (0.25 ml, 0.5 mmol) and 2-[1-({5-chloro-2-[(2-methylpropyl)oxy]phenyl}methyl)-5-methyl-1H-pyrazol-3-yl]-1H-benzimidazole-5-carbaldehyde (85 mg, 0.2 mmol) in tetrahydrofuran (3 ml). After 3 hours at room temperature the solution was diluted with ethyl acetate/water and the organic phase dried (magnesium sulphate), evaporated and purified by chromatography on silica gel eluting with methanol/dichloromethane (1:3). The product was dissolved in dichloromethane (5 ml), 1M hydrogen chloride in ether (2 ml) added and the solution evaporated to dryness. The residue was triturated with ether to give the title compound as a white solid (71 mg). LC/MS: Rt=2.17, [MH]⁺ 452.3.

The following examples were prepared by a similar method to that described for ({2-[1-({5-chloro-2-[(2-methylpropyl)oxy]phenyl}methyl)-5-methyl-1H-pyrazol-3-yl]-1H-benzimidazol-5-yl}methyl)dimethylamine dihydrochloride above.

| Example | Structure | Name | Data |
|---|---|---|---|
| 379 | 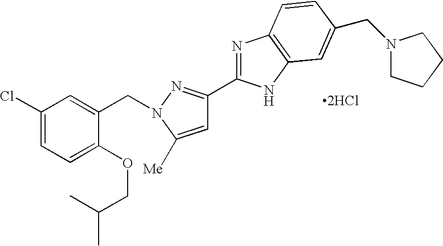 | 2-[1-({5-Chloro-2-[(2- methylpropyl)oxy]phenyl}meth- yl)-5-methyl-1H-pyrazol-3- yl]-5-(1-pyrrolidinylmethyl)- 1H-benzimidazole | LC/MS Rt = 2.19 [MH]⁺ 478.3 |
| 380 | 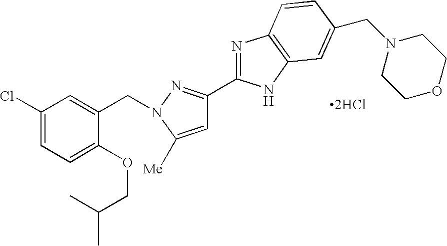 | 2-[1-({5-Chloro-2-[(2- methylpropyl)oxy]phenyl}meth- yl)-5-methyl-1H-pyrazol-3- yl]-5-(4-morpholinylmethyl)- 1H-benzimidazole | LC/MS Rt = 2.19 [MH]⁺ 494.3 |
| 381 | 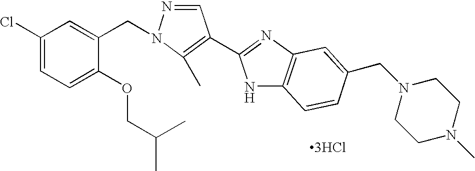 | 2-[1-({5-Chloro-2-[(2- methylpropyl)oxy]phenyl}meth- yl)-5-methyl-1H-pyrazol-4- yl]-5-[(4-methyl-1- piperazinyl)methyl]-1H- benzimidazole trihydrochloride | LC/MS Rt = 2.04 [MH]⁺ 507.3 |
| 382 | 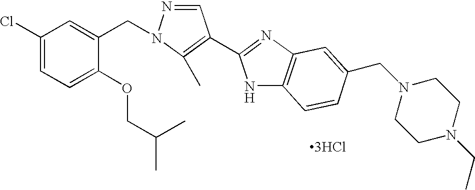 | 2-[1-({5-Chloro-2-[(2- methylpropyl)oxy]phenyl}meth- yl)-5-methyl-1H-pyrazol-4- yl]-5-[(4-ethyl-1- piperazinyl)methyl]-1H- benzimidazole trihydrochloride | LC/MS Rt = 2.03 [MH]⁺ 521.4 |

### Example 383

### ({2-[1-({5-Chloro-2-[(2-methylpropyl)oxy]phenyl}methyl)-5-methyl-1H-pyrazol-4-yl]-1H-benzimidazol-5-yl}methyl)methylamine dihydrochloride

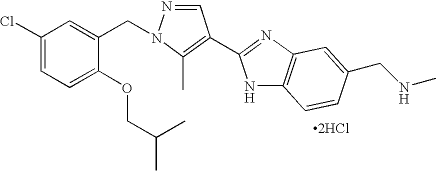

A solution of 2-[1-({5-chloro-2-[(2-methylpropyl)oxy]phenyl}methyl)-5-methyl-1H-pyrazol-4-yl]-1H-benzimidazole-5-carbaldehyde (70 mg, 0.165 mmol) and 33% methylamine in ethanol (0.2 ml, 2.1 mmol) in tetrahydrofuran (3 ml) was stirred at room temperature for 1 hour and sodium borohydride (38 mg, 1 mmol) added. The mixture was stirred at room temperature for 2 hours, diluted with ethyl acetate/water and the organic phase dried (magnesium sulphate), evaporated and purified by flash chromatography eluting with methanol/dichloromethane (1:19) to remove impurities then with methanol/dichloromethane (2:1). The product was dissolved in dichloromethane (5 ml), 1M hydrogen chloride in ether (2 ml) added and the solution evaporated to dryness. The residue was triturated with ether to give the title compound as a white solid (45 mg). LC/MS: Rt=2.16, [MH]⁺ 438.3

### [1-({5-Chloro-2-[(phenylmethyl)oxy]phenyl}methyl)-5-methyl-1H-pyrazol-3-yl]methanol

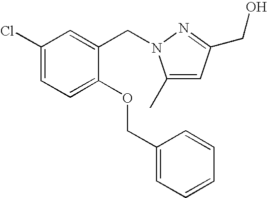

Ethyl 1-({5-chloro-2-[(phenylmethyl)oxy]phenyl}methyl)-5-methyl-1H-pyrazole-3-carboxylate (1.15 g, 3 mmol) was dissolved in tetrahydrofuran (10 ml) and cooled to 0° C. under argon with stirring. 1M Lithium aluminium hydride in diethyl ether (3 ml, 3 mmol) was added at 0-5° C. over 10 minutes and the reaction stirred at 0° C. for 1 h. Water (2 ml) was carefully added. When effervescence had ceased, diethyl ether and water were added and the organic layer washed with brine, dried (MgSO₄) and evaporated. The residue was purified by flash chromatography, eluting with 5-30% ethyl acetate in hexane to give the title compound as a yellow oil (600 mg).

¹H NMR (CDCl₃) δ: 1.98 (1H, m), 2.14 (3H, s), 4.65 (2H, d), 5.09 (2H, s), 5.24 (2H, s), 6.07 (1H, s), 6.59 (1H, d), 6.85 (1H, d), 7.16 (1H, dd), 7.35 (5H, m).

### 1-({5-Chloro-2-[(phenylmethyl)oxy]phenyl}methyl)-5-methyl-1H-pyrazole-3-carbaldehyde

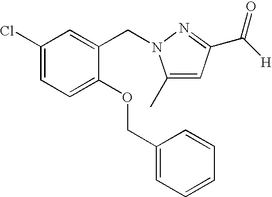

[1-({5-Chloro-2-[(phenylmethyl)oxy]phenyl}methyl)-5-methyl-1H-pyrazol-3-yl]methanol (600 mg, 1.75 mmol) was dissolved in dichloromethane (10 ml) and Dess-Martin periodinane (817 mg, 1.93 mmol) added. The mixture was stirred at room temperature for 2 h then washed with 5% sodium thiosulphate solution and water, dried (MgSO₄) and evaporated. The residue was purified by flash chromatography, eluting with 5-30% ethyl acetate in hexane to give the title compound as a yellow oil (375 mg).

¹H NMR (CDCl₃) δ: 2.16 (3H, s), 5.08 (2H, s), 5.30 (2H, s), 6.59 (1H, d), 6.74 (1H, d) 6.89 (1H, d), 7.21 (1H, dd), 7.37 (5H, m), 9.92 (1H, s).

### 1-({5-Chloro-2-[(2-methylpropyl)oxy]phenyl}methyl)-5-methyl-1H-pyrazole-3-carbaldehyde

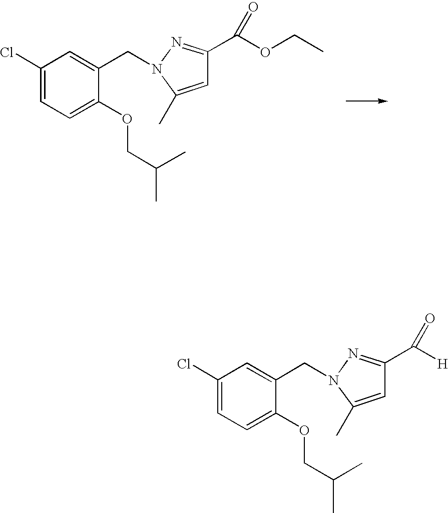

Ethyl 1-({5-chloro-2-[(2-methylpropyl)oxy]phenyl}methyl)-5-methyl-1H-pyrazole-3-carboxylate (0.577 g, 1.65 mmol) was dissolved in tetrahydrofuran-ethanol (1:1, 3.6 ml) and cooled to 0° C. 2M Lithium borohydride in tetrahydrofuran (3.6 ml, 1.8 mmol) was added and the mixture stirred at room temperature for 22 hours. Further tetrahydrofuran-ethanol (1:1, 3.6 ml) and 2M lithium borohydride (3.6 ml, 1.8 mmol) were added and the mixture heated at 70° C. for 4 hours. Upon cooling to room temperature, wet tetrahydrofuran was added and the mixture was stirred for 5 minutes. Ethyl acetate and 2M hydrochloric acid were added. The layers were separated and the organic phase was washed with saturated sodium bicarbonate, dried (Na₂SO₄), filtered and concentrated to give a residue which was dissolved in dichloromethane (7.7 ml) and cooled to 0° C. Dess-Martin periodinane (0.65 g, 1.53 mmol) was added. Stirring was continued for 2.5 hours. The mixture was then diluted with dichloromethane and washed with saturated sodium bicarbonate containing sodium thiosulphate, dried (Na₂SO₄), filtered and concentrated. The residue was purified by chromatography on silica gel with hexane containing ethyl acetate (10-20%) as eluent, to give the title compound (0.327 g) as a white solid. LC/MS Rt 3.53 [MH+] 307, 309.

### Example 384

### 1-({5-Chloro-2-[(2-methylpropyl)oxy]phenyl}methyl)-3-(1H-imidazol-2-yl)-5-methyl-1H-pyrazole

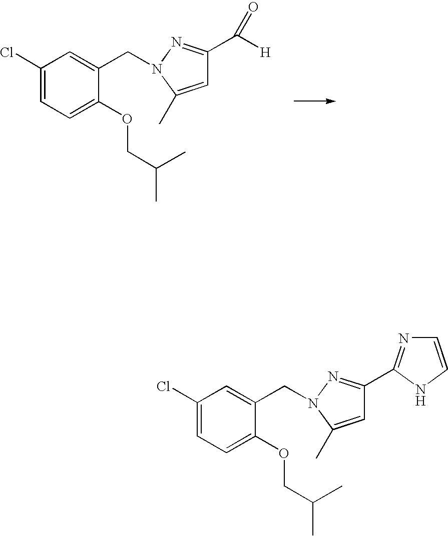

1-({5-Chloro-2-[(2-methylpropyl)oxy]phenyl}methyl)-5-methyl-1H-pyrazole-3-carbaldehyde (0.105 g, 0.34 mmol), ammonium acetate (0.98 g, 12.73 mmol) and 40% aqueous glyoxal (0.100 g, 0.68 mmol) were heated in acetic acid at 100° C. with microwave irradiation for 10 minutes, followed by a further 30 minutes at the same temperature. The solution was then heated at 150° C. with microwave irradiation for 1 hour. Upon cooling to room temperature, the mixture was slowly poured into saturated sodium bicarbonate and then extracted with ethyl acetate, which was dried (Na₂SO₄) and evaporated. The crude product was purified by MDAP to give the title compound (0.003 g). LC/MS Rt 2.28 min [MH⁺] 345, 347.

### Example 385

### 2-[1-({5-Chloro-2-[(phenylmethyl)oxy]phenyl}methyl)-5-methyl-1H-pyrazol-3-yl]-1H-benzimidazole

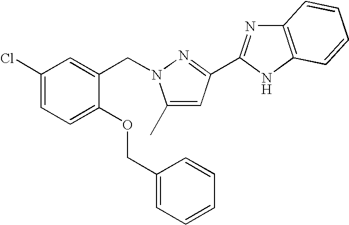

1-({5-Chloro-2-[(phenylmethyl)oxy]phenyl}methyl)-5-methyl-1H-pyrazole-3-carbaldehyde (68 mg, 0.2 mmol), sodium bisulphite (24 mg, 0.23 mmol) and 1,2 phenylenediamine (22 mg, 0.2 mmol) were dissolved in methanol (2 ml) and the solution heated at reflux for 1 h. After cooling, diethyl ether and water were added and the organic phase washed with brine, dried (MgSO₄) and evaporated. The residue was purified by flash chromatography, eluting with 5-30% ethyl acetate in hexane and the product triturated with diethyl ether to give the title compound as a cream powder (30 mg). LC/MS Rt 2.57 min, [MH]⁺ 429, 431.

### Example 386

### 2-[1-({5-Chloro-2-[(2-methylpropyl)oxy]phenyl}methyl)-5-methyl-1H-pyrazol-3-yl]-1H-benzimidazole

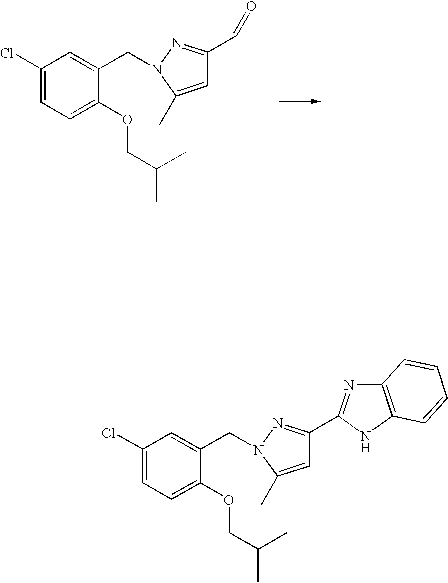

1-({5-Chloro-2-[(2-methylpropyl)oxy]phenyl}methyl)-5-methyl-1H-pyrazole-3-carbaldehyde (0.097 g, 0.32 mmol), 1,2-phenylenediamine (0.038 g, 0.35 mmol) and NaHSO₃ (0.037 g, 0.35 mmol) were heated in methanol (2 ml) at 70° C. with microwave irradiation for 10 minutes. Upon cooling to room temperature, the mixture was diluted with ethyl acetate and washed with saturated sodium bicarbonate, dried (Na₂SO₄), filtered and evaporated. The residue was purified by MDAP to give the title compound (0.024 g).

LC/MS Rt 2.62 [MH⁺] 395, 397

### Example 387

### N-[2,2-Bis(ethyloxy)ethyl]-1-({5-chloro-2-[(2-methylpropyl)oxy]phenyl}methyl)-5-methyl-1H-pyrazole-3-carboxamide

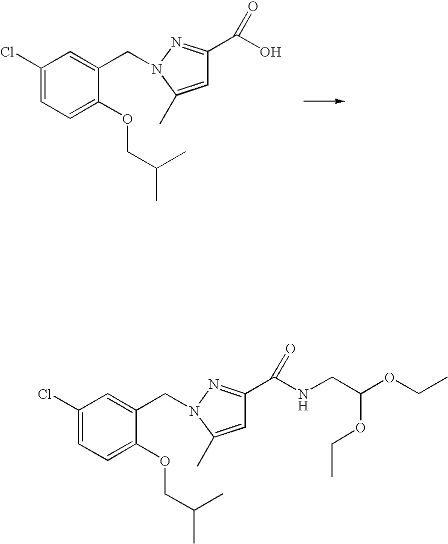

1-({5-Chloro-2-[(2-methylpropyl)oxy]phenyl}methyl)-5-methyl-1H-pyrazole-3-carboxylic acid (0.276 g, 0.86 mmol) was dissolved in dichloromethane (1.7 ml) Thionyl chloride (0.31 ml, 4.29 mmol) was added and the mixture was heated at reflux for 4.5 hours. Upon cooling to room temperature the solvent was evaporated and the crude residue was dissolved in dichloromethane (2.1 ml) and triethylamine (0.24 ml, 1.72 mmol) then cooled to 0° C. Aminoacetaldehyde diethyl acetal (0.19 ml, 1.29 mmol) in dichloromethane (2.2 ml) was added slowly. The mixture was left to stir overnight then evaporated. The residue was purified by chromatography on silica gel with hexane containing ethyl acetate (20-40%) as eluent, to give the title compound (0.311 g).

### Example 388

### 2-[1-({5-Chloro-2-[(2-methylpropyl)oxy]phenyl}methyl)-5-methyl-1H-pyrazol-3-yl]-1,3-oxazole

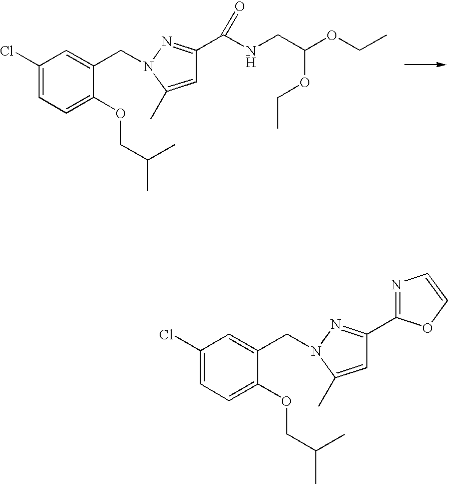

N-[2,2-Bis(ethyloxy)ethyl]-1-({5-chloro-2-[(2-methylpropyl)oxy]phenyl}methyl)-5-methyl-1H-pyrazole-3-carboxamide (0.101 g, 0.23 mmol) was stirred in a mixture of tetrahydrofuran (0.9 ml) and 2M hydrochloric acid (0.45 ml) at room temperature overnight. The mixture was diluted with ethyl acetate and washed with saturated sodium bicarbonate, dried (Na₂SO₄), filtered and concentrated to give 1-({5-chloro-2-[(2-methylpropyl)oxy]phenyl}methyl)-5-methyl-N-(2-oxoethyl)-1H-pyrazole-3-carboxamide which was dissolved in dichloromethane (1.5 ml) and added to a solution of triethylamine (0.13 ml, 0.92 mmol), iodine (0.12 g, 0.46 mmol) and triphenylphosphine (0.12 g, 0.46 mmol) in dichloromethane (1.5 ml) at room temperature. After stirring for 30 minutes the solvent was evaporated and the residue was purified by chromatography on silica gel with hexane containing ethyl acetate (10-30%) as eluent, to give the title compound (0.20 g).

LC/MS Rt 3.40 [MH⁺] 346, 348.

### Example 389

### 2-[1-({5-Bromo-2-[(phenylmethyl)oxy]phenyl}methyl)-5-methyl-1H-pyrazol-3-yl]-1,3-oxazole

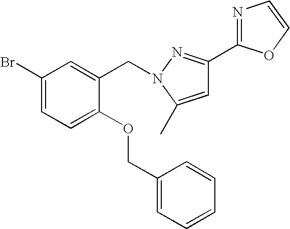

1-({5-Bromo-2-[(phenylmethyl)oxy]phenyl}methyl)-5-methyl-1H-pyrazole-3-carboxylic acid (0.159 g, 0.40 mmol) was heated at reflux in dichloromethane (0.8 ml) containing thionyl chloride (0.15 ml, 2.15 mmol) for 2.5 hours. Upon cooling to room temperature, the mixture was concentrated and the residue dissolved in dichloromethane (1 ml) containing triethylamine (0.11 ml, 0.79 mmol) and cooled to 0° C. Aminoacetaldehyde diethyl acetal (0.09 ml, 0.98 mmol) in dichloromethane (1.0 ml) was added slowly. The mixture was stirred for 1.5 hours, during which time it was allowed to warm to room temperature. The mixture was then diluted with dichloromethane and 2M hydrochloric acid and stirred vigorously. The organic layer was separated and stirred vigorously with saturated sodium bicarbonate. The dichloromethane solution was separated and dried (Na₂SO₄) filtered and evaporated to give a residue which was dissolved in tetrahydrofuran (1.6 ml) and stirred at room temperature with 2M hydrochloric acid overnight. The mixture was diluted with ethyl acetate and saturated sodium bicarbonate. The layers were separated and the organic phase was dried (Na₂SO₄), filtered and concentrated. The residue was dissolved in dichloromethane (1.5 ml) and added to a stirred solution of triphenylphosphine (0.210 g, 0.8 mmol), iodine (0.203 g, 0.8 mmol) and triethylamine (0.21 ml, 1.5 mmol) in dichloromethane (3 ml) at room temperature. After 15 minutes the reaction was quenched with 2M hydrochloric acid. The layers were separated and the organic phase was washed with saturated sodium bicarbonate containing sodium thiosulphate, dried (Na₂SO₄), filtered and concentrated. The residue was purified by chromatography on silica gel with hexane containing ethyl acetate (10-30%) as eluent, to give the title compound (0.036 g) as a white solid. LC/MS Rt 3.32 min [MH+] 424, 426.

### Example 390

### 1-({5-Chloro-2-[(2-methylpropyl)oxy]phenyl}methyl)-5-methyl-N-(1,2,3,4-tetrahydro-6-isoquinolinyl)-1H-pyrazole-3-carboxamide hydrochloride

### (a) 1,1-Dimethylethyl 6-({[1-({5-chloro-2-[(2-methylpropyl)oxy]phenyl}methyl)-5-methyl-1H-pyrazol-3-yl]carbonyl}amino)-3,4-dihydro-2(1H)-isoquinolinecarboxylate

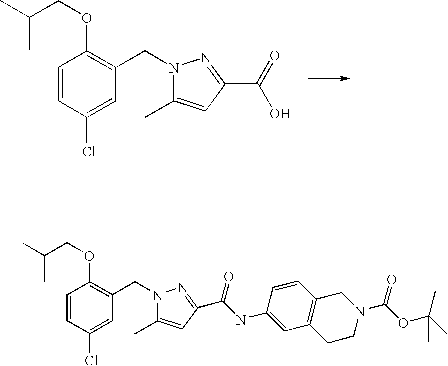

To a solution of 1-({5-chloro-2-[(2-methylpropyl)oxy]phenyl}methyl)-5-methyl-1H-pyrazole-3-carboxylic acid (140 mg, 0.43 mmol), N-(3-dimethylaminopropyl)-N′-ethylcarbodiimide hydrochloride (124 mg, 0.65 mmol), 1-hydroxybenzotriazole hydrate (88 mg, 0.65 mmol) and triethylamine (65 mg, 0.65 mmol) in DCM (5 mL) was added 1,1-dimethylethyl 6-amino-3,4-dihydro-2(1H)-isoquinolinecarboxylate (108 mg, 0.43 mmol) and the mixture was stirred at room temperature for 5 hours. The organic phase was then washed with HCl (1N), NaOH (1N) and brine, dried over Na₂SO₄, filtered and evaporated under reduced pressure. The residue was purified by flash column chromatography eluting with CH₂Cl₂/MeOH: 99/1 to give the title compound as a white solid (40 mg). LC/MS: m/z 553 (M+H)⁺, Rt: 4.28 min.

### (b) 1-({5-Chloro-2-[(2-methylpropyl)oxy]phenyl}methyl)-5-methyl-N-(1,2,3,4-tetrahydro-6-isoquinolinyl)-1H-pyrazole-3-carboxamide hydrochloride

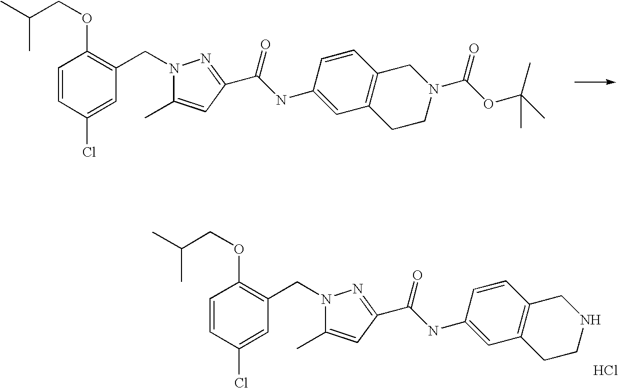

To a solution of 1,1-dimethylethyl 6-({[1-({5-chloro-2-[(2-methylpropyl)oxy]phenyl}methyl)-5-methyl-1H-pyrazol-3-yl]carbonyl}amino)-3,4-dihydro-2(1H)-isoquinolinecarboxylate (35 mg, 0.06 mmol) in DCM (3 mL) was added trifluoroacetic acid (0.5 mL) and the mixture was stirred at room temperature for 2 hours and evaporated to dryness under reduced pressure. The mixture was basified with a saturated solution of NaHCO₃ and extracted with AcOEt. The organic phase was washed with brine, dried over Na₂SO₄, filtered and evaporated under reduced pressure. The residue was dissolved in diethyl ether and 1N HCl in diethyl ether was added. The resulting precipitate was filtered and washed with diisopropyl ether to give the title compound as a pale yellow solid (25 mg). LC/MS: m/z 453 (M+H)⁺, Rt: 3.21 min.

### Example 391

### 1-({5-Chloro-2-[(2-methylpropyl)oxy]phenyl}methyl)-N-[4-(hydroxyl-methyl)phenyl]-5-methyl-1H-pyrazole-3-carboxamide

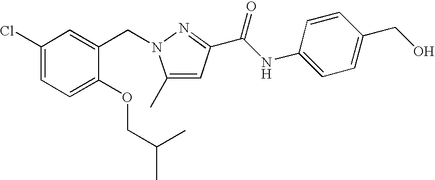

A solution of 1-({5-chloro-2-[(2-methylpropyl)oxy]phenyl}methyl)-5-methyl-1H-pyrazole-3-carboxylic acid (1.94 g), 4-aminobenzyl alcohol (739 mg), N-(3-dimethylaminopropyl)-N′-ethylcarbodiimide hydrochloride (1.24 g) and 1-hydroxybenzotriazole hydrate (975 mg) in dichloromethane (25 ml) and dimethylformamide (25 ml) was stirred at ambient temperature for 5 hours. Ethyl acetate (120 ml) was added and the mixture washed with water (120 ml), saturated sodium bicarbonate solution (45 ml) and brine (2×35 ml), dried (MgSO₄) and concentrated in vacuo. The residue was recrystallised from ethanol to afford the title compound (1.72 g, 67%) as a white solid. LC/MS [MH+]=428/430, RT=3.30 min.

### Example 392

### 1-({5-Chloro-2-[(2-methylpropyl)oxy]phenyl}methyl)-5-methyl-N-{4-[(methylamino)methyl]phenyl}-1H-pyrazole-3-carboxamide hydrochloride

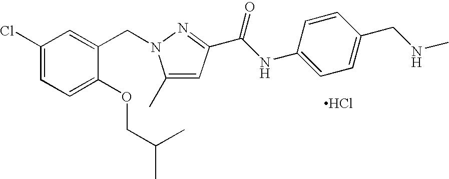

### (a). 1-({5-Chloro-2-[(2-methylpropyl)oxy]phenyl}methyl)-N-(4-formyl-phenyl)-5-methyl-1H-pyrazole-3-carboxamide

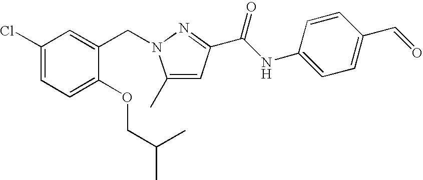

To a solution/suspension of 1-({5-chloro-2-[(2-methylpropyl)oxy]phenyl}-methyl)-N-[4-(hydroxylmethyl)phenyl]-5-methyl-1H-pyrazole-3-carboxamide (1.68 g) in dry dichloromethane (20 ml) was added under argon Dess-Martin periodinane (1.67 g) and the mixture stirred at ambient temperature for 1.5 hours. It was washed with saturated sodium bicarbonate solution (30 ml) and the aqueous layer extracted with dichloromethane (2×10 ml). The combined dichloromethane layers were washed with brine (15 ml), dried (MgSO₄) and concentrated in vacuo. The residue was purified by SP4 Biotage chromatography using 5 to 40% ethyl acetate in hexane to afford the title compound (1.47 g, 88%) as a white foamy solid. LC/MS [MH+]=426/438, RT=3.68 min.

### (b). 1-({5-Chloro-2-[(2-methylpropyl)oxy]phenyl}methyl)-5-methyl-N-{4-[(methylamino)methyl]phenyl}-1H-pyrazole-3-carboxamide hydrochloride

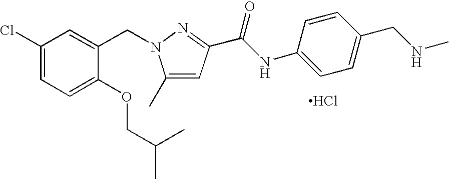

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added 33% methylamine in ethanol (88 μl) and the solution stirred under argon for 1 hour. Sodium triacetoxyborohydride (68 mg) and acetic acid (10 μl) were added and stirring continued for 2 hours. Further aliquots of 33% methylamine in ethanol (3×150 μl) and sodium triacetoxyborohydride (68 mg) were added and stirring continued for 2 hours. Ethyl acetate (12 ml) and brine (12 ml) were added and the aqueous layer separated and extracted with ethyl acetate (5 ml). The combined organic extracts were dried (MgSO₄) and concentrated in vacuo. The residue was purified by mass-directed autopreparation then dissolved in dichloromethane (5 ml). 1M hydrogen chloride in ether (5 ml) was added and solvent removed in vacuo. The residue was washed with hexane and dried in vacuo to afford the title compound (24.9 mg, 33%) as a pale cream solid.

LC/MS [MH+]=441/443, RT=2.38 min.

### Example 393

### 1-({5-Chloro-2-[(2-methylpropyl}oxy]phenyl}methyl)-N-{4-[(ethylamino)methyl]phenyl}-5-methyl-1H-pyrazole-3-carboxamide hydrochloride

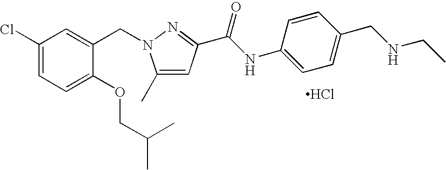

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added 2M ethylamine in tetrahydrofuran (48 μl) and the solution stirred under argon for 1 hour. Sodium triacetoxyborohydride (68 mg) and acetic acid (10 μl) were added and stirring continued for 2 hours. Further aliquots of 2M ethylamine in tetrahydrofuran (60 μl) and sodium triacetoxyborohydride (68 mg) were added and stirring continued for 2 hours. Ethyl acetate (12 ml) and brine (12 ml) were added and the aqueous layer separated and extracted with ethyl acetate (5 ml). The combined organic extracts were dried (MgSO₄) and concentrated in vacuo. The residue was purified by mass-directed autopreparation then dissolved in dichloromethane (5 ml). 1M hydrogen chloride in ether (5 ml) was added and solvent removed in vacuo. The residue was washed with hexane and dried in vacuo to afford the title compound (24.3 mg, 31%) as a pale cream solid.

LC/MS [MH+]=455/457, RT=2.42 min.

### Example 394

### 1-({5-Chloro-2-[(2-methylpropyl)oxy]phenyl}methyl)-N-{4-[(dimethylamino)methyl]phenyl}-5-methyl-1H-pyrazole-3-carboxamide hydrochloride

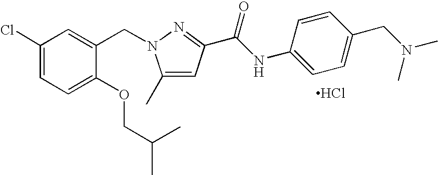

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added 33% dimethylamine in ethanol (128 μl) and the solution stirred under argon for 1 hour. Sodium triacetoxyborohydride (68 mg) and acetic acid (10 μl) were added and stirring continued for 2 hours. Ethyl acetate (12 ml) and brine (12 ml) were added and the aqueous layer separated and extracted with ethyl acetate (5 ml). The combined organic extracts were dried (MgSO₄) and concentrated in vacuo. The residue was purified by mass-directed autopreparation then dissolved in dichloromethane (5 ml). 1M hydrogen chloride in ether (5 ml) was added and solvent removed in vacuo. The residue was washed with hexane and dried in vacuo to afford the title compound (44.4 mg, 57%) as a pale cream solid. LC/MS [MH+]=455/457, RT=2.40 min.

### Example 395

### 1-({5-Chloro-2-[(2-methylpropyl)oxy]phenyl}methyl)-5-methyl-N-(4-{[(1-methylethyl)amino]methyl}phenyl)-1H-pyrazole-3-carboxamide hydrochloride

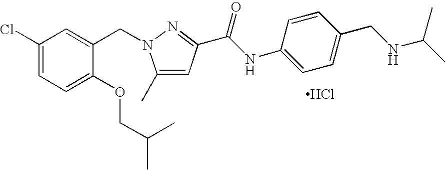

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added isopropylamine (28 μl) and the solution stirred under argon for 1 hour. Sodium triacetoxyborohydride (68 mg) and acetic acid (10 μl) were added and stirring continued for 2 hours. Further aliquots of isopropylamine (20 μl) and sodium triacetoxyborohydride (68 mg) were added and stirring continued for 2 hours. Ethyl acetate (12 ml) and brine (12 ml) were added and the aqueous layer separated and extracted with ethyl acetate (5 ml). The combined organic extracts were dried (MgSO₄) and concentrated in vacuo. The residue was purified by mass-directed autopreparation then dissolved in dichloromethane (5 ml). 1M hydrogen chloride in ether (5 ml) was added and solvent removed in vacuo. The residue was washed with hexane and dried in vacuo to afford the title compound (9.0 mg, 11%) as a pale buff solid. LC/MS [MH+]=469/471, RT=2.46 min.

### Example 396

### 1-({5-Chloro-2-[(2-methylpropyl)oxy]phenyl}methyl)-5-methyl-N-[4-(1-pyrrolidinylmethyl)phenyl]-1H-pyrazole-3-carboxamide hydrochloride

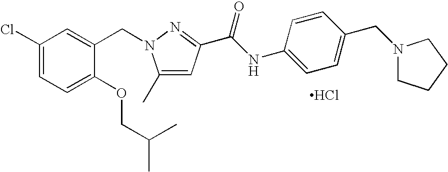

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added pyrrolidine (27 μl) and the solution stirred under argon for 1 hour. Sodium triacetoxyborohydride (68 mg) and acetic acid (10 μl) were added and stirring continued for 2 hours. Ethyl acetate (12 ml) and brine (12 ml) were added and the aqueous layer separated and extracted with ethyl acetate (5 ml). The combined organic extracts were dried (MgSO₄) and concentrated in vacuo. The residue was purified by mass-directed autopreparation then dissolved in dichloromethane (5 ml). 1M hydrogen chloride in ether (5 ml) was added and solvent removed in vacuo. The residue was washed with hexane and dried in vacuo to afford the title compound (42.3 mg, 51%) as a pale cream solid.

LC/MS [MH+]=481/483, RT=2.48 min.

### Example 397

### 1-({5-Chloro-2-[(2-methylpropyl)oxy]phenyl}methyl)-5-methyl-N-[4-(1-piperidinylmethyl)phenyl]-1H-pyrazole-3-carboxamide hydrochloride

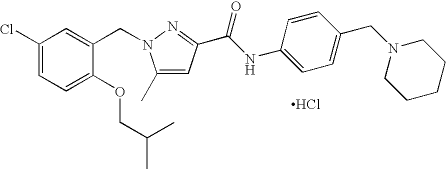

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added piperidine (32 μl) and the solution stirred under argon for 1 hour. Sodium triacetoxyborohydride (68 mg) and acetic acid (10 μl) were added and stirring continued for 2 hours. Further aliquots of piperidine (15 μl) and sodium triacetoxyborohydride (68 mg) were added and stirring continued for 2 hours. Ethyl acetate (12 ml) and brine (12 ml) were added and the aqueous layer separated and extracted with ethyl acetate (5 ml). The combined organic extracts were dried (MgSO₄) and concentrated in vacuo. The residue was purified by mass-directed autopreparation then dissolved in dichloromethane (5 ml). 1M hydrogen chloride in ether (5 ml) was added and solvent removed in vacuo. The residue was washed with hexane and dried in vacuo to afford the title compound (20.3 mg, 24%) as a pale cream solid. LC/MS [MH+]=495/497, RT=2.53 min.

### Example 398

### 1-({5-Chloro-2-[(2-methylpropyl)oxy]phenyl}methyl)-5-methyl-N-[4-(4-morpholinylmethyl)phenyl]-1H-pyrazole-3-carboxamide hydrochloride

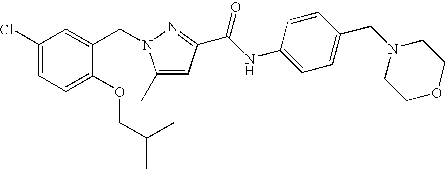

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added morpholine (28 μl) and the solution stirred under argon for 1 hour. Sodium triacetoxyborohydride (68 mg) and acetic acid (10 μl) were added and stirring continued for 2 hours. Further aliquots of morpholine (28 μl) and sodium triacetoxyborohydride (68 mg) were added and stirring continued for 2 hours. Ethyl acetate (12 ml) and brine (12 ml) were added and the aqueous layer separated and extracted with ethyl acetate (5 ml). The combined organic extracts were dried (MgSO₄) and concentrated in vacuo. The residue was purified by mass-directed autopreparation then dissolved in dichloromethane (5 ml). 1M hydrogen chloride in ether (5 ml) was added and solvent removed in vacuo. The residue was washed with hexane and dried in vacuo to afford the title compound (47.6 mg, 56%) as a pale cream solid. LC/MS [MH+]=497/499, RT=2.43 min.

### Example 399

### 1-({5-Chloro-2-[(2-methylpropyl)oxy]phenyl}methyl)-5-methyl-N-{4-[(3-oxo-1-piperazinyl)methyl]phenyl}-1H-pyrazole-3-carboxamide hydrochloride

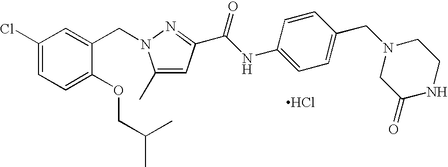

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added piperazin-2-one (32 mg) and the solution stirred under argon for 1 hour. Sodium triacetoxyborohydride (68 mg) and acetic acid (10 μl) were added and stirring continued for 2 hours. Further aliquots of piperazin-2-one (50 mg) and sodium triacetoxyborohydride (68 mg) were added and stirring continued for 2 hours. Ethyl acetate (12 ml) and brine (12 ml) were added and the aqueous layer separated and extracted with ethyl acetate (5 ml). The combined organic extracts were dried (MgSO₄) and concentrated in vacuo. The residue was purified by mass-directed autopreparation then dissolved in dichloromethane (5 ml). 1M hydrogen chloride in ether (5 ml) was added and solvent removed in vacuo. The residue was recrystallised from isopropanol and dried in vacuo to afford the title compound (52.8 mg, 61%) as an off-white solid. LC/MS [MH+]=510/512, RT=2.44 min.

### Example 400

### 1-({5-Chloro-2-[(2-methylpropyl)oxy]phenyl}methyl)-N-{4-[(4-hydroxy-1-piperidinyl)methyl]phenyl}-5-methyl-1H-pyrazole-3-carboxamide hydrochloride

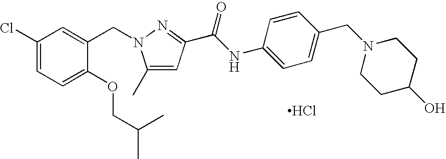

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added 4-piperidinol (32.5 mg) and sodium triacetoxyborohydride (68 mg) and the solution stirred at ambient temperature for 2 hours. Further aliquots of 4-piperidinol (32.5 mg) and sodium triacetoxyborohydride (68 mg) were added and stirring continued for 2 hours at 50°. Ethyl acetate (12 ml) and brine (12 ml) were added and the organic layer separated, dried (MgSO₄) and concentrated in vacuo. The residue was purified by mass-directed autopreparation then dissolved in dichloromethane (5 ml).1M hydrogen chloride in ether (1 ml) was added and solvent removed in vacuo. The residue was washed with hexane and dried in vacuo to afford the title compound (38.1 mg, 47%) as a pale cream solid.

LC/MS [MH+]=511/513, RT=2.34 min.

### Example 401

### 1-({5-Chloro-2-[(2-methylpropyl)oxy]phenyl}methyl)-N-(4-{[(3R)-3-hydroxy-1-pyrrolidinyl]methyl}phenyl)-5-methyl-1H-pyrazole-3-carboxamide hydrochloride

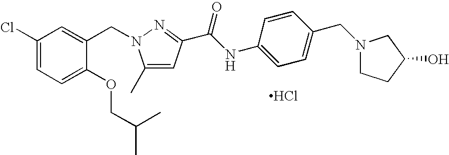

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added (R)-(+)-3-pyrrolidinol (27 μl) and sodium triacetoxyborohydride (68 mg) and the solution stirred at ambient temperature for 2 hours. Ethyl acetate (12 ml) and brine (12 ml) were added and the organic layer separated, dried (MgSO₄) and concentrated in vacuo. The residue was purified by mass-directed autopreparation then dissolved in dichloromethane (5 ml). 1M hydrogen chloride in ether (1 ml) was added and solvent removed in vacuo. The residue was washed with hexane and dried in vacuo to afford the title compound (31.9 mg, 40%) as a pale cream solid. LC/MS [MH+]=497/499, RT=2.35 min.

### Example 402

### 1-({5-Chloro-2-[(2-methylpropyl)oxy]phenyl}methyl)-N-(4-{[(3S)-3-hydroxy-1-pyrrolidinyl]methyl}phenyl)-5-methyl-1H-pyrazole-3-carboxamide hydrochloride

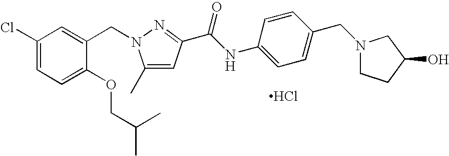

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added (S)-(−)-3-pyrrolidinol (27 μl) and sodium triacetoxyborohydride (68 mg) and the solution stirred at ambient temperature for 2 hours. Ethyl acetate (12 ml) and brine (12 ml) were added and the organic layer separated, dried (MgSO₄) and concentrated in vacuo. The residue was purified by mass-directed autopreparation then dissolved in dichloromethane (5 ml). 1M hydrogen chloride in ether (1 ml) was added and solvent removed in vacuo. The residue was washed with hexane and dried in vacuo to afford the title compound (48.6 mg, 61%) as a pale cream solid. LC/MS [MH+]=497/499, RT=2.35 min.

### Example 403

### 1-({5-chloro-2-[(2-methylpropyl)oxy]phenyl}methyl)-N-(4-{[(2-hydroxyethyl)amino]methyl}phenyl)-5-methyl-1H-pyrazole-3-carboxamide hydrochloride

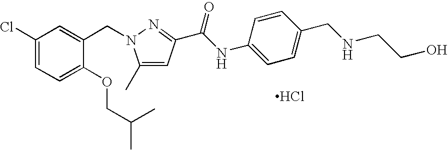

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added ethanolamine (19.5 μl) and sodium triacetoxyborohydride (68 mg) and the solution stirred at ambient temperature for 2 hours. Further aliquots of ethanolamine (32.5 mg) and sodium triacetoxyborohydride (68 mg) were added with acetic acid (0.5 ml) and stirring continued for 2 hours under reflux. Ethyl acetate (12 ml) and brine (12 ml) were added and the organic layer separated, dried (MgSO₄) and concentrated in vacuo. The residue was purified by mass-directed autopreparation then dissolved in dichloromethane (5 ml). 1M hydrogen chloride in ether (1 ml) was added and solvent removed in vacuo. The residue was washed with hexane and dried in vacuo to afford the title compound (31.2 mg, 41%) as a pale cream solid. LC/MS [MH+]=471/473, RT=2.32 min.

### Example 404

### N-(4-{[Bis(2-hydroxyethyl)amino]methyl}phenyl)-1-({5-chloro-2-[(2-methylpropyl)oxy]phenyl}methyl)-5-methyl-1H-pyrazole-3-carboxamide hydrochloride

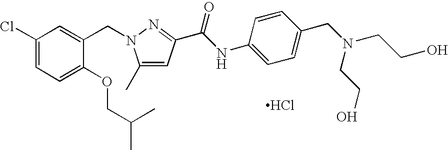

To a solution of 1-({5-chloro-2-[(2-methylpropyl)oxy]phenyl}methyl)-N-(4-formylphenyl)-5-methyl-1H-pyrazole-3-carboxamide (68 mg) in dry tetrahydrofuran (2.5 ml) was added diethanolamine (31 μl) and sodium triacetoxyborohydride (68 mg) and the solution stirred at ambient temperature for 2 hours. Further aliquots of diethanolamine (135 mg) and sodium triacetoxyborohydride (270 mg) were added with acetic acid (0.5 ml) and stirring continued for 4 hours under reflux. Ethyl acetate (12 ml) and brine (12 ml) were added and the organic layer separated, dried (MgSO₄) and concentrated in vacuo. The residue was purified by mass-directed autopreparation then dissolved in dichloromethane (5 ml). 1M hydrogen chloride in ether (1 ml) was added and solvent removed in vacuo. The residue was washed with hexane and dried in vacuo to afford the title compound (18.1 mg, 22%) as a pale cream solid. LC/MS [MH+]=515/517, RT=2.30 min.

### Example 405

### 1-({5-Chloro-2-[(2-methylpropyl)oxy]phenyl}methyl)-N-[4-(hydroxyl-methyl)-3-(methyloxy)phenyl]-5-methyl-1H-pyrazole-3-carboxamide

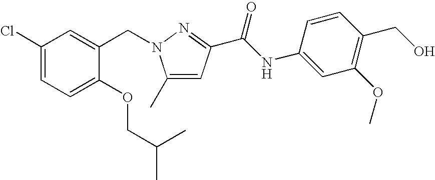

### (a). Methyl 4-({[1-({5-chloro-2-[(2-methylpropyl)oxy]phenyl}methyl)-5-methyl-1H-pyrazol-3-yl]carbonyl}amino)-2-(methyloxy)benzoate

A solution of 1-({5-chloro-2-[(2-methylpropyl)oxy]phenyl}methyl)-5-methyl-1H-pyrazole-3-carboxylic acid (807 mg), methyl 4-amino-2-methoxybenzoate (453 mg), N-(3-dimethylaminopropyl)-N′-ethylcarbodiimide hydrochloride (516 mg) and 1-hydroxybenzotriazole hydrate (406 mg) in dichloromethane (10 ml) and dimethylformamide (10 ml) was stirred at ambient temperature for 24 hours. Ethyl acetate (60 ml) was added and the mixture washed with water (60 ml), saturated sodium bicarbonate solution (25 ml) and brine (2×20 ml), dried (MgSO₄) and concentrated in vacuo. The residue was purified by SP4 Biotage chromatography using 5 to 40% ethyl acetate in hexane to afford the title compound (738 mg, 61%) as a white foamy solid.

LC/MS [MH+]=486/488, RT=3.70 min.

### (b). 1-({5-Chloro-2-[(2-methylpropyl)oxy]phenyl}methyl)-N-[4-(hydroxyl-methyl)-3-(methyloxy)phenyl]-5-methyl-1H-pyrazole-3-carboxamide

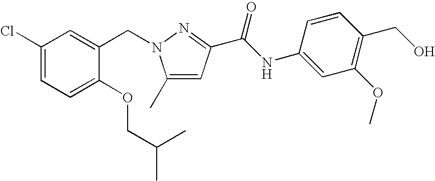

To a solution of methyl 4-({[1-({5-chloro-2-[(2-methylpropyl)oxy]phenyl}-methyl)-5-methyl-1H-pyrazol-3-yl]carbonyl}amino)-2-(methyloxy)benzoate (736 mg) in tetrahydrofuran (20 ml) was added dropwise with ice-cooling under argon a 1M solution of lithium aluminium hydride in tetrahydrofuran (2.9 ml) and the mixture stirred at ambient temperature for 1.5 hours. It was quenched with a few mis of methanol and water, and ethyl acetate (30 ml) and 2M sodium hydroxide solution (25 ml) added. The organic layer was separated and washed with water (10 ml), dried (MgSO₄) and concentrated in vacuo. The residue was triturated with cold ether and dried in vacuo to afford the title compound (485 mg, 70%) as a white solid. LC/MS [MH+]=458/460, RT=3.38 min.

### Example 406

### 1-({5-Chloro-2-[(2-methylpropyl)oxy]phenyl}methyl)-5-methyl-N-[4-[(methylamino)methyl]-3-(methyloxy)phenyl]-1H-pyrazole-3-carboxamide hydrochloride

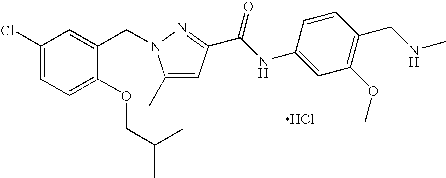

### (a). 1-({5-Chloro-2-[(2-methylpropyl)oxy]phenyl}methyl)-N-[4-formyl-3-(methyloxy)phenyl]-5-methyl-1H-pyrazole-3-carboxamide

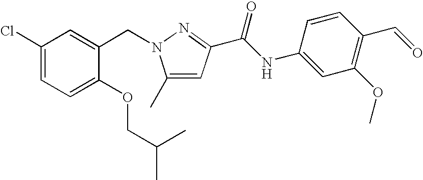

To a solution of 1-({5-chloro-2-[(2-methylpropyl)oxy]phenyl}methyl)-N-[4-(hydroxylmethyl)-3-(methyloxy)phenyl]-5-methyl-1H-pyrazole-3-carboxamide (457 mg) in dry dichloromethane (7.5 ml) was added under argon Dess-Martin periodinane (424 mg) and the mixture stirred at ambient temperature for 2 hours. It was washed with saturated sodium bicarbonate solution (10 ml) and brine (10 ml) and the aqueous layer extracted with dichloromethane (2×5 ml). The combined dichloromethane layers were dried (MgSO₄) and concentrated in vacuo. The residue was purified by SP4 Biotage chromatography using 8 to 66% ethyl acetate in hexane to afford the title compound (335 mg, 74%) as a white solid. LC/MS [MH+]=456/458, RT=3.75 min.

### (b). 1-({5-Chloro-2-[(2-methylpropyl)oxy]phenyl}methyl)-5-methyl-N-[4-[(methylamino)methyl]-3-(methyloxy)phenyl]-1H-pyrazole-3-carboxamide hydrochloride

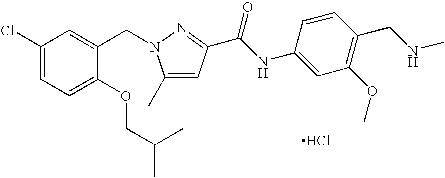

To a solution of 1-({5-chloro-2-[(2-methylpropyl)oxy]phenyl}methyl)-N-[4-formyl-3-(methyloxy)phenyl]-5-methyl-1H-pyrazole-3-carboxamide (55 mg) in dry tetrahydrofuran (2.5 ml) was added 33% methylamine in ethanol (23 μl) and the solution stirred at ambient temperature for ½ hour. Sodium triacetoxyborohydride (54 mg) was added and stirring continued for 3 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (3 ml). 1M hydrogen chloride in ether (5 ml) was added and solvent removed in vacuo. The residue was washed with ether and dried in vacuo to afford the title compound (13.2 mg, 23%) as a white solid. LC/MS [MH+]=471/473, RT=2.44 min.

### Example 407

### 1-({5-Chloro-2-[(2-methylpropyl)oxy]phenyl}methyl)-N-[4-[(ethylamino)-methyl]-3-(methyloxy)phenyl]-5-methyl-1H-pyrazole-3-carboxamide hydrochloride

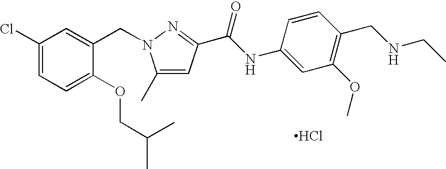

To a solution of 1-({5-chloro-2-[(2-methylpropyl)oxy]phenyl}methyl)-N-[4-formyl-3-(methyloxy)phenyl]-5-methyl-1H-pyrazole-3-carboxamide (55 mg) in dry tetrahydrofuran (2.5 ml) was added 2M ethylamine in tetrahydrofuran (12.5 μl) and the solution stirred at ambient temperature for ½ hour. Sodium triacetoxyborohydride (54 mg) was added and stirring continued for 3 hours. Further aliquots of 2M ethylamine in tetrahydrofuran (25 μl) and sodium triacetoxyborohydride (75 mg) were added and stirring continued for 2 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (3 ml). 1M hydrogen chloride in ether (5 ml) was added and solvent removed in vacuo. The residue was washed with ether and dried in vacuo to afford the title compound (16.1 mg, 27%) as a white solid.

LC/MS [MH+]=485/487, RT=2.53 min.

### Example 408

### 1-({5-Chloro-2-[(2-methylpropyl)oxy]phenyl}methyl)-5-methyl-N-[3-(methyloxy)-4-(1-pyrrolidinylmethyl)phenyl]-1H-pyrazole-3-carboxamide hydrochloride

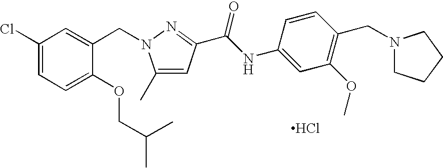

To a solution of 1-({5-chloro-2-[(2-methylpropyl)oxy]phenyl}methyl)-N-[4-formyl-3-(methyloxy)phenyl]-5-methyl-1H-pyrazole-3-carboxamide (55 mg) in dry tetrahydrofuran (2.5 ml) was added pyrrolidine (21 μl) and the solution stirred at ambient temperature for ½ hour. Sodium triacetoxyborohydride (54 mg) was added and stirring continued for 3 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (3 ml). 1M hydrogen chloride in ether (5 ml) was added and solvent removed in vacuo. The residue was washed with ether and dried in vacuo to afford the title compound (28.1 mg, 46%) as a pale cream solid.

LC/MS [MH+]=511/513, RT=2.59 min.

### Example 409

### 1-({5-Chloro-2-[(2-methylpropyl)oxy]phenyl}methyl)-5-methyl-N-[3-(methyloxy)-4-(4-morpholinylmethyl)phenyl]-1H-pyrazole-3-carboxamide hydrochloride

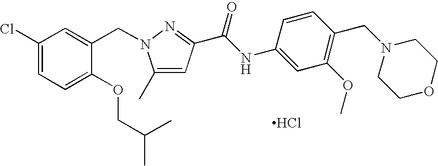

To a solution of 1-({5-chloro-2-[(2-methylpropyl)oxy]phenyl}methyl)-N-[4-formyl-3-(methyloxy)phenyl]-5-methyl-1H-pyrazole-3-carboxamide (55 mg) in dry tetrahydrofuran (2.5 ml) was added morpholine (22 μl) and the solution stirred at ambient temperature for ½ hour. Sodium triacetoxyborohydride (54 mg) was added and stirring continued for 3 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (3 ml). 1M hydrogen chloride in ether (5 ml) was added and solvent removed in vacuo. The residue was washed with ether and dried in vacuo to afford the title compound (35.3 mg, 55%) as a pale cream solid.

LC/MS [MH+]=527/529, RT=2.52 min.

### Example 410

### 1-({5-Chloro-2-[(2-methylpropyl)oxy]phenyl}methyl)-N-[4-[(4-hydroxy-1-piperidinyl)methyl]-3-(methyloxy)phenyl]-5-methyl-1H-pyrazole-3-carboxamide hydrochloride

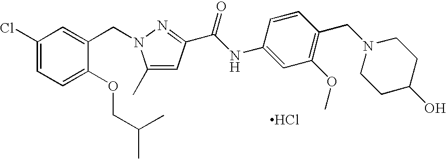

To a solution of 1-({5-chloro-2-[(2-methylpropyl)oxy]phenyl}methyl)-N-[4-formyl-3-(methyloxy)phenyl]-5-methyl-1H-pyrazole-3-carboxamide (55 mg) in dry tetrahydrofuran (2.5 ml) was added 4-piperidinol (25 mg) and the solution stirred at ambient temperature for ½ hour. Sodium triacetoxyborohydride (54 mg) was added and stirring continued for 3 hours. Further aliquots of 4-piperidinol (50 mg) and sodium triacetoxyborohydride (75 mg) were added and stirring continued for 2 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (3 ml). 1M hydrogen chloride in ether (5 ml) was added and solvent removed in vacuo. The residue was washed with ether and dried in vacuo to afford the title compound (18.9 mg, 29%) as a pale cream solid. LC/MS [MH+]=541/543, RT=2.43 min.

### Example 411

### 1-({5-Chloro-2-[(2-methylpropyl)oxy]phenyl}methyl)-N-[4-(hydroxyl-methyl)-2-(methyloxy)phenyl]-5-methyl-1H-pyrazole-3-carboxamide

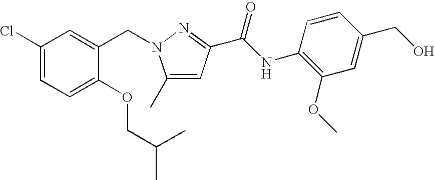

### (a). 1-({[1-({5-Chloro-2-[(2-methylpropyl)oxy]phenyl}methyl)-5-methyl-1H-pyrazol-3-yl]carbonyl}oxy)-1H-1,2,3-benzotriazole

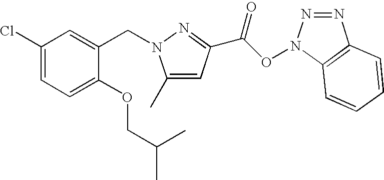

A solution of 1-({5-chloro-2-[(2-methylpropyl)oxy]phenyl}methyl)-5-methyl-1H-pyrazole-3-carboxylic acid (1.94 g), methyl 6-aminonicotinate (913 mg), N-(3-dimethylaminopropyl)-N′-ethylcarbodiimide hydrochloride (1.24 g) and 1-hydroxybenzotriazole hydrate (975 mg) in dichloromethane (25 ml) and dimethylformamide (25 ml) was stirred at ambient temperature for 16 hours. Ethyl acetate (120 ml) was added and the mixture washed with water (120 ml), saturated sodium bicarbonate solution (45 ml) and brine (2×35 ml), dried (MgSO₄) and concentrated in vacuo. The residue was purified by Biotage chromatography using 3:1 hexane:ethyl acetate to afford the title compound (1.59 g, 60%) as a white foamy solid. LC/MS [MH+]=440/442, RT=3.74 min.

### (b). 1-({5-Chloro-2-[(2-methylpropyl)oxy]phenyl}methyl)-N-[4-(hydroxyl-methyl)-2-(methyloxy)phenyl]-5-methyl-1H-pyrazole-3-carboxamide

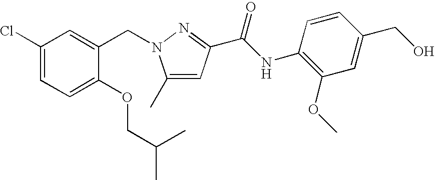

A solution of 1-({[1-({5-chloro-2-[(2-methylpropyl)oxy]phenyl}methyl)-5-methyl-1H-pyrazol-3-yl]carbonyl}oxy)-1H-1,2,3-benzotriazole (470 mg) and [4-amino-3-(methyloxy)phenyl]methanol (153 mg, Ref.: Fujisawa Patent WO9711069 (1997), p. 40) in dry dichloromethane (5 ml) was stirred at ambient temperature for 4 hours. It was concentrated in vacuo and ethyl acetate (10 ml) added. The solution washed with saturated sodium bicarbonate solution (5 ml) and brine (2×5 ml), dried (MgSO₄) and concentrated in vacuo. The residue was purified by SP4 Biotage chromatography using 12 to 100% ethyl acetate in hexane to afford the title compound (345 mg, 75%) as an off-white solid. LC/MS [MH+]=458/460, RT=3.51 min.

### Example 412

### 1-({5-Chloro-2-[(2-methylpropyl)oxy]phenyl}methyl)-5-methyl-N-[4-[(methylamino)methyl]-2-(methyloxy)phenyl]-1H-pyrazole-3-carboxamide hydrochloride

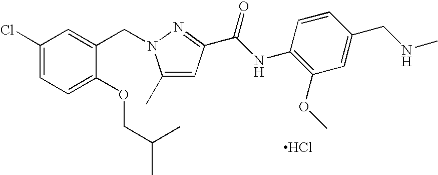

### (a). 1-({5-Chloro-2-[(2-methylpropyl)oxy]phenyl}methyl)-N-[4-formyl-2-(methyloxy)phenyl]-5-methyl-1H-pyrazole-3-carboxamide

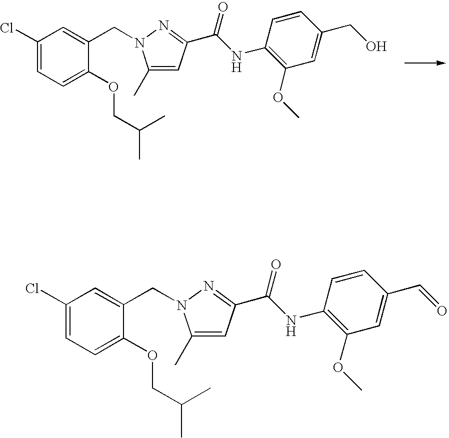

To a solution of 1-({5-chloro-2-[(2-methylpropyl)oxy]phenyl}methyl)-N-[4-(hydroxylmethyl)-2-(methyloxy)phenyl]-5-methyl-1H-pyrazole-3-carboxamide (317 g) in dry dichloromethane (5 ml) was added under argon Dess-Martin periodinane (294 mg) and the mixture stirred at ambient temperature for 1.5 hours. It was washed with saturated sodium bicarbonate solution (6 ml) and the aqueous layer extracted with dichloromethane (2×4 ml). The combined dichloromethane layers were dried (MgSO₄) and concentrated in vacuo. The residue was purified by SP4 Biotage chromatography using 8 to 66% ethyl acetate in hexane to afford the title compound (237 mg, 75%) as a pale buff solid.

LC/MS [MH+]=456/458, RT=3.92 min.

### (b). 1-({5-Chloro-2-[(2-methylpropyl)oxy]phenyl}methyl)-5-methyl-N-[4-[(methylamino)methyl]-2-(methyloxy)phenyl]-1H-pyrazole-3-carboxamide hydrochloride

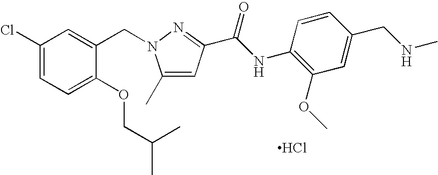

To a solution of 1-({5-chloro-2-[(2-methylpropyl)oxy]phenyl}methyl)-N-[4-formyl-2-(methyloxy)phenyl]-5-methyl-1H-pyrazole-3-carboxamide (59 mg) in dry tetrahydrofuran (2.5 ml) was added 33% methylamine in ethanol (60 μl) and the solution stirred at ambient temperature for 1 hour. Sodium triacetoxyborohydride (60 mg) was added and stirring continued for 2 hours. A further aliquot of 33% methylamine in ethanol (120 μl) was added and after stirring for a further hour sodium triacetoxyborohydride (60 mg) was added and stirring continued for 2 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (4 ml). 1M hydrogen chloride in ether (3 ml) was added and solvent removed in vacuo. The residue was washed with hexane and dried in vacuo to afford the title compound (11.8 mg, 19%) as a pale buff solid. LC/MS [MH+]=440/442 (loss of H₂NCH₃), RT=2.49 min.

### Example 413

### 1-({5-Chloro-2-[(2-methylpropyl)oxy]phenyl}methyl)-5-methyl-N-[2-(methyloxy)-4-(1-pyrrolidinylmethyl)phenyl]-1H-pyrazole-3-carboxamide hydrochloride

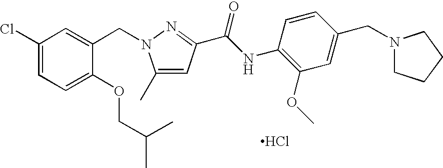

To a solution of 1-({5-chloro-2-[(2-methylpropyl)oxy]phenyl}methyl)-N-[4-formyl-2-(methyloxy)phenyl]-5-methyl-1H-pyrazole-3-carboxamide (59 mg) in dry tetrahydrofuran (2.5 ml) was added pyrrolidine (25 μl) and the solution stirred at ambient temperature for 1 hour. Sodium triacetoxyborohydride (60 mg) was added and stirring continued for 2 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (4 ml). 1M hydrogen chloride in ether (3 ml) was added and solvent removed in vacuo. The residue was washed with hexane and dried in vacuo to afford the title compound (30.2 mg, 46%) as a pale cream solid.

LC/MS [MH+]=511/513, RT=2.59 min.

### Example 414

### 1-({5-Chloro-2-[(2-methylpropyl)oxy]phenyl}methyl)-5-methyl-N-[2-(methyloxy)-4-(4-morpholinylmethyl)phenyl]-1H-pyrazole-3-carboxamide hydrochloride

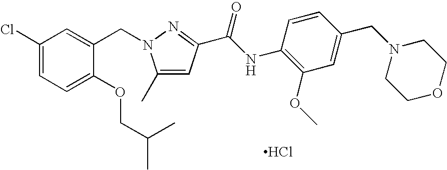

To a solution of 1-({5-chloro-2-[(2-methylpropyl)oxy]phenyl}methyl)-N-[4-formyl-2-(methyloxy)phenyl]-5-methyl-1H-pyrazole-3-carboxamide (59 mg) in dry tetrahydrofuran (2.5 ml) was added morpholine (26 μl) and the solution stirred at ambient temperature for 1 hour. Sodium triacetoxyborohydride (60 mg) was added and stirring continued for 2 hours. A further aliquot of morpholine (25 μl) was added and after stirring for a further hour sodium triacetoxyborohydride (60 mg) was added and stirring continued for 2 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (4 ml). 1M hydrogen chloride in ether (3 ml) was added and solvent removed in vacuo. The residue was washed with hexane and dried in vacuo to afford the title compound (35.3 mg, 52%) as a pale cream solid.

LC/MS [MH+]=527/529, RT=2.55 min.

### Example 415

### 1-({5-Chloro-2-[(2-methylpropyl)oxy]phenyl}methyl)-N-[4-[(4-hydroxy-1-piperidinyl)methyl]-2-(methyloxy)phenyl]-5-methyl-1H-pyrazole-3-carboxamide hydrochloride

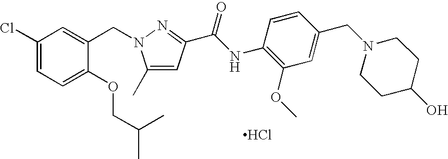

To a solution of 1-({5-chloro-2-[(2-methylpropyl)oxy]phenyl}methyl)-N-[4-formyl-2-(methyloxy)phenyl]-5-methyl-1H-pyrazole-3-carboxamide (59 mg) in dry tetrahydrofuran (2.5 ml) was added 4-piperidinol (35 mg) and the solution stirred at ambient temperature for 1 hour. Sodium triacetoxyborohydride (60 mg) was added and stirring continued for 2 hours. A further aliquot of 4-piperidinol (35 mg) was added and after stirring for a further hour sodium triacetoxyborohydride (60 mg) was added and stirring continued for 2 hours. Ethyl acetate (12 ml) and brine (12 ml) were added and the aqueous layer separated and extracted with ethyl acetate (10 ml). The combined organic extracts were dried (MgSO₄) and concentrated in vacuo. The residue was purified by mass-directed autopreparation then dissolved in dichloromethane (4 ml). 1M hydrogen chloride in ether (3 ml) was added and solvent removed in vacuo. The residue was washed with hexane and dried in vacuo to afford the title compound (28.7 mg, 41%) as a pale cream solid.

LC/MS [MH+]=541/543, RT=2.50 min.

### Example 416

### 1-({5-Chloro-2-[(2-methylpropyl)oxy]phenyl}methyl)-N-[5-(hydroxymethyl)-2-pyridinyl]-5-methyl-1H-pyrazole-3-carboxamide

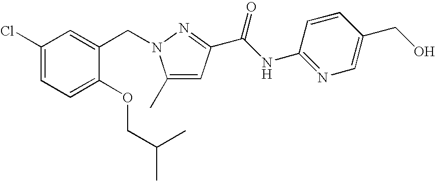

### (a). Methyl 6-({[1-({5-chloro-2-[(2-methylpropyl)oxy]phenyl}methyl)-5-methyl-1H-pyrazol-3-yl]carbonyl}amino)-3-pyridinecarboxylate

A solution of 1-({[1-({5-chloro-2-[(2-methylpropyl)oxy]phenyl}methyl)-5-methyl-1H-pyrazol-3-yl]carbonyl}oxy)-1H-1,2,3-benzotriazole (533 mg), methyl 6-aminonicotinate (184 mg) and 4-dimethylaminopyridine (150 mg) in dry dichloromethane (7.5 ml) was stirred at ambient temperature for 6 days. It was concentrated in vacuo and ethyl acetate (40 ml) added. The solution washed with water (20 ml), saturated sodium bicarbonate solution (20 ml) and brine (20 ml), dried (MgSO₄) and concentrated in vacuo. The residue was purified by SP4 Biotage chromatography using 5 to 40% ethyl acetate in hexane to afford the title compound (206 mg, 37%) as a white solid. LC/MS [MH+]=457/459, RT=3.99 min.

### (b). 1-({5-Chloro-2-[(2-methylpropyl)oxy]phenyl}methyl)-N-[5-(hydroxymethyl)-2-pyridinyl]-5-methyl-1H-pyrazole-3-carboxamide

To a solution of methyl 6-({[1-({5-chloro-2-[(2-methylpropyl)oxy]phenyl}-methyl)-5-methyl-1H-pyrazol-3-yl]carbonyl}amino)-3-pyridinecarboxylate (206 mg) in tetrahydrofuran (7 ml) was added dropwise with ice-cooling under argon a 1M solution of lithium aluminium hydride in tetrahydrofuran (0.9 ml) and the mixture stirred at ambient temperature for 2 hours. It was quenched with a few mis of methanol and water, and ethyl acetate (10 ml) and 2M sodium hydroxide solution (8 ml) added. The organic layer was separated and washed with brine (5 ml), dried (MgSO₄) and concentrated in vacuo. The residue was purified by mass-directed autopreparation to afford the title compound (32 mg, 17%) as a pale cream solid. LC/MS [MH+]=429/431, RT=3.20 min.

### Example 417

### 6-({[1-({5-Chloro-2-[(2-methylpropyl)oxy]phenyl}methyl)-5-methyl-1H-pyrazol-3-yl]carbonyl}amino)-3-pyridinecarboxylic acid

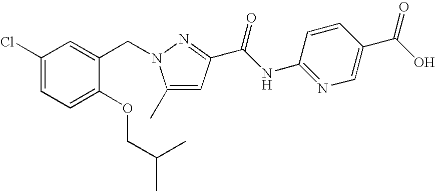

A solution of 1-({5-chloro-2-[(2-methylpropyl)oxy]phenyl}methyl)-5-methyl-1H-pyrazole-3-carboxylic acid (161 mg) and thionyl chloride (182 μl) in dry dichloromethane (1.5 ml) was stirred under reflux for 2.5 hours. It was concentrated in vacuo and traces of thionyl chloride removed by azeotroping with toluene.

The acid chloride was dissolved in dichloromethane (1.5 ml) and the solution added dropwise to a solution of methyl 6-aminonicotinate (68 mg) and triethylamine (63 μl) in dry dichloromethane (1.5 ml). The solution was stirred at ambient temperature for 2 hours and washed with 2M sodium hydroxide solution (4 ml). The aqueous layer was washed with dichloromethane (4 ml) and the combined organic layers washed with brine (4 ml), dried (MgSO₄) and concentrated in vacuo. The residue was dissolved in ethanol (5 ml) and 2M sodium hydroxide solution (1 ml), and the solution stirred at ambient temperature for 5 hours and concentrated in vacuo. Water (4 ml) was added and the solution acidified with concentrated hydrochloric acid. The mixture was extracted with dichloromethane (2×5 ml) and the combined extracts dried (MgSO₄) and concentrated in vacuo. The residue was purified by mass-directed autopreparation to afford the title compound (57.4 mg, 29%) as a white solid. LC/MS [MH+]=443/445, RT=3.47 min.

### Example 418

### 1-({5-Chloro-2-[(2-methylpropyl)oxy]phenyl}methyl)-N-{6-[(dimethylamino)methyl]-3-pyridinyl}-5-methyl-1H-pyrazole-3-carboxamide hydrochloride

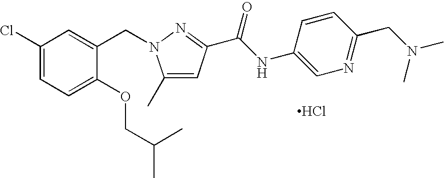

### (a). 1-({5-Chloro-2-[(2-methylpropyl)oxy]phenyl}methyl)-N-(6-cyano-3-pyridinyl)-5-methyl-1H-pyrazole-3-carboxamide

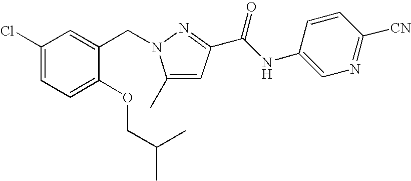

A solution of 1-({[1-({5-chloro-2-[(2-methylpropyl)oxy]phenyl}methyl)-5-methyl-1H-pyrazol-3-yl]carbonyl}oxy)-1H-1,2,3-benzotriazole (440 mg), 5-amino-2-cyanopyridine (119 mg) and 4-dimethylaminopyridine (125 mg) in dry dichloromethane (5 ml) was stirred at ambient temperature for 30 hours. It was concentrated in vacuo and ethyl acetate (10 ml) added. The solution washed with saturated sodium bicarbonate solution (5 ml) and brine (2×4 ml), dried (MgSO₄) and concentrated in vacuo. The residue was purified by SP4 Biotage chromatography using 12 to 100% ethyl acetate in hexane to afford the title compound (221 mg, 52%) as a pale buff solid. LC/MS [MH+]=424/426, RT=3.60 min.

### (b). N-[6-(Aminomethyl)-3-pyridinyl]-1-({5-chloro-2-[(2-methylpropyl)oxy]-phenyl}methyl)-5-methyl-1H-pyrazole-3-carboxamide hydrochloride

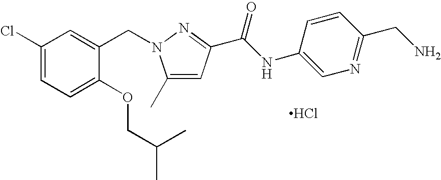

To a solution/suspension of 1-({5-Chloro-2-[(2-methylpropyl)oxy]phenyl}-methyl)-N-(6-cyano-3-pyridinyl)-5-methyl-1H-pyrazole-3-carboxamide (221 mg) in methanol (15 ml) and concentrated hydrochloric acid (0.25 ml) was added 10% palladium on carbon (100 mg) and the mixture hydrogenated at atmospheric pressure for 4 hours. Catalyst was filtered off and washed with methanol, and the filtrate concentrated in vacuo. The residue was washed with ether and dried in vacuo afford the title compound (241 mg, 99%) as a pale buff solid. LC/MS [MH+]=428/430, RT=2.26 min.

### (c). 1-({5-Chloro-2-[(2-methylpropyl)oxy]phenyl}methyl)-N-{6-[(dimethylamino)methyl]-3-pyridinyl}-5-methyl-1H-pyrazole-3-carboxamide hydrochloride

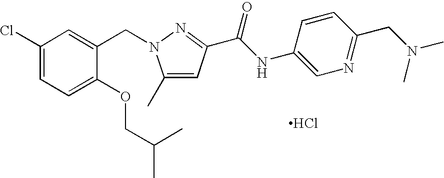

To a solution of N-[6-(aminomethyl)-3-pyridinyl]-1-({5-chloro-2-[(2-methylpropyl)oxy]phenyl}methyl)-5-methyl-1H-pyrazole-3-carboxamide hydrochloride (104 mg) in dry tetrahydrofuran (6 ml) was added 37% aqueous formaldehyde (40 μl) and the solution stirred under argon for ½ hour. Sodium triacetoxyborohydride (106 mg) was added and stirring continued for 1 hour. Further aliquots of 37% aqueous formaldehyde (15 μl) and sodium triacetoxyborohydride (40 mg) were added and stirring continued for 2 hours. Ethyl acetate (20 ml) and brine (20 ml) were added and the aqueous layer separated and extracted with ethyl acetate (15 ml). The combined organic extracts were dried (MgSO₄) and concentrated in vacuo. The residue was purified by mass-directed autopreparation then dissolved in dichloromethane (2 ml). 1M hydrogen chloride in ether (4 ml) was added and solvent removed in vacuo. The residue was washed with hexane and dried in vacuo to afford the title compound (36.8 mg, 31%) as a pale cream solid.

LC/MS [MH+]=456/458, RT=2.34 min.

### Example 419

### 1-({5-Chloro-2-[(2-methylpropyl)oxy]phenyl}methyl)-N-{6-[(diethylamino)methyl]-3-pyridinyl}-5-methyl-1H-pyrazole-3-carboxamide hydrochloride

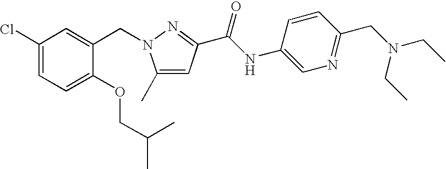

To a solution of N-[6-(aminomethyl)-3-pyridinyl]-1-({5-chloro-2-[(2-methylpropyl)oxy]phenyl}methyl)-5-methyl-1H-pyrazole-3-carboxamide hydrochloride (93 mg) in dry tetrahydrofuran (5 ml) was added acetaldehyde (23 μl) and the solution stirred under argon for ½ hour. Sodium triacetoxyborohydride (87 mg) was added and stirring continued for 1 hour. Ethyl acetate (20 ml) and brine (20 ml) were added and the aqueous layer separated and extracted with ethyl acetate (15 ml). The combined organic extracts were dried (MgSO₄) and concentrated in vacuo. The residue was purified by mass-directed autopreparation then dissolved in dichloromethane (3 ml). 1M hydrogen chloride in ether (4 ml) was added and solvent removed in vacuo. The residue was washed with hexane and dried in vacuo to afford the title compound (32.7 mg, 31%) as a pale cream solid.

LC/MS [MH+]=484/486, RT=2.38 min.

### Example 420

### 1-({5-Chloro-2-[(2-methylpropyl)oxy]phenyl}methyl)-5-methyl-N-[5-(1-pyrrolidinylmethyl)-2-pyridinyl]-1H-pyrazole-3-carboxamide hydrochloride

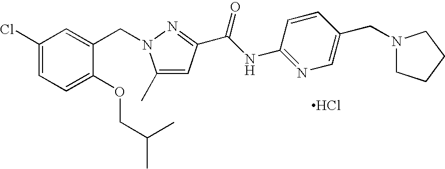

### (a). 5-(1-Pyrrolidinylmethyl)-2-pyridinamine

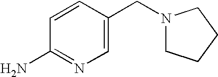

A mixture of 5-(chloromethyl)-2-pyridinamine (90 mg, Ref.: Array Biopharma Patent WO2005/095931 (2005), p. 38), pyrrolidine (83.5 μl), potassium carbonate (138 mg) and acetonitrile (2.5 ml) was stirred under reflux for 18 hours and allowed to cool. Water (15 ml) was added and the mixture extracted with dichloromethane (15 ml then 10 ml). The combined organic extracts were dried (MgSO₄) and concentrated in vacuo to afford the title compound (34.1 mg, 38%) as a pale cream solid.

### (b). 1-({5-Chloro-2-[(2-methylpropyl)oxy]phenyl}methyl)-5-methyl-N-[5-(1-pyrrolidinylmethyl)-2-pyridinyl]-1H-pyrazole-3-carboxamide hydrochloride

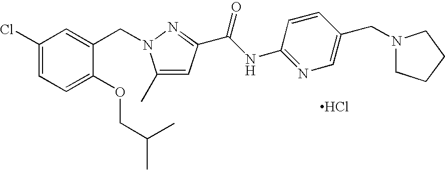

A solution of 1-({5-chloro-2-[(2-methylpropyl)oxy]phenyl}methyl)-5-methyl-1H-pyrazole-3-carboxylic acid (124 mg) and thionyl chloride (140 μl) in dry dichloromethane (1.5 ml) was stirred under reflux for 2 hours. It was concentrated in vacuo and traces of thionyl chloride removed by azeotroping with toluene.

The acid chloride was dissolved in dichloromethane (1.5 ml) and the solution added dropwise to a solution of 5-(1-pyrrolidinylmethyl)-2-pyridinamine (34.1 mg) and triethylamine (54 μl) in dry dichloromethane (1.5 ml). The solution was stirred at ambient temperature for 3 hours. Pyrrolidine (50 μl) was added and the mixture stirred for 2 hours. Dichloromethane was removed in vacuo and replaced with tetrahydrofuran (3 ml). Pyrrolidine (100 μl) was added and the mixture stirred at reflux for 3 days. Tetrahydrofuran was removed in vacuo and ethyl acetate (5 ml) added, and the solution washed with saturated sodium bicarbonate solution (5 ml) and brine (5 ml), dried (MgSO₄) and concentrated in vacuo. The residue was purified by mass-directed autopreparation then dissolved in dichloromethane (3 ml). 1M hydrogen chloride in ether (4 ml) was added and solvent removed in vacuo. The residue was washed with hexane and dried in vacuo at 40° to afford the title compound (19.1 mg, 19%) as a pale cream solid.

LC/MS [MH+]=482/484, RT=2.38 min.

The following intermediates were prepared in a similar manner to the method described for 4-ethenyl-2-fluorobenzoic acid from the appropriate intermediates:

| | Compound name | Data |
|---|---|---|
| 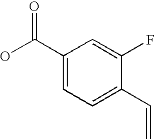 | 4-ethenyl-3- fluorobenzoic acid | LC/MS Rt = 2.46 min [MH+] 165 |
| 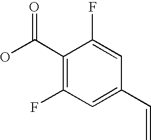 | 4-ethenyl-2,6- difluorobenzoic acid | LC/MS Rt = 2.15 min [MH+] 185 |

The following examples were prepared in a similar manner to the method described for N-[1-({5-Chloro-2-[(2-methylpropyl)oxy]phenyl}methyl)-5-methyl-1H-pyrazol-3-yl]-4-ethenyl-2-fluorobenzamide (Example 206) from the appropriate intermediates:

| Example | Structure | Compound name | Data |
|---|---|---|---|
| 421 | 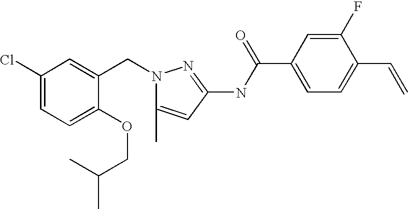 | N-[1-({5-chloro-2-[(2- methylpropyl)oxy]phenyl}methyl)- 5-methyl-1H-pyrazol-3-yl]-4- ethenyl-3-fluorobenzamide | LC/MS Rt = 3.91 min [MH+] 442, 444 |
| 422 | 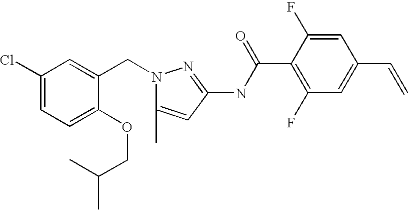 | N-[1-({5-chloro-2-[(2- methylpropyl)oxy]phenyl}methyl)- 5-methyl-1H-pyrazol-3-yl]-4- ethenyl-2,6-difluorobenzamide | LC/MS Rt = 3.74 min [MH+] 460, 462 |
| 423 | 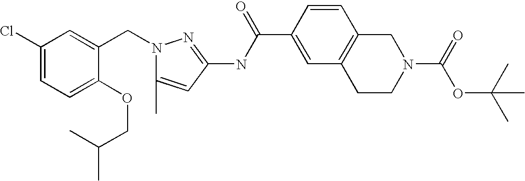 | 1,1-dimethylethyl 6-({[1-({5- chloro-2-[(2-methylpropyl)oxy] phenyl}methyl)-5-methyl-1H- pyrazol-3-yl]amino}carbonyl)-3,4- dihydro-2(1H)- isoquinolinecarboxylate | LC/MS Rt = 4.03 min [MH+] 553, 555 |
| | 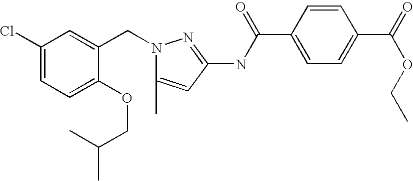 | ethyl 4-({[1-({5-chloro-2-[(2- methylpropyl)oxy]phenyl}methyl)- 5-methyl-1H-pyrazol-3- yl]amino}carbonyl)benzoate | LC/MS Rt = 3.67 min [MH+] 456, 458 |
| 424 | 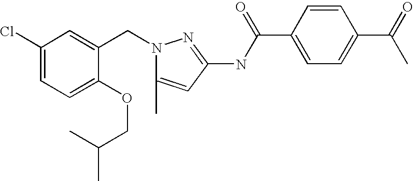 | 4-acetyl-N-[1-({5-chloro-2-[(2- methylpropyl)oxy]phenyl}methyl)- 5-methyl-1H-pyrazol-3- yl]benzamide | LC/MS Rt = 3.59 min [MH+] 440, 442 |

The following examples were prepared in a similar manner to N-[1-({5-chloro-2-[(2-methylpropyl)oxy]phenyl}methyl)-5-methyl-1H-pyrazol-3-yl]-2-fluoro-4-formylbenzamide (Example 207) from the appropriate intermediates:

| Example | | Compound name | Data |
|---|---|---|---|
| 426 | 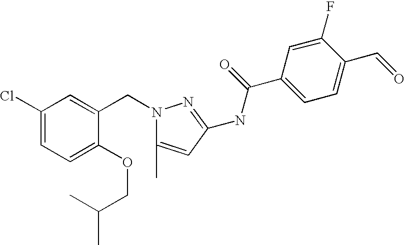 | N-[1-({5-chloro-2-[(2- methylpropyl)oxy]phenyl}meth- yl)-5-methyl-1H-pyrazol- 3-yl]-3-fluoro-4- formylbenzamide | LC/MS Rt = 3.60 min [MH+] 444, 446 |
| 425 | 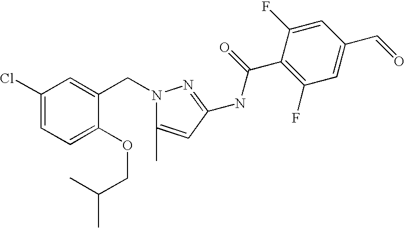 | N-[1-({5-chloro-2-[(2- methylpropyl)oxy]phenyl}meth- yl)-5-methyl-1H-pyrazol- 3-yl]-2,6-difluoro-4- formylbenzamide | LC/MS Rt = 3.50 min [MH+] 462, 464 |

### Example 427

### N-[1-({5-chloro-2-[(2-methylpropyl)oxy]phenyl}methyl)-5-methyl-1H-pyrazol-3-yl]-4-[(ethylamino)methyl]-2-fluorobenzamide hydrochloride

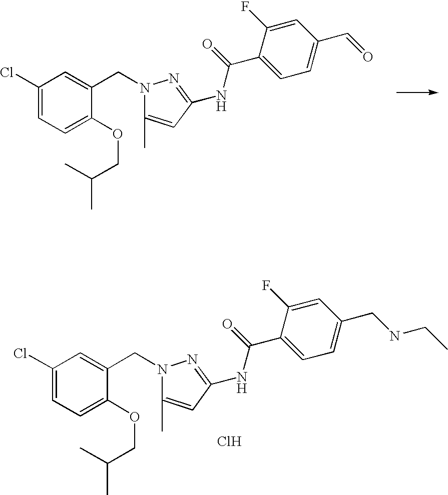

N-[1-({5-chloro-2-[(2-methylpropyl)oxy]phenyl}methyl)-5-methyl-1H-pyrazol-3-yl]-2-fluoro-4-formylbenzamide (0.054 g, 0.12 mmol), acetic acid (0.008 ml, 0.13 mmol), 2M ethylamine in THF (0.12 ml, 0.24 mmol) and sodium triacetoxyborohydride (0.077 g, 0.36 mmol) were stirred in dichloromethane (3.5 ml) for 3 hours and evaporated to dryness. The residue was redissolved in methanol and purified on an SCX ion exchange cartridge and further purified by MDAP. The residue was stirred in 1M hydrogen chloride in diethyl ether (1 ml) dried to yield the title compound (0.025 g). LC/MS Rt=2.42 min [MH+] 473, 475

The following examples were prepared in a similar manner to that described for N-[1-({5-chloro-2-[(2-methylpropyl)oxy]phenyl}methyl)-5-methyl-1H-pyrazol-3-yl]-4-[(dimethylamino)methyl]benzamide hydrochloride from the appropriate intermediates:

| Example | Structure | Compound name | Data |
|---|---|---|---|
| 428 | 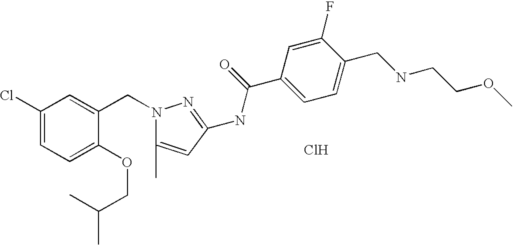 | N-[1-({5-chloro-2-[(2- methylpropyl)oxy]phenyl}methyl)- 5-methyl-1H-pyrazol-3-yl]-4-({[2- (methyloxy)ethyl]amino}methyl)- 2-fluorobenzamide hydrochloride | LC/MS Rt = 2.45 min [MH+] 503, 505 |
| 429 | 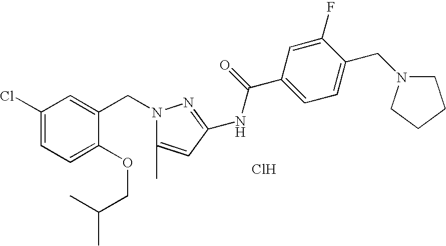 | N-[1-({5-chloro-2-[(2- methylpropyl)oxy]phenyl}methyl)- 5-methyl-1H-pyraozl-3-yl]-4-(1- pyrrolidinylmethyl)-2- fluorobenzamide hydrochlroride | LC/MS Rt = 2.45 min [MH+] 499, 501 |
| 430 | 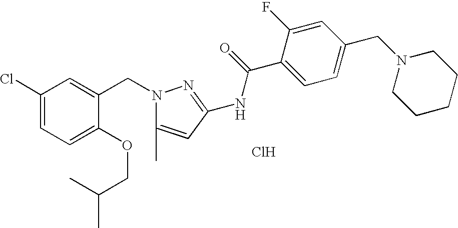 | N-[1-({5-chloro-2-[(2- methylpropyl)oxy]phenyl}methyl)- 5-methyl-1H-pyrazol-3-yl]-4-(1- piperidinylmethyl)-2- fluorobenzamide hydrochloride | LC/MS Rt = 2.53 min [MH+] 513, 515 |
| 431 | 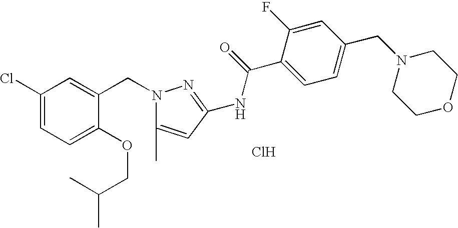 | N-[1-({5-chloro-2-[(2- methylpropyl)oxy]phenyl}methyl)- 5-methyl-1H-pyraozl-3-yl]-4-(4- morpholinylmethyl)-2- fluorobenzamide hydrochloride | LC/MS Rt = 2.48 min [MH+] 515, 517 |

### Example 432

### 4-(aminomethyl)-N-[1-({5-chloro-2-[(2-methylpropyl)oxy]phenyl}methyl)-5-methyl-1H-pyrazol-3-yl]-2-fluorobenzamide hydrochloride

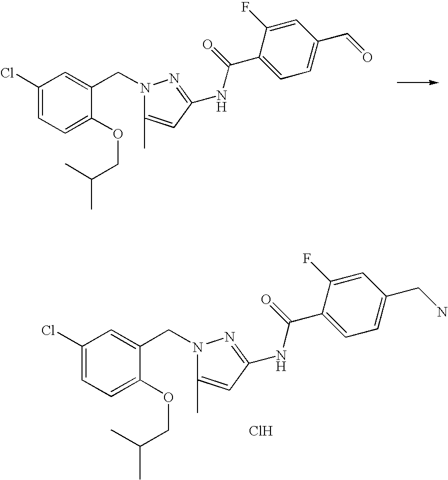

4-(aminomethyl)-N-[1-({5-chloro-2-[(2-methylpropyl)oxy]phenyl}methyl)-5-methyl-1H-pyrazol-3-yl]-2-fluorobenzamide hydrochloride was isolated from the synthesis of example SPIRAL during purification on the MDAP. The residue was stirred in 1M hydrogen chloride in diethyl ether (1 ml) and dried to yield the title compound (0.016 g).

LC/MS Rt=3.36 min [MH+] 446, 448.

### Example 433

### N-[1-({5-chloro-2-[(2-methylpropyl)oxy]phenyl}methyl)-5-methyl-1H-pyrazol-3-yl]-3-fluoro-4{[(1-methylethyl)amino]methyl}benzamide hydrochloride

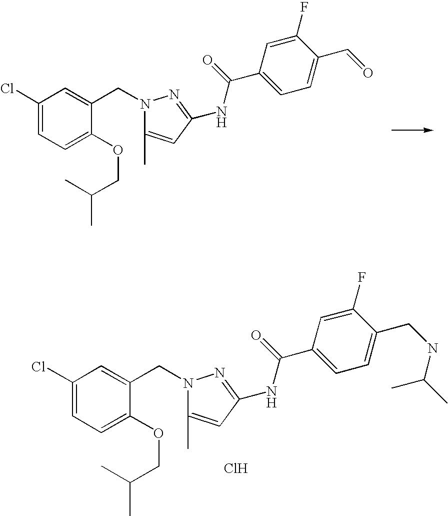

N-[1-({5-chloro-2-[(2-methylpropyl)oxy]phenyl}methyl)-5-methyl-1H-pyrazol-3-yl]-3-fluoro-4-formylbenzamide (0.1 g, 0.23 mmol), acetic acid (0.013 ml, 0.23 mmol), isopropylamine (0.038 ml, 0.46 mmol) and sodium triacetoxyborohydride (0.143 g, 0.69 mmol) were stirred in dichloromethane (3.5 ml) for 3 hours and evaporated to dryness. The residue was redissolved in methanol and purified on an SCX ion exchange cartridge and further purified by MDAP. The residue was stirred in 1M hydrogen chloride in diethyl ether (1 ml) dried to yield the title compound (0.071 g). LC/MS Rt=2.45 min [MH+] 487, 489.

The following examples were prepared in a similar manner to that described for N-[1-({5-chloro-2-[(2-methylpropyl)oxy]phenyl}methyl)-5-methyl-1H-pyrazol-3-yl]-3-fluoro-4-{[(1-methylethyl)amino]methyl}benzamide hydrochloride from the appropriate intermediates:

| Example | Structure | Compound name | Data |
|---|---|---|---|
| 434 | 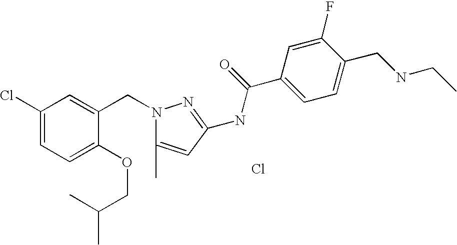 | N-[1-({5-chloro-2-[(2- methylpropyl)oxy] phenyl}methyl)-5- methyl-1H-pyrazol-3-yl]-4- [(ethylamino)methyl]-3- fluorobenzamide hydrochloride | LC/MS Rt = 2.43 mins [MH+] 473, 475 |
| 435 | 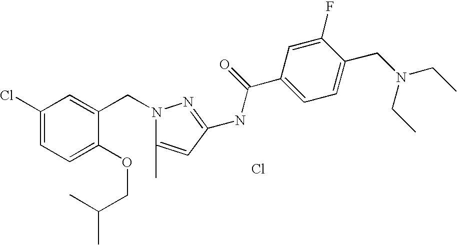 | N-[1-({5-chloro-2-[(2- methylpropyl)oxy] phenyl}methyl)-5- methyl-1H-pyrazol-3-yl]-4- [(diethylamino)methyl]-3- fluorobenzamide hydrochloride | LC/MS Rt = 2.49 mins [MH+] 501, 503 |
| 436 | 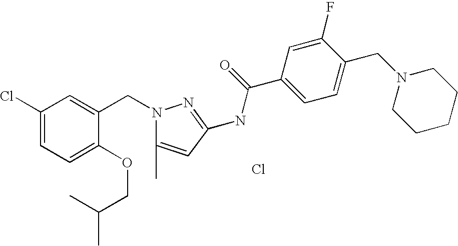 | N-[1-({5-chloro-2-[(2- methylpropyl)oxy] phenyl}methyl)-5- methyl-1H-pyrazol-3-yl]-3- fluoro-4-(1- piperidinylmethyl)benzamide hydrochloride | LC/MS Rt = 2.49 mins [MH+] 501, 503 |
| 437 | 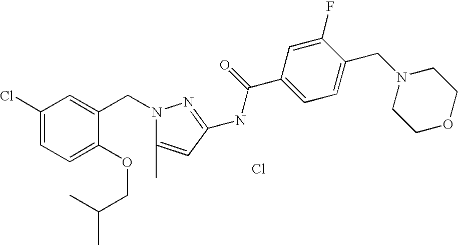 | N-[1-({5-chloro-2-[(2- methylpropyl)oxy] phenyl}methyl)-5- methyl-1H-pyrazol-3-yl]-3- fluoro-4-(4- morpholinylmethyl)benzamide hydrochloride | LC/MS Rt = 2.46 mins [MH+] 515, 517 |
| 438 | 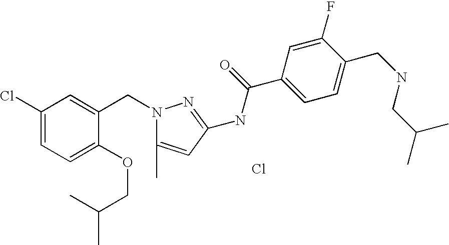 | N-[1-({5-chloro-2-[(2- methylpropyl)oxy] phenyl}methyl)-5- methyl-1H-pyrazol-3- yl]-3-fluoro-4- {[(2- methylpropyl)amino] methyl}benzamide hydrochloride | LC/MS Rt = 2.53 mins [MH+] 501, 503 |
| 439 | 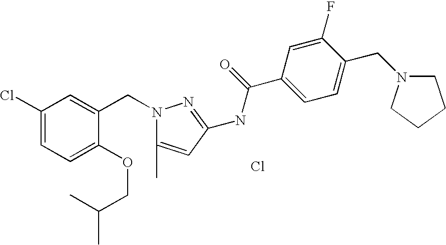 | N-[1-({5-chloro-2-[(2- methylpropyl)oxy] phenyl}methyl)-5- methyl-1H-pyrazol-3- yl]-3-fluoro-4-(1- pyrrolidinylmethyl)benzamide hydrochloride | LC/MS Rt = 2.46 mins [MH+] 499, 501 |

### Example 440

### N-[1-({5-chloro-2-[(2-methylpropyl)oxy]phenyl}methyl)-5-methyl-1H-pyrazol-3-yl]-2,6-difluoro-4-{[(1-methylethyl)amino]methyl}benzamide hydrochloride

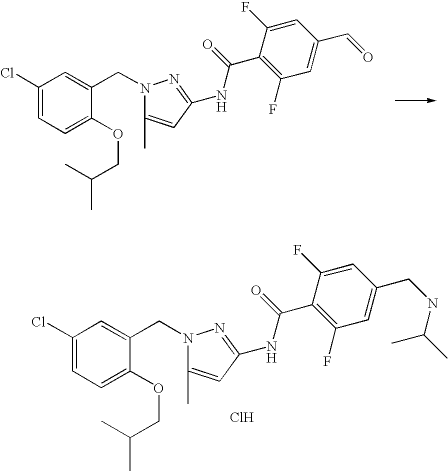

N-[1-({5-chloro-2-[(2-methylpropyl)oxy]phenyl}methyl)-5-methyl-1H-pyrazol-3-yl]-2,6-difluoro-4-{[(1-methylethyl)amino]methyl}benzamide hydrochloride (0.1 g, 0.23 mmol), acetic acid (0.013 ml, 0.23 mmol), isopropylamine (0.038 ml, 0.46 mmol) and sodium triacetoxyborohydride (0.143 g, 0.69 mmol) were stirred in dichloromethane (3.5 ml) for 3 hours and evaporated to dryness. The residue was redissolved in methanol and purified on an SCX ion exchange cartridge and further purified by MDAP. The residue was redissolved in dichloromethane, stirred in 1M hydrogen chloride in diethyl ether (1 ml) and dried to yield the title compound (0.074 g). LC/MS Rt=2.45 min [MH+] 505, 507.

The following examples were prepared in a similar manner to that described for N-[1-({5-chloro-2-[(2-methylpropyl)oxy]phenyl}methyl)-5-methyl-1H-pyrazol-3-yl]-2,6-difluoro-4-{[(1-methylethyl)amino]methyl}benzamide hydrochloride from the appropriate intermediates:

| Example | Structure | Compound name | Data |
|---|---|---|---|
| 441 | 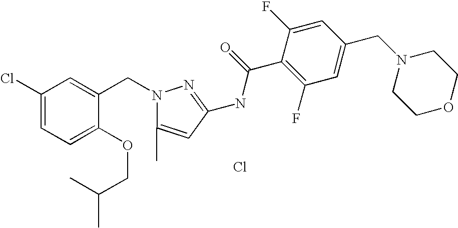 | N-[1-({5-chloro-2-[(2- methylpropyl)oxy]phenyl}methyl)-5- methyl-1H-pyrazol-3-yl]-2,6- difluoro-4-(4- morpholinylmethyl)benzamide hydrochloride | LC/MS Rt = 2.50 min [MH+] 533, 535 |
| 442 | 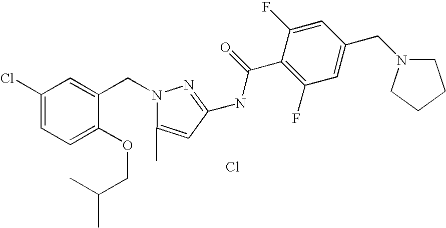 | N-[1-({5-chloro-2-[(2- methylpropyl)oxy]phenyl}methyl)-5- methyl-1H-pyrazol-3-yl]-2,6- difluoro-4-(1- pyrrolidinylmethyl)benzamide hydrochloride | LC/MS Rt = 2.45 min [MH+] 517, 519 |

### 2-(1,1-dimethylethyl)6-methyl 3,4-dihydro-2,6(1H)-isoquinolinedicarboxylate

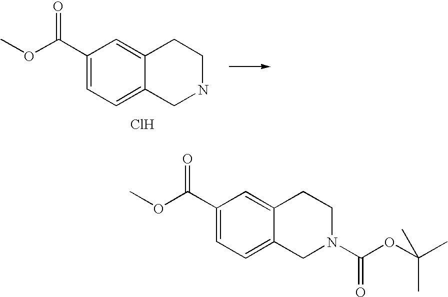

Methyl 1,2,3,4-tetrahydro-6-isoquinolinecarboxylate hydrochloride (3.071 g, 13.5 mmol), BOC-anhydride (4.42 g, 20.3 mmol) and triethylamine (3.76 ml, 27.0 mol) were stirred in DCM (70 ml) under argon for 18 hours, evaporated to dryness, redissolved in ethyl acetate, washed with saturated sodium bicarbonate twice, once with brine and dried (MgSO₄). The organic solution was filtered, evaporated to a solid and purified by flash chromatography using ethyl acetate and hexane to yield the title compound (3.6 g)

LC/MS Rt=3.24 min [MH+] 236

### 2-{[(1,1-dimethylethyl)oxy]carbonyl}-1,2,3,4-tetrahydro-6-isoquinolinecarboxylic acid

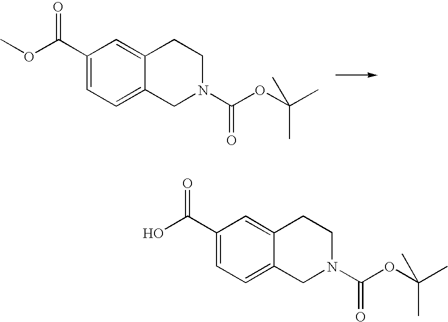

2-(1,1-dimethylethyl)6-methyl 3,4-dihydro-2,6(1H)-isoquinolinedicarboxylate (1.5 g, 5.15 mmol) and 2M sodium hydroxide (5 ml) were stirred in methanol (50 ml) for four days, evaporated to a solid and redissolved in ethyl acetate. The organic layer was washed three times with 2M hydrochoric acid, once with brine, dried (MgSO₄), filtered and evaporated to yield the title compound (1.37 g). LC/MS Rt=2.67 min [MH+] 276.

### Example 443

### N-[1-({5-chloro-2-[(2-methylpropyl)oxy]phenyl}methyl)-5-methyl-1H-pyrazol-3-yl]-1,2,3,4-tetrahydro-6-isoquinolinecarboxamide hHydrochloride

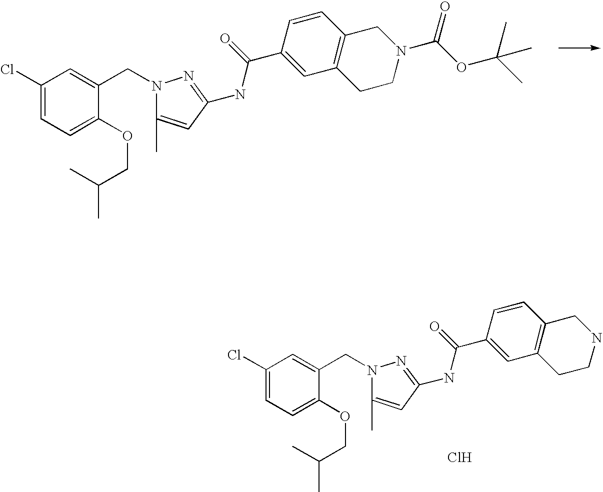

1,1-dimethylethyl 6-({[1-({5-chloro-2-[(2-methylpropyl)oxy]phenyl}methyl)-5-methyl-1H-pyrazol-3-yl]amino}carbonyl)-3,4-dihydro-2(H)-isoquinolinecarboxylate (1.25 g, 2.3 mmol) was stirred in 4M hydrogen chloride in dioxane (20 ml) for 2 hours and the reaction evaporated to dryness to yield the title compound (1.14 g).

LC/MS Rt=2.39 min [MH+] 453 and 455

### Example 444

### N-[1-({5-chloro-2-[(2-methylpropyl)oxy]phenyl}methyl)-5-methyl-1H-pyrazol-3-yl]-2-ethyl-1,2,3,4-tetrahydro-6-isoquinolinecarboxamide hydrochloride

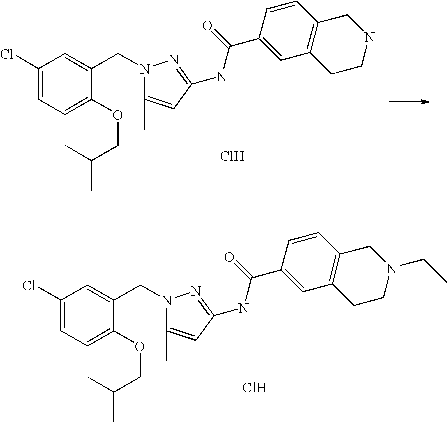

N-[1-({5-chloro-2-[(2-methylpropyl)oxy]phenyl}methyl)-5-methyl-1H-pyrazol-3-yl]-1,2,3,4-tetrahydro-6-isoquinolinecarboxamide hydrochloride (0.1 g, 0.20 mmol), acetic acid (0.012 ml, 0.20 mmol) and acetaldehyde (0.022 ml, 0.40 mmol) were stirred in dichloromethane (5 ml) for 64 hours. Sodium triacetoxyborohydride (0.13 g, 0.60 mmol) was added to the reaction with dichloromethane (5 ml), the reaction stirred for 24 hours and evaporated to dryness. The residue was redissolved in methanol and purified on an SCX ion exchange cartridge, evaporated to dryness, further purified using flash chromatography eluting with 2M ammonia in methanol and dichloromethane and evaporated to dryness. The residue was redissolved in dichloromethane, stirred in 1M hydrogen chloride in diethyl ether (1 ml) and dried to yield the title compound (0.061 g) LC/MS Rt=2.36 min [MH+] 495 and 497

The following examples were prepared in a similar manner to that described for N-[1-({5-chloro-2-[(2-methylpropyl)oxy]phenyl}methyl)-5-methyl-1H-pyrazol-3-yl]-2-ethyl-1,2,3,4-tetrahydro-6-isoquinolinecarboxamide hydrochloride from the appropriate intermediates:

| Example | Structure | Compound name | Data |
|---|---|---|---|
| 445 | 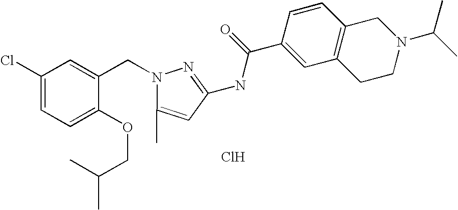 | N-[1-({5-chloro-2-[(2- methylpropyl)oxy]phenyl}methyl)- 5-methyl-1H-pyrazol-3-yl]-2-(1- methylethyl)-1,2,3,4-tetrahydro-6- isoquinolinecarboxamide hydrochloride | LC/MS Rt = 2.43 min [MH+] 495, 497 |
| 446 | 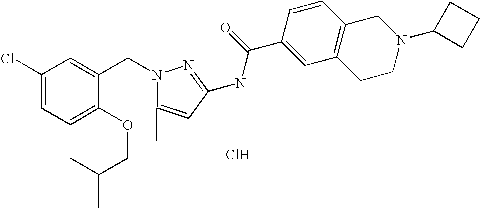 | N-[1-({5-chloro-2-[(2- methylpropyl)oxy]phenyl}methyl)- 5-methyl-1H-pyrazol-3-yl]-2- cyclobutyl-1,2,3,4-tetrahydro-6- isoquinolinecarboxamide hydrochloride | LC/MS Rt = 2.48 min [MH+] 507, 509 |
| 447 | 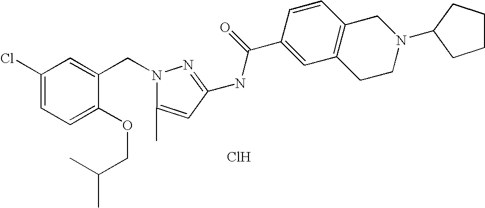 | N-[1-({5-chloro-2-[(2- methylpropyl)oxy]phenyl}methyl)- 5-methyl-1H-pyrazol-3-yl]-2- cyclopentyl-1,2,3,4-tetrahydro-6- isoquinolinecarboxamide hydrochloride | LC/MS Rt = 2.53 min [MH+] 521, 523 |
| 448 | 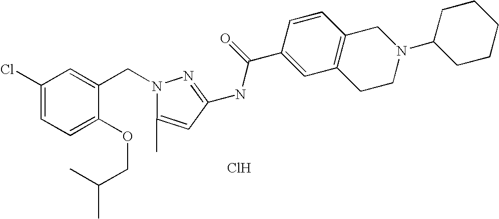 | N-[1-({5-chloro-2-[(2- methylpropyl)oxy]phenyl}methyl)- 5-methyl-1H-pyrazol-3-yl]-2- cyclohexyl-1,2,3,4-tetrahydro-6- isoquinolinecarboxamide hydrochloride | LC/MS Rt = 2.60 min [MH+] 535, 537 |
| 449 | 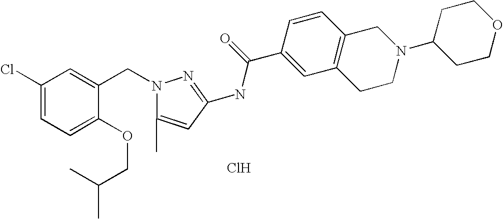 | N-[1-({5-chloro-2-[(2- methylpropyl)oxy]phenyl}methyl)- 5-methyl-1H-pyrazol-3-yl]-2- (tetrahydro-2H-pyran-4-yl)- 1,2,3,4-tetrahydro-6- isoquinolinecarboxamide hydrochloride | LC/MS Rt = 2.43 min [MH+] 537, 539 |

### Example 450

### N-[1-({5-chloro-2-[(2-methylpropyl)oxy]phenyl}methyl)-5-methyl-1H-pyrazol-3-yl]-4-(hydroxymethyl)benzamide

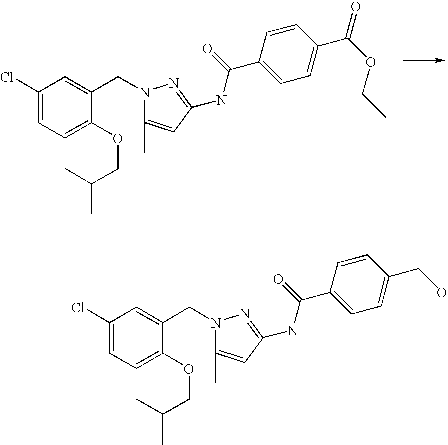

N-[1-({5-chloro-2-[(2-methylpropyl)oxy]phenyl}methyl)-5-methyl-1H-pyrazol-3-yl]-2-(tetrahydro-2H-pyran-4-yl)-1,2,3,4-tetrahydro-6-isoquinolinecarboxamide hydrochloride (0.49 g, 1.1 mmol) was dissolved in THF (5 ml) and added dropwise to a 0.2M solution of lithium aluminium hydride in THF (5 ml) under argon with stirring at 0° C. The reaction mixture was stirred for an hour, quenched with water (1 ml) in THF (4 ml), diluted with ethyl acetate, the organic layer washed twice with 2M hydrochloric acid, once with water, once with brine, dried (MgSO₄), filtered, evaporated to a colourless oil and purified using flash chromatography eluting with ethyl acetate hexane to yield the title compound (0.361 g) LC/MS Rt=3.20 min [MH+] 428 and 430

### Example 451

### N-[1-({5-chloro-2-[(2-methylpropyl)oxy]phenyl}methyl)-5-methyl-1H-pyrazol-3-yl]-{1-[(2-methylpropyl)amino]ethyl}benzamide hydrochloride

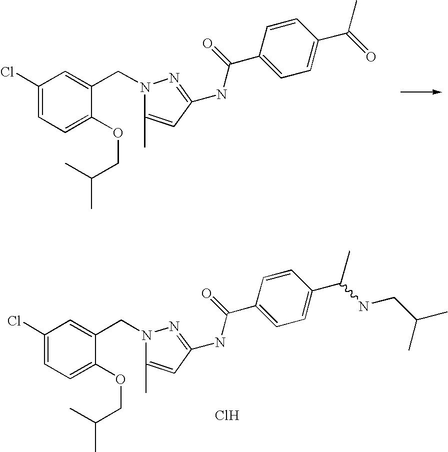

4-acetyl-N-[1-({5-chloro-2-[(2-methylpropyl)oxy]phenyl}methyl)-5-methyl-1H-pyrazol-3-yl]benzamide (0.1 g, 0.23 mmol), isobutylamine (0.045 ml, 0.46 mol) and acetic acid (0.013 ml, 0.23 mol) were stirred in dichloromethane (2 ml) for 1 hour, sodium triacetoxyborohydride (0.143 g, 0.69 mmol) added and the reaction allowed to stir for 20 hours and evaporated to dryness. The residue was redissolved in methanol and purified on an SCX ion exchange cartridge and further purified by MDAP. The residue was redissolved in dichloromethane, stirred in 1M hydrogen chloride in diethyl ether (1 ml) and dried to yield the title compound (0.039 g). LC/MS Rt=2.64 min [MH+] 497, 499.

The following example was prepared in a similar manner to that described for N-[1-({5-chloro-2-[(2-methylpropyl)oxy]phenyl}methyl)-5-methyl-1H-pyrazol-3-yl]-4-{1-[(2-methylpropyl)amino]ethyl}benzamide hydrochloride from the appropriate intermediates:

| Example | Structure | Compound name | Data |
|---|---|---|---|
| 452 | 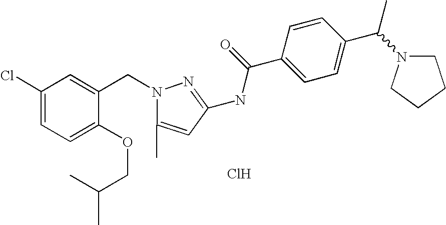 | N-[1-({5-chloro-2-[(2- methylpropyl)oxy]phenyl}methyl)- 5-methyl-1H-pyrazol-3-yl]-4-[1-(1- pyrrolidinyl)ethyl]benzamide hydrochloride | LC/MS Rt = 2.55 min [MH+] 495, 497 |

### Example 453

### N-[1-({5-chloro-2-[(2-methylpropyl)oxy]phenyl}methyl)-5-methyl-1H-pyrazol-3-yl]-4-(2-pyrrolidinyl)benzamide hydrochloride

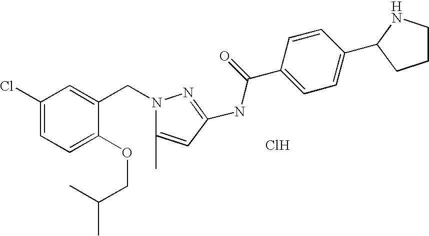

### (a) Methyl 4-[4-({[(1,1-dimethylethyl)oxy]carbonyl}amino)butanoyl]benzoate

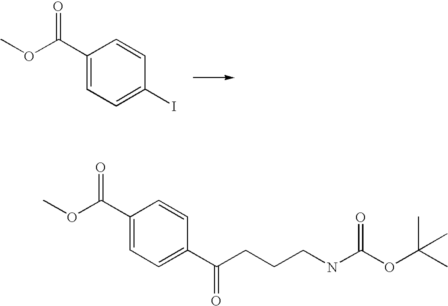

Methyl 4-iodobenzoate (2.0 g, 7.6 mmol) was stirred in tetrahydrofuran (50 ml), cooled to −60° C. under argon, 0.4M isopropyl magnesium chloride in THF (4.18 ml, 8.4 mmol) added dropwise, stirred for half an hour, 1,1-dimethylethyl 2-oxo-1-pyrrolidinecarboxylate (1.56 ml, 9.1 mmol) and the reaction allowed to come to room temperature. The reaction was quenched with 2M hydrochloric acid (5 ml), the THF removed by evaporation, redissolved in ethyl acetate, washed twice with water, once with brine, dried (MgSO₄), filtered, evaporated to dryness and purified by flash chromatography to yield the title compound (1.59 g). LC/MS Rt=2.97 min [MH+] 322.

### (b) 4-(3,4-dihydro-2H-pyrrol-5-yl)benzoic acid

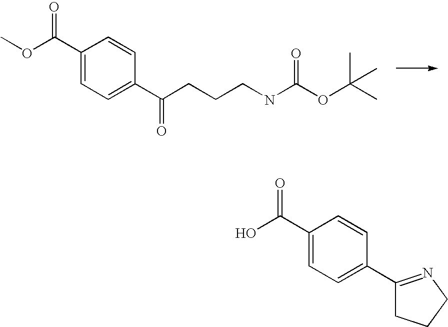

Methyl 4-[4-({[(1,1-dimethylethyl)oxy]carbonyl}amino)butanoyl]benzoate (1.5 g, 4.7 mmol) and 5M hydrochloric acid (4.5 ml) were stirred in tetrahydrofuran (10 ml) at 70° C. for 1 and a half hours, allowed to cool, azetroped with ethanol and purified on an SCX cartridge to yield the title compound (0.271 g). LC/MS Rt=0.93 min [MH+] 190.

### (c) N-[1-({5-chloro-2-[(2-methylpropyl)oxy]phenyl}methyl)-5-methyl-1H-pyrazol-3-yl]-4-(3,4-dihydro-2H-pyrrol-5-yl)benzamide

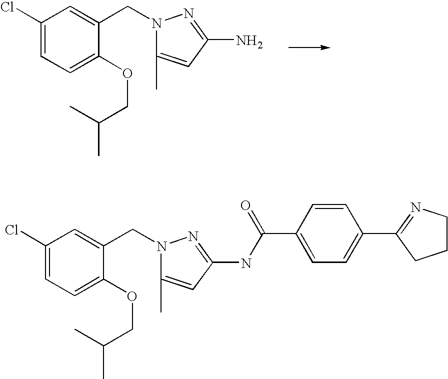

4-(3,4-dihydro-2H-pyrrol-5-yl)benzoic acid (0.097 g, 0.51 mmol), EDAC (0.098 g, 0.51 mmol) and 1-hydroxy-7-azabenzotriazole (0.069 g, 0.51 mmol) were stirred in dichloromethane (5 ml) for 45 minutes, 1-({5-chloro-2-[(2-methylpropyl)oxy]phenyl}methyl)-5-methyl-1H-pyrazol-3-amine (0.1 g, 0.34 mmol) added and allowed to react for 20 hours. The reaction mixture was diluted with ethyl acetate, washed twice with saturated sodium bicarbonate, once with brine, dried (MgSO₄), evaporated to dryness and purified by column chromatography eluting with 2M ammonia in methanol and dichloromethane and further purified by trituration with ether to yield the title compound (0.035 g)

LC/MS Rt=2.57 min [MH+] 465, 467

### (d) Example 453

### N-[1-({5-chloro-2-[(2-methylpropyl)oxy]phenyl}methyl)-5-methyl-1H-pyrazol-3-yl]-4-(2-pyrrolidinyl)benzamide hydrochloride

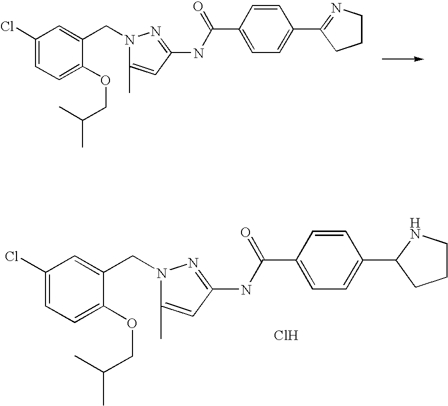

N-[1-({5-chloro-2-[(2-methylpropyl)oxy]phenyl}methyl)-5-methyl-1H-pyrazol-3-yl]-4-(3,4-dihydro-2H-pyrrol-5-yl)benzamide (0.035 g, 0.073 mmol), acetic acid (0.005 ml, 0.080 mmol) and sodium triacetoxyborohydride (0.031 g, 0.14 mmol) were stirred in dichloromethane (2 ml) for 5 days. The reaction mixture was purified on an SCX cartridge, evaporated to dryness, redissolved in dichloromethane and treated with 1M hydrogen chloride in ether (0.5 ml) and evaporated to dryness to yield the title compound (0.035 g).

LCMS Rt=2.41 min [MH+] 467, 469.

### Example 454

### 2-[1-({5-chloro-2-[(2-methylpropyl)oxy]phenyl}methyl)-5-methyl-1H-pyrazol-3-yl]-1H-isoindole-1,3(2H)-dione

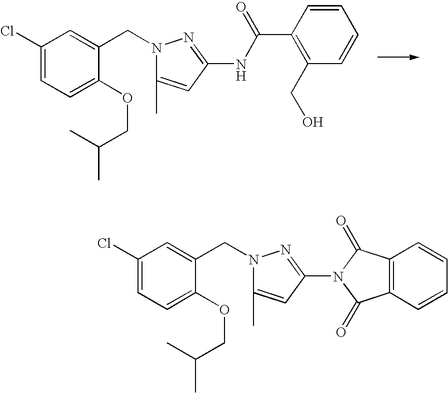

N-[1-({5-chloro-2-[(2-methylpropyl)oxy]phenyl}methyl)-5-methyl-1H-pyrazol-3-yl]-2-(hydroxymethyl)benzamide (200 mg, 0.47 mmol) and Dess-Martin periodinane (240 mg, 0.56 mmol) were stirred in dichloromethane (2 ml) for 3 hours. More Dess-Martin periodinane (240 mg, 0.56 mmol) was added and the reaction stood overnight. The reaction mixture was diluted with ethyl acetate, washed with a 1:1 mixture of 10% sodium thiosulphate solution and saturated sodium bicarbonate twice, once with brine, dried (sodium carbonate), purified by silica gel chromatography eluting with ethyl acetate and hexane and evaporated to give the title compound (68 mg).

LCMS Rt=3.70 min [MH+] 424, 426

### Example 455

### N-[1-({5-bromo-2-[(2-methylpropyl)oxy]phenyl}methyl)-5-methyl-1H-pyrazol-3-yl]-4-[(ethylamino)methyl]benzamide hydrochloride

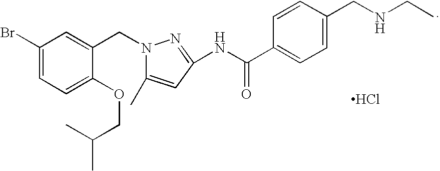

### (a) 2-(trimethylsilyl)ethyl[1-({5-bromo-2-[(2-methylpropyl)oxy]phenyl}methyl)-5-methyl-1H-pyrazol-3-yl]carbamate

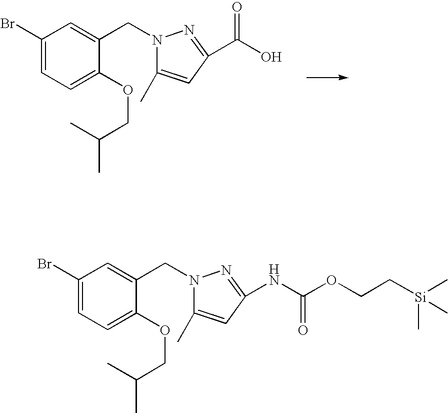

1-({5-bromo-2-[(2-methylpropyl)oxy]phenyl}methyl)-5-methyl-1H-pyrazole-3-carboxylic acid (6.00 g, 16.31 mmol) was stirred in dry toluene (35 ml) under an atmosphere of argon. Diphenylphosphoryl azide (3.86 ml, 17.94 mmol), Et₃N (2.72 ml, 19.57 mmol) and 2(trimethylsilyl)ethanol (3.51 ml, 24.47 mmol) was added and the reaction mixture was heated to 100° C. for 2 hours. It was then cooled to room temperature and was evaporated to a pale brown oil. This was partitioned between EtOAc and water. The aqueous layer was washed again with EtOAc. The organics were dried over MgSO₄, filtered and concentrated under reduced pressure to give a brown oil. The residue was chromatographed [SiO₂, Hexane:EtOAc (4:1)] to give pure product. (4.63 g, 59% yield)

LCMS Rt=4.16 [MH⁺] 482, 484

### (b) 1-({5-bromo-2-[(2-methylpropyl)oxy]phenyl}methyl)-5-methyl-1H-pyrazol-3-amine

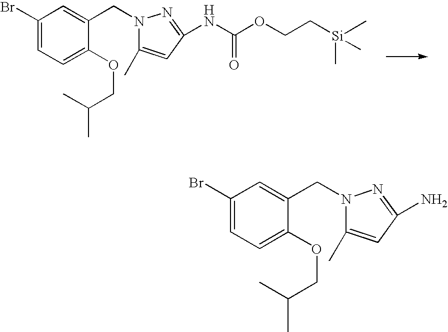

A solution of 2-(trimethylsilyl)ethyl[1-({5-bromo-2-[(2-methylpropyl)oxy]phenyl}methyl)-5-methyl-1H-pyrazol-3-yl]carbamate (3.53 g, 7.29 mmol) and tetrabutylammonium fluoride (4.22 ml, 14.59 mmol) in THF (15 ml) was stirred at 50° C. for 2 hours. After this time, the solution was allowed to cool to room temperature and the solvent was evaporated leaving a yellow oil. This was partitioned between Et₂O and water. The aqueous layer was run off and the organic layer was washed with water (×2) and brine. The organic layer was dried over Na₂SO₄ and concentrated under reduced pressure to give an off-white solid. (1.98 g, 80% yield). LCMS Rt=2.82 [MH⁺] 338, 340.

### (c) Methyl 4-({[1-({5-bromo-2-[(2-methylpropyl)oxy]phenyl}methyl)-5-methyl-1H-pyrazol-3-yl]amino}carbonyl)benzoate

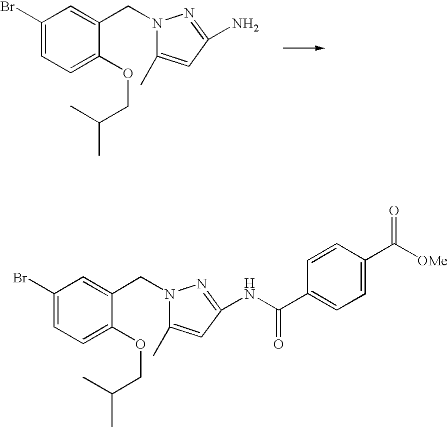

A solution of monomethyl terephthalate (1.58 g, 8.76 mmol) and N-(3-Dimethylaminopropyl)-N′-ethylcarboiimidehydrochloride (1.68 g, 8.76 mmol) was stirred at room temperature in DCM (169 ml) under an atmosphere of argon. After 5 mins, 1-Hydroxy-7-azabenzotriazole (1.19 g, 8.76 mmol) was added and the solution was stirred for 1 hour. 1-({5-bromo-2-[(2-methylpropyl)oxy]phenyl}methyl)-5-methyl-1H-pyrazol-3-amine (1.98 g, 5.84 mmol) was then added and the reaction mixture was stirred at room temperature for 3 days. The solvent was then evaporated leaving a pale yellow solid. The residue was partitioned between EtOAc and water. The aqueous layer was washed with EtOAc. The organics were dried over MgSO₄, filtered and concentrated under reduced pressure to give a pale yellow solid. The crude product was triturated with EtOAc and the 1-Hydroxy-7-azabenzotriazole precipitated out. The ester product was then washed again with EtOAc to remove any traces of 1-Hydroxy-7-azabenzotriazole. The solvent was concentrated under reduced pressure to give a pale yellow solid. (1.41 g, 48% yield). LCMS Rt=3.72 [MH⁺] 500, 502.

### (d) N-[1-({5-bromo-2-[(2-methylpropyl)oxy]phenyl}methyl)-5-methyl-1H-pyrazol-3-yl]-4-(hydroxymethyl)benzamide

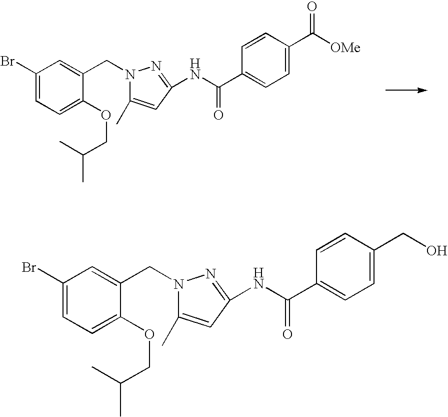

18.00 ml THF was cooled to 0° C. under an atmosphere of argon. Lithium aluminium hydride (2.81 ml, 2.81 mmol) was then added and the solution was stirred at 0° C. for 5 mins. Methyl 4-({[1-({5-bromo-2-[(2-methylpropyl)oxy]phenyl}methyl)-5-methyl-1H-pyrazol-3-yl]amino}carbonyl)benzoate (1.41 g, 2.81 mmol) was dissolved in 10.00 ml THF and this was added drop wise to the lithium aluminium hydride solution. The reaction mixture was stirred at 0° C. for 2 hours. It was then allowed to warm to room temperature and was stirred for 45 mins. A further 1.41 ml (1.41 mmol, 0.5 eq) lithium aluminium hydride was added to the solution at 0° C. and the reaction mixture was stirred at 0° C. for 15 mins. A pale pink precipitate formed. The reaction mixture was then allowed to warm to room temperature and was stirred for 30 mins. The solution was cooled to 0° C. and was carefully quenched with cold water. The solution was then allowed to warm to room temperature and the solvent was evaporated under reduced pressure to give a pale yellow solid. The residue was partitioned between EtOAc and water. The organic layer was then washed again with water. The organics were dried over MgSO₄, filtered and concentrated under reduced pressure to give a pale yellow solid. The aqueous layer was extracted again with EtOAc. The organics were dried over MgSO₄, filtered and concentrated under reduced pressure to give a pale yellow solid. (0.86 g, 65% yield). LCMS Rt=3.28 [MH⁺]472, 474

### (e) N-[1-({5-bromo-2-[(2-methylpropyl)oxy]phenyl}methyl)-5-methyl-1H-pyrazol-3-yl]-4-formylbenzamide

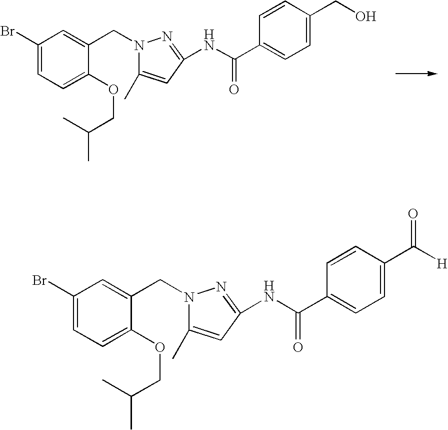

A solution of N-[1-({5-bromo-2-[(2-methylpropyl)oxy]phenyl}methyl)-5-methyl-1H-pyrazol-3-yl]-4-(hydroxymethyl)benzamide (0.278 g, 0.59 mmol) in dry DCM (3.48 ml) was stirred at 0° C. under argon for 10 mins. Dess-martin periodinane (0.25 g, 0.59 mmol) was added and the reaction mixture was stirred at room temperature for 1 hour. The reaction was quenched with 10% sodium thiosulphate (17 ml) and NaHCO₃ (sat. aq. Soln., 17 ml). The reaction mixture was extracted twice with DCM. The organics were dried over MgSO₄, filtered and concentrated under reduced pressure to give a pale brown oil. The residue was used directly in the next step.

### Standard Procedure 2

### Example 455

### N-[1-({5-bromo-2-[(2-methylpropyl)oxy]phenyl}methyl)-5-methyl-1H-pyrazol-3-yl]-4-[(ethylamino)methyl]benzamide

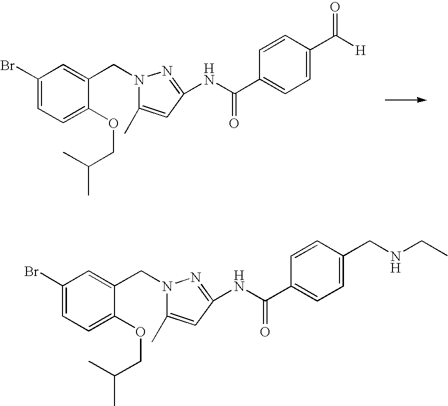

A solution of N-[1-({5-bromo-2-[(2-methylpropyl)oxy]phenyl}methyl)-5-methyl-1H-pyrazol-3-yl]-4-formylbenzamide (0.139 g, 0.295 mmol) was stirred in DCM (1.74 ml) at room temperature under an atmosphere of argon. Sodium triacetoxyborohydride (0.063 g, 0.395 mmol), acetic acid (16.89 μl, 0.295 mmol) and ethylamine (19.3 μl, 0.295 mmol) were added and the reaction mixture was stirred at room temperature overnight. The solvent was then evaporated under reduced pressure and the residue was partitioned between EtOAc and water. The organic layer was washed with NaHCO₃ (sat. aq. Soln.). The organics were dried over MgSO₄, filtered and concentrated under reduced pressure to give crude product. The residue was purified using MDAP. (0.029 g, 19% yield) t=2.29 [MH⁺] 499, 501.

The following compounds were prepared using appropriate intermediates using similar methods to that described in Standard Procedure 2:

| Example | Structure | Name | Data |
|---|---|---|---|
| 456 | 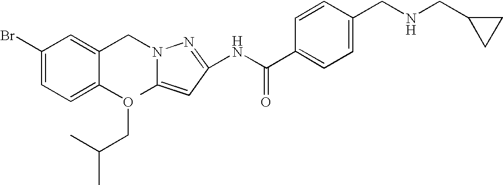 | N-[1-({5-bromo-2-[(2- methylpropyl)oxy]phenyl} methyl)-5-methyl-1H-pyrazol-3-yl]- 4-{[(cyclopropylmethyl) amino]methyl}benzamide | t = 2.41 [MH⁺] 525, 527 |
| 457 | 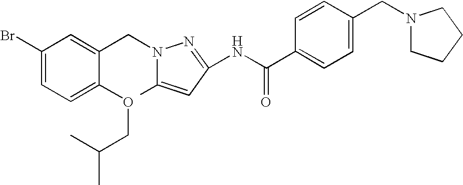 | N-[1-({5-bromo-2-[(2- methylpropyl)oxy]phenyl}methyl)-5- methyl-1H-pyrazol-3-yl]-4-(1- pyrrolidinylmethyl)benzamide | t = 2.44 [MH⁺] 525, 527 |
| 458 | 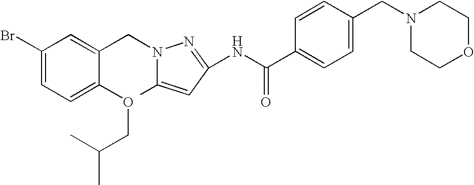 | N-[1-({5-bromo-2-[(2- methylpropyl)oxy]phenyl}methyl)-5- methyl-1H-pyrazol-3-yl]-4-(4- morpholinylmethyl)benzamide | t = 2.42 [MH⁺] 541, 543 |

### Example 459

### N-[1-({5-bromo-2-[(2-methylpropyl)oxy]phenyl}methyl)-5-methyl-1H-pyrazol-3-yl]-4-(1-piperidinylmethyl)benzamide

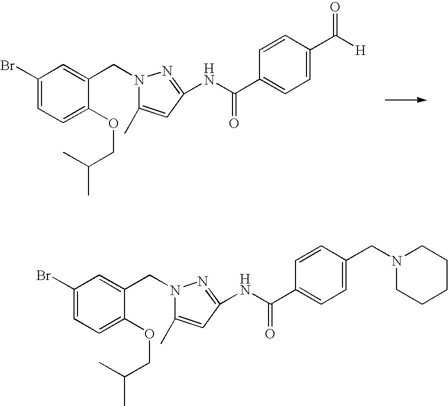

A solution of N-[1-({5-bromo-2-[(2-methylpropyl)oxy]phenyl}methyl)-5-methyl-1H-pyrazol-3-yl]-4-formylbenzamide (0.099 g, 0.21 mmol), piperidine (20.74 μl, 0.21 mmol), Sodium triacetoxyborohydride (0.045 g, 0.21 mmol) and acetic acid (12.02 μl, 0.21 mmol) in DCM (1.24 ml) was stirred at room temperature overnight under an atmosphere of argon. LCMS showed 75% product. The reaction mixture was diluted with DCM and washed with water. The aqueous layer was washed with DCM. The organics formed an off-white precipitate. This was concentrated under reduced pressure and azeotroped with toluene to give a pale brown solid. The residue was purified using MDAP. (0.044 g, 39% yield) t=2.51 [MH⁺] 539, 541

### Standard Procedure 3

### Example 455

### HCl: N-[1-({5-bromo-2-[(2-methylpropyl)oxy]phenyl}methyl)-5-methyl-1H-pyrazol-3-yl]-4-[(ethylamino)methyl]benzamide hydrochloride

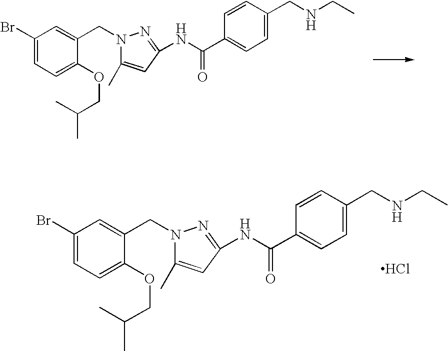

A solution of N-[1-({5-bromo-2-[(2-methylpropyl)oxy]phenyl}methyl)-5-methyl-1H-pyrazol-3-yl]-4-[(ethylamino)methyl]benzamide (0.029 g, 0.058 mmol) was stirred in 1M HCl in Et₂O (0.83 ml) for 15 mins. The solvent was then evaporated under reduced pressure. (0.025 g, 81% yield). t=2.36 [MH+] 499, 501.

The following compounds were prepared using appropriate intermediates using similar methods to that described in Standard Procedure 3:

| Example | Structure | Name | Data |
|---|---|---|---|
| 456 HCl | 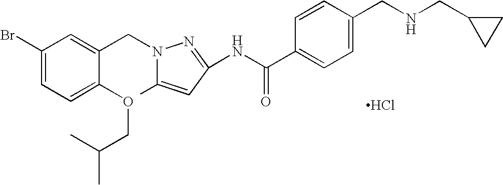 | N-[1-({5-bromo-2-[(2- methylpropyl)oxy]phenyl} methyl)-5-methyl-1H-pyrazol-3- yl]-4-{[(cyclopropylmethyl) amino]methyl}benzamide hydrochloride | LCMS Rt = 2.43 [MH⁺] 525, 527 |

### Standard Procedure 4

### Example 459

### HCl: N-[1-({5-bromo-2-[(2-methylpropyl)oxy]phenyl}methyl)-5-methyl-1H-pyrazol-3-yl]-4-(1-piperidinylmethyl)benzamide hydrochloride

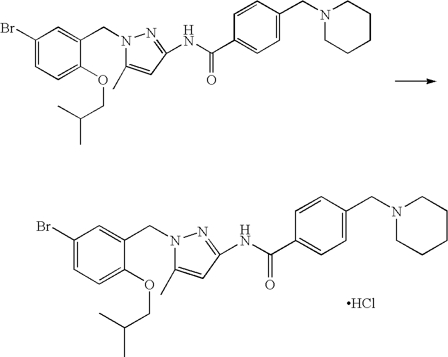

A solution of N-[1-({5-bromo-2-[(2-methylpropyl)oxy]phenyl}methyl)-5-methyl-1H-pyrazol-3-yl]-4-(1-piperidinylmethyl)benzamide (0.044 g, 0.081 mmol) in 4M HCl in Dioxane was stirred for 1 hour. The solvent was then evaporated under reduced pressure. (0.024 g, 51% yield). LCMS Rt=2.55 [MH+] 539, 541.

The following compounds were prepared using appropriate intermediates using similar methods to that described in Standard Procedure 4:

| Example | Structure | Name | Data |
|---|---|---|---|
| 457 HCl | 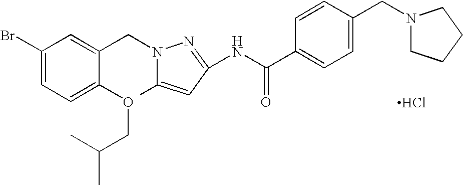 | N-[1-({5-bromo-2-[(2- methylpropyl)oxy]phenyl}methyl)- 5-methyl-1H-pyrazol-3-yl]- 4-(1-pyrrolidinylmethyl) benzamide hydrochloride | LCMS Rt = 2.48 [MH⁺] 525, 527 |
| 458 HCl | 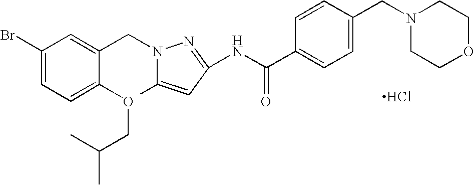 | N-[1-({5-bromo-2-[(2- methylpropyl)oxy]phenyl} methyl)-5-methyl-1H- pyrazol-3-yl]-4-(4- morpholinylmethyl) benzamide hydrochloride | LCMS Rt = 2.45 [MH⁺] 541, 543 |

### Example 460

### 4-piperidinylmethyl[1-({5-bromo-2-[(phenylmethyl)oxy]phenyl}methyl)-5-methyl-1H-pyrazol-3-yl]carbamate hydrochloride

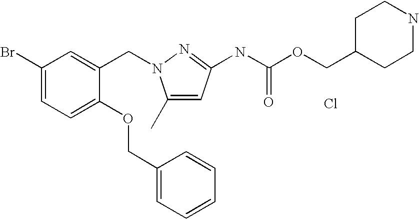

### (a) 1,1-dimethylethyl 4-{[({[1-({5-bromo-2-[(phenylmethyl)oxy]phenyl}methyl)-5-methyl-1H-pyrazol-3-yl]amino}carbonyl)oxy]methyl}-1-piperidinecarboxylate

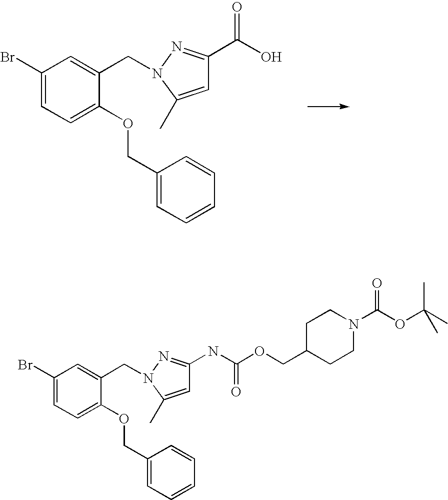

Diphenylphosphoryl azide (DPPA) (0.222 ml, 1.03 mmol, 1.1 eq) added to 1-({5-bromo-2-[(phenylmethyl)oxy]phenyl}methyl)-5-methyl-1H-pyrazole-3-carboxylic acid (376.0 mg, 0.94 mmol) in PhMe (0.94 mL, 1M) containing TEA (0.156 mL, 1.12 mmol, 1.2 eq). Mixture heated at 80° C. for 15 min (homogenous) then [(1,1-dimethylethyl)oxy][4-(hydroxymethyl)-1-piperidinyl]methanol (1.013 g, (4.71 mmol) added. Heating continued at 80° C. for further 4 hrs then cooled to rt. Mixture diluted with EtOAc and washed sequentially with 2M HCl and sat. bicarb., dried (Na₂SO₄), filtered and concentrated. Purified by chromatography on silica gel (50 g SPE), eluted with hexane+EtOAc (15-20-25-30-35-40-45%) to give the title compound (407 mg, 71%) containing approximately 7% impurity by ¹H NMR.

LCMS: Rt 3.82 min, [ES+] 615.

### (b) 4-piperidinylmethyl[1-({5-bromo-2-[(Phenylmethyl)oxy]phenyl}methyl)-5-methyl-1H-pyrazol-3-yl]carbamate

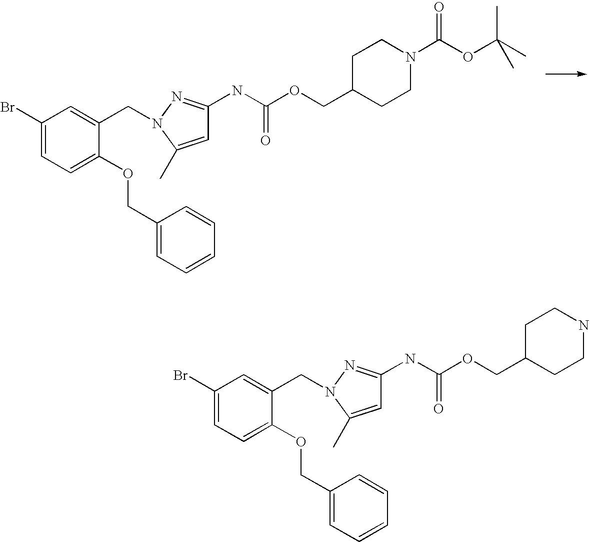

4M HCl in dioxane (4 mL) added to solid 1,1-dimethylethyl 4-{[({[1-({5-bromo-2-[(phenylmethyl)oxy]phenyl}methyl)-5-methyl-1H-pyrazol-3-yl]amino}carbonyl)oxy]methyl}-1-piperidinecarboxylate (404.0 mg, 0.66 mmol) all dissolved. Stirred at rt for 3 h then evaporated. Et₂O added and evaporated to give a solid. Residue dissolved in MeOH and loaded onto an SCX (5 g) column. Eluted with MeOH then 2M NH₃ in MeOH. Evaporated to give a white solid, 4-piperidinylmethyl[1-({5-bromo-2-[(phenylmethyl)oxy]phenyl}methyl)-5-methyl-1H-pyrazol-3-yl]carbamate, 328.3 mg (97%). LCMS: Rt 2.42 min, [ES+] 515.

### (c) 4-piperidinylmethyl[1-({5-bromo-2-[(phenylmethyl)oxy]phenyl}methyl)-5-methyl-1H-pyrazol-3-yl]carbamate hydrochloride

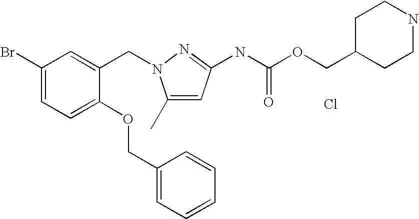

1M HCl in Et₂O (2 mL) added to solid 4-piperidinylmethyl[1-({5-bromo-2-[(phenylmethyl)oxy]phenyl}methyl)-5-methyl-1H-pyrazol-3-yl]carbamate (44.3 mg, 0.09 mmol). Not soluble. DCM added to aid solubility. Stirred for ˜4 h. Evaporated to give a white solid, 4-piperidinylmethyl[1-({5-bromo-2-[(phenylmethyl)oxy]phenyl}methyl)-5-methyl-1H-pyrazol-3-yl]carbamate hydrochloride. LCMS: Rt 2.45 min, [ES+] 515.

### Example 461

### 4-piperidinylmethyl{1-[(5-chloro-2{[(2,4-difluorophenyl)methyl]oxy}phenyl)methyl]-5-methyl-1H-pyrazol-3-yl}carbamate hydrochloride

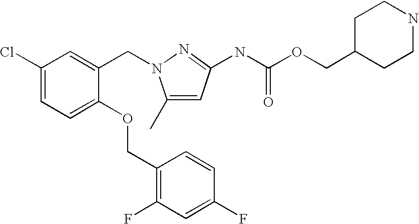

### (a) 1,1-dimethylethyl 4-({[({1-[(5-chloro-2-{[(2,4-difluorophenyl)methyl]oxy}phenyl)methyl]-5-methyl-1H-pyrazol-3-yl}amino)carbonyl]oxy}methyl)-1-piperidinecarboxylate

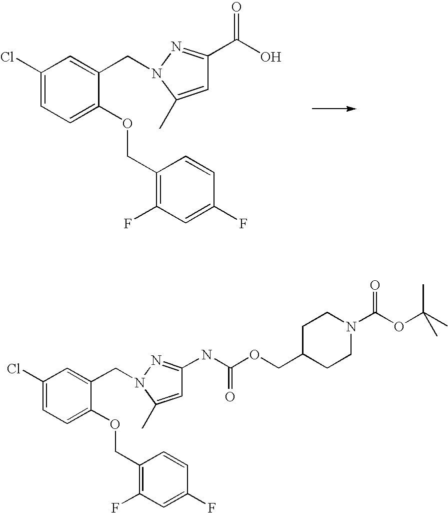

1-[(5-Chloro-2-{[(2,4-difluorophenyl)methyl]oxy}phenyl)methyl]-5-methyl-1H-pyrazole-3-carboxylic acid (695.4 mg, 1.77 mmol), TEA (296 uL, 2.13 mmol, 1.2 eq) and diphenylphosphoryl azide (DPPA) (420 uL, 1.95 mmol, 1.1 eq) in PhMe (1.8 mL, 1M) heated at 80° C. for 15 min then [(1,1-dimethylethyl)oxy][4-(hydroxymethyl)-1-piperidinyl]methanol (1.032 g, (4.80 mmol, 2.7 eq) added. Heating continued at 80° C. for further 5.5 hrs then cooled to room temp., diluted with EtOAc and washed sequentially with 2M HCl and sat. bicarb., dried (Na₂SO₄), filtered and concentrated. Loaded onto 50 g SPE silica cartridge and eluted with hexane+EtOAc (20-50%) to give the title compound (772.3 mg). 94% pure by LCMS. Re-purified on silica gel (20 g SPE) with hexane+EtOAc (10-20-30%) to give the title compound—still impurity present by TLC. LCMS: Rt 3.77 min, [ES+] 605, 607; [ES-] 1603, 604.

### (b) 4-piperidinylmethyl{1-[(5-chloro-2-{[(2,4-difluorophenyl)methyl]oxy}phenyl)methyl]-5-methyl-1H-pyrazol-3-yl}carbamate

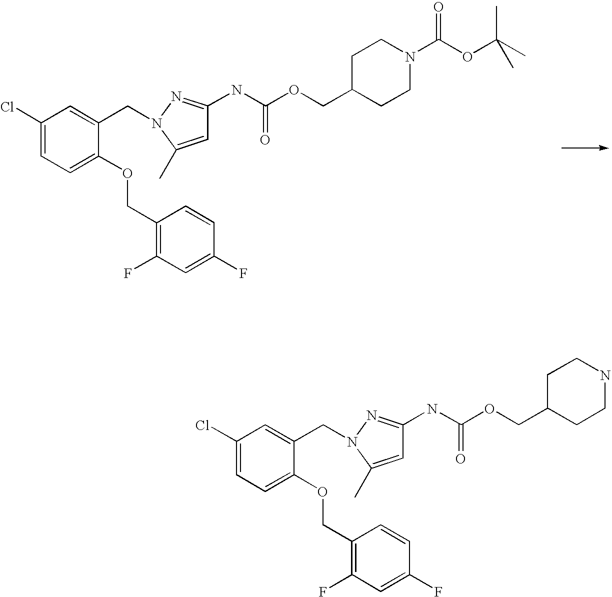

4M HCl in dioxane (6.5 mL) added to solid 1,1-dimethylethyl 4-({[({1-[(5-chloro-2-{[(2,4-difluorophenyl)methyl]oxy}phenyl)methyl]-5-methyl-1H-pyrazol-3-yl}amino)carbonyl]oxy}methyl)-1-piperidinecarboxylate (642.8 mg). Stirred at rt for 2 h then evaporated. Purified on SCX cartridge with MeOH then 2M NH₃ in MeOH to give the title compound (476.0 mg, white solid). LCMS: Rt 2.45 min, [ES+] 505, 507 (93% pure).

### (c) 4-Piperidinylmethyl {1-[(5-chloro-2-{[(2,4-difluorophenyl)methyl]oxyl}phenyl)methyl]-5-methyl-1H-pyrazol-3-yl}carbamate hydrochloride

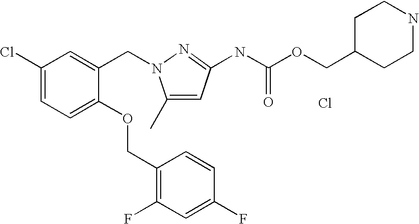

4-piperidinylmethyl {1-[(5-chloro-2-{[(2,4-difluorophenyl)methyl]oxy}phenyl)methyl]-5-methyl-1H-pyrazol-3-yl}carbamate (142.5 mg, 0.28 mmol) was dissolved in DCM (˜4 mL) and tested with 1M HCl in Et₂O (˜3 mL). Stirred for ˜4 h then evaporated to give the title compound as a white solid. LCMS: Rt 2.44 min, [ES+] 505, 507.

### Example 462

### 4-piperidinylmethyl [({5-chloro-2-[(2-methylpropyl)oxy]phenyl}methyl)-5-methyl-1H-pyrazol-3-yl]carbamate hydrochloride

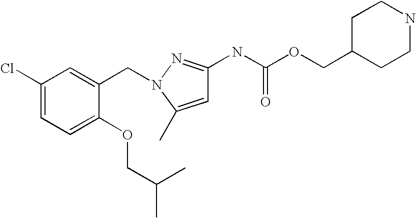

### (a) 1,1-dimethylethyl 4-{[({[1-({5-chloro-2-[(2-methylpropyl)oxy]phenyl}methyl)-5-methyl-1H-pyrazol-3-yl]amino}carbonyl)oxy]methyl}-1-piperidinecarboxylate

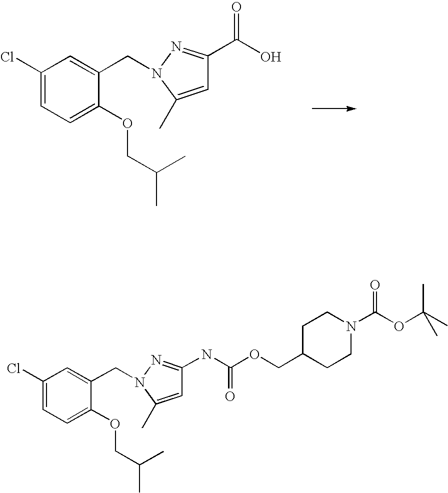

1-({5-Chloro-2-[(2-methylpropyl)oxy]phenyl}methyl)-5-methyl-1H-pyrazole-3-carboxylic acid (506.8 mg, 1.57 mmol), TEA (262 uL) and diphenylphosphoryl azide (DPPA) (372 uL) heated in PhMe (1.6 mL, 1M) at 80° C. for ˜15 min then [(1,1-dimethylethyl)oxy][4-(hydroxymethyl)-1-piperidinyl]methanol (989.5 mg) added. Continued heating for further 18 hrs. Cooled to room temp and diluted with EtOAc and washed sequentially with 2M HCl and sat. bicarb., dried (Na₂SO₄), filtered and concentrated. Residue purified on silica gel (50 g SPE), eluted with hexane+EtOAc (10-30-40%) to give the title compound (505.7 mg, 60%) as a white foam. LCMS: Rt 3.84 min, [ES+] 535, 537 (97% pure).

### (b) 4-piperidinylmethyl[1-({5-chloro-2-[(2-methylpropyl)oxy]phenyl}methyl)-5-methyl-1H-pyrazol-3-yl]carbamate

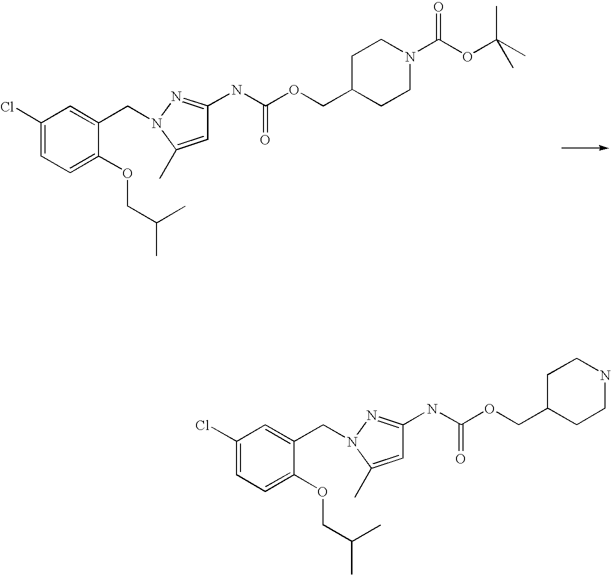

4M HCl in dioxane (5 mL) added to solid 1,1-dimethylethyl 4-{[({[1-({5-chloro-2-[(2-methyl propyl)oxy]phenyl}methyl)-5-methyl-1H-pyrazol-3-yl]amino}carbonyl)oxy]methyl}-1-piperidinecarboxylate (494.1 mg) (all dissolved). Stirred for 2 h then evaporated. Residue dissolved in MeOH and loaded onto an SCX cartridge. Eluted with MeOH then 2M NH₃ in MeOH. Required fractions evaporated to the title compound (379.0 mg, 94%).

LCMS: Rt 2.44 min, [ES+] 435, 437

### (c) 4-piperidinylmethyl[1-({5-chloro-2-[(2-methylpropyl)oxy]phenyl}methyl)-5-methyl-1H-pyrazol-3-yl]carbamate hydrochloride

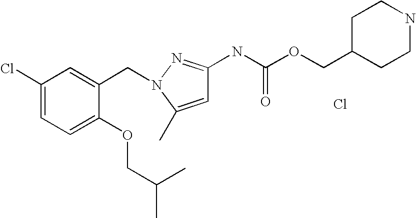

4M HCl in dioxane (2 mL) added to solid 4-piperidinylmethyl[1-({5-chloro-2-[(2-methylpropyl)oxy]phenyl}methyl)-5-methyl-1H-pyrazol-3-yl]carbamate (61.2 mg). Stirred at rt for 15mins then evaporated to give the title compound. LCMS: Rt 2.30 min, [ES+] 435, 437

### Example 463

### (1-propyl-4-piperidinyl)methyl [1-({5-chloro-2-[(2-methylpropyl)oxy]phenyl}methyl)-5-methyl-1H-pyrazol-3-yl]carbamate hydrochloride

### (a) (1-propyl-4-piperidinyl)methyl[1-({5-chloro-2-[(2-methylpropyl)oxy]phenyl}methyl)-5-methyl-1H-pyrazol-3-yl]carbamate

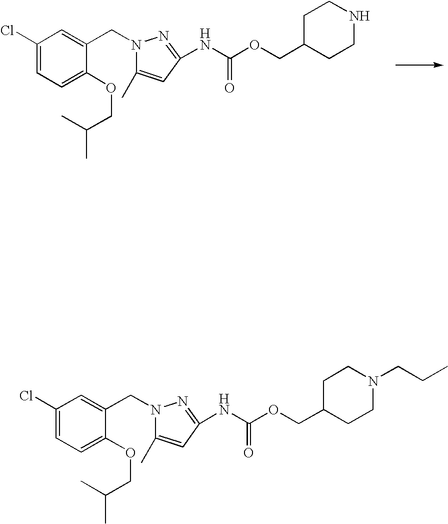

A solution of 4-piperidinylmethyl[1-({5-chloro-2-[(2-methylpropyl)oxy]phenyl}methyl)-5-methyl-1H-pyrazol-3-yl]carbamate (0.090 g, 0.21 mmol) and propylaldehyde (15.15 μl, 0.21 mmol) was stirred in DCM (1.2 ml) at room temperature for 45 mins. After this time, sodium triactoxyborohydride (0.045 g, 0.21 mmol) and acetic acid (12.02 μl, 0.21 mmol) was added and the solution was stirred overnight. The solvent was evaporated under reduced pressure and the residue was partitioned between EtOAc and water. The aqueous layer was washed with EtOAc. The combined organics were dried over MgSO₄, filtered and concentrated under reduced pressure to give crude product. The residue was purified using MDAP to give pure product. (0.029 g, 29% yield). Rt=2.56mins, [MH+] 477, 479.

### (b) (1-propyl-4-piperidinyl)methyl[1-({5-chloro-2-[(2-methylpropyl)oxy]phenyl}methyl)-5-methyl-1H-pyrazol-3-yl]carbamate Hydrochloride

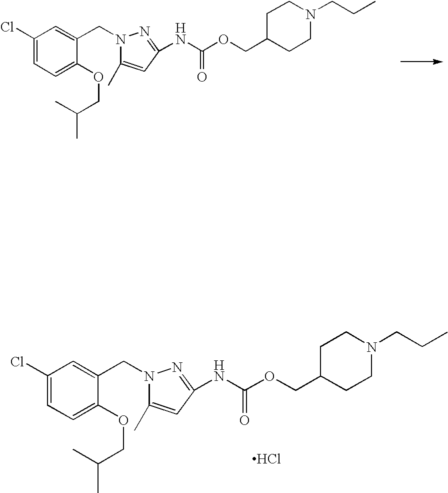

A solution of (1-propyl-4-piperidinyl)methyl[1-({5-chloro-2-[(2-methylpropyl)-oxy]phenyl}methyl)-5-methyl-1H-pyrazol-3-yl]carbamate (0.029 g, 0.061) in HCl in Et₂O (1M) (0.87 ml) was stirred at room temperature under an atmosphere of argon for 1 hour. This was then concentrated under reduced pressure. (0.021 g, 68% yield). t=2.40 [MH⁺] 477, 479

### Standard Procedure 5

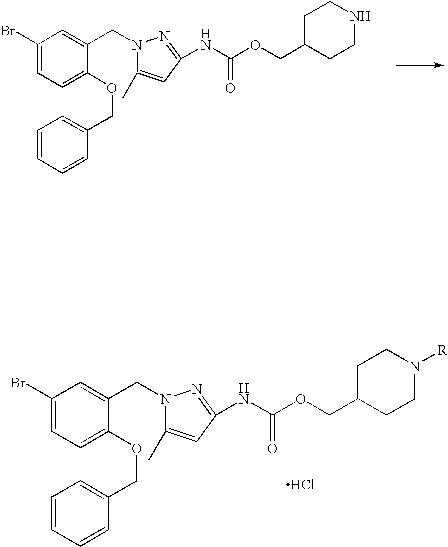

4-piperidinylmethyl[1-({5-bromo-2-[(phenylmethyl)oxy]phenyl}methyl)-5-methyl-1H-pyrazol-3-yl]carbamate (0.085 g, 0.166 mmol) was stirred in DCM (2 mL) and AcOH (0.009 mL, 1 eq.) added. The relevant aldehyde or ketone (0.024 mL, 2 eq.) was then added. The mixtures were stirred for 2 hours then allowed to stand over the weekend. The mixtures were purified on SCX cartridges (3×MeOH, 3×2M NH₃ in MeOH). The basic fractions were evaporated to colourless glasses and columned with DCM:2M NH₃ in MeOH (19:1) on a Biotage 12+M silica column. The product containing fractions were evaporated to dryness, redissolved in DCM and treated with 1M HCl in Et₂O (0.5 mL). These were evaporated to white powders.

The following Examples were prepared using appropriate intermediates using similar methods to that described in Standard Procedure 5:

| | | | LCMS |
|---|---|---|---|
| Example | Structure | Name | Data |
| 464 | 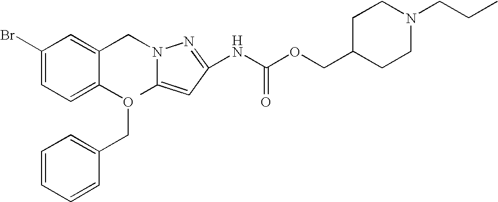 | (1-propyl-4- piperidinyl)methyl[1-({5- bromo-2- [(phenylmethyl)oxy]phenyl} methyl)-5-methyl-1H- pyrazol-3-yl]carbamate hydrochloride | Rt 2.42 min [MH+] 555 & 557 |
| 465 | 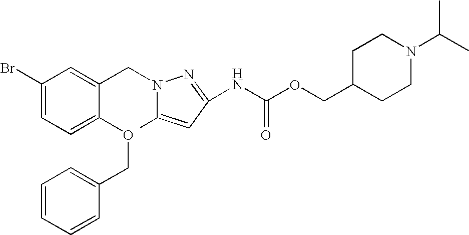 | [1-(1-methylethyl)-4- piperidinyl]methyl[1-({5- bromo-2- [(phenylmethyl)oxy]phenyl} methyl)-5-methyl-1H- pyrazol-3-yl]carbamate | Rt 2.40 min [MH+] |

### Ethyl 4-[({[1-({5-chloro-2-[(2-methylpropyl)oxy]phenyl}methyl)-5-methyl-1H-pyrazol-3-yl]amino}carbonyl)oxy]cyclohexanecarboxylate

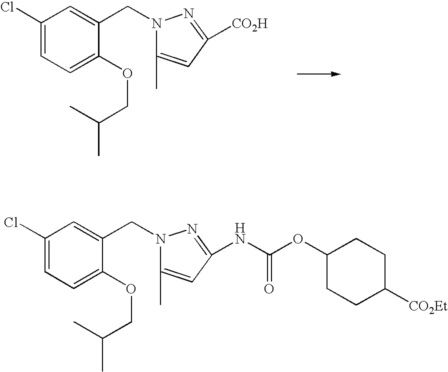

A solution of 1-({5-chloro-2-[(2-methylpropyl)oxy]phenyl}methyl)-5-methyl-1H-pyrazole-3-carboxylic acid (1.00 g, 3.11 mmol) in dry PhCH₃ (6.0 ml) was stirred at room temperature under an atmosphere of argon. Et₃N (0.649 ml, 4.67 mmol), DPPA (0.803 ml, 3.73 mmol) and ethyl-4-hydroxy-cyclohexane carboxylate (mixture of cis and trans 98%) (5.349 g, 31.10 mmol) were added to the stirred solution. The solution was heated to 80° C. for 18 hours. After this time, the solution was allowed to cool to room temperature and then it was diluted with EtOAc (˜50 ml). Organics were washed with water (3×100 ml). The combined organics were dried over MgSO₄, filtered and concentrated under reduced pressure to give a yellow oil. The residue was chromatographed [SiO₂, Hexane: EtOAc, 20-50%] to give a yellow oil (1.04 g, 68% yield). t=3.80 mins, [MH]⁺ 492, 494.

### 4-(Hydroxymethyl)cyclohexyl[({5-chloro-2-[(2-methylpropyl)oxy]phenyl}methyl)-5-methyl-1H-pyrazol-3-yl]carbamate

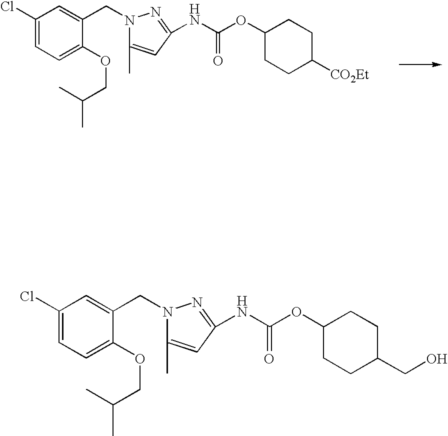

A solution of ethyl 4-[({[1-({5-chloro-2-[(2-methylpropyl)oxy]phenyl}methyl)-5-methyl-1H-pyrazol-3-yl]amino}carbonyl)oxy]cyclohexanecarboxylate (1.04 g, 2.12 mmol) in dry THF (5.0 ml) was added dropwise to a stirred solution of LiAlH₄ (1M in THF, 2.12 ml, 2.12 mmol) in dry THF (10.0 ml) at −10° C., under an atmosphere of argon. The mixture was stirred for 4 hours at 0° C. Additional LiAlH₄ (1M in THF, 0.2 ml, 0.13 mmol) was added to the solution and the reaction monitored using LC/MS. Water was added dropwise to quench excess LiAlH₄. Solution was diluted with EtOAc (150 ml), and washed with water (2×100 ml). Organics were dried over MgSO₄, filtered and concentrated under reduced pressure to give a yellow oil (0.810 g, 85% yield). t=3.33 mins, [MH]⁺ 450, 452.

### 4-Formylcyclohexyl[1-({5-chloro-2-[(2-methylpropyl)oxy]phenyl}methyl)-5-methyl-1H-pyrazol-3-yl]carbamate

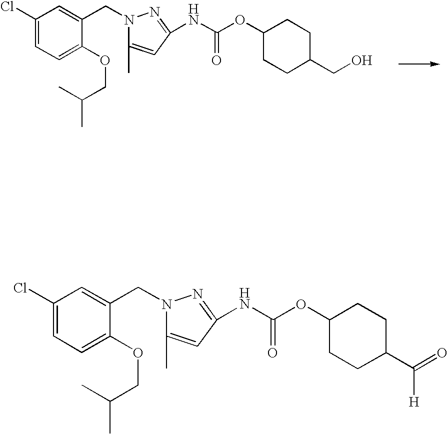

A solution of 4-(hydroxymethyl)cyclohexyl[1-({5-chloro-2-[(2-methylpropyl)oxy]phenyl}methyl)-5-methyl-1H-pyrazol-3-yl]carbamate (0.300 g, 0.67 mmol) in dry DCM (4.0 ml) was stirred at 0° C. under an atmosphere of argon. Dess-Martin periodinane (0.284 g, 0.67 mmol) was added to the stirred solution, and the resulting mixture allowed to warm to room temperature (2 hours). The reaction was quenched by addition of Na₂S₂O₃ (10% aq. soln., 20 ml) and NaHCO₃ (saturated aq. soln., 20 ml). The mixture was extracted with DCM (2×30 ml). The combined organics were dried over MgSO₄, filtered and concentrated under reduced pressure to give a brown oil.

### Standard Procedure 6

### Example 466

### 4-[(ethylamino)methyl]cyclohexyl[1-({5-chloro-2-[(2-methylpropyl)oxy]phenyl}methyl)-5-methyl-1H-pyrazol-3-yl]carbamate

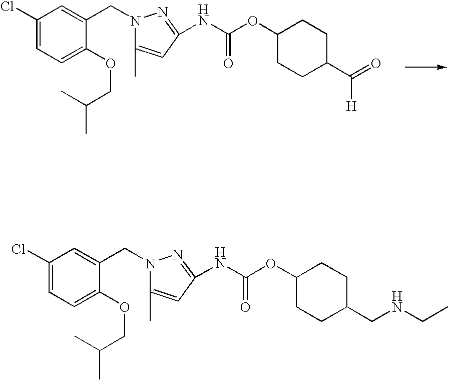

A solution of 4-formylcyclohexyl[1-({5-chloro-2-[(2-methylpropyl)oxy]phenyl}methyl)-5-methyl-1H-pyrazol-3-yl]carbamate (0.075 g, 0.68 mmol) in dry DCM (6.0 ml) was stirred at room temperature under an atmosphere of argon. AcOH (0.01 ml, 0.17 mmol) and NaBH(OAc)₃ (0.043 g, 0.20 mmol) were added to the solution. Ethylamine (2M in THF, 0.2 ml, 0.20 mmol) was added to the stirred solution. The solution was stirred for 3 hours at room temperature under an atmosphere of argon. Reaction was quenched by addition of water. Organics were extracted into DCM (3×30 ml). the combined organics were dried over MgSO₄, filtered and concentrated under reduced pressure to give a yellow oil. The residue was purified using MDAP to give product (0.02 g). t=2.41 mins [MH]⁺ 477, 479.

The following Examples were prepared using appropriate intermediates using similar methods to that described in Standard Procedure 62:

| Example | Structure | Name | LCMS Data |
|---|---|---|---|
| 467 | 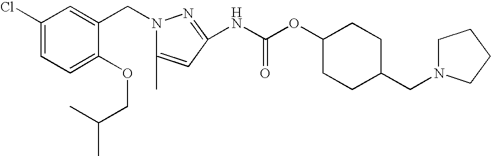 | 4-(1- pyrrolidinylmethyl)cyclohexyl [1-({5-chloro-2-[(2- methylpropyl)oxy]phenyl} methyl)-5-methyl-1H-pyrazol-3- yl]carbamate | Rt = 2.45 mins, [MH]⁺ 503, 505 |
| 468 | 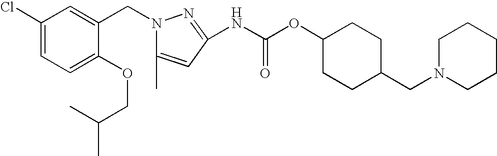 | 4-(1- piperidinylmethyl)cyclohexyl [1-({5-chloro-2-[(2- methylpropyl)oxy]phenyl} methyl)-5-methyl-1H-pyrazol-3- yl]carbamate | |
| 469 | 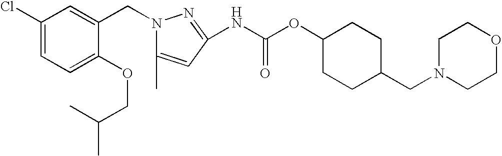 | 4-(4- morpholinylmethyl)cyclohexyl [1-({5-chloro-2-[(2- methylpropyl)oxy]phenyl} methyl)-5-methyl-1H-pyrazol-3- yl]carbamate | Rt = 2.38 mins, [MH]⁺ 519, 521 |

### Standard Procedure 7

### Example 466

### HCl: 4-[(ethylamino)methyl]cyclohexyl[1-({5-chloro-2-[(2-methylpropyl)oxy]phenyl}methyl)-5-methyl-1H-pyrazol-3-yl]carbamate hydrochloride

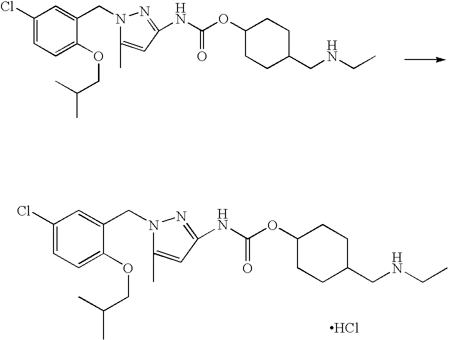

A mixture of 4-[(ethylamino)methyl]cyclohexyl[1-({5-chloro-2-[(2-methylpropyl)oxy]phenyl}-methyl)-5-methyl-1H-pyrazol-3-yl]carbamate (0.02 g, 0.04 mmol) in a solution of HCl in Et₂O (1.0 ml) was stirred for 10 minutes at room temperature. A precipitate formed. Solvent was removed under reduced pressure to give product (0.01 μg).

LCMS Rt=2.61 mins [MH]⁺ 477, 479 (isomeric salts separate on LC, t=2.24 and 2.61 mins only t=2.61 mins shows molecular ion).

The following Examples were prepared using appropriate intermediates using similar methods to that described in Standard Procedure 7:

| Example | Structure | Name | LCMS Data |
|---|---|---|---|
| 467 HCl | 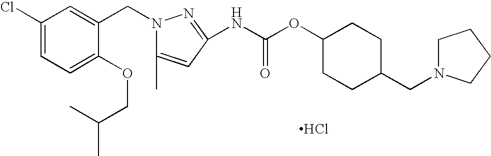 | 4-(1- pyrrolidinylmethyl)cyclohexyl [1-({5-chloro-2-[(2- methylpropyl)oxy]phenyl}methyl)- 5-methyl-1H-pyrazol-3-yl] carbamate hydrochloride | Rt = 2.66 mins, [MH]⁺ 503, 505 |
| 468 HCl | 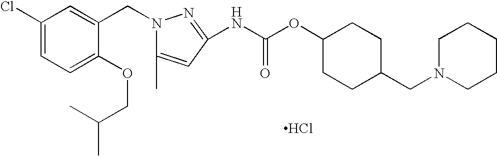 | 4-(1-piperidinylmethyl)cyclohexyl [1-({5-chloro-2-[(2- methylpropyl)oxy]phenyl}methyl)- 5-methyl-1H-pyrazol-3-yl] carbamate hydrochloride | Rt = 2.47 mins, [MH]⁺ 517, 519 |
| 469 HCl | 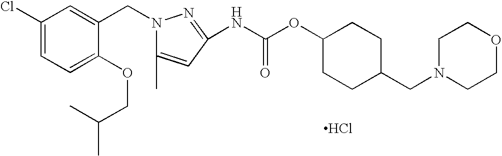 | 4-(4- morpholinylmethyl)cyclohexyl [1-({5-chloro-2-[(2- methylpropyl)oxy]phenyl}methyl)- 5-methyl-1H-pyrazol-3-yl] carbamate hydrochloride | Rt = 2.37 mins, [MH]⁺ 519, 521 |

### Example 470

### 2-[1-({5-chloro-2-[(2-methylpropyl)oxy]phenyl}methyl)-5-methyl-1H-pyrazol-3-yl][1,2,4]triazolor[1,5-a]pyridine

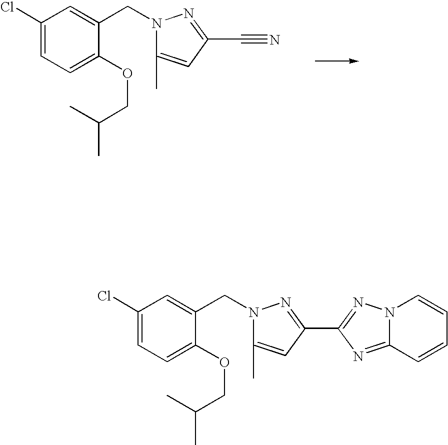

1-({5-chloro-2-[(2-methylpropyl)oxy]phenyl}methyl)-5-methyl-1H-pyrazole-3-carbonitrile (0.1 g, 0.296 mmol), DBU (0.09 ml, 0.59 mmol) and 1-aminopyridinium iodide (0.072 g, 0.33 g) were stirred in toluene at room temperature for 2 hours and 70° C. for 65 hours, DMF (2 ml) added and the reaction heated at 120° C. for 27 hours. The reaction was allowed to cool, diluted with ethyl acetate, washed twice with 2M hydrochloric acid, once with brine, dried (MgSO₄), filtered, evaporated to dryness and purified on the MDAP. The residue was treated with 1M hydrogen chloride in ether (0.5 ml) and evaporated to yield the title compound (0.008 g). LC/MS Rt=3.33 min [MH+] 430, 432.

The following Examples were prepared using appropriate starting materials using a similar procedure to that used to prepare Example 378.

| Example | Structure | Name | Data |
|---|---|---|---|
| 471 | 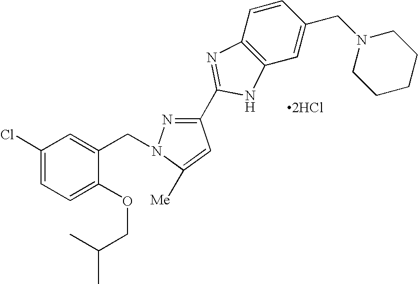 | 2-[1-({5-Chloro-2-[(2- methylpropyl)oxy]phenyl} methyl)-5-methyl-1H-pyrazol-3- yl]-5-(1-piperidinylmethyl)-1H- benzimidazole dihydrochloride | LC/MS Rt = 2.27 [MH]⁻ 490.17 |
| 472 | 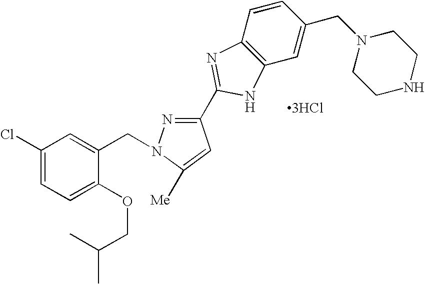 | 2-[1-({5-Chloro-2-[(2- methylpropyl)oxy]phenyl} methyl)-5-methyl-1H-pyrazol-3- yl]-5-(1-piperazinylmethyl)- 1H-benzimidazol trihydrochloride | LC/MS Rt = 2.01 [MH]⁻ 491.18, 493.2 |

The following Examples were prepared from appropriate starting materials using a similar procedure to that used to prepare Example 375.

| Example | Structure | Name | Data |
|---|---|---|---|
| 473 | 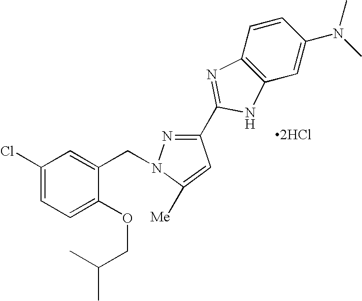 | 2-[1-({5-Chloro-2-[(2- methylpropyl)oxy]phenyl} methyl)-5-methyl-1H- pyrazol-3-yl]-N,N- dimethyl-1H- benzimidazol-5-amine dihydrochloride | LC/MS Rt = 2.57 [MH]⁻ 436.23, 438.25 |
| 474 | 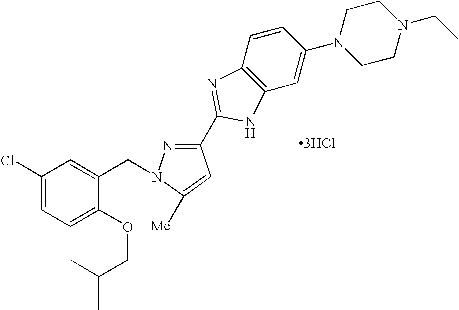 | 2-[1-({5-Chloro-2-[(2- methylpropyl)oxy]phenyl} methyl)-5-methyl-1H- pyrazol-3-yl]-5-(4-ethyl-1- piperazinyl)-1H- benzimidazole trihydrochloride | LC/MS Rt = 2.07 [MH]⁻ 505.25, 507.24 |

### Methyl 2-[1-({5-chloro-2-[(phenylmethyl)oxy]phenyl}methyl)-5-methyl-1H-pyrazol-3-yl]-1H-benzimidazole-5-carboxylate

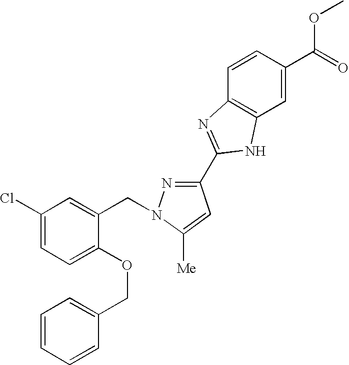

1-({5-Chloro-2-[(phenylmethyl)oxy]phenyl}methyl)-5-methyl-1H-pyrazole-3-carboxylic acid (3.57 g, 10 mmol) and methyl 3,4-diaminobenzoate (1.66 g, 10 mmol) were stirred in phosphorus oxychloride (10 ml) at 100° C. for 2 hours. The mixture was poured into ice-water and extracted with ethyl acetate, the insoluble material filtered and the filtrate washed with water and evaporated. The residue and the insoluble solid were combined and stirred in ethyl acetate: diethyl ether (1:1; 50 ml) and the purple solid filtered and dried. (3.47 g) LC/MS Rt 3.21, [MH]⁺ 487, 489.

### {2-[1-({5-Chloro-2-[(phenylmethyl)oxy]phenyl}methyl)-5-methyl-1H-pyrazol-3-yl]-1H-benzimidazol-5-yl}methanol

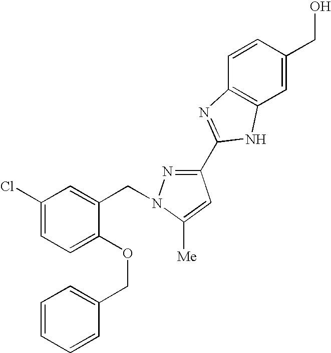

Methyl 2-[1-({5-chloro-2-[(phenylmethyl)oxy]phenyl}methyl)-b-methyl-1H-pyrazol-3-yl]-1H-benzimidazole-5-carboxylate (3.47 g, 7.13 mmol) was dissolved in THF (30 ml) and 1M lithium aluminium hydride in diethyl ether (7.48 ml, 7.48 mmol) added over 10 minutes. The mixture was stirred at room temperature for 1 hour. Water (5 ml) was added cautiously, followed by ethyl acetate and water. The mixture was filtered through Kieselguhr and the organic layer separated, dried (MgSO₄) and evaporated to a brown solid (1.85 g). The solid was redissolved in THF (15 ml) and 1M lithium aluminium hydride in diethyl ether (3.8 ml, 3.8 mmol) added over 10 minutes. The mixture was stirred at room temperature for 1 hour. Water (5 ml) was added cautiously, followed by ethyl acetate and water. The mixture was filtered through Kieselguhr and the organic layer separated, dried (MgSO₄) and evaporated to a brown solid which was triturated with diethyl ether, filtered and dried (603 mg). LC/MS Rt 2.37, [MH]⁺ 459, 461

### 2-[1-({5-Chloro-2-[(phenylmethyl)oxy]phenyl}methyl)-5-methyl-1H-pyrazol-3-yl]-1H-benzimidazole-5-carbaldehyde

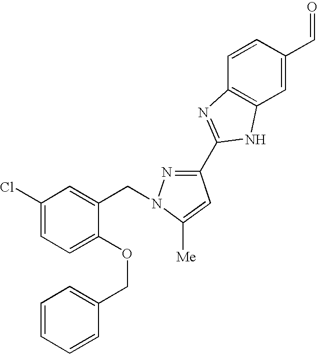

{2-[1-({5-Chloro-2-[(phenylmethyl)oxy]phenyl}methyl)-5-methyl-1H-pyrazol-3-yl]-1H-benzimidazol-5-yl}methanol (600 mg, 1.31 mmol) was dissolved in dichloromethane (10 ml) and Dess-Martin periodinane (596 mg, 1.44 mmol) added. The mixture was stirred at room temperature for 2 hours. The suspension was washed with 5% sodium thiosulfate solution and water, dried (MgSO₄) and evaporated. The residue was triturated with diethyl ether and the beige solid filtered and dried (592 mg). LC/MS Rt 3.16, [MH]⁺ 457, 459.

### Example 475

### ({2-[1-({5-Chloro-2-[(phenylmethyl)oxy]phenyl}methyl)-5-methyl-1H-pyrazol-3-yl]-1H-benzimidazol-5-yl}methyl)dimethylamine dihydrochloride

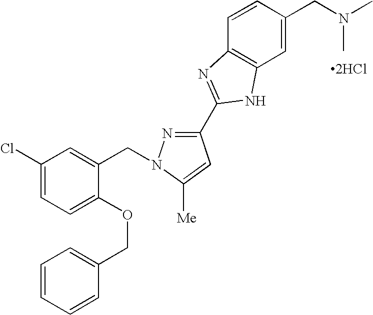

2-[1-({5-Chloro-2-[(phenylmethyl)oxy]phenyl}methyl)-5-methyl-1H-pyrazol-3-yl]-1H-benzimidazole-5-carbaldehyde (85 mg, 0.19 mmol) sodium triacetoxyborohydride (124 mg, 0.57 mmol) and 5.6M dimethylamine in ethanol (68 ul, 0.38 mmol) were stirred in THF (2 ml) under argon at room temperature for 16 hours. Ethyl acetate and water were added and the organic layer dried (MgSO₄) and evaporated. The residue was purified by flash chromatography, eluting with 2-20% methanol in dichloromethane. The product was dissolved in dichloromethane (1 ml) and 1M HCl in diethyl ether (1 ml) added. The solvent was evaporated and the residue triturated with diethyl ether. The product was collected by decantation and dried in a stream of argon (24 mg). LC/MS Rt 2.24, [MH]⁺ 486, 488.

The following examples were prepared from appropriate starting materials using a similar method to that described for ({2-[1-({5-chloro-2-[(phenylmethyl)oxy]phenyl}methyl)-5-methyl-1H-pyrazol-3-yl]-1H-benzimidazol-5-yl}methyl)dimethylamine dihydrochloride above.

| Example | Structure | Name | Data |
|---|---|---|---|
| 476 | 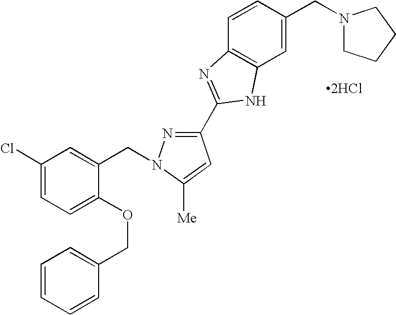 | 2-[1-({5-Chloro-2- [(phenylmethyl)oxy]phenyl} methyl)-5-methyl-1H-pyrazol- 3-yl]-5-(1-pyrrolidinylmethyl)- 1H-benzimidazole dihydrochloride | LC/MS Rt = 2.27 [MH]⁺ 512 |
| 477 | 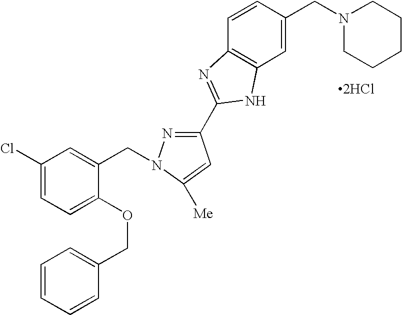 | 2-[1-({5-Chloro-2- [(phenylmethyl)oxy]phenyl} methyl)-5-methyl-1H-pyrazol- 3-yl]-5-(1-piperidinylmethyl)- 1H-benzimidazole dihydrochloride | LC/MS Rt = 2.28 [MH]⁺ 526 |
| 478 | 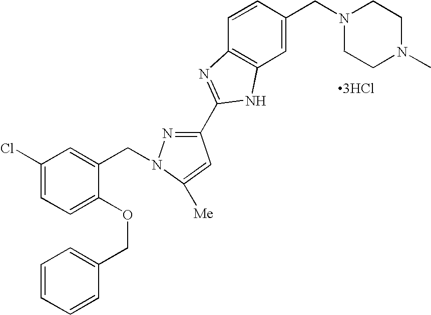 | 2-[1-({5-Chloro-2- [(phenylmethyl)oxy]phenyl} methyl)-5-methyl-1H-pyrazol- 3-yl]-5-[(4-methyl-1- piperazinyl)methyl]-1H- benzimidazole trihydrochloride | LC/MS Rt = 2.08 [MH]⁺ 539, 541 |
| 479 | 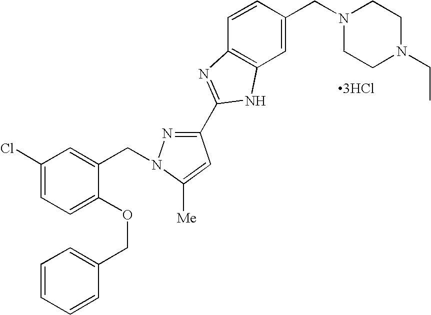 | 2-[1-({5-Chloro-2- [(phenylmethyl)oxy]phenyl} methyl)-5-methyl-1H-pyrazol- 3-yl]-5-[(4-ethyl-1- piperazinyl)methyl]-1H- benzimidazole trihydrochloride | LC/MS Rt = 2.12 [MH]⁺ 553, 555 |

### Example 480

### 1-({5-Chloro-2-[(phenylmethyl)oxy]phenyl}methyl)-5-methyl-3-(2-phenyl-1H-imidazol-4-yl)-1H-pyrazole hydrochloride

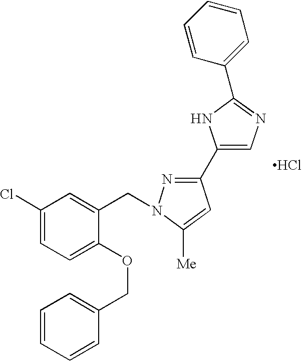

Benzenecarboximidamide (18 mg, 0.115 mmol) and potassium bicarbonate (46 mg, 0.46 mmol) were stirred in THF (1.6 ml) and water (0.4 ml) and heated to reflux. 2-Bromo-1-[1-({5-chloro-2-[(phenylmethyl)oxy]phenyl}methyl)-5-methyl-1H-pyrazol-3-yl]ethanone (50 mg, 0.115 mmol) in THF (0.4 ml) was added dropwise over 15 minutes. The mixture was heated at reflux for 1 hour, then evaporated. The residue was partitioned between ethyl acetate and water and the organic layer washed with water, dried (MgSO₄) and evaporated. The residue was purified by flash chromatography, eluting with 2% methanol in dichloromethane. The product was dissolved in dichloromethane (0.5 ml) and 1M HCl in diethyl ether (0.5 ml) added. The solid was filtered, washed with diethyl ether and dried (33 mg). LC/MS Rt 2.62, [MH]⁺ 455, 457.

The following examples were prepared from appropriate starting materials using a similar method to that described for 1-({5-chloro-2-[(phenylmethyl)oxy]phenyl}methyl)-5-methyl-3-(2-phenyl-1H-imidazol-4-yl)-1H-pyrazole hydrochloride above.

| Example | Structure | Name | Data |
|---|---|---|---|
| 481 | 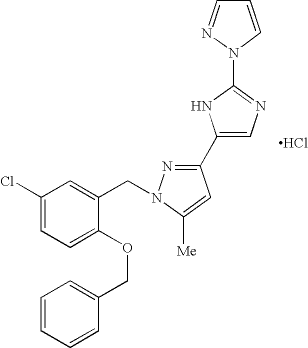 | 1-({5-Chloro-2- [(phenylmethyl)oxy]phenyl} methyl)-5-methyl-3-[2-(1H- pyrazol-1-yl)-1H-imidazol-4- yl]-1H-pyrazole hydrochloride | LC/MS Rt = 3.24 [MH]⁺ 445, 447 |
| 482 | 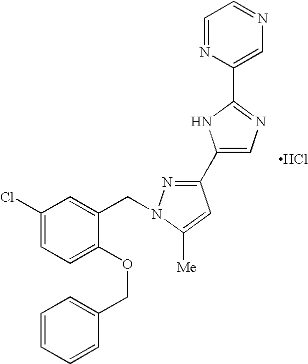 | 2-{4-[1-({5-chloro-2- [(phenylmethyl)oxy]phenyl} methyl)-5-methyl-1H-pyrazol- 3-yl]-1H-imidazol-2- yl}pyrazine | LC/MS Rt = 2.99 [MH]⁺ 457, 459 |
| 483 | 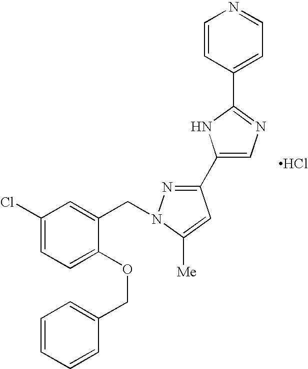 | 4-{4-[1-({5-Chloro-2- [(phenylmethyl)oxy]phenyl} methyl)-5-methyl-1H-pyrazol- 3-yl]-1H-imidazol-2- yl}pyridine hydrochloride | LC/MS Rt = 2.58 [MH]⁺ 456, 458 |
| 484 | 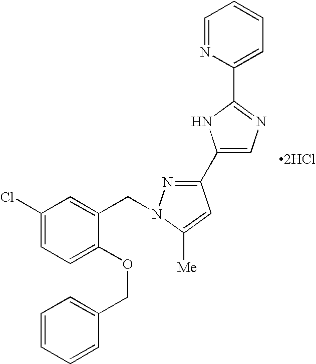 | 2-{4-[1-({5-Chloro-2- [(phenylmethyl)oxy]phenyl} methyl)-5-methyl-1H-pyrazol- 3-yl]-1H-imidazol-2- yl}pyridine dihydrochloride | LC/MS Rt = 2.73 [MH]⁺ 456, 458 |
| 485 | 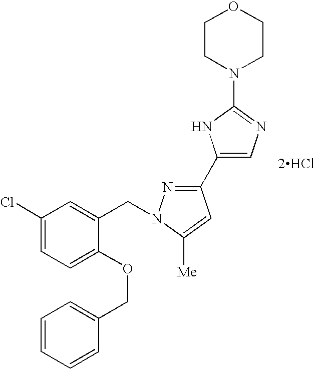 | 4-{4-[1-({5-Chloro-2- [(phenylmethyl)oxy]phenyl} methyl)-5-methyl-1H-pyrazol- 3-yl]-1H-imidazol-2- yl}morpholine dihydrochloride | LC/MS Rt = 2.51 [MH]⁺ 464, 466 |

### Example 486

### 1-({5-chloro-2-[(2-methylpropyl)oxy]phenyl}methyl)-N-(6-ethenyl-3-pyridinyl)-5-methyl-1H-pyrazole-3-carboxamide

### (a) 1-({5-chloro-2-[(2-methylpropyl)oxy]phenyl}methyl)-5-methyl-1H-pyrazole-3-carbonyl chloride

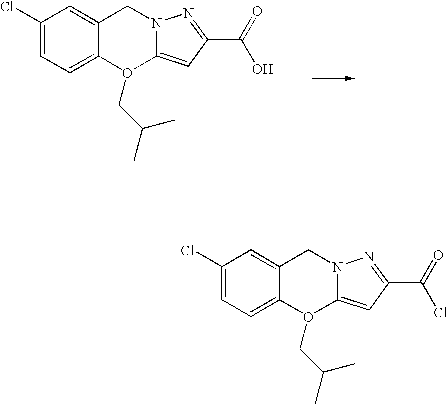

A mixture of 1-({5-chloro-2-[(2-methylpropyl)oxy]phenyl}methyl)-5-methyl-1H-pyrazole-3-carboxylic acid (1.10 g, 3.42 mmol) and thionyl chloride (1.25 ml, 17.10 mmol) was stirred at 60° C. for 1.5 hours under an atmosphere of argon. The reaction was monitored by LC-MS. After this time, the reaction mixture was allowed to cool to room temperature and concentrated under reduced pressure to give 1-({5-chloro-2-[(2-methylpropyl)oxy]phenyl}-methyl)-5-methyl-1H-pyrazole-3-carbonyl chloride (1.16 g, 100%).

Rt=3.82 [MH⁺]: 341, 343, 345

The residue was used immediately without further purification in the next stage.

### (b) 1-({5-chloro-2-[(2-methylpropyl)oxy]phenyl}methyl)-N-(6-ethenyl-3-pyridinyl)-5-methyl-1H-pyrazole-3-carboxamide

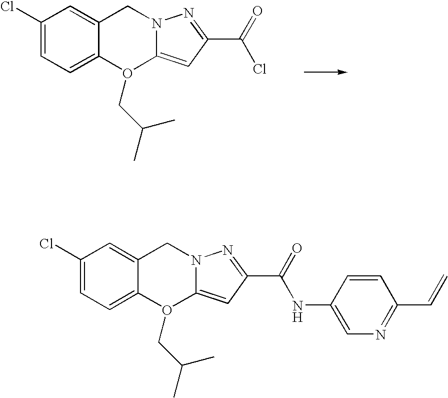

A solution of 1-({5-chloro-2-[(2-methylpropyl)oxy]phenyl}methyl)-5-methyl-1H-pyrazole-3-carbonyl chloride (1.16 g, 3.42 mmol) in dry DCM (7 ml) was stirred at room temperature under an atmosphere of argon. 6-ethenyl-3-pyridinamine (0.382 g, 3.18 mmol) and triethylamine (0.57 ml, 4.09 mmol) were added. Fuming and colour change from yellow to red solution observed. Reaction mixture stirred at room temperature under an atmosphere of argon for 1.5 hours. The reaction was monitored by LC-MS. After this time, further triethylamine (0.57 ml, 4.09 mmol) was added and the reaction mixture was stirred for 72 hours (over the weekend) at room temperature. Further triethylamine (0.57 ml, 4.09 mmol) and dry DCM (2 ml) were added. Reaction mixture was left stirring at room temperature for further 2 hours. After this time, the reaction mixture was concentrated under reduced. The resulting residue was stirred in EtOH (20 ml) and 2M sodium hydroxide (9 ml) at room temperature for 1 hour. Reaction mixture was concentrated under reduced pressure. The residue was partitioned between EtOAc and NaHCO₃ (sat. aq sol). The aqueous layer was washed with further EtOAc and brine was added to encourage separation. The combined organics were dried over magnesium sulfate, filtered and concentrated under reduced pressure to give crude product, brown oil (1.04 g). The residue was purified using column chromatography SiO₂, 6%-60% EtOAc in hexane for 30 min to give impure product (0.500 g). Column purification repeated but with eluent of 30%-50% EtOAc in hexane over 30 minutes to give 1-({5-chloro-2-[(2-methylpropyl)oxy]phenyl}methyl)-N-(6-ethenyl-3-pyridinyl)-5-methyl-1H-pyrazole-3-carboxamide (400 mg, 28%, 80% purity).

LCMS Rt=3.38 [MH⁺]: 425, 427

### 5-ethenyl-2-pyridinamine

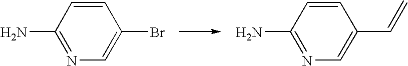

A solution of 5-bromo-2-pyridinamine (2.00 g, 11.56 mmol), vinyl boronic anhydride pyridine complex, VBAP (4.17 g, 17.34 mmol) and potassium carbonate (12.78 g, 92.5 mmol) in EtOH (58 ml) and PhCH₃ (58 ml) was purged with argon for 30 minutes. Pd(PPh₃)₄ (0.670 g, 0.580 mmol) was then added and the reaction mixture stirred at 80° C. for 20 hours under an atmosphere of argon. The reaction was monitored by LC-MS. After this time, the reaction mixture was allowed to cool to room temperature and partitioned between EtOAc and water. The organics were washed with further water and were dried over magnesium sulfate, filtered and concentrated under reduced pressure to give crude product. The crude product was purified on the Horizion, C18 column, 5%-100% acetonitrile in water for 30 minutes to give 5-ethenyl-2-pyridinamine (0.317 g, 23%)

LCMS Rt=0.84 [MH⁺]: 121

### 1-({5-chloro-2-[(2-methylpropyl)oxy]phenyl}methyl)-N-(5-ethenyl-2-pyridinyl)-5-methyl-1H-pyrazole-3-carboxamide

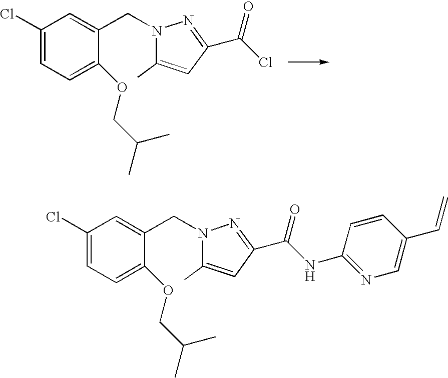

A solution of 1-({5-chloro-2-[(2-methylpropyl)oxy]phenyl}methyl)-5-methyl-1H-pyrazole-3-carbonyl chloride (1.80 g, 5.28 mmol) in dry DCM (11 ml) was stirred at room temperature under an atmosphere of argon. 5-ethenyl-2-pyridinamine (0.317 g, 2.64 mmol) was added. Reaction mixture stirred at room temperature under an atmosphere of argon for 2 hours. The reaction was monitored by LC-MS. After this time, triethylamine (0.73 ml, 5.28 mmol) was added and the reaction mixture was stirred for 72 hours (over the weekend) at room temperature. Further triethylamine (0.73 ml, 5.28 mmol) was added and the reaction mixture was left stirring at room temperature for 1 hour. The reaction mixture was concentrated under reduced pressure. The resulting residue was stirred in EtOH (20 ml) and 2M sodium hydroxide (9 ml) at room temperature for 1 hour. After this time, the reaction mixture was concentrated under reduced pressure. The residue was partitioned between EtOAc and NaHCO₃ (sat. aq sol). Brine was added to encourage separation. Organics were dried over magnesium sulfate, filtered and concentrated under reduced pressure to give crude product, brown solid (1.17 g). The residue was purified using column chromatography SiO₂, 5%-40% EtOAc in hexane for 30 min to give 1-({5-chloro-2-[(2-methylpropyl)oxy]phenyl}methyl)-N-(5-ethenyl-2-pyridinyl)-5-methyl-1H-pyrazole-3-carboxamide (0.778 g, 69%). LCMS Rt=4.00 [MH+]: 425, 427.

### 1-({5-chloro-2-[(2-methylpropyl)oxy]phenyl}methyl)-N-(5-formyl-2-pyridinyl)-5-methyl-1H-pyrazole-3-carboxamide

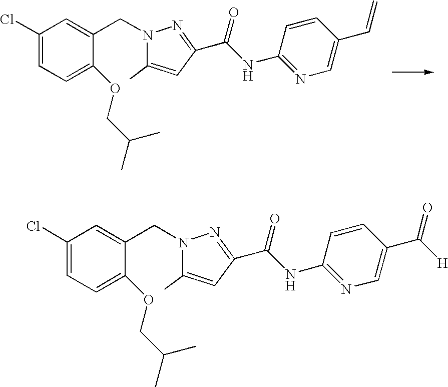

A mixture of 1-({5-chloro-2-[(2-methylpropyl)oxy]phenyl}methyl)-N-(5-ethenyl-2-pyridinyl)-5-methyl-1H-pyrazole-3-carboxamide (0.786 g, 1.85 mmol), OsO₄ (200 μl, 2.5 wt %), NalO₄ (0.990 g, 4.63 mmol) in THF (3.7 ml) and water (3.7 ml) was stirred at room temperature for 2.5 hours. The reaction was monitored by TLC (50% EtOAc in hexane). After this time, the reaction mixture was partitioned between DCM and water until solid dissolved. Aqueous layer was washed with further DCM. The combined organics were dried over sodium sulfate, filtered and concentrated under reduced pressure to give a white foam. The residue was purified using column chromatography SiO₂, 20%-50% EtOAc in hexane to give 1-({5-chloro-2-[(2-methylpropyl)oxy]phenyl}methyl)-N-(5-formyl-2-pyridinyl)-5-methyl-1H-pyrazole-3-carboxamide (0.400 g, 51%). LCMS Rt=3.72 [MH+]: 427, 429.

### Example 487

### 1-({5-chloro-2-[(2-methylpropyl)oxy]phenyl}methyl)-5-methyl-N-[5-(4-morpholinylmethyl)-2-pyridinyl]-1H-pyrazole-3-carboxamide hydrochloride salt

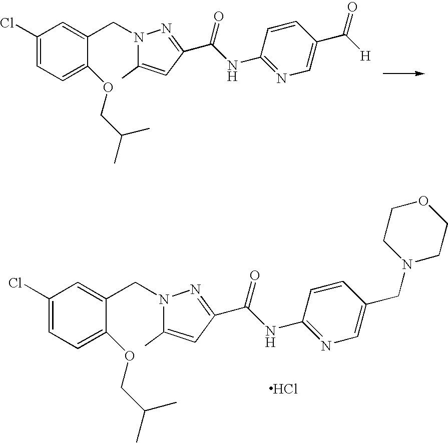

A solution of 1-({5-chloro-2-[(2-methylpropyl)oxy]phenyl}methyl)-N-(5-formyl-2-pyridinyl)-5-methyl-1H-pyrazole-3-carboxamide (0.100 g, 0.234 mmol) and morpholine (24.6 μl, 0.581 mmol) in dry DCM (0.8 ml) was stirred at room temperature under an atmosphere of argon for 3 hours. After this time, NaBH(OAc)₃ (0.050 g, 0.234 mmol) and acetic acid (0.1 ml) were added and the reaction mixture was stirred at room temperature for 1 hour. The reaction was monitored by LC-MS. After this time, further morpholine (24.6 μl, 0.581 mmol) was added and the reaction mixture stirred for further 2 hours. Further NaBH(OAc)₃ (0.050 g, 0.234 mmol), acetic acid (0.1 ml) and morpholine (24.6 μl, 0.581 mmol) were added and stirred for 17 hours. After this time, the reaction mixture was partitioned between DCM (20 ml) and 2M sodium hydroxide (2 ml). The organics were washed with further water and dried over magnesium sulfate, filtered and concentrated under reduced pressure (0.136 g). The residue was purified using column chromatography SiO₂, 12%-100% EtOAc in hexane for 20 minutes. The resulting residue was then stirred in HCl in 1,4 dioxane (1M, 5 ml) at room temperature for 10 minutes. The solvent was evaporated under reduced pressure to give a white solid, 1-({5-chloro-2-[(2-methylpropyl)oxy]phenyl}methyl)-5-methyl-N-[5-(4-morpholinylmethyl)-2-pyridinyl]-1H-pyrazole-3-carboxamide (0.091 g, 73%). t=2.46 [MH⁺]: 498, 500.

### Standard Procedure 8

### Example 488

### 1-({5-chloro-2-[(2-methylpropyl)oxy]phenyl}methyl)-5-methyl-N-5-[(methylamino)methyl]-2-pyridinyl)-1H-pyrazole-3-carboxamide hydrochloride salt

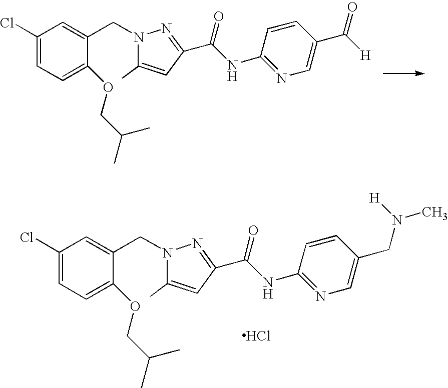

A solution of 1-({5-chloro-2-[(2-methylpropyl)oxy]phenyl}methyl)-N-(5-formyl-2-pyridinyl)-5-methyl-1H-pyrazole-3-carboxamide (0.150 g, 0.352 mmol) and methylamine (52.0 μl, 0.422 mmol) in dry DCM (3.5 ml) was stirred at room temperature under an atmosphere of argon for 30 minutes. After this time, NaBH(OAc)₃ (0.089 g, 0.422 mmol) and acetic acid (0.1 ml) were added and the reaction mixture was stirred at room temperature for 1 hour. The reaction was monitored by LC-MS. After this time, further methylamine (52.0 μl, 0.422 mmol) was added and the reaction mixture stirred for 1 hour. Further methylamine (52.0 μl, 0.422 mmol) and NaBH(OAc)₃ (0.089 g, 0.422 mmol) were added and stirred for 17 hours (overnight). After this time, the reaction mixture was partitioned between DCM (20 ml) and 2M sodium hydroxide (5 ml). The aqueous layer was washed with further DCM and brine was added to encourage separation. The combined organics were washed with water and dried over magnesium sulfate, filtered and concentrated under reduced pressure. The residue was purified using column chromatography SiO₂: 3 column vol 50% EtOAc in Hexane, 2 column vol 100% EtOAc followed by 3 column vol 10% methanolic ammonia solution in 1:1 EtOAc:Hexane. The resulting residue was then stirred in HCl in 1,4 dioxane (1M) at room temperature for 10 minutes. The solvent was evaporated under reduced pressure to give a white solid, 1-({5-chloro-2-[(2-methylpropyl)oxy]phenyl}methyl)-5-methyl-N-{5-[(methylamino)methyl]-2-pyridinyl}-1H-pyrazole-3-carboxamide (0.076 g, 49%). LCMS Rt=2.39 [MH⁺]: 442.

In some cases, further amine (1.2 equiv) and NaBH(OAc)₃ (0.089 g, 0.422 mmol) were added in order to progress the reaction to completion.

The following Example was prepared from appropriate intermediates using a similar method to that described in Standard Procedure 8:

| Example | Structure | Name | LCMS Data |
|---|---|---|---|
| 488 | 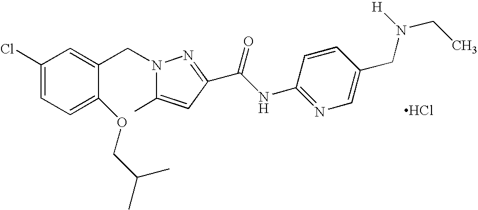 | 1-({5-chloro-2-[(2- methylpropyl)oxy]phenyl} methyl)-N-{5- [(ethylamino)methyl]-2- pyridinyl}-5-methyl-1H- pyrazole-3-carboxamide hydrochloride salt | Rt = 2.45, [MH⁺]: 456 |

### N-(5-bromo-2-pyrazinyl)-1-({5-chloro-2-[(2-methylpropyl)oxy]phenyl}methyl)-5-methyl-1H-pyrazole-3-carboxamide

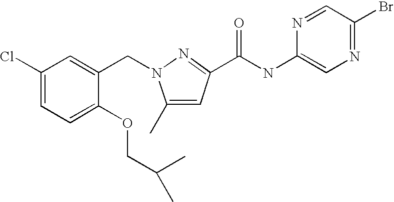

1-({5-Chloro-2-[(2-methylpropyl)oxy]phenyl}methyl)-5-methyl-1H-pyrazole-3-carboxylic acid (1.2 g, 3.72 mmol) was treated with dichloromethane (20 ml), cooled at 0° C., oxalyl chloride (811 μl, 9.3 mmol) was added followed by a drop of DMF. The reaction mixture under argon was stirred at 0° C. for 5 more minutes then let warm to room temperature and stirred for 1 hr. The solvent was evaporated; the residue was dissolved with H₂O and extracted with EtOAc (×3); the combined organic phases were dried (MgSO₄) and evaporated. The residue was purified on the Flash Master II using a gradient of ethyl acetate in hexane (040%) to give the title compound as white solid (1.54 g)

LC/MS Rt=4.01 min, [MH⁺] 480.1, 482.1.

### 1-({5-Chloro-2-[(2-methylpropyl)oxy]phenyl}methyl)-N-(5-ethenyl-2-pyrazinyl)-5=methyl-1H-pyrazole-3-carboxamide

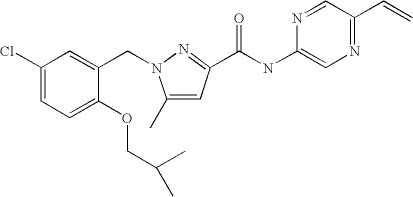

A mixture of N-(5-bromo-2-pyrazinyl)-1-({5-chloro-2-[(2-methylpropyl)oxy]phenyl}methyl)-5-methyl-1H-pyrazole-3-carboxamide (1.54 g, 3.22 mmol), vinylboronic anhydride pyridine complex (852 mg, 3.54 mmol), potassium carbonate (1.77 g, 12.87 mmol) and Pd(PPh₃)₄ (371 mg, 0.32 mmol) were heated at 80° C. in a 1:1 toluene/ethanol mixture (30 ml) under argon for 3 hr. The mixture was then cooled, evaporated and the residue treated with water and extracted with EtOAc (×2). The combined extracts were dried (MgSO₄) and evaporated, the residue was purified on the Flash Master II using a gradient of ethyl acetate in hexane (0-30%) to give the title compound as yellow solid (1.21 g).

LC/MS Rt=3.89 min, [MH⁺] 426.2, 428.2, 429.2

### 1-({5-Chloro-2-[(2-methylpropyl)oxy]phenyl}methyl)-N-(5-formyl-2-pyrazinyl)-5-methyl-1H-pyrazole-3-carboxamide

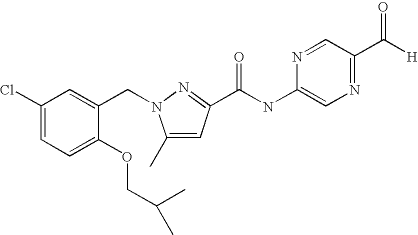

Two drops of osmium tetroxide (2.5 wt. % in 2-propanol) were added to 1-({5-Chloro-2-[(2-methylpropyl)oxy]phenyl}methyl)-N-(5-ethenyl-2-pyrazinyl)-5-methyl-1H-pyrazole-3-carboxamide (1.21 g, 2.84 mmol) in a 1:1 tetrahydrofuran/water mixture (20 ml) with sodium (meta)periodate (1.52 g, 7.1 mmol). The resulting solution was stirred at room temperature for 6 hr; diluted with DCM/H₂O; after the extraction the organic phase was washed with Na₂S₂O₃ solution. Evaporation of the solvent gave a solid that was triturated with ether to give the title compound as an off-white solid (1.1 g).

LC/MS Rt=3.86 min, [MH⁺] 428.1, 430.1, [MH] 426.1, 428.1

### Example 490

### 1-({5-chloro-2-[(2-methylpropyl)oxy]phenyl}methyl)-5-methyl-N-[5-(1-piperidinylmethyl)-2-pyrazinyl]-1H-pyrazole-3-carboxamide hydrochloride

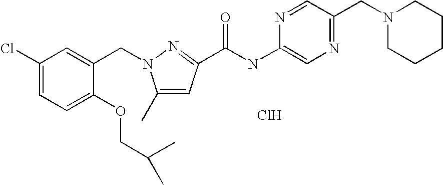

1-({5-Chloro-2-[(2-methylpropyl)oxy]phenyl}methyl)-N-(5-formyl-2-pyrazinyl)-5-methyl-1H-pyrazole-3-carboxamide (120 mg, 0.28 mmol) was dissolved in tetrahydrofuran (6 ml), sodium triacetoxyborohydride (119 mg, 0.56 mmol) and piperidine (55 μl, 0.56 mmol) were added. The reaction mixture was stirred under argon at room temperature for 18 hr; was then quenched with H₂O and extracted with EtOAc (×2). The combined extracts were dried (MgSO₄) and evaporated; the residue was purified on a silica cartridge using first EtOAc to remove impurities and then DCM/MeOH gradient. The residue was dissolved in methanol and 1M HCl in ether (1 ml) was added, the mixture was stirred for 10 minutes and evaporated to give a solid that was triturated with diethyl ether/ethyl acetate mixture to afford the title compound (99 mg). LC/MS Rt=2.43 min, [MH+] 497.2, 500.2

The following Examples were prepared from appropriate starting materials using a similar procedure to that described for the preparation Example 490

| Example | Structure | Name | Data |
|---|---|---|---|
| 491 | 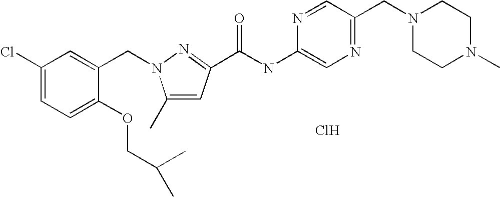 | 1-({5-Chloro-2-[(2- methylpropyl)oxy]phenyl}methyl)- 5-methyl-N-{5-[(4-methyl-1- piperazinyl)methyl]-2-pyrazinyl}- 1H-pyrazole-3-carboxamide hydrochloride | LC/MS Rt = 2.33 min, [MH⁺] 512.2, 514.1, 515.2 |
| 492 | 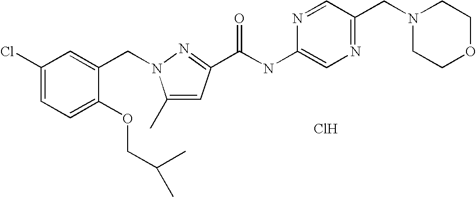 | 1-({5-Chloro-2-[(2- methylpropyl)oxy]phenyl}methyl)- 5-methyl-N-[5-(4- morpholinylmethyl)-2-pyrazinyl]- 1H-pyrazole-3-carboxamide hydrochloride | LC/MS Rt = 2.42 min, [MH⁺] 499.1, 501.1 |
| 493 | 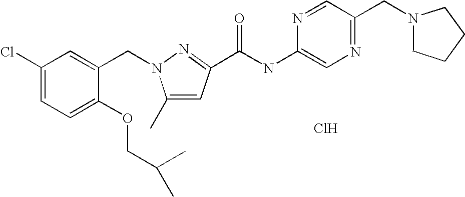 | 1-({5-Chloro-2-[(2- methylpropyl)oxy]phenyl}methyl)- 5-methyl-N-[5-(1- pyrrolidinylmethyl)-2-pyrazinyl]- 1H-pyrazole-3-carboxamide hydrochloride | LC/MS Rt = 2.39 min, [MH⁺] 483.1, 486.1 |
| 494 | 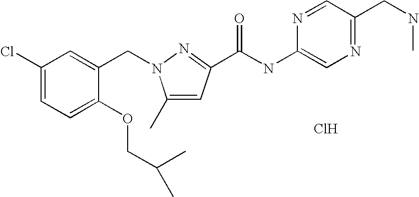 | 1-({5-Chloro-2-[(2- methylpropyl)oxy]phenyl}methyl)- 5-methyl-N-{5- [(methylamino)methyl]-2- pyrazinyl}-1H-pyrazole-3- carboxamide hydrochloride | LC/MS Rt = 2.34 min, [MH⁺] 443.1, 445.1, 446.1 |
| 495 | 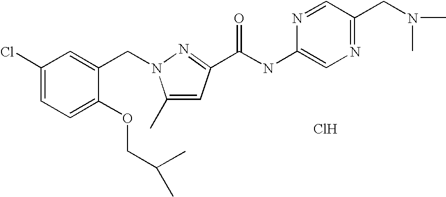 | 1-({5-Chloro-2-[(2- methylpropyl)oxy]phenyl}methyl)- N-{5-[(dimethylamino)methyl]- 2-pyrazinyl}-5-methyl-1H- pyrazole-3-carboxamide hydrochloride | LC/MS Rt = 2.48 min, [MH⁺] 457.1, 459.1 |

### Example 496

### 2-[1-({5-Chloro-2-[(2-methylpropyl)oxy]phenyl}methyl)-5-methyl-1H-pyrazol-3-yl]-5-(4-morpholinyl)-1H-benzimidazole dihydrochloride

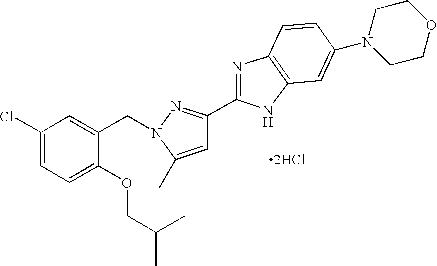

A mixture of 4-(4-morpholinyl)-1,2-benzenediamine (70 mg, 0.36 mmol) and 1-({5-chloro-2-[(2-methylpropyl)oxy]phenyl}methyl)-5-methyl-1H-pyrazole-3-carboxylic acid (108 mg, 0.33 mmol) in phosphorus oxychloride (2 ml) was stirred and heated at 100° C. for 2 hours. The resulting solution was poured onto ice and diluted with water, ethyl acetate and 2M sodium hydroxide solution. The organic phase was dried (magnesium sulphate), evaporated and purified on a Biotage column eluting with ethyl acetate. The product was dissolved in dichloromethane and treated with 1M hydrogen chloride in ether. After evaporation the residue was triturated with ether to give the title compound as a dark coloured solid (16 mg). LC/MS: Rt=2.57, [MH]⁺ 480.24, 482.26

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

### Biological Activity at EP₁ and EP₃ Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable (pCIN; BioTechniques 20 (1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (plC₅₀) may then be estimated.

### Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10s burst at full setting), centrifuged at 48,000×g for 20mins and the pellet containing the membrane fraction is washed (optional) three times by suspension and centrifugation at 48,000×g for 20mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

### Bioloqical Activity at TP Receptor

To determine if a compound has agonist or antagonist activity at the TP receptor a functional calcium mobilisation assay may be performed. Briefly, the antagonist properties of compounds are assessed by their ability to inhibit the mobilisation of intracellular calcium ([Ca²⁺]ᵢ) in response to activation of TP receptors by the stable TXA₂ mimetic U46619 (9,11-dideoxy-11α,9α-epoxy-methanoprostaglandin F2α; commercially available from e.g Sigma-Aldrich). Increasing concentrations of antagonist reduce the amount of calcium that a given concentration of U46619 can mobilise. The net effect is to displace the U46619 concentration-effect curve. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software. The agonist activity of the compounds are determined by their ability to cause an increase in intracellular mobilisation in the absence of U46619.

The human TP calcium mobilisation assay utilises Chinese hamster ovary-K1 (CHO-K1) cells into which a stable (pCIN; BioTechniques 20 (1996): 102-110) vector containing TP cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 100 μg/ml puromycin.

For assay, cells are harvested using a proprietary reagent that dislodges cells such as Versene. Cells are re-suspended in a suitable quantity of fresh culture media for introduction into a 96-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of U46619 are then added to the plate in order to assess the antagonist properties of the compounds.

The data so generated may be analysed by means of a computerised curve-fitting routine. The concentration of compound that elicits a half-maximal inhibition of the calcium mobilisation induced by U46619 (pIC₅₀) may then be estimated, and the percentage activation caused by the compounds directly can be used to determine if there is any agonism present.

### Results

The compounds of examples 1496 were tested in the binding assay for the human prostanoid EP₁ receptor. The results are expressed as pIC₅₀ values. A pIC₅₀ is the negative logarithm₁₀ of the IC₅₀. The results given are averages of a number of experiments. The compounds of the Examples had a pIC₅₀ value ≧6. More particularly, the compounds of examples 6-9, 30, 76, 109, 110, 118, 126, 132, 133, 136, 137, 154, 160, 313, 319, 346, 350, 353, 355, 356, 372, 373, 375, 377, 381, 382, 392-399 and 473 exhibited a pIC₅₀ value ≧8.0.

The compounds of examples 1-3, 6, 8-14, 23-113, 120-116, 174-180, 182-200, 202, 204-220, 227, 229-252, 254-259, 261-273, 275-317, 320-327, 329-332, 334-389, 391-417, 419, 421-425, 427-442, 444-470, 471-475, 477-479 and 481-496 (free bases or sodium salts) were tested in the human EP₁ calcium mobilisation assay. The results are expressed as functional pKᵢ values. A functional pKi is the negative logarithm₁₀ of the antagonist dissociation constant as determined in the human EP₁ calcium mobilisation assay. The results given are averages of a number of experiments. The compounds of examples 6, 9, 43, 51, 56, 59-61, 65, 66, 97, 98, 103, 104, 120-129, 139, 140, 142, 144, 145, 148, 149, 249, 284, 288, 290-292, 294, 296, 297, 300, 304, 306, 338-340, 359, 368, 371, 376, 385, 412, 435, 436, 438, 440, 481-483 and 485 exhibited a functional pKi value <6. The remaining compounds of the Examples tested exhibited a functional pKi value ≧6

The compounds of examples 1-9, 12-14, 23-69, 71-113, 120-166, 174-180, 182-220, 227, 229-252, 254-259, 261-273, 275-319, 321-399, 402, 405-410, 412-416, 419-442, 444-470, and 472-496. (free bases or sodium salts) were tested in the human EP₃ calcium mobilisation assay. The results are expressed as functional pKᵢ values. A functional pKi is the negative logarithm₁₀ of the antagonist dissociation constant as determined in the human EP₃ calcium mobilisation assay. The results given are averages of a number of experiments. Compounds of Examples 50, 52, 75, 95, 180, 183, 184, 187, 227, 364 and 387 exhibited a functional pKi value of >6.5 and ≦6.8. All other compounds tested exhibited a functional pKi value of ≦6.5. The compounds of examples 1-11, 13, 15-24, 27-34, 35, 38, 39, 43, 45, 47, 49, 51, 55, 57, 58, 60-72, 74, 75, 77-83, 85, 86, 88, 89, 93, 94, 97, 98-145, 147-175, 178, 179, 181, 182, 185, 186, 190-192, 195, 199, 202, 204, 206, 208-226, 228, 230, 231-241, 243-246, 248-250, 253-258, 260-262, 265, 266, 268, 274, 280-284, 286, 287, 290-308, 310, 312, 313, 315, 316, 319, 320, 322, 327, 328, 330, 332-344, 346, 348, 349-354, 356-358, 360, 365-372, 376-379, 381-383, 385, 386, 388-394, 396, 399427, 430, 433, 435-449, 452-455, 457, 458, 460, 462-466, 468-485, 489-491 and 493-496 were inactive.

No toxicological effects were observed in these tests.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

1. A compound of formula (I): 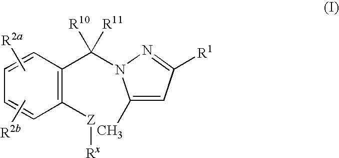
wherein:
Z is O, S, SO or SO₂;
R^{x} is optionally substituted C₂₋₁₀alkyl, optionally substituted C₂₋₁₀alkenyl, optionally substituted C₂₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CONR³R⁴, NR³CO₂R⁵, NR³COR⁶, OCONR³R¹, tetrazolyl, oxazolin-2-yl, oxazol-2-yl, benzoxazol-2-yl, pyrrolidinonyl, isoindoledionyl, dihydroisoindolonyl, or optionally substituted SO₂NHCOaryl; or R¹ is optionally substituted imidazolyl or optionally substituted 1,2,4-triazolyl wherein optionally the imidazole or 1,2,4-triazole ring is fused to give an optionally substituted bicyclic or tricyclic ring system;
or R¹ is 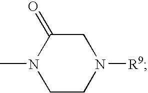
R^{2a} and R^{2b} are independently selected from hydrogen, halo, CN, SO₂alkyl, SR³, NO₂, optionally substituted alkyl, and optionally substituted alkoxy;
R³ is hydrogen or C₁₋₄alkyl;
R⁴ is hydrogen, OH, optionally substituted alkyl, optionally substituted aryl, optionally substituted heterocyclyl, optionally substituted bicyclic heterocyclyl, optionally substituted CQ^{c}Q^{d}aryl, optionally substituted CQ^{c}Q^{d}heterocyclyl, optionally substituted CQ^{c}Q^{d}bicyclic heterocyclyl, or SO₂R⁸;
R⁵ is C₁₋₄alkyl optionally substituted by SiMe₃, SO₂C₁₋₄alkyl, OC₁₋₄alkyl, N(C₁₋₄alkyl)₂, CO₂C₁₋₄alkyl, or CF₃; cyclohexyl substituted by CH₂NHC₁₋₄alkyl, CH₂pyrrolidinyl, CH₂morpholinyl or CH₂piperidinyl; phenyl; CQ^{c}Q^{d}phenyl; CQ^{c}Q^{d}pyridyl; CQ^{c}Q^{d}thienyl; CQ^{c}Q^{d}tetrahydrofuryl; CQ^{c}Q^{d}furyl; CQ^{c}Q^{d}piperidinyl optionally substituted by C₁₋₄alkyl; CH₂CH₂pyrrolidinonyl; CQ^{c}Q^{d}CH₂morpholinyl; tetrahydropyranyl; tetrahydrofuryl; 2-pyrrolidinon-4-yl; tetrahydrothienyl-1,1-dioxide; piperidin-4-yl optionally substituted on the 1-position by CO₂C₁₋₄alkyl; or dihydroindenyl;
R⁶ is alkyl, optionally substituted aryl, optionally substituted heterocyclyl, optionally substituted bicyclic heterocyclyl, optionally substituted CQ^{c}Q^{d}-Y-aryl, optionally substituted CQ^{c}Q^{d}-Y-heterocyclyl or optionally substituted CQ^{c}Q^{d}-Y-bicyclic heterocyclyl;
R⁷ is optionally substituted alkyl, optionally substituted alkenyl, optionally substituted aryl, or optionally substituted CQ^{c}Q^{d}aryl;
R⁸ is optionally substituted alkyl, optionally substituted aryl or optionally substituted heterocyclyl;
R⁹ is alkyl optionally substituted by OH, CN, OC₁₋₃alkyl, CONH₂, CONHC₁₋₄alkyl, or SO₂phenyl; alkenyl; optionally substituted CQ^{c}Q^{d}-Y-aryl; optionally substituted CQ^{c}Q^{d}-Y-heterocyclyl; or optionally substituted CQ^{c}Q^{d}-Y-bicyclic heterocyclyl;
R¹⁰ and R¹¹ are independently selected from hydrogen, fluorine and alkyl; or R¹⁰ and R¹¹ together with the carbon to which they are attached form a cycloalkyl ring, optionally containing up to one heteroatom selected from O, S, NH or N-alkyl;
Y is CH₂ or a bond;
Q^{a} and Q^{b} are each independently selected from hydrogen, CH₃ and fluorine; and
Q^{c} and Q^{d} are each independently selected from hydrogen, CH₃ and fluorine;
and derivatives thereof;
provided that:
R⁹ is not optionally substituted CH₂furan or optionally substituted CH₂imidazole;
when R^{x} is 2-methylpropyl, then R⁵ is not 1-methylethyl;
when R^{x} is optionally substituted CH₂cyclopropyl, then R⁹ is not 2-methylpropyl, CH₂cyclopropyl, CH₂cyclobutyl, CH₂CH₂OCH₃ or CH₂CH₂OH;
when R^{x} is CH₂tetrahydropyranyl or CH₂CH₂N(CH₃)₂, then R⁹ is not 2-methylpropyl.
when R¹ is benzimidazolyl it is unsubstituted on the 1-position; and
when R¹ is benzimidazole optional substituents on the 4 or 7 position are selected from CH₂OH or CO₂H.

2. A compound according to claim 1 wherein Z is O; R^{2a} is hydrogen; R^{2b} is Cl or Br and is positioned 1,4-relative to the Z substituent; and 1,3-relative to the pyrazole moiety; and R¹⁰ and R¹¹ are each hydrogen.

3. A compound according to claim 1 selected from the compounds of Examples 1 to 496 or a derivative thereof.

4. A pharmaceutical composition comprising a compound according to claim 1 or a pharmaceutically acceptable derivative thereof together with a pharmaceutical carrier and/or excipient.

5-6. (canceled)

7. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

8. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable derivative thereof.

9. A method of treating a human or animal subject suffering from inflammatory pain, neuropathic pain or visceral pain which method comprises administering to said subject an effective amount of a compound according to claim 1 or a pharmaceutically acceptable derivative thereof.

10-12. (canceled)

13. A compound of formula (I) according to claim 1 selected from:
N-[1-({5-chloro-2-[(2-methylpropyl)oxy]phenyl}methyl)-5-methyl-1H-pyrazol-3-yl]-1,2,3,4-tetrahydro-6-isoquinolinecarboxamide,
1-({5-chloro-2-[(2-methylpropyl)oxy]phenyl}methyl)-5-methyl-N-(1,2,3,4-tetrahydro-6-isoquinolinyl)-1H-pyrazole-3-carboxamide,
N-[1-({5-chloro-2-[(2-methylpropyl)oxy]phenyl}methyl)-5-methyl-1H-pyrazol-3-yl]-2-ethyl-1,2,3,4-tetrahydro-6-isoquinolinecarboxamide,
N-[1-({5-chloro-2-[(2-methylpropyl)oxy]phenyl}methyl)-5-methyl-1H-pyrazol-3-yl]-2-(1-methylethyl)-1,2,3,4-tetrahydro-6-isoquinolinecarboxamide,
N-[1-({5-chloro-2-[(2-methylpropyl)oxy]phenyl}methyl)-5-methyl-1H-pyrazol-3-yl]-2-cyclobutyl-1,2,3,4-tetrahydro-6-isoquinolinecarboxamide,
N-[1-({5-chloro-2-[(2-methylpropyl)oxy]phenyl}methyl)-5-methyl-1H-pyrazol-3-yl]-2-cyclopentyl-1,2,3,4-tetrahydro-6-isoquinolinecarboxamide,
N-[1-({5-chloro-2-[(2-methylpropyl)oxy]phenyl}methyl)-5-methyl-1H-pyrazol-3-yl]-2-cyclohexyl-1,2,3,4-tetrahydro-6-isoquinolinecarboxamide, and
N-[1-({5-chloro-2-[(2-methylpropyl)oxy]phenyl}methyl)-5-methyl-1H-pyrazol-3-yl]-2-(tetrahydro-2H-pyran-4-yl)-1,2,3,4-tetrahydro-6-isoquinolinecarboxamide,
or a pharmaceutically acceptable salt thereof.

14. A compound of formula (I) according to claim 1 which is N-[1-({5-chloro-2-[(2-methylpropyl)oxy]phenyl}methyl)-5-methyl-1H-pyrazol-3-yl]-1,2,3,4-tetrahydro-6-isoquinolinecarboxamide hydrochloride.

15. A compound of formula (I) according to claim 1 which is 1-({5-chloro-2-[(2-methylpropyl)oxy]phenyl}methyl)-5-methyl-N-(1,2,3,4-tetrahydro-6-isoquinolinyl)-1H-pyrazole-3-carboxamide hydrochloride.

16. A method of treating a human or animal subject suffering from cardiovascular disease or complications of type I diabetes which method comprises administering to said subject an effective amount of a compound according to claim 1 or a pharmaceutically acceptable derivative thereof.

17. A pharmaceutical composition comprising a compound according to claim 13 or a pharmaceutically acceptable derivative thereof together with a pharmaceutical carrier and/or excipient.

18. A method of treating a human or animal subject suffering from a condition which is mediated by the action of PGE₂ at EP₁ receptors which comprises administering to said subject an effective amount of a compound according to claim 13 or a pharmaceutically acceptable derivative thereof.

19. A method of treating a human or animal subject suffering from a pain, or an inflammatory, immunological, bone, neurodegenerative or renal disorder, which method comprises administering to said subject an effective amount of a compound according to claim 13 or a pharmaceutically acceptable derivative thereof.

20. A method of treating a human or animal subject suffering from inflammatory pain, neuropathic pain or visceral pain which method comprises administering to said subject an effective amount of a compound according to claim 13 or a pharmaceutically acceptable derivative thereof.

21. A method of treating a human or animal subject suffering from cardiovascular disease or complications of type I diabetes which method comprises administering to said subject an effective amount of a compound according to claim 13 or a pharmaceutically acceptable derivative thereof.
